SEQUENCE LISTING (1833 sequences)

SEQ ID NO: 1 - DNA, 17 bp, Artificial Sequence
  oligonucleotide
aggctgtgcgtgctatt

SEQ ID NO: 2 - DNA, 24 bp, Artificial Sequence
  oligonucleotide
ccatcaaggtggagcaatctgcgg

SEQ ID NO: 3 - DNA, 20 bp, Artificial Sequence
  oligonucleotide
ttgaccttggtctggatgag

SEQ ID NO: 4 - DNA, 20 bp, Artificial Sequence
  oligonucleotide
catcaaggtggagcaatctg

SEQ ID NO: 5 - DNA, 24 bp, Artificial Sequence
  oligonucleotide
tgctcgacctcatccagaccaagg

SEQ ID NO: 6 - DNA, 20 bp, Artificial Sequence
  oligonucleotide
ctcctggaccacatagttga

SEQ ID NO: 7 - DNA, 19 bp, Artificial Sequence
  oligonucleotide
gtgttggcagagctgaaag

SEQ ID NO: 8 - DNA, 24 bp, Artificial Sequence
  oligonucleotide
actttgtacggaaggctgtgcgtg

SEQ ID NO: 9 - DNA, 20 bp, Artificial Sequence
  oligonucleotide
cagattgctccaccttgatg

SEQ ID NO: 10 - DNA, 20 bp, Artificial Sequence
  oligonucleotide
gcattcttggcatctgtatt

SEQ ID NO: 11 - DNA, 24 bp, Artificial Sequence
  oligonucleotide
tggtggtggtgtccatttggcttt

SEQ ID NO: 12 - DNA, 18 bp, Artificial Sequence
  oligonucleotide
gtggctggcttgtaaatg

SEQ ID NO: 13 - DNA, 24 bp, Artificial Sequence
  oligonucleotide
tggtggtggtgtccatttggcttt

SEQ ID NO: 14 - DNA, 21 bp, Artificial Sequence
  oligonucleotide
ggcattcttggcatctgtatt

SEQ ID NO: 15 - DNA, 22 bp, Artificial Sequence
  oligonucleotide
gcttgtaaatgactcccttgtt

SEQ ID NO: 16 - DNA, 18 bp, Artificial Sequence
  oligonucleotide
tattgtggtggtggtgtc

SEQ ID NO: 17 - DNA, 26 bp, Artificial Sequence
  oligonucleotide
aagggagtcatttacaagccagccac

SEQ ID NO: 18 - DNA, 18 bp, Artificial Sequence
  oligonucleotide
gcctcagtctccatcttg

SEQ ID NO: 19 - DNA, 17 bp, Artificial Sequence
  oligonucleotide
tggcattcttggcatct

SEQ ID NO: 20 - DNA, 24 bp, Artificial Sequence
  oligonucleotide
tggtggtggtgtccatttggcttt

SEQ ID NO: 21 - DNA, 20 bp, Artificial Sequence
  oligonucleotide
gactcccttgttatcacctt

SEQ ID NO: 22 - DNA, 19 bp, Artificial Sequence
  oligonucleotide
cacaatgcacagacaactc

SEQ ID NO: 23 - DNA, 24 bp, Artificial Sequence
  oligonucleotide
tgccaatgtgctggaagatgaccc

SEQ ID NO: 24 - DNA, 18 bp, Artificial Sequence
  oligonucleotide
tgtagacgtgaggtaggt

SEQ ID NO: 25 - DNA, 18 bp, Artificial Sequence
  oligonucleotide
cttacccagatgccacaa

SEQ ID NO: 26 - DNA, 24 bp, Artificial Sequence
  oligonucleotide
cggtggaaggaaggatggcagaga

SEQ ID NO: 27 - DNA, 18 bp, Artificial Sequence
  oligonucleotide
ggtcatcttccagcacat

SEQ ID NO: 28 - DNA, 18 bp, Artificial Sequence
  oligonucleotide
ggatggcagagacattga

SEQ ID NO: 29 - DNA, 24 bp, Artificial Sequence
  oligonucleotide
tgccaatgtgctggaagatgaccc

SEQ ID NO: 30 - DNA, 17 bp, Artificial Sequence
  oligonucleotide
ccttcctcgctgtagac

SEQ ID NO: 31 - DNA, 17 bp, Artificial Sequence
  oligonucleotide
tgctctgaccaccaagt

SEQ ID NO: 32 - DNA, 24 bp, Artificial Sequence
  oligonucleotide
accctgacctcgtcctacaactgc

SEQ ID NO: 33 - DNA, 17 bp, Artificial Sequence
  oligonucleotide
tctccgtagggctggta

SEQ ID NO: 34 - DNA, 17 bp, Artificial Sequence
  oligonucleotide
tccgtagggctggtaca

SEQ ID NO: 35 - DNA, 18 bp, Artificial Sequence
  oligonucleotide
ggtccggaaagcactcaa

SEQ ID NO: 36 - DNA, 23 bp, Artificial Sequence
  oligonucleotide
tcctacaactgccccagccccta

SEQ ID NO: 37 - DNA, 17 bp, Artificial Sequence
  oligonucleotide
cctgaccctgctctgac

SEQ ID NO: 38 - DNA, 24 bp, Artificial Sequence
  oligonucleotide
accctgacctcgtcctacaactgc

SEQ ID NO: 39 - DNA, 17 bp, Artificial Sequence
  oligonucleotide
gtacagccgcccatctg

SEQ ID NO: 40 - DNA, 18 bp, Artificial Sequence
  oligonucleotide
tctgaccaccaagtccac

SEQ ID NO: 41 - DNA, 24 bp, Artificial Sequence
  oligonucleotide
cctgacctcgtcctacaactgccc

SEQ ID NO: 42 - DNA, 17 bp, Artificial Sequence
  oligonucleotide
tggtgctgtgcagagag

SEQ ID NO: 43 - DNA, 19 bp, Artificial Sequence
  oligonucleotide
cctcgtttgacatccacta

SEQ ID NO: 44 - DNA, 24 bp, Artificial Sequence
  oligonucleotide
acacagtgtcctggctcaggtacc

SEQ ID NO: 45 - DNA, 17 bp, Artificial Sequence
  oligonucleotide
tgcctctccactttgac

SEQ ID NO: 46 - DNA, 19 bp, Artificial Sequence
  oligonucleotide
ggtacctcgtttgacatcc

SEQ ID NO: 47 - DNA, 24 bp, Artificial Sequence
  oligonucleotide
acacagtgtcctggctcaggtacc

SEQ ID NO: 48 - DNA, 17 bp, Artificial Sequence
  oligonucleotide
aaagacctgcctctcca

SEQ ID NO: 49 - DNA, 23 bp, Artificial Sequence
  oligonucleotide
gtacctcgtttgacatccactat

SEQ ID NO: 50 - DNA, 20 bp, Artificial Sequence
  oligonucleotide
cgtcgtcagcctctgccctg

SEQ ID NO: 51 - DNA, 20 bp, Artificial Sequence
  oligonucleotide
acctgcctctccactttgac

SEQ ID NO: 52 - DNA, 23 bp, Artificial Sequence
  oligonucleotide
acactgtgtcggtgccctgccag

SEQ ID NO: 53 - DNA, 20 bp, Artificial Sequence
  oligonucleotide
aagacctgcctctccacttt

SEQ ID NO: 54 - DNA, 23 bp, Artificial Sequence
  oligonucleotide
gaatggtacctcgtttgacatcc

SEQ ID NO: 55 - DNA, 19 bp, Artificial Sequence
  oligonucleotide
gtcacctacaacacagaca

SEQ ID NO: 56 - DNA, 24 bp, Artificial Sequence
  oligonucleotide
cgtttgagtccatgcccaatcccg

SEQ ID NO: 57 - DNA, 19 bp, Artificial Sequence
  oligonucleotide
cacgtccgtagaaaggtag

SEQ ID NO: 58 - DNA, 17 bp, Artificial Sequence
  oligonucleotide
tgagtccatgcccaatc

SEQ ID NO: 59 - DNA, 24 bp, Artificial Sequence
  oligonucleotide
acacagctggcgccgaatgtatac

SEQ ID NO: 60 - DNA, 20 bp, Artificial Sequence
  oligonucleotide
gtccgtagaaaggtagttgt

SEQ ID NO: 61 - DNA, 18 bp, Artificial Sequence
  oligonucleotide
cgagggccggtatacatt

SEQ ID NO: 62 - DNA, 21 bp, Artificial Sequence
  oligonucleotide
cacgtccgtagaaaggtagtt

SEQ ID NO: 63 - DNA, 23 bp, Artificial Sequence
  oligonucleotide
agctgtgtgactgcctgtcccta

SEQ ID NO: 64 - DNA, 18 bp, Artificial Sequence
  oligonucleotide
gagggccggtatacattc

SEQ ID NO: 65 - DNA, 24 bp, Artificial Sequence
  oligonucleotide
tttctacggacgtgggatcctgca

SEQ ID NO: 66 - DNA, 18 bp, Artificial Sequence
  oligonucleotide
ctgtcacctcttggttgt

SEQ ID NO: 67 - DNA, 19 bp, Artificial Sequence
  oligonucleotide
cttcgatgatgggcttact

SEQ ID NO: 68 - DNA, 24 bp, Artificial Sequence
  oligonucleotide
catgtgaaccagctccctgtctgc

SEQ ID NO: 69 - DNA, 18 bp, Artificial Sequence
  oligonucleotide
ggagggtcaaatccacaa

SEQ ID NO: 70 - DNA, 22 bp, Artificial Sequence
  oligonucleotide
caactgggcgaagagggtgcca

SEQ ID NO: 71 - DNA, 20 bp, Artificial Sequence
  oligonucleotide
agcttcgatgatgggcttac

SEQ ID NO: 72 - DNA, 20 bp, Artificial Sequence
  oligonucleotide
cctgatcatggagggtcaaa

SEQ ID NO: 73 - DNA, 18 bp, Artificial Sequence
  oligonucleotide
ggagctggttcacatgat

SEQ ID NO: 74 - DNA, 24 bp, Artificial Sequence
  oligonucleotide
agggtcaaatccacaaagcctggc

SEQ ID NO: 75 - DNA, 18 bp, Artificial Sequence
  oligonucleotide
ctagccaggcacattcta

SEQ ID NO: 76 - DNA, 18 bp, Artificial Sequence
  oligonucleotide
gatgggcttactgaccaa

SEQ ID NO: 77 - DNA, 24 bp, Artificial Sequence
  oligonucleotide
catgtgaaccagctccctgtctgc

SEQ ID NO: 78 - DNA, 19 bp, Artificial Sequence
  oligonucleotide
ctgatcatggagggtcaaa

SEQ ID NO: 79 - DNA, 18 bp, Artificial Sequence
  oligonucleotide
gaggttcttgccagatga

SEQ ID NO: 80 - DNA, 25 bp, Artificial Sequence
  oligonucleotide
tgtgttggctgtggaaagaaaggct

SEQ ID NO: 81 - DNA, 20 bp, Artificial Sequence
  oligonucleotide
ttggagaattccgtgaagta

SEQ ID NO: 82 - DNA, 26 bp, Artificial Sequence
  oligonucleotide
tctctgggaattgtgttggctgtgga

SEQ ID NO: 83 - DNA, 22 bp, Artificial Sequence
  oligonucleotide
ggagaattccgtgaagtaacag

SEQ ID NO: 84 - DNA, 20 bp, Artificial Sequence
  oligonucleotide
aagaggttcttgccagatga

SEQ ID NO: 85 - DNA, 19 bp, Artificial Sequence
  oligonucleotide
gatgacaatggccacaatg

SEQ ID NO: 86 - DNA, 25 bp, Artificial Sequence
  oligonucleotide
tgtgttggctgtggaaagaaaggct

SEQ ID NO: 87 - DNA, 18 bp, Artificial Sequence
  oligonucleotide
cttcttgggttgagtggt

SEQ ID NO: 88 - DNA, 18 bp, Artificial Sequence
  oligonucleotide
ccgttgacaagaggttct

SEQ ID NO: 89 - DNA, 28 bp, Artificial Sequence
  oligonucleotide
ccagagaaaccaagaagagcattgtggc

SEQ ID NO: 90 - DNA, 18 bp, Artificial Sequence
  oligonucleotide
ccacagccaacacaattc

SEQ ID NO: 91 - DNA, 17 bp, Artificial Sequence
  oligonucleotide
ggtcagtgtgccctcag

SEQ ID NO: 92 - DNA, 18 bp, Artificial Sequence
  oligonucleotide
ggacgagcatggctacat

SEQ ID NO: 93 - DNA, 24 bp, Artificial Sequence
  oligonucleotide
acccaagtttcctcctccctgtcc

SEQ ID NO: 94 - DNA, 18 bp, Artificial Sequence
  oligonucleotide
cttcacgcggaaatacac

SEQ ID NO: 95 - DNA, 24 bp, Artificial Sequence
  oligonucleotide
acccaagtttcctcctccctgtcc

SEQ ID NO: 96 - DNA, 18 bp, Artificial Sequence
  oligonucleotide
gatggtgatctcgttgga

SEQ ID NO: 97 - DNA, 17 bp, Artificial Sequence
  oligonucleotide
agcatggctacatctcc

SEQ ID NO: 98 - DNA, 24 bp, Artificial Sequence
  oligonucleotide
tgcttcacgcggaaatacacgctg

SEQ ID NO: 99 - DNA, 17 bp, Artificial Sequence
  oligonucleotide
gaggaggaaacttgggt

SEQ ID NO: 100 - DNA, 17 bp, Artificial Sequence
  oligonucleotide
gacgagcatggctacat

SEQ ID NO: 101 - DNA, 24 bp, Artificial Sequence
  oligonucleotide
tgcttcacgcggaaatacacgctg

SEQ ID NO: 102 - DNA, 17 bp, Artificial Sequence
  oligonucleotide
acagggaggaggaaact

SEQ ID NO: 103 - DNA, 20 bp, Artificial Sequence
  oligonucleotide
acgaggacctctacatcatc

SEQ ID NO: 104 - DNA, 24 bp, Artificial Sequence
  oligonucleotide
aagcagtgtcacccagctctggat

SEQ ID NO: 105 - DNA, 17 bp, Artificial Sequence
  oligonucleotide
ccacacaccagcacttg

SEQ ID NO: 106 - DNA, 17 bp, Artificial Sequence
  oligonucleotide
ccaactgcgaccgcaac

SEQ ID NO: 107 - DNA, 18 bp, Artificial Sequence
  oligonucleotide
gtcttccggtccacacac

SEQ ID NO: 108 - DNA, 24 bp, Artificial Sequence
  oligonucleotide
caagcagtgtcacccagctctgga

SEQ ID NO: 109 - DNA, 17 bp, Artificial Sequence
  oligonucleotide
ctggccgcttcacagag

SEQ ID NO: 110 - DNA, 24 bp, Artificial Sequence
  oligonucleotide
tgatgtagaggtcctcgtgggtgc

SEQ ID NO: 111 - DNA, 18 bp, Artificial Sequence
  oligonucleotide
cagagctgggtgacactg

SEQ ID NO: 112 - DNA, 17 bp, Artificial Sequence
  oligonucleotide
gcaacggcaacttccac

SEQ ID NO: 113 - DNA, 24 bp, Artificial Sequence
  oligonucleotide
aagcagtgtcacccagctctggat

SEQ ID NO: 114 - DNA, 17 bp, Artificial Sequence
  oligonucleotide
gtcttccggtccacaca

SEQ ID NO: 115 - DNA, 18 bp, Artificial Sequence
  oligonucleotide
tgcttagacgctggattt

SEQ ID NO: 116 - DNA, 26 bp, Artificial Sequence
  oligonucleotide
ccctcaacgttagcttcaccaacagg

SEQ ID NO: 117 - DNA, 19 bp, Artificial Sequence
  oligonucleotide
tcgtagtcgaggtcatagt

SEQ ID NO: 118 - DNA, 18 bp, Artificial Sequence
  oligonucleotide
tctctgaaaggctctcct

SEQ ID NO: 119 - DNA, 24 bp, Artificial Sequence
  oligonucleotide
tgcagctgcttagacgctggattt

SEQ ID NO: 120 - DNA, 19 bp, Artificial Sequence
  oligonucleotide
tcctgttggtgaagctaac

SEQ ID NO: 121 - DNA, 17 bp, Artificial Sequence
  oligonucleotide
gacccgcttctctgaaa

SEQ ID NO: 122 - DNA, 24 bp, Artificial Sequence
  oligonucleotide
tgcagctgcttagacgctggattt

SEQ ID NO: 123 - DNA, 19 bp, Artificial Sequence
  oligonucleotide
aggtcatagttcctgttgg

SEQ ID NO: 124 - DNA, 19 bp, Artificial Sequence
  oligonucleotide
ccggatgacatcagagcta

SEQ ID NO: 125 - DNA, 26 bp, Artificial Sequence
  oligonucleotide
tctcagaaagcagaggctcagagctt

SEQ ID NO: 126 - DNA, 22 bp, Artificial Sequence
  oligonucleotide
aatgcaaatatcagtgttcgtc

SEQ ID NO: 127 - DNA, 20 bp, Artificial Sequence
  oligonucleotide
ctcagagcttggagacagac

SEQ ID NO: 128 - DNA, 30 bp, Artificial Sequence
  oligonucleotide
aggattctatctgcttactgctacagacct

SEQ ID NO: 129 - DNA, 20 bp, Artificial Sequence
  oligonucleotide
gcaaggaggaggagaagaat

SEQ ID NO: 130 - DNA, 24 bp, Artificial Sequence
  oligonucleotide
aagcagaggctcagagcttggaga

SEQ ID NO: 131 - DNA, 21 bp, Artificial Sequence
  oligonucleotide
caacagccttctcaattttct

SEQ ID NO: 132 - DNA, 23 bp, Artificial Sequence
  oligonucleotide
cccattaaatgcaaatatcagtg

SEQ ID NO: 133 - DNA, 18 bp, Artificial Sequence
  oligonucleotide
tcagagcttggagacaga

SEQ ID NO: 134 - DNA, 28 bp, Artificial Sequence
  oligonucleotide
aaggattctatctgcttactgctacaga

SEQ ID NO: 135 - DNA, 24 bp, Artificial Sequence
  oligonucleotide
gagaagaattccttaacacatagg

SEQ ID NO: 136 - DNA, 20 bp, Artificial Sequence
  oligonucleotide
tgctcaacatgacgcttgtc

SEQ ID NO: 137 - DNA, 21 bp, Artificial Sequence
  oligonucleotide
gccatgaggaagtggttgaaa

SEQ ID NO: 138 - DNA, 24 bp, Artificial Sequence
  oligonucleotide
aagccgtgtcatgttcctgctggt

SEQ ID NO: 139 - DNA, 24 bp, Artificial Sequence
  oligonucleotide
gagttcctctgtatgaaagtattg

SEQ ID NO: 140 - DNA, 25 bp, Artificial Sequence
  oligonucleotide
tggaagggctacgaagtcaaaccca

SEQ ID NO: 141 - DNA, 20 bp, Artificial Sequence
  oligonucleotide
atgtagcttgacctcatctc

SEQ ID NO: 142 - DNA, 20 bp, Artificial Sequence
  oligonucleotide
tggcagatcccacaggagtt

SEQ ID NO: 143 - DNA, 30 bp, Artificial Sequence
  oligonucleotide
agcttcaagttagccaagaagagttcctct

SEQ ID NO: 144 - DNA, 24 bp, Artificial Sequence
  oligonucleotide
agcccttccaaaggaattgtatta

SEQ ID NO: 145 - DNA, 20 bp, Artificial Sequence
  oligonucleotide
agttagccaagaagagttcc

SEQ ID NO: 146 - DNA, 25 bp, Artificial Sequence
  oligonucleotide
tggaagggctacgaagtcaaaccca

SEQ ID NO: 147 - DNA, 21 bp, Artificial Sequence
  oligonucleotide
agctctctaatgtagcttgac

SEQ ID NO: 148 - DNA, 21 bp, Artificial Sequence
  oligonucleotide
ccaagaagagttcctctgtat

SEQ ID NO: 149 - DNA, 25 bp, Artificial Sequence
  oligonucleotide
tggaagggctacgaagtcaaaccca

SEQ ID NO: 150 - DNA, 20 bp, Artificial Sequence
  oligonucleotide
cttgacctcatctcctcaaa

SEQ ID NO: 151 - DNA, 17 bp, Artificial Sequence
  oligonucleotide
cagatcaccctcctcaa

SEQ ID NO: 152 - DNA, 23 bp, Artificial Sequence
  oligonucleotide
cctggacatcctgatcctgcgga

SEQ ID NO: 153 - DNA, 17 bp, Artificial Sequence
  oligonucleotide
cgagaaggtcatggtgt

SEQ ID NO: 154 - DNA, 17 bp, Artificial Sequence
  oligonucleotide
ttcaccaccctcaccat

SEQ ID NO: 155 - DNA, 24 bp, Artificial Sequence
  oligonucleotide
agccttgaggagggtgatctggtc

SEQ ID NO: 156 - DNA, 19 bp, Artificial Sequence
  oligonucleotide
agaaggtcatggtgtcctg

SEQ ID NO: 157 - DNA, 17 bp, Artificial Sequence
  oligonucleotide
gtgtaccgcgtgcagat

SEQ ID NO: 158 - DNA, 21 bp, Artificial Sequence
  oligonucleotide
cattaagactgtggagttcgc

SEQ ID NO: 159 - DNA, 23 bp, Artificial Sequence
  oligonucleotide
accagatcaccctcctcaaggct

SEQ ID NO: 160 - DNA, 17 bp, Artificial Sequence
  oligonucleotide
ctgcctgcctggacatc

SEQ ID NO: 161 - DNA, 24 bp, Artificial Sequence
  oligonucleotide
cgagaaggtcatggtgtcctgctc

SEQ ID NO: 162 - DNA, 18 bp, Artificial Sequence
  oligonucleotide
catctgggtccggttcag

SEQ ID NO: 163 - DNA, 18 bp, Artificial Sequence
  oligonucleotide
gctatggccctgaagatt

SEQ ID NO: 164 - DNA, 24 bp, Artificial Sequence
  oligonucleotide
acaaagacgagcaggactaaacga

SEQ ID NO: 165 - DNA, 22 bp, Artificial Sequence
  oligonucleotide
acctaactaggttctgaatgaa

SEQ ID NO: 166 - DNA, 19 bp, Artificial Sequence
  oligonucleotide
aaacagtttcctgctatgg

SEQ ID NO: 167 - DNA, 24 bp, Artificial Sequence
  oligonucleotide
ccctgaagattcctgccaagagaa

SEQ ID NO: 168 - DNA, 18 bp, Artificial Sequence
  oligonucleotide
tagtcctgctcgtctttg

SEQ ID NO: 169 - DNA, 20 bp, Artificial Sequence
  oligonucleotide
agattcctgccaagagaata

SEQ ID NO: 170 - DNA, 24 bp, Artificial Sequence
  oligonucleotide
acaaagacgagcaggactaaacga

SEQ ID NO: 171 - DNA, 21 bp, Artificial Sequence
  oligonucleotide
ggatacctaactaggttctga

SEQ ID NO: 172 - DNA, 24 bp, Artificial Sequence
  oligonucleotide
acaaagacgagcaggactaaacga

SEQ ID NO: 173 - DNA, 25 bp, Artificial Sequence
  oligonucleotide
tggatacctaactaggttctgaatg

SEQ ID NO: 174 - DNA, 25 bp, Artificial Sequence
  oligonucleotide
ctgccaagagaatatttggtgataa

SEQ ID NO: 175 - DNA, 20 bp, Artificial Sequence
  oligonucleotide
tgcaggtcctcactttaatc

SEQ ID NO: 176 - DNA, 24 bp, Artificial Sequence
  oligonucleotide
aaacacggtgggccaaaggatgaa

SEQ ID NO: 177 - DNA, 20 bp, Artificial Sequence
  oligonucleotide
ctttgtcagcagtcacattg

SEQ ID NO: 178 - DNA, 23 bp, Artificial Sequence
  oligonucleotide
ggtcctcactttaatcctctatc

SEQ ID NO: 179 - DNA, 24 bp, Artificial Sequence
  oligonucleotide
aaacacggtgggccaaaggatgaa

SEQ ID NO: 180 - DNA, 19 bp, Artificial Sequence
  oligonucleotide
accatctttgtcagcagtc

SEQ ID NO: 181 - DNA, 17 bp, Artificial Sequence
  oligonucleotide
gtgcaggtcctcacttt

SEQ ID NO: 182 - DNA, 24 bp, Artificial Sequence
  oligonucleotide
aaacacggtgggccaaaggatgaa

SEQ ID NO: 183 - DNA, 17 bp, Artificial Sequence
  oligonucleotide
gcccaagtctccaacat

SEQ ID NO: 184 - DNA, 18 bp, Artificial Sequence
  oligonucleotide
cctcccagtgtgcaaata

SEQ ID NO: 185 - DNA, 24 bp, Artificial Sequence
  oligonucleotide
acgtccctccagaagaggagtgtg

SEQ ID NO: 186 - DNA, 19 bp, Artificial Sequence
  oligonucleotide
ccctgcagaagtgtctaaa

SEQ ID NO: 187 - DNA, 20 bp, Artificial Sequence
  oligonucleotide
ccctggtgcttctatcctaa

SEQ ID NO: 188 - DNA, 20 bp, Artificial Sequence
  oligonucleotide
atccctgcagaagtgtctaa

SEQ ID NO: 189 - DNA, 21 bp, Artificial Sequence
  oligonucleotide
accatcgacgtccctccagaa

SEQ ID NO: 190 - DNA, 21 bp, Artificial Sequence
  oligonucleotide
ggtgcttctatcctaatacca

SEQ ID NO: 191 - DNA, 24 bp, Artificial Sequence
  oligonucleotide
agacacttctgcagggatctgcct

SEQ ID NO: 192 - DNA, 18 bp, Artificial Sequence
  oligonucleotide
tctgggactaatcaccgt

SEQ ID NO: 193 - DNA, 20 bp, Artificial Sequence
  oligonucleotide
cctggtgcttctatcctaat

SEQ ID NO: 194 - DNA, 24 bp, Artificial Sequence
  oligonucleotide
acgtccctccagaagaggagtgtg

SEQ ID NO: 195 - DNA, 17 bp, Artificial Sequence
  oligonucleotide
gatccctgcagaagtgt

SEQ ID NO: 196 - DNA, 17 bp, Artificial Sequence
  oligonucleotide
ggacatgagaggcacac

SEQ ID NO: 197 - DNA, 24 bp, Artificial Sequence
  oligonucleotide
tctccctcctctgcctcctctcaa

SEQ ID NO: 198 - DNA, 17 bp, Artificial Sequence
  oligonucleotide
ggcaggtacatggtgtc

SEQ ID NO: 199 - DNA, 19 bp, Artificial Sequence
  oligonucleotide
tcctctgcctcctctcaaa

SEQ ID NO: 200 - DNA, 23 bp, Artificial Sequence
  oligonucleotide
cagctgtgcccgacaccatgtac

SEQ ID NO: 201 - DNA, 17 bp, Artificial Sequence
  oligonucleotide
cagccatccagcaccag

SEQ ID NO: 202 - DNA, 17 bp, Artificial Sequence
  oligonucleotide
acaccgaagacccacct

SEQ ID NO: 203 - DNA, 24 bp, Artificial Sequence
  oligonucleotide
tctccctcctctgcctcctctcaa

SEQ ID NO: 204 - DNA, 17 bp, Artificial Sequence
  oligonucleotide
catggtgtcgggcacag

SEQ ID NO: 205 - DNA, 17 bp, Artificial Sequence
  oligonucleotide
catggtgtcgggcacag

SEQ ID NO: 206 - DNA, 18 bp, Artificial Sequence
  oligonucleotide
gagaggcacaccgaagac

SEQ ID NO: 207 - DNA, 20 bp, Artificial Sequence
  oligonucleotide
ttctccctcctctgcctcct

SEQ ID NO: 208 - DNA, 18 bp, Artificial Sequence
  oligonucleotide
tggattctggcggtattg

SEQ ID NO: 209 - DNA, 25 bp, Artificial Sequence
  oligonucleotide
ttgggcattctggacaacttggacc

SEQ ID NO: 210 - DNA, 19 bp, Artificial Sequence
  oligonucleotide
ggaagggcatttgaagaac

SEQ ID NO: 211 - DNA, 21 bp, Artificial Sequence
  oligonucleotide
catgactgggtccaagttgtc

SEQ ID NO: 212 - DNA, 20 bp, Artificial Sequence
  oligonucleotide
ggattctggcggtattgact

SEQ ID NO: 213 - DNA, 30 bp, Artificial Sequence
  oligonucleotide
tcagagtctgatatcctgttgggcattctg

SEQ ID NO: 214 - DNA, 20 bp, Artificial Sequence
  oligonucleotide
gcggtattgactcttcagat

SEQ ID NO: 215 - DNA, 28 bp, Artificial Sequence
  oligonucleotide
tctgatatcctgttgggcattctggaca

SEQ ID NO: 216 - DNA, 18 bp, Artificial Sequence
  oligonucleotide
gaacatgactgggtccaa

SEQ ID NO: 217 - DNA, 18 bp, Artificial Sequence
  oligonucleotide
ctggcggtattgactctt

SEQ ID NO: 218 - DNA, 25 bp, Artificial Sequence
  oligonucleotide
ttgggcattctggacaacttggacc

SEQ ID NO: 219 - DNA, 20 bp, Artificial Sequence
  oligonucleotide
ggcatttgaagaacatgact

SEQ ID NO: 220 - DNA, 19 bp, Artificial Sequence
  oligonucleotide
ccattgagagggtgtcaga

SEQ ID NO: 221 - DNA, 26 bp, Artificial Sequence
  oligonucleotide
tggattcttcggatgctgacgattgc

SEQ ID NO: 222 - DNA, 22 bp, Artificial Sequence
  oligonucleotide
cgctgtgatatctcatcaagta

SEQ ID NO: 223 - DNA, 20 bp, Artificial Sequence
  oligonucleotide
gagagggtgtcagaggcaat

SEQ ID NO: 224 - DNA, 24 bp, Artificial Sequence
  oligonucleotide
gcgctgtgatatctcatcaagtag

SEQ ID NO: 225 - DNA, 26 bp, Artificial Sequence
  oligonucleotide
cgtcagcatccgaagaatccagacct

SEQ ID NO: 226 - DNA, 18 bp, Artificial Sequence
  oligonucleotide
gtgtcagaggcaatcgtc

SEQ ID NO: 227 - DNA, 18 bp, Artificial Sequence
  oligonucleotide
cgtcagctgccgtcagat

SEQ ID NO: 228 - DNA, 34 bp, Artificial Sequence
  oligonucleotide
cagacctttttgctacttgatgagatatcacagc

SEQ ID NO: 229 - DNA, 19 bp, Artificial Sequence
  oligonucleotide
gccattgagagggtgtcag

SEQ ID NO: 230 - DNA, 18 bp, Artificial Sequence
  oligonucleotide
tatcacagcgcaaccgtc

SEQ ID NO: 231 - DNA, 28 bp, Artificial Sequence
  oligonucleotide
gcatccgaagaatccagacctttttgct

SEQ ID NO: 232 - DNA, 21 bp, Artificial Sequence
  oligonucleotide
cctgatctgtggagatgaagc

SEQ ID NO: 233 - DNA, 19 bp, Artificial Sequence
  oligonucleotide
gcgcacaggtacttctgtt

SEQ ID NO: 234 - DNA, 22 bp, Artificial Sequence
  oligonucleotide
tggaagctgcaaggtcttcttc

SEQ ID NO: 235 - DNA, 19 bp, Artificial Sequence
  oligonucleotide
gcttctgggtgtcactatg

SEQ ID NO: 236 - DNA, 19 bp, Artificial Sequence
  oligonucleotide
cgcacaggtacttctgttt

SEQ ID NO: 237 - DNA, 25 bp, Artificial Sequence
  oligonucleotide
tgtggaagctgcaaggtcttcttca

SEQ ID NO: 238 - DNA, 20 bp, Artificial Sequence
  oligonucleotide
ctgatctgtggagatgaagc

SEQ ID NO: 239 - DNA, 21 bp, Artificial Sequence
  oligonucleotide
ggtacttctgtttcccttcag

SEQ ID NO: 240 - DNA, 25 bp, Artificial Sequence
  oligonucleotide
tgtggaagctgcaaggtcttcttca

SEQ ID NO: 241 - DNA, 20 bp, Artificial Sequence
  oligonucleotide
gctgaagggaaacagaagta

SEQ ID NO: 242 - DNA, 23 bp, Artificial Sequence
  oligonucleotide
tgcgccagcagaaatgattgcac

SEQ ID NO: 243 - DNA, 20 bp, Artificial Sequence
  oligonucleotide
gaagacgacaagatggacaa

SEQ ID NO: 244 - DNA, 20 bp, Artificial Sequence
  oligonucleotide
gcatttatggtgcctgattc

SEQ ID NO: 245 - DNA, 19 bp, Artificial Sequence
  oligonucleotide
caggaacagggtttgacag

SEQ ID NO: 246 - DNA, 23 bp, Artificial Sequence
  oligonucleotide
agtgagctgccctttgatgctca

SEQ ID NO: 247 - DNA, 19 bp, Artificial Sequence
  oligonucleotide
tgattcctgcatggtcagt

SEQ ID NO: 248 - DNA, 22 bp, Artificial Sequence
  oligonucleotide
acaaccctgctgccctgtcaaa

SEQ ID NO: 249 - DNA, 19 bp, Artificial Sequence
  oligonucleotide
ctcatcggtcaggaacagg

SEQ ID NO: 250 - DNA, 19 bp, Artificial Sequence
  oligonucleotide
tgatgctcatgcccacatc

SEQ ID NO: 251 - DNA, 19 bp, Artificial Sequence
  oligonucleotide
gcagacgcttctcctcatc

SEQ ID NO: 252 - DNA, 22 bp, Artificial Sequence
  oligonucleotide
tgctgccctgtcaaaccctgtt

SEQ ID NO: 253 - DNA, 18 bp, Artificial Sequence
  oligonucleotide
ccagagcaggcaccgtag

SEQ ID NO: 254 - DNA, 20 bp, Artificial Sequence
  oligonucleotide
agcagacgcttctcctcatc

SEQ ID NO: 255 - DNA, 22 bp, Artificial Sequence
  oligonucleotide
caaccctgctgccctgtcaaac

SEQ ID NO: 256 - DNA, 19 bp, Artificial Sequence
  oligonucleotide
gaggcagctggagaagttt

SEQ ID NO: 257 - DNA, 20 bp, Artificial Sequence
  oligonucleotide
gcagcttgtggtacaaggag

SEQ ID NO: 258 - DNA, 24 bp, Artificial Sequence
  oligonucleotide
tatgccgactgctacatgaaccgg

SEQ ID NO: 259 - DNA, 19 bp, Artificial Sequence
  oligonucleotide
tggcttccagatgctgtat

SEQ ID NO: 260 - DNA, 19 bp, Artificial Sequence
  oligonucleotide
tggtacaaggagccatcaa

SEQ ID NO: 261 - DNA, 22 bp, Artificial Sequence
  oligonucleotide
aaccgggaggagttctgggaga

SEQ ID NO: 262 - DNA, 19 bp, Artificial Sequence
  oligonucleotide
aggcagctggagaagtttg

SEQ ID NO: 263 - DNA, 19 bp, Artificial Sequence
  oligonucleotide
gcagcttgtggtacaagga

SEQ ID NO: 264 - DNA, 22 bp, Artificial Sequence
  oligonucleotide
aaccgggaggagttctgggaga

SEQ ID NO: 265 - DNA, 19 bp, Artificial Sequence
  oligonucleotide
aacactttgaagccaggtc

SEQ ID NO: 266 - DNA, 20 bp, Artificial Sequence
  oligonucleotide
caaacatctcccagaactcc

SEQ ID NO: 267 - DNA, 22 bp, Artificial Sequence
  oligonucleotide
tgccgactgctacatgaaccgg

SEQ ID NO: 268 - DNA, 19 bp, Artificial Sequence
  oligonucleotide
caacatcaccgtactgcat

SEQ ID NO: 269 - DNA, 20 bp, Artificial Sequence
  oligonucleotide
gagtccttggaactggattg

SEQ ID NO: 270 - DNA, 22 bp, Artificial Sequence
  oligonucleotide
tgaagctcaccgagacggcgat

SEQ ID NO: 271 - DNA, 20 bp, Artificial Sequence
  oligonucleotide
catgtgaagctcaccgagac

SEQ ID NO: 272 - DNA, 22 bp, Artificial Sequence
  oligonucleotide
ggaactggattgaaggtcgaaa

SEQ ID NO: 273 - DNA, 23 bp, Artificial Sequence
  oligonucleotide
ctcgagacttaccagagccacaa

SEQ ID NO: 274 - DNA, 17 bp, Artificial Sequence
  oligonucleotide
agaagtttgtccgcagc

SEQ ID NO: 275 - DNA, 19 bp, Artificial Sequence
  oligonucleotide
gagtccttggaactggatt

SEQ ID NO: 276 - DNA, 32 bp, Artificial Sequence
  oligonucleotide
tcgagacttaccagagccacaaaatttaattc

SEQ ID NO: 277 - DNA, 19 bp, Artificial Sequence
  oligonucleotide
cgtactgcatgtgaagctc

SEQ ID NO: 278 - DNA, 21 bp, Artificial Sequence
  oligonucleotide
gaactggattgaaggtcgaaa

SEQ ID NO: 279 - DNA, 25 bp, Artificial Sequence
  oligonucleotide
tttgtggctctggtaagtctcgagc

SEQ ID NO: 280 - DNA, 19 bp, Artificial Sequence
  oligonucleotide
aacggaaaggagagggata

SEQ ID NO: 281 - DNA, 19 bp, Artificial Sequence
  oligonucleotide
tttgactgcagagatgtgg

SEQ ID NO: 282 - DNA, 21 bp, Artificial Sequence
  oligonucleotide
aaatccacctggaaggccgct

SEQ ID NO: 283 - DNA, 20 bp, Artificial Sequence
  oligonucleotide
tgaagatggaggcattatcc

SEQ ID NO: 284 - DNA, 20 bp, Artificial Sequence
  oligonucleotide
aacagtagaaatccacctgg

SEQ ID NO: 285 - DNA, 25 bp, Artificial Sequence
  oligonucleotide
ggagaaccaaacggaaaggagaggg

SEQ ID NO: 286 - DNA, 22 bp, Artificial Sequence
  oligonucleotide
acggaaaggagagggatattca

SEQ ID NO: 287 - DNA, 22 bp, Artificial Sequence
  oligonucleotide
gtcaaacatccttccaccacag

SEQ ID NO: 288 - DNA, 26 bp, Artificial Sequence
  oligonucleotide
acagtagaaatccacctggaaggccg

SEQ ID NO: 289 - DNA, 20 bp, Artificial Sequence
  oligonucleotide
acggaaaggagagggatatt

SEQ ID NO: 290 - DNA, 19 bp, Artificial Sequence
  oligonucleotide
ccacagtgaatgcatcctt

SEQ ID NO: 291 - DNA, 21 bp, Artificial Sequence
  oligonucleotide
aaatccacctggaaggccgct

SEQ ID NO: 292 - DNA, 23 bp, Artificial Sequence
  oligonucleotide
gggattacacaaagagagtgata

SEQ ID NO: 293 - DNA, 22 bp, Artificial Sequence
  oligonucleotide
tcatcttcagggccatcagcgc

SEQ ID NO: 294 - DNA, 22 bp, Artificial Sequence
  oligonucleotide
gggagacataacttcatcagag

SEQ ID NO: 295 - DNA, 19 bp, Artificial Sequence
  oligonucleotide
ccagagtgcagtccaattc

SEQ ID NO: 296 - DNA, 24 bp, Artificial Sequence
  oligonucleotide
gggattacacaaagagagtgatac

SEQ ID NO: 297 - DNA, 24 bp, Artificial Sequence
  oligonucleotide
tggccctgaagatgatggagctct

SEQ ID NO: 298 - DNA, 19 bp, Artificial Sequence
  oligonucleotide
ggcgatgcctattgtgtag

SEQ ID NO: 299 - DNA, 20 bp, Artificial Sequence
  oligonucleotide
tcagagagctccatcatctt

SEQ ID NO: 300 - DNA, 23 bp, Artificial Sequence
  oligonucleotide
gggccatcagcgctatctgaaca

SEQ ID NO: 301 - DNA, 21 bp, Artificial Sequence
  oligonucleotide
gagagtgatactcatgctgtt

SEQ ID NO: 302 - DNA, 22 bp, Artificial Sequence
  oligonucleotide
caattcgcatcttgataggttc

SEQ ID NO: 303 - DNA, 22 bp, Artificial Sequence
  oligonucleotide
gcgctgatggccctgaagatga

SEQ ID NO: 304 - DNA, 21 bp, Artificial Sequence
  oligonucleotide
tcgaaccagaggagttcttgc

SEQ ID NO: 305 - DNA, 21 bp, Artificial Sequence
  oligonucleotide
agcacacatgtcattggacag

SEQ ID NO: 306 - DNA, 25 bp, Artificial Sequence
  oligonucleotide
cccaggagtcttcagtgtgtgagcc

SEQ ID NO: 307 - DNA, 19 bp, Artificial Sequence
  oligonucleotide
aggagtcttcagtgtgtga

SEQ ID NO: 308 - DNA, 20 bp, Artificial Sequence
  oligonucleotide
acaacatgaactctgtcacc

SEQ ID NO: 309 - DNA, 24 bp, Artificial Sequence
  oligonucleotide
tgtcattggacaggagatggaggc

SEQ ID NO: 310 - DNA, 19 bp, Artificial Sequence
  oligonucleotide
gtgtgtgagcctccatctc

SEQ ID NO: 311 - DNA, 20 bp, Artificial Sequence
  oligonucleotide
ccagcacacaacatgaactc

SEQ ID NO: 312 - DNA, 27 bp, Artificial Sequence
  oligonucleotide
ctgtccaatgacatgtgtgctagagct

SEQ ID NO: 313 - DNA, 19 bp, Artificial Sequence
  oligonucleotide
catcgaaccagaggagttc

SEQ ID NO: 314 - DNA, 20 bp, Artificial Sequence
  oligonucleotide
gaactctgtcaccttctcag

SEQ ID NO: 315 - DNA, 23 bp, Artificial Sequence
  oligonucleotide
tgtgagcctccatctcctgtcca

SEQ ID NO: 316 - DNA, 20 bp, Artificial Sequence
  oligonucleotide
gaaccagaggagttcttgac

SEQ ID NO: 317 - DNA, 19 bp, Artificial Sequence
  oligonucleotide
tgaacttggtcaccttctg

SEQ ID NO: 318 - DNA, 23 bp, Artificial Sequence
  oligonucleotide
ccaatgacgtgtgtgcgcaagtt

SEQ ID NO: 319 - DNA, 21 bp, Artificial Sequence
  oligonucleotide
gtgtggacctccatgttattt

SEQ ID NO: 320 - DNA, 19 bp, Artificial Sequence
  oligonucleotide
acacagcatgaacttggtc

SEQ ID NO: 321 - DNA, 23 bp, Artificial Sequence
  oligonucleotide
tgtgcgcaagttcaccctcagaa

SEQ ID NO: 322 - DNA, 19 bp, Artificial Sequence
  oligonucleotide
gatgctgtgaaggtcatgg

SEQ ID NO: 323 - DNA, 21 bp, Artificial Sequence
  oligonucleotide
cacacactgaagtttctttgg

SEQ ID NO: 324 - DNA, 23 bp, Artificial Sequence
  oligonucleotide
actcctctggttcaatgctgccc

SEQ ID NO: 325 - DNA, 19 bp, Artificial Sequence
  oligonucleotide
gcagcattgaaccagagga

SEQ ID NO: 326 - DNA, 22 bp, Artificial Sequence
  oligonucleotide
gcacacacgtcattggaaataa

SEQ ID NO: 327 - DNA, 27 bp, Artificial Sequence
  oligonucleotide
cccaaagaaacttcagtgtgtggacct

SEQ ID NO: 328 - DNA, 20 bp, Artificial Sequence
  oligonucleotide
gcctataaaggagctcaacc

SEQ ID NO: 329 - DNA, 19 bp, Artificial Sequence
  oligonucleotide
ccgtgacagaccatcaaat

SEQ ID NO: 330 - DNA, 22 bp, Artificial Sequence
  oligonucleotide
tcggattggcaccctggagttg

SEQ ID NO: 331 - DNA, 19 bp, Artificial Sequence
  oligonucleotide
gaacaacatcacggaagtg

SEQ ID NO: 332 - DNA, 19 bp, Artificial Sequence
  oligonucleotide
catcaaatgctcccaactc

SEQ ID NO: 333 - DNA, 22 bp, Artificial Sequence
  oligonucleotide
acacctgctttccacacggacc

SEQ ID NO: 334 - DNA, 18 bp, Artificial Sequence
  oligonucleotide
acacggaccgcctataaa

SEQ ID NO: 335 - DNA, 20 bp, Artificial Sequence
  oligonucleotide
ccatcaaatgctcccaactc

SEQ ID NO: 336 - DNA, 22 bp, Artificial Sequence
  oligonucleotide
agctcaacctggcaggcaatcg

SEQ ID NO: 337 - DNA, 19 bp, Artificial Sequence
  oligonucleotide
cacacggaccgcctataaa

SEQ ID NO: 338 - DNA, 20 bp, Artificial Sequence
  oligonucleotide
ttgctcaggcgaagagttag

SEQ ID NO: 339 - DNA, 22 bp, Artificial Sequence
  oligonucleotide
tcggattggcaccctggagttg

SEQ ID NO: 340 - DNA, 21 bp, Artificial Sequence
  oligonucleotide
gcattattggcatgggaacag

SEQ ID NO: 341 - DNA, 22 bp, Artificial Sequence
  oligonucleotide
ccaccatctcagggttgtttag

SEQ ID NO: 342 - DNA, 25 bp, Artificial Sequence
  oligonucleotide
cgcctacatcctaactcgatttgct

SEQ ID NO: 343 - DNA, 20 bp, Artificial Sequence
  oligonucleotide
cattattggcatgggaacag

SEQ ID NO: 344 - DNA, 19 bp, Artificial Sequence
  oligonucleotide
agggttcacgttgataagg

SEQ ID NO: 345 - DNA, 24 bp, Artificial Sequence
  oligonucleotide
aaacaaccctgagatggtggaggg

SEQ ID NO: 346 - DNA, 19 bp, Artificial Sequence
  oligonucleotide
tgaaatgcttcctggagtc

SEQ ID NO: 347 - DNA, 21 bp, Artificial Sequence
  oligonucleotide
cagggttgtttagagcaaatc

SEQ ID NO: 348 - DNA, 22 bp, Artificial Sequence
  oligonucleotide
ttggcatgggaacaggagcagg

SEQ ID NO: 349 - DNA, 19 bp, Artificial Sequence
  oligonucleotide
cttcctggagtccttcaac

SEQ ID NO: 350 - DNA, 20 bp, Artificial Sequence
  oligonucleotide
accatctcagggttgtttag

SEQ ID NO: 351 - DNA, 22 bp, Artificial Sequence
  oligonucleotide
ttggcatgggaacaggagcagg

SEQ ID NO: 352 - DNA, 18 bp, Artificial Sequence
  oligonucleotide
cttctgcggctgcttagg

SEQ ID NO: 353 - DNA, 19 bp, Artificial Sequence
  oligonucleotide
cagagaccgagccagaaag

SEQ ID NO: 354 - DNA, 23 bp, Artificial Sequence
  oligonucleotide
aaacacttcaggcgcccttccaa

SEQ ID NO: 355 - DNA, 19 bp, Artificial Sequence
  oligonucleotide
cagagaccgagccagaaag

SEQ ID NO: 356 - DNA, 19 bp, Artificial Sequence
  oligonucleotide
cacctgagtgtgggagaag

SEQ ID NO: 357 - DNA, 22 bp, Artificial Sequence
  oligonucleotide
aacacttcaggcgcccttccaa

SEQ ID NO: 358 - DNA, 19 bp, Artificial Sequence
  oligonucleotide
gagccagaaaggcacttgg

SEQ ID NO: 359 - DNA, 21 bp, Artificial Sequence
  oligonucleotide
tctccaactcgatcacctgag

SEQ ID NO: 360 - DNA, 22 bp, Artificial Sequence
  oligonucleotide
aacacttcaggcgcccttccaa

SEQ ID NO: 361 - DNA, 21 bp, Artificial Sequence
  oligonucleotide
ggtcttatctgttggactctg

SEQ ID NO: 362 - DNA, 20 bp, Artificial Sequence
  oligonucleotide
ctgaacttcctctccaactc

SEQ ID NO: 363 - DNA, 22 bp, Artificial Sequence
  oligonucleotide
aacacttcaggcgcccttccaa

SEQ ID NO: 364 - DNA, 22 bp, Artificial Sequence
  oligonucleotide
tcgaactgtgactgtctaaatg

SEQ ID NO: 365 - DNA, 19 bp, Artificial Sequence
  oligonucleotide
ctgccctccgaatttcttt

SEQ ID NO: 366 - DNA, 23 bp, Artificial Sequence
  oligonucleotide
aacattcactggtgcaactgccc

SEQ ID NO: 367 - DNA, 19 bp, Artificial Sequence
  oligonucleotide
gttccatcgaactgtgact

SEQ ID NO: 368 - DNA, 20 bp, Artificial Sequence
  oligonucleotide
cgaatttctttgggcagttg

SEQ ID NO: 369 - DNA, 26 bp, Artificial Sequence
  oligonucleotide
tggaggaacatgtgtgtccaacaagt

SEQ ID NO: 370 - DNA, 20 bp, Artificial Sequence
  oligonucleotide
gtgcaactgcccaaagaaat

SEQ ID NO: 371 - DNA, 19 bp, Artificial Sequence
  oligonucleotide
gacagtggcagagttccag

SEQ ID NO: 372 - DNA, 22 bp, Artificial Sequence
  oligonucleotide
aggaaaggccagcactgacacc

SEQ ID NO: 373 - DNA, 19 bp, Artificial Sequence
  oligonucleotide
actgcccaaagaaattcgg

SEQ ID NO: 374 - DNA, 19 bp, Artificial Sequence
  oligonucleotide
ctggcctttcctcggtaaa

SEQ ID NO: 375 - DNA, 31 bp, Artificial Sequence
  oligonucleotide
cagcactgtgaaatagataagtcaaaaacct

SEQ ID NO: 376 - DNA, 19 bp, Artificial Sequence
  oligonucleotide
tggtgatgatggtgatggt

SEQ ID NO: 377 - DNA, 19 bp, Artificial Sequence
  oligonucleotide
cagggcatcttctctcctc

SEQ ID NO: 378 - DNA, 22 bp, Artificial Sequence
  oligonucleotide
acaagctgtctgcacggtcctt

SEQ ID NO: 379 - DNA, 20 bp, Artificial Sequence
  oligonucleotide
agccactacaagctgtctgc

SEQ ID NO: 380 - DNA, 19 bp, Artificial Sequence
  oligonucleotide
tgacactgtgctctccgag

SEQ ID NO: 381 - DNA, 24 bp, Artificial Sequence
  oligonucleotide
agagaagatgccctgtcctcagaa

SEQ ID NO: 382 - DNA, 19 bp, Artificial Sequence
  oligonucleotide
gtctgcacggtccttcatc

SEQ ID NO: 383 - DNA, 19 bp, Artificial Sequence
  oligonucleotide
gattccgttgcctgacact

SEQ ID NO: 384 - DNA, 25 bp, Artificial Sequence
  oligonucleotide
agaagatgccctgtcctcagaagga

SEQ ID NO: 385 - DNA, 19 bp, Artificial Sequence
  oligonucleotide
atcatcgtggtggtgatga

SEQ ID NO: 386 - DNA, 19 bp, Artificial Sequence
  oligonucleotide
ctgaggacagggcatcttc

SEQ ID NO: 387 - DNA, 22 bp, Artificial Sequence
  oligonucleotide
acaagctgtctgcacggtcctt

SEQ ID NO: 388 - DNA, 22 bp, Artificial Sequence
  oligonucleotide
cagcgatggttacattgaatac

SEQ ID NO: 389 - DNA, 19 bp, Artificial Sequence
  oligonucleotide
cgaagagtggcctgaatag

SEQ ID NO: 390 - DNA, 28 bp, Artificial Sequence
  oligonucleotide
acaacctgccttccttaactctttctgc

SEQ ID NO: 391 - DNA, 19 bp, Artificial Sequence
  oligonucleotide
ggcaggttgtccttctatt

SEQ ID NO: 392 - DNA, 21 bp, Artificial Sequence
  oligonucleotide
tcatcctggcttgaagataaa

SEQ ID NO: 393 - DNA, 24 bp, Artificial Sequence
  oligonucleotide
tgtactgcatgctgtttgtggcac

SEQ ID NO: 394 - DNA, 21 bp, Artificial Sequence
  oligonucleotide
atgtcgagggaatgcagaaag

SEQ ID NO: 395 - DNA, 24 bp, Artificial Sequence
  oligonucleotide
caggttgtccttctattcaggcca

SEQ ID NO: 396 - DNA, 24 bp, Artificial Sequence
  oligonucleotide
caggttgtccttctattcaggcca

SEQ ID NO: 397 - DNA, 21 bp, Artificial Sequence
  oligonucleotide
gagggaatgcagaaagagtta

SEQ ID NO: 398 - DNA, 22 bp, Artificial Sequence
  oligonucleotide
gaagataaagtgccacaaacag

SEQ ID NO: 399 - DNA, 25 bp, Artificial Sequence
  oligonucleotide
aggcaggttgtccttctattcaggc

SEQ ID NO: 400 - DNA, 21 bp, Artificial Sequence
  oligonucleotide
ctctaaaggtactgcacatga

SEQ ID NO: 401 - DNA, 20 bp, Artificial Sequence
  oligonucleotide
ctttggctttggctagaaac

SEQ ID NO: 402 - DNA, 29 bp, Artificial Sequence
  oligonucleotide
acagccttcatttctctgaacatactgcc

SEQ ID NO: 403 - DNA, 21 bp, Artificial Sequence
  oligonucleotide
gctagccggttatttcataga

SEQ ID NO: 404 - DNA, 23 bp, Artificial Sequence
  oligonucleotide
ctttcattgatctgtcccataag

SEQ ID NO: 405 - DNA, 31 bp, Artificial Sequence
  oligonucleotide
tgacagccttcatttctctgaacatactgcc

SEQ ID NO: 406 - DNA, 23 bp, Artificial Sequence
  oligonucleotide
ccagtatacaggtacaactacag

SEQ ID NO: 407 - DNA, 22 bp, Artificial Sequence
  oligonucleotide
taaggcagtatgttcagagaaa

SEQ ID NO: 408 - DNA, 28 bp, Artificial Sequence
  oligonucleotide
tgctagccggttatttcatagacactct

SEQ ID NO: 409 - DNA, 24 bp, Artificial Sequence
  oligonucleotide
actctaaaggtactgcacatgatc

SEQ ID NO: 410 - DNA, 22 bp, Artificial Sequence
  oligonucleotide
ctttggctttggctagaaactc

SEQ ID NO: 411 - DNA, 29 bp, Artificial Sequence
  oligonucleotide
aaaacagctctggaaaatgacagccttca

SEQ ID NO: 412 - DNA, 19 bp, Artificial Sequence
  oligonucleotide
ggagcctgagcttatgaat

SEQ ID NO: 413 - DNA, 23 bp, Artificial Sequence
  oligonucleotide
gaagtgaaagtcagatggtttag

SEQ ID NO: 414 - DNA, 22 bp, Artificial Sequence
  oligonucleotide
ttggtggaggagaagggttggc

SEQ ID NO: 415 - DNA, 20 bp, Artificial Sequence
  oligonucleotide
tatgaatgccaacccttctc

SEQ ID NO: 416 - DNA, 19 bp, Artificial Sequence
  oligonucleotide
ccctttccgatcactttca

SEQ ID NO: 417 - DNA, 22 bp, Artificial Sequence
  oligonucleotide
aatcaaccttggcccgtcgtcc

SEQ ID NO: 418 - DNA, 20 bp, Artificial Sequence
  oligonucleotide
caggagcctgagcttatgaa

SEQ ID NO: 419 - DNA, 22 bp, Artificial Sequence
  oligonucleotide
gatggtttagcatgaggattgg

SEQ ID NO: 420 - DNA, 23 bp, Artificial Sequence
  oligonucleotide
tcagcaaatcaaccttggcccgt

SEQ ID NO: 421 - DNA, 20 bp, Artificial Sequence
  oligonucleotide
cttgaagatctcccaacctc

SEQ ID NO: 422 - DNA, 21 bp, Artificial Sequence
  oligonucleotide
caaggttgatttgctgagaag

SEQ ID NO: 423 - DNA, 22 bp, Artificial Sequence
  oligonucleotide
ttggtggaggagaagggttggc

SEQ ID NO: 424 - DNA, 20 bp, Artificial Sequence
  oligonucleotide
atgaaactgaacacaagtgc

SEQ ID NO: 425 - DNA, 21 bp, Artificial Sequence
  oligonucleotide
aggctatacagcgtaaagaaa

SEQ ID NO: 426 - DNA, 27 bp, Artificial Sequence
  oligonucleotide
ctgtaccacagtgatgcctgttcttca

SEQ ID NO: 427 - DNA, 23 bp, Artificial Sequence
  oligonucleotide
ctgttcttcaaaagcagtggttt

SEQ ID NO: 428 - DNA, 17 bp, Artificial Sequence
  oligonucleotide
tggcggcttgagttcaa

SEQ ID NO: 429 - DNA, 25 bp, Artificial Sequence
  oligonucleotide
ttacgctgtatagcctgcggggtca

SEQ ID NO: 430 - DNA, 19 bp, Artificial Sequence
  oligonucleotide
cggatccaccagctttatg

SEQ ID NO: 431 - DNA, 20 bp, Artificial Sequence
  oligonucleotide
tttgaagaacaggcatcact

SEQ ID NO: 432 - DNA, 24 bp, Artificial Sequence
  oligonucleotide
acacaagtgccggcaatgtcgata

SEQ ID NO: 433 - DNA, 20 bp, Artificial Sequence
  oligonucleotide
cttgaagatctcccaacctc

SEQ ID NO: 434 - DNA, 21 bp, Artificial Sequence
  oligonucleotide
caaggttgatttgctgagaag

SEQ ID NO: 435 - DNA, 22 bp, Artificial Sequence
  oligonucleotide
ttggtggaggagaagggttggc

SEQ ID NO: 436 - DNA, 20 bp, Artificial Sequence
  oligonucleotide
cccactgaagaagacaactg

SEQ ID NO: 437 - DNA, 19 bp, Artificial Sequence
  oligonucleotide
tgcaggtctagtacctctg

SEQ ID NO: 438 - DNA, 22 bp, Artificial Sequence
  oligonucleotide
atctgttgcaggaggctcaggc

SEQ ID NO: 439 - DNA, 20 bp, Artificial Sequence
  oligonucleotide
aacaggcagaagaggatctg

SEQ ID NO: 440 - DNA, 19 bp, Artificial Sequence
  oligonucleotide
aggactcatgcagccttac

SEQ ID NO: 441 - DNA, 22 bp, Artificial Sequence
  oligonucleotide
tactagacctgcaggaccgcga

SEQ ID NO: 442 - DNA, 21 bp, Artificial Sequence
  oligonucleotide
agaagaggatctgttgcagga

SEQ ID NO: 443 - DNA, 19 bp, Artificial Sequence
  oligonucleotide
cacatgggtcacagcaagg

SEQ ID NO: 444 - DNA, 21 bp, Artificial Sequence
  oligonucleotide
tcgctgcgtaaggctgcatga

SEQ ID NO: 445 - DNA, 19 bp, Artificial Sequence
  oligonucleotide
ccttggcagaggtactaga

SEQ ID NO: 446 - DNA, 19 bp, Artificial Sequence
  oligonucleotide
attgaagaagcggcagtag

SEQ ID NO: 447 - DNA, 21 bp, Artificial Sequence
  oligonucleotide
aggtgccttgctgtgacccat

SEQ ID NO: 448 - DNA, 18 bp, Artificial Sequence
  oligonucleotide
cctttcttggcccttgtt

SEQ ID NO: 449 - DNA, 19 bp, Artificial Sequence
  oligonucleotide
aaggttgctttgccatttg

SEQ ID NO: 450 - DNA, 24 bp, Artificial Sequence
  oligonucleotide
tgactctcggactgagaaacgcaa

SEQ ID NO: 451 - DNA, 19 bp, Artificial Sequence
  oligonucleotide
atcgcatcatcgcggtatt

SEQ ID NO: 452 - DNA, 22 bp, Artificial Sequence
  oligonucleotide
gagtcagagtcagacatttggg

SEQ ID NO: 453 - DNA, 22 bp, Artificial Sequence
  oligonucleotide
cgccctttcttggcccttgttc

SEQ ID NO: 454 - DNA, 20 bp, Artificial Sequence
  oligonucleotide
ggcctgttctatagcatctt

SEQ ID NO: 455 - DNA, 20 bp, Artificial Sequence
  oligonucleotide
gtgtacatgaagtccaggag

SEQ ID NO: 456 - DNA, 26 bp, Artificial Sequence
  oligonucleotide
tcctgagatcaaccctgagggattct

SEQ ID NO: 457 - DNA, 18 bp, Artificial Sequence
  oligonucleotide
gagccgtgagcagtttag

SEQ ID NO: 458 - DNA, 22 bp, Artificial Sequence
  oligonucleotide
gatcacactaaggttgcatttc

SEQ ID NO: 459 - DNA, 21 bp, Artificial Sequence
  oligonucleotide
aaacggtcctcatggcctgca

SEQ ID NO: 460 - DNA, 21 bp, Artificial Sequence
  oligonucleotide
gtcgagacatcttgactgatg

SEQ ID NO: 461 - DNA, 19 bp, Artificial Sequence
  oligonucleotide
gctatagaacaggccactg

SEQ ID NO: 462 - DNA, 24 bp, Artificial Sequence
  oligonucleotide
tcattgttgtgagccgtgagcagt

SEQ ID NO: 463 - DNA, 24 bp, Artificial Sequence
  oligonucleotide
gtgatgttcttctcaaccttaatc

SEQ ID NO: 464 - DNA, 20 bp, Artificial Sequence
  oligonucleotide
ttatgggctctaaactgctc

SEQ ID NO: 465 - DNA, 27 bp, Artificial Sequence
  oligonucleotide
actgatgttgtcattgttgtgagccgt

SEQ ID NO: 466 - DNA, 19 bp, Artificial Sequence
  oligonucleotide
atgagtctggacggagtag

SEQ ID NO: 467 - DNA, 21 bp, Artificial Sequence
  oligonucleotide
ctctccaatgctatgggtatc

SEQ ID NO: 468 - DNA, 22 bp, Artificial Sequence
  oligonucleotide
tcgctcgcagacaccacaagat

SEQ ID NO: 469 - DNA, 20 bp, Artificial Sequence
  oligonucleotide
cccatagcattggagagaaa

SEQ ID NO: 470 - DNA, 20 bp, Artificial Sequence
  oligonucleotide
acttgaccaatcccatatcc

SEQ ID NO: 471 - DNA, 25 bp, Artificial Sequence
  oligonucleotide
acagctcacagtctgaggaagatga

SEQ ID NO: 472 - DNA, 22 bp, Artificial Sequence
  oligonucleotide
cacaagataccaacagagcttc

SEQ ID NO: 473 - DNA, 24 bp, Artificial Sequence
  oligonucleotide
gctttcaacttctttccttctttc

SEQ ID NO: 474 - DNA, 25 bp, Artificial Sequence
  oligonucleotide
acagctcacagtctgaggaagatga

SEQ ID NO: 475 - DNA, 21 bp, Artificial Sequence
  oligonucleotide
agagcttctgaaacagatacc

SEQ ID NO: 476 - DNA, 22 bp, Artificial Sequence
  oligonucleotide
gctttcaacttctttccttctt

SEQ ID NO: 477 - DNA, 25 bp, Artificial Sequence
  oligonucleotide
acagctcacagtctgaggaagatga

SEQ ID NO: 478 - DNA, 21 bp, Artificial Sequence
  oligonucleotide
tccttcatctccaagtttctc

SEQ ID NO: 479 - DNA, 20 bp, Artificial Sequence
  oligonucleotide
tgggaaccagtgatgtttat

SEQ ID NO: 480 - DNA, 22 bp, Artificial Sequence
  oligonucleotide
agcgacagctgcagaccttcag

SEQ ID NO: 481 - DNA, 17 bp, Artificial Sequence
  oligonucleotide
cgacagctgcagacctt

SEQ ID NO: 482 - DNA, 19 bp, Artificial Sequence
  oligonucleotide
gttgatgcgaatacaacgg

SEQ ID NO: 483 - DNA, 23 bp, Artificial Sequence
  oligonucleotide
caggagctgctggcagaacatta

SEQ ID NO: 484 - DNA, 19 bp, Artificial Sequence
  oligonucleotide
acatcactggttcccagaa

SEQ ID NO: 485 - DNA, 19 bp, Artificial Sequence
  oligonucleotide
gcctgtccaatcagaggat

SEQ ID NO: 486 - DNA, 23 bp, Artificial Sequence
  oligonucleotide
acaacggtaacccgatcccttgc

SEQ ID NO: 487 - DNA, 20 bp, Artificial Sequence
  oligonucleotide
atcgggttaccgttgtattc

SEQ ID NO: 488 - DNA, 19 bp, Artificial Sequence
  oligonucleotide
ctgactgctcagtccaatc

SEQ ID NO: 489 - DNA, 23 bp, Artificial Sequence
  oligonucleotide
cctctgattggacaggcagcaca

SEQ ID NO: 490 - DNA, 19 bp, Artificial Sequence
  oligonucleotide
gagaagtgcggagattcaa

SEQ ID NO: 491 - DNA, 19 bp, Artificial Sequence
  oligonucleotide
ttctcacacagacgtttcc

SEQ ID NO: 492 - DNA, 22 bp, Artificial Sequence
  oligonucleotide
acgttactcaggtgcgggcatt

SEQ ID NO: 493 - DNA, 19 bp, Artificial Sequence
  oligonucleotide
gtgctgaatgaggaacaga

SEQ ID NO: 494 - DNA, 19 bp, Artificial Sequence
  oligonucleotide
agttcttgaccgctttctt

SEQ ID NO: 495 - DNA, 24 bp, Artificial Sequence
  oligonucleotide
acgtctgtgtgagaacattgccgg

SEQ ID NO: 496 - DNA, 19 bp, Artificial Sequence
  oligonucleotide
ctccggaacaacagccttc

SEQ ID NO: 497 - DNA, 23 bp, Artificial Sequence
  oligonucleotide
catcattggcagtgttgaatctc

SEQ ID NO: 498 - DNA, 23 bp, Artificial Sequence
  oligonucleotide
tattggatgctgtgctccagggc

SEQ ID NO: 499 - DNA, 21 bp, Artificial Sequence
  oligonucleotide
aacactgccaatgatgataac

SEQ ID NO: 500 - DNA, 19 bp, Artificial Sequence
  oligonucleotide
acagacgtttcctctgttc

SEQ ID NO: 501 - DNA, 23 bp, Artificial Sequence
  oligonucleotide
cgggcattctatgtgaacgtgct

SEQ ID NO: 502 - DNA, 18 bp, Artificial Sequence
  oligonucleotide
cgatgacgtggtcaagat

SEQ ID NO: 503 - DNA, 18 bp, Artificial Sequence
  oligonucleotide
cttccaaatgccgtcaaa

SEQ ID NO: 504 - DNA, 24 bp, Artificial Sequence
  oligonucleotide
ttgcagaaccagaagggacacaca

SEQ ID NO: 505 - DNA, 20 bp, Artificial Sequence
  oligonucleotide
ccagaagggacacacagttt

SEQ ID NO: 506 - DNA, 19 bp, Artificial Sequence
  oligonucleotide
aaagagggcagacagcaag

SEQ ID NO: 507 - DNA, 22 bp, Artificial Sequence
  oligonucleotide
aaggccagcttcaccaccttca

SEQ ID NO: 508 - DNA, 19 bp, Artificial Sequence
  oligonucleotide
gcacaccaaggagatcgac

SEQ ID NO: 509 - DNA, 20 bp, Artificial Sequence
  oligonucleotide
cccttctggttctgcaatca

SEQ ID NO: 510 - DNA, 23 bp, Artificial Sequence
  oligonucleotide
tggtcaaccgcgaccctaaacac

SEQ ID NO: 511 - DNA, 19 bp, Artificial Sequence
  oligonucleotide
cgatgacgtggtcaagatt

SEQ ID NO: 512 - DNA, 25 bp, Artificial Sequence
  oligonucleotide
aactgtgtgtcccttctggttctgc

SEQ ID NO: 513 - DNA, 19 bp, Artificial Sequence
  oligonucleotide
cacagtgaaggtggtgaag

SEQ ID NO: 514 - DNA, 20 bp, Artificial Sequence
  oligonucleotide
acaggttcttggctcttatg

SEQ ID NO: 515 - DNA, 22 bp, Artificial Sequence
  oligonucleotide
tgcagtcttcttcaactattct

SEQ ID NO: 516 - DNA, 30 bp, Artificial Sequence
  oligonucleotide
cctaaacatttgtcttgcattggagtctgt

SEQ ID NO: 517 - DNA, 19 bp, Artificial Sequence
  oligonucleotide
ttgctggctgctagaatag

SEQ ID NO: 518 - DNA, 22 bp, Artificial Sequence
  oligonucleotide
gtcagaagcagtacatttacac

SEQ ID NO: 519 - DNA, 26 bp, Artificial Sequence
  oligonucleotide
tccatccactcatgatgtgatccgga

SEQ ID NO: 520 - DNA, 20 bp, Artificial Sequence
  oligonucleotide
acaggttcttggctcttatg

SEQ ID NO: 521 - DNA, 22 bp, Artificial Sequence
  oligonucleotide
tgcagtcttcttcaactattct

SEQ ID NO: 522 - DNA, 25 bp, Artificial Sequence
  oligonucleotide
acatttgtcttgcattggagtctgt

SEQ ID NO: 523 - DNA, 19 bp, Artificial Sequence
  oligonucleotide
tggatcttgcactgaaact

SEQ ID NO: 524 - DNA, 21 bp, Artificial Sequence
  oligonucleotide
ctccaatgcaagacaaatgtt

SEQ ID NO: 525 - DNA, 28 bp, Artificial Sequence
  oligonucleotide
caccttcataagagccaagaacctgtcc

SEQ ID NO: 526 - DNA, 19 bp, Artificial Sequence
  oligonucleotide
agagccaacagaacagaag

SEQ ID NO: 527 - DNA, 19 bp, Artificial Sequence
  oligonucleotide
tcgagctgtttacgtttga

SEQ ID NO: 528 - DNA, 23 bp, Artificial Sequence
  oligonucleotide
aaatgccggttctgtggagcaga

SEQ ID NO: 529 - DNA, 22 bp, Artificial Sequence
  oligonucleotide
caaacgtaaacagctcgaatta

SEQ ID NO: 530 - DNA, 20 bp, Artificial Sequence
  oligonucleotide
tccatgaagtcagcgatatg

SEQ ID NO: 531 - DNA, 28 bp, Artificial Sequence
  oligonucleotide
acatcactgcttgatgaagcaaggaaga

SEQ ID NO: 532 - DNA, 19 bp, Artificial Sequence
  oligonucleotide
aagaagcctggcctcagaa

SEQ ID NO: 533 - DNA, 25 bp, Artificial Sequence
  oligonucleotide
tcatgtatatcttccttgcttcatc

SEQ ID NO: 534 - DNA, 30 bp, Artificial Sequence
  oligonucleotide
tcttaattcgagctgtttacgtttgacgtc

SEQ ID NO: 535 - DNA, 19 bp, Artificial Sequence
  oligonucleotide
cggttctgtggagcagacg

SEQ ID NO: 536 - DNA, 24 bp, Artificial Sequence
  oligonucleotide
cttcatcaagcagtgatgtatctg

SEQ ID NO: 537 - DNA, 23 bp, Artificial Sequence
  oligonucleotide
cctggcctcagaagacgtcaaac

SEQ ID NO: 538 - DNA, 18 bp, Artificial Sequence
  oligonucleotide
gctgctgtggaagaaact

SEQ ID NO: 539 - DNA, 21 bp, Artificial Sequence
  oligonucleotide
gccctttgtctgacagaatta

SEQ ID NO: 540 - DNA, 22 bp, Artificial Sequence
  oligonucleotide
tgccagtaccacttctccgagc

SEQ ID NO: 541 - DNA, 21 bp, Artificial Sequence
  oligonucleotide
atccgcaaacgattaattctg

SEQ ID NO: 542 - DNA, 20 bp, Artificial Sequence
  oligonucleotide
tccatactgctctttccttg

SEQ ID NO: 543 - DNA, 24 bp, Artificial Sequence
  oligonucleotide
acaaagggcagctggattggaaga

SEQ ID NO: 544 - DNA, 19 bp, Artificial Sequence
  oligonucleotide
aagaaactctgccagtacc

SEQ ID NO: 545 - DNA, 22 bp, Artificial Sequence
  oligonucleotide
acatcggacaagtttgaaatac

SEQ ID NO: 546 - DNA, 23 bp, Artificial Sequence
  oligonucleotide
agcggcagatccgcaaacgatta

SEQ ID NO: 547 - DNA, 19 bp, Artificial Sequence
  oligonucleotide
ctgctgtggaagaaactct

SEQ ID NO: 548 - DNA, 19 bp, Artificial Sequence
  oligonucleotide
cttgggtaacatcggacaa

SEQ ID NO: 549 - DNA, 23 bp, Artificial Sequence
  oligonucleotide
agcggcagatccgcaaacgatta

SEQ ID NO: 550 - DNA, 21 bp, Artificial Sequence
  oligonucleotide
gtgccctacttcaaggataag

SEQ ID NO: 551 - DNA, 19 bp, Artificial Sequence
  oligonucleotide
cttgccagttccctcattc

SEQ ID NO: 552 - DNA, 22 bp, Artificial Sequence
  oligonucleotide
aactccatccggcacaacctgt

SEQ ID NO: 553 - DNA, 19 bp, Artificial Sequence
  oligonucleotide
caacctgtcactgcatagt

SEQ ID NO: 554 - DNA, 19 bp, Artificial Sequence
  oligonucleotide
ttgatgatccaccaagagc

SEQ ID NO: 555 - DNA, 22 bp, Artificial Sequence
  oligonucleotide
tgcgggtccagaatgagggaac

SEQ ID NO: 556 - DNA, 19 bp, Artificial Sequence
  oligonucleotide
acaaacggctcactctgtc

SEQ ID NO: 557 - DNA, 19 bp, Artificial Sequence
  oligonucleotide
tgttgctgtcgcccttatc

SEQ ID NO: 558 - DNA, 22 bp, Artificial Sequence
  oligonucleotide
tacgagtggatggtgcgttgcg

SEQ ID NO: 559 - DNA, 21 bp, Artificial Sequence
  oligonucleotide
cgtgccctacttcaaggataa

SEQ ID NO: 560 - DNA, 18 bp, Artificial Sequence
  oligonucleotide
tcattctggacccgcatg

SEQ ID NO: 561 - DNA, 24 bp, Artificial Sequence
  oligonucleotide
cggctggaagaactccatccggca

SEQ ID NO: 562 - DNA, 19 bp, Artificial Sequence
  oligonucleotide
ggtgacgaatccacattca

SEQ ID NO: 563 - DNA, 19 bp, Artificial Sequence
  oligonucleotide
tcaccgttcagggagatta

SEQ ID NO: 564 - DNA, 22 bp, Artificial Sequence
  oligonucleotide
tgccgggaaagtcgctacatgg

SEQ ID NO: 565 - DNA, 22 bp, Artificial Sequence
  oligonucleotide
acgaatccacattcatctcaat

SEQ ID NO: 566 - DNA, 20 bp, Artificial Sequence
  oligonucleotide
gatccatgtagcgactttcc

SEQ ID NO: 567 - DNA, 29 bp, Artificial Sequence
  oligonucleotide
aaggatcctgccttaagtcaacttatttg

SEQ ID NO: 568 - DNA, 22 bp, Artificial Sequence
  oligonucleotide
cctgccttaagtcaacttattt

SEQ ID NO: 569 - DNA, 19 bp, Artificial Sequence
  oligonucleotide
tcattcagatgccatcacc

SEQ ID NO: 570 - DNA, 22 bp, Artificial Sequence
  oligonucleotide
tgccgggaaagtcgctacatgg

SEQ ID NO: 571 - DNA, 21 bp, Artificial Sequence
  oligonucleotide
cacattcatctcaatggaagg

SEQ ID NO: 572 - DNA, 21 bp, Artificial Sequence
  oligonucleotide
cagggagattaatcactggaa

SEQ ID NO: 573 - DNA, 22 bp, Artificial Sequence
  oligonucleotide
tgccgggaaagtcgctacatgg

SEQ ID NO: 574 - DNA, 19 bp, Artificial Sequence
  oligonucleotide
gacccagtatgccatcttt

SEQ ID NO: 575 - DNA, 20 bp, Artificial Sequence
  oligonucleotide
ggcatctgtctggacataaa

SEQ ID NO: 576 - DNA, 21 bp, Artificial Sequence
  oligonucleotide
tttcggatgaacgccggacct

SEQ ID NO: 577 - DNA, 20 bp, Artificial Sequence
  oligonucleotide
ctggatccaatctcagtgtc

SEQ ID NO: 578 - DNA, 19 bp, Artificial Sequence
  oligonucleotide
caggtagtgggtgatgttg

SEQ ID NO: 579 - DNA, 22 bp, Artificial Sequence
  oligonucleotide
aagtggaaaccaccctccgacc

SEQ ID NO: 580 - DNA, 22 bp, Artificial Sequence
  oligonucleotide
ggccaagagtgacatcatttat

SEQ ID NO: 581 - DNA, 23 bp, Artificial Sequence
  oligonucleotide
ggtggtttccacttcagaataat

SEQ ID NO: 582 - DNA, 17 bp, Artificial Sequence
  oligonucleotide
aacccctctgtgcccct

SEQ ID NO: 583 - DNA, 19 bp, Artificial Sequence
  oligonucleotide
ccatctttgtgaagaccct

SEQ ID NO: 584 - DNA, 22 bp, Artificial Sequence
  oligonucleotide
aatctgggatgatgagttagac

SEQ ID NO: 585 - DNA, 22 bp, Artificial Sequence
  oligonucleotide
tccagacagatgccaccaaccc

SEQ ID NO: 586 - DNA, 19 bp, Artificial Sequence
  oligonucleotide
catctgcgcctttgtttag

SEQ ID NO: 587 - DNA, 19 bp, Artificial Sequence
  oligonucleotide
gtgctttggctttgttgag

SEQ ID NO: 588 - DNA, 23 bp, Artificial Sequence
  oligonucleotide
cccggcacacaacacttggtttg

SEQ ID NO: 589 - DNA, 18 bp, Artificial Sequence
  oligonucleotide
gcacacaacacttggttt

SEQ ID NO: 590 - DNA, 21 bp, Artificial Sequence
  oligonucleotide
ctgttctcgttccaaattctc

SEQ ID NO: 591 - DNA, 30 bp, Artificial Sequence
  oligonucleotide
agcacacatcaagaaacttgaagaagctga

SEQ ID NO: 592 - DNA, 20 bp, Artificial Sequence
  oligonucleotide
ccaaagcacacatcaagaaa

SEQ ID NO: 593 - DNA, 20 bp, Artificial Sequence
  oligonucleotide
ttcgtattcgttccatctcc

SEQ ID NO: 594 - DNA, 22 bp, Artificial Sequence
  oligonucleotide
aaagccagcaccagctcgagaa

SEQ ID NO: 595 - DNA, 19 bp, Artificial Sequence
  oligonucleotide
tgattccactaggaccaga

SEQ ID NO: 596 - DNA, 22 bp, Artificial Sequence
  oligonucleotide
tctttcagcttcttcaagtttc

SEQ ID NO: 597 - DNA, 23 bp, Artificial Sequence
  oligonucleotide
cccggcacacaacacttggtttg

SEQ ID NO: 598 - DNA, 17 bp, Artificial Sequence
  oligonucleotide
agcggcttcagcagatc

SEQ ID NO: 599 - DNA, 17 bp, Artificial Sequence
  oligonucleotide
gcctggagcccagatac

SEQ ID NO: 600 - DNA, 22 bp, Artificial Sequence
  oligonucleotide
actagcagcatgttgagccggg

SEQ ID NO: 601 - DNA, 19 bp, Artificial Sequence
  oligonucleotide
gcactagcagcatgttgag

SEQ ID NO: 602 - DNA, 19 bp, Artificial Sequence
  oligonucleotide
cgttgatgtgaggttccag

SEQ ID NO: 603 - DNA, 20 bp, Artificial Sequence
  oligonucleotide
tccaggcagaagcacagcct

SEQ ID NO: 604 - DNA, 19 bp, Artificial Sequence
  oligonucleotide
cgacctgccctacgactac

SEQ ID NO: 605 - DNA, 21 bp, Artificial Sequence
  oligonucleotide
ggtgacgttcaggttgttcac

SEQ ID NO: 606 - DNA, 22 bp, Artificial Sequence
  oligonucleotide
tggaacctcacatcaacgcgca

SEQ ID NO: 607 - DNA, 19 bp, Artificial Sequence
  oligonucleotide
cctggaacctcacatcaac

SEQ ID NO: 608 - DNA, 19 bp, Artificial Sequence
  oligonucleotide
cctcctggtacttctcctc

SEQ ID NO: 609 - DNA, 22 bp, Artificial Sequence
  oligonucleotide
tcaggttgttcacgtaggccgc

SEQ ID NO: 610 - DNA, 19 bp, Artificial Sequence
  oligonucleotide
ctctctgtgtggctgtaac

SEQ ID NO: 611 - DNA, 20 bp, Artificial Sequence
  oligonucleotide
gggctgttcatactctcttc

SEQ ID NO: 612 - DNA, 23 bp, Artificial Sequence
  oligonucleotide
accaacgagctgaagcagatgca

SEQ ID NO: 613 - DNA, 21 bp, Artificial Sequence
  oligonucleotide
ggctgtaacttacgtgtactt

SEQ ID NO: 614 - DNA, 20 bp, Artificial Sequence
  oligonucleotide
gggtcccaataactgtcatc

SEQ ID NO: 615 - DNA, 23 bp, Artificial Sequence
  oligonucleotide
accaacgagctgaagcagatgca

SEQ ID NO: 616 - DNA, 18 bp, Artificial Sequence
  oligonucleotide
cctgcagtctctctgtgt

SEQ ID NO: 617 - DNA, 20 bp, Artificial Sequence
  oligonucleotide
gccacttttggagtacttgt

SEQ ID NO: 618 - DNA, 24 bp, Artificial Sequence
  oligonucleotide
taccaacgagctgaagcagatgca

SEQ ID NO: 619 - DNA, 19 bp, Artificial Sequence
  oligonucleotide
gctgatcgtgatcttcaca

SEQ ID NO: 620 - DNA, 20 bp, Artificial Sequence
  oligonucleotide
gagtacttgtcctgcatctg

SEQ ID NO: 621 - DNA, 22 bp, Artificial Sequence
  oligonucleotide
tgcagtctctctgtgtggctgt

SEQ ID NO: 622 - DNA, 22 bp, Artificial Sequence
  oligonucleotide
tccagtacaactttacctacaa

SEQ ID NO: 623 - DNA, 19 bp, Artificial Sequence
  oligonucleotide
agcacatctgcaggataat

SEQ ID NO: 624 - DNA, 22 bp, Artificial Sequence
  oligonucleotide
caggcatcagaccagcttgcca

SEQ ID NO: 625 - DNA, 19 bp, Artificial Sequence
  oligonucleotide
tgctacatggagcttgttc

SEQ ID NO: 626 - DNA, 19 bp, Artificial Sequence
  oligonucleotide
cgaaggagaaaggcaatga

SEQ ID NO: 627 - DNA, 23 bp, Artificial Sequence
  oligonucleotide
ccaactgcagagatggcagctga

SEQ ID NO: 628 - DNA, 21 bp, Artificial Sequence
  oligonucleotide
ggctgtattccagtacaactt

SEQ ID NO: 629 - DNA, 20 bp, Artificial Sequence
  oligonucleotide
cagttggatcagtctggaaa

SEQ ID NO: 630 - DNA, 22 bp, Artificial Sequence
  oligonucleotide
caggcatcagaccagcttgcca

SEQ ID NO: 631 - DNA, 19 bp, Artificial Sequence
  oligonucleotide
caccaaagcagccataaac

SEQ ID NO: 632 - DNA, 21 bp, Artificial Sequence
  oligonucleotide
ctccatgtagcaaacagtcta

SEQ ID NO: 633 - DNA, 22 bp, Artificial Sequence
  oligonucleotide
caggcatcagaccagcttgcca

SEQ ID NO: 634 - DNA, 20 bp, Artificial Sequence
  oligonucleotide
cctcggtgtcctacttcaaa

SEQ ID NO: 635 - DNA, 19 bp, Artificial Sequence
  oligonucleotide
acttctgttcctcgcagac

SEQ ID NO: 636 - DNA, 22 bp, Artificial Sequence
  oligonucleotide
aagatcgtcgccacctggatgc

SEQ ID NO: 637 - DNA, 22 bp, Artificial Sequence
  oligonucleotide
cttcaaatgtgtgcagaaggag

SEQ ID NO: 638 - DNA, 19 bp, Artificial Sequence
  oligonucleotide
gaagcggtccaggtagttc

SEQ ID NO: 639 - DNA, 22 bp, Artificial Sequence
  oligonucleotide
tgcgaggaacagaagtgcgagg

SEQ ID NO: 640 - DNA, 18 bp, Artificial Sequence
  oligonucleotide
catgcggaagatcgtcgc

SEQ ID NO: 641 - DNA, 17 bp, Artificial Sequence
  oligonucleotide
gacctcctcctcgcact

SEQ ID NO: 642 - DNA, 25 bp, Artificial Sequence
  oligonucleotide
ctggatgctggaggtctgcgaggaa

SEQ ID NO: 643 - DNA, 22 bp, Artificial Sequence
  oligonucleotide
gtgtcctacttcaaatgtgtgc

SEQ ID NO: 644 - DNA, 19 bp, Artificial Sequence
  oligonucleotide
cctcctcgcacttctgttc

SEQ ID NO: 645 - DNA, 22 bp, Artificial Sequence
  oligonucleotide
aagatcgtcgccacctggatgc

SEQ ID NO: 646 - DNA, 20 bp, Artificial Sequence
  oligonucleotide
taccagatggatgtgaaccc

SEQ ID NO: 647 - DNA, 20 bp, Artificial Sequence
  oligonucleotide
ccgtgatctgtcaccacata

SEQ ID NO: 648 - DNA, 22 bp, Artificial Sequence
  oligonucleotide
tgccacctgcgtgaagaagtgt

SEQ ID NO: 649 - DNA, 22 bp, Artificial Sequence
  oligonucleotide
gatgcattccttgtaaccaata

SEQ ID NO: 650 - DNA, 22 bp, Artificial Sequence
  oligonucleotide
atttcttcaccaataccctttg

SEQ ID NO: 651 - DNA, 23 bp, Artificial Sequence
  oligonucleotide
tcctgctttcaaacgggtctgga

SEQ ID NO: 652 - DNA, 21 bp, Artificial Sequence
  oligonucleotide
gagtgaaatcaggaacatctc

SEQ ID NO: 653 - DNA, 20 bp, Artificial Sequence
  oligonucleotide
cttgttgattctcagacgtg

SEQ ID NO: 654 - DNA, 25 bp, Artificial Sequence
  oligonucleotide
atcgacaagaaggcacctgactttg

SEQ ID NO: 655 - DNA, 21 bp, Artificial Sequence
  oligonucleotide
cagtcaattgcccatcaaaca

SEQ ID NO: 656 - DNA, 22 bp, Artificial Sequence
  oligonucleotide
gtgagctgttgcaaataatctt

SEQ ID NO: 657 - DNA, 24 bp, Artificial Sequence
  oligonucleotide
cacagccaccaaccacactaacga

SEQ ID NO: 658 - DNA, 23 bp, Artificial Sequence
  oligonucleotide
gtgtgacagtaagctaacctttg

SEQ ID NO: 659 - DNA, 19 bp, Artificial Sequence
  oligonucleotide
gctcaacttccgggtagaa

SEQ ID NO: 660 - DNA, 28 bp, Artificial Sequence
  oligonucleotide
agacagattctacatgcgcacaaatccc

SEQ ID NO: 661 - DNA, 20 bp, Artificial Sequence
  oligonucleotide
cgatgcagccatttctgata

SEQ ID NO: 662 - DNA, 22 bp, Artificial Sequence
  oligonucleotide
ggaaagtcttcagctatttgct

SEQ ID NO: 663 - DNA, 30 bp, Artificial Sequence
  oligonucleotide
ctggagatttgatgagaagagaaattccat

SEQ ID NO: 664 - DNA, 19 bp, Artificial Sequence
  oligonucleotide
catccacaccctaggtttc

SEQ ID NO: 665 - DNA, 22 bp, Artificial Sequence
  oligonucleotide
ctccagtatttgtcctctacaa

SEQ ID NO: 666 - DNA, 25 bp, Artificial Sequence
  oligonucleotide
tcgatgcagccatttctgataagga

SEQ ID NO: 667 - DNA, 19 bp, Artificial Sequence
  oligonucleotide
ccctaggtttccctccaac

SEQ ID NO: 668 - DNA, 22 bp, Artificial Sequence
  oligonucleotide
gctccatggaatttctcttctc

SEQ ID NO: 669 - DNA, 25 bp, Artificial Sequence
  oligonucleotide
tcgatgcagccatttctgataagga

SEQ ID NO: 670 - DNA, 22 bp, Artificial Sequence
  oligonucleotide
gtagaggacaaatactggagat

SEQ ID NO: 671 - DNA, 19 bp, Artificial Sequence
  oligonucleotide
gagtcaatccctggaaagt

SEQ ID NO: 672 - DNA, 22 bp, Artificial Sequence
  oligonucleotide
ccatggagccaggctttcccaa

SEQ ID NO: 673 - DNA, 20 bp, Artificial Sequence
  oligonucleotide
tggagacctgagaaccaatc

SEQ ID NO: 674 - DNA, 19 bp, Artificial Sequence
  oligonucleotide
acccgagtgtaaccatagc

SEQ ID NO: 675 - DNA, 23 bp, Artificial Sequence
  oligonucleotide
aggcagctggcagaggaatacct

SEQ ID NO: 676 - DNA, 19 bp, Artificial Sequence
  oligonucleotide
ggagacctgagaaccaatc

SEQ ID NO: 677 - DNA, 19 bp, Artificial Sequence
  oligonucleotide
gactctccacgcatctctg

SEQ ID NO: 678 - DNA, 23 bp, Artificial Sequence
  oligonucleotide
aggcagctggcagaggaatacct

SEQ ID NO: 679 - DNA, 20 bp, Artificial Sequence
  oligonucleotide
agaaccaatctcaccgacag

SEQ ID NO: 680 - DNA, 21 bp, Artificial Sequence
  oligonucleotide
ccagagatttcgactctccac

SEQ ID NO: 681 - DNA, 23 bp, Artificial Sequence
  oligonucleotide
tggttacactcgggtggcagaga

SEQ ID NO: 682 - DNA, 17 bp, Artificial Sequence
  oligonucleotide
tccacccttgtgctctt

SEQ ID NO: 683 - DNA, 18 bp, Artificial Sequence
  oligonucleotide
actctccacgcatctctg

SEQ ID NO: 684 - DNA, 23 bp, Artificial Sequence
  oligonucleotide
aaccaatctcaccgacaggcagc

SEQ ID NO: 685 - DNA, 19 bp, Artificial Sequence
  oligonucleotide
tgtgtgggttagacttgtg

SEQ ID NO: 686 - DNA, 19 bp, Artificial Sequence
  oligonucleotide
gtaacggcttcgggaatag

SEQ ID NO: 687 - DNA, 20 bp, Artificial Sequence
  oligonucleotide
aaccagggcaactctggccg

SEQ ID NO: 688 - DNA, 19 bp, Artificial Sequence
  oligonucleotide
tgtgtgggttagacttgtg

SEQ ID NO: 689 - DNA, 19 bp, Artificial Sequence
  oligonucleotide
cacagctcatgtctgatga

SEQ ID NO: 690 - DNA, 23 bp, Artificial Sequence
  oligonucleotide
ccgaagccgttacctcgaatgca

SEQ ID NO: 691 - DNA, 19 bp, Artificial Sequence
  oligonucleotide
ctcagagaagaccaacagg

SEQ ID NO: 692 - DNA, 19 bp, Artificial Sequence
  oligonucleotide
cttcgggaataggtgacag

SEQ ID NO: 693 - DNA, 23 bp, Artificial Sequence
  oligonucleotide
acttgtgcaaccagggcaactct

SEQ ID NO: 694 - DNA, 19 bp, Artificial Sequence
  oligonucleotide
gaggttgtgtgtgggttag

SEQ ID NO: 695 - DNA, 19 bp, Artificial Sequence
  oligonucleotide
tgatgagccacaggaaatg

SEQ ID NO: 696 - DNA, 23 bp, Artificial Sequence
  oligonucleotide
acttgtgcaaccagggcaactct

SEQ ID NO: 697 - DNA, 20 bp, Artificial Sequence
  oligonucleotide
ttgacagtccaccaacttac

SEQ ID NO: 698 - DNA, 21 bp, Artificial Sequence
  oligonucleotide
ggaggaagggctctagtataa

SEQ ID NO: 699 - DNA, 26 bp, Artificial Sequence
  oligonucleotide
aagctgggaagccttctctaacctct

SEQ ID NO: 700 - DNA, 22 bp, Artificial Sequence
  oligonucleotide
gtgaataacattcccttccttc

SEQ ID NO: 701 - DNA, 21 bp, Artificial Sequence
  oligonucleotide
tagccatagtcagcattgtaa

SEQ ID NO: 702 - DNA, 29 bp, Artificial Sequence
  oligonucleotide
ccagatcacatttgattgacagtccacca

SEQ ID NO: 703 - DNA, 20 bp, Artificial Sequence
  oligonucleotide
tgtgttgacatccagatcac

SEQ ID NO: 704 - DNA, 20 bp, Artificial Sequence
  oligonucleotide
taggagaggttagagaaggc

SEQ ID NO: 705 - DNA, 28 bp, Artificial Sequence
  oligonucleotide
ccaccaacttacaatgctgactatggct

SEQ ID NO: 706 - DNA, 22 bp, Artificial Sequence
  oligonucleotide
ccaacttacaatgctgactatg

SEQ ID NO: 707 - DNA, 19 bp, Artificial Sequence
  oligonucleotide
caatcatcaggcacaggag

SEQ ID NO: 708 - DNA, 26 bp, Artificial Sequence
  oligonucleotide
aagctgggaagccttctctaacctct

SEQ ID NO: 709 - DNA, 18 bp, Artificial Sequence
  oligonucleotide
tgtctctgtgcccatgat

SEQ ID NO: 710 - DNA, 20 bp, Artificial Sequence
  oligonucleotide
cagttccaggatgtcgtagt

SEQ ID NO: 711 - DNA, 22 bp, Artificial Sequence
  oligonucleotide
actgagttcaccacgcccgatg

SEQ ID NO: 712 - DNA, 19 bp, Artificial Sequence
  oligonucleotide
cgcctcttccacaaatcag

SEQ ID NO: 713 - DNA, 21 bp, Artificial Sequence
  oligonucleotide
tccaggatgtcgtagtaatgg

SEQ ID NO: 714 - DNA, 22 bp, Artificial Sequence
  oligonucleotide
atgggcacagagacagtgctgc

SEQ ID NO: 715 - DNA, 18 bp, Artificial Sequence
  oligonucleotide
tggctcagaccaacaagt

SEQ ID NO: 716 - DNA, 20 bp, Artificial Sequence
  oligonucleotide
cagcaatgaacatgctgagg

SEQ ID NO: 717 - DNA, 24 bp, Artificial Sequence
  oligonucleotide
actacgacatcctggaactgccct

SEQ ID NO: 718 - DNA, 20 bp, Artificial Sequence
  oligonucleotide
ccacaaatcagacggcagca

SEQ ID NO: 719 - DNA, 20 bp, Artificial Sequence
  oligonucleotide
gtcgtagtaatggccatcgg

SEQ ID NO: 720 - DNA, 25 bp, Artificial Sequence
  oligonucleotide
cccatgatggctcagaccaacaagt

SEQ ID NO: 721 - DNA, 19 bp, Artificial Sequence
  oligonucleotide
ccagagagacaccagagaa

SEQ ID NO: 722 - DNA, 19 bp, Artificial Sequence
  oligonucleotide
gaggtcggaattgcagaag

SEQ ID NO: 723 - DNA, 24 bp, Artificial Sequence
  oligonucleotide
tctggcatcctgttgttgctgtgg

SEQ ID NO: 724 - DNA, 20 bp, Artificial Sequence
  oligonucleotide
cccagagagacaccagagaa

SEQ ID NO: 725 - DNA, 19 bp, Artificial Sequence
  oligonucleotide
gctatcagccacagcaaca

SEQ ID NO: 726 - DNA, 20 bp, Artificial Sequence
  oligonucleotide
cctggcttctggcatcctgt

SEQ ID NO: 727 - DNA, 19 bp, Artificial Sequence
  oligonucleotide
agagacaccagagaaccca

SEQ ID NO: 728 - DNA, 17 bp, Artificial Sequence
  oligonucleotide
gtgggacacaggtgcag

SEQ ID NO: 729 - DNA, 24 bp, Artificial Sequence
  oligonucleotide
tctggcatcctgttgttgctgtgg

SEQ ID NO: 730 - DNA, 17 bp, Artificial Sequence
  oligonucleotide
catcgccgcagatccag

SEQ ID NO: 731 - DNA, 18 bp, Artificial Sequence
  oligonucleotide
tcagccacagcaacaaca

SEQ ID NO: 732 - DNA, 24 bp, Artificial Sequence
  oligonucleotide
cccagagagacaccagagaaccca

SEQ ID NO: 733 - DNA, 22 bp, Artificial Sequence
  oligonucleotide
aaactcatgttcaagacagaag

SEQ ID NO: 734 - DNA, 19 bp, Artificial Sequence
  oligonucleotide
caagcaagggttcaaagac

SEQ ID NO: 735 - DNA, 26 bp, Artificial Sequence
  oligonucleotide
tggagaatgtcagtctgagtcaggcc

SEQ ID NO: 736 - DNA, 19 bp, Artificial Sequence
  oligonucleotide
gtctacctcccgccataaa

SEQ ID NO: 737 - DNA, 21 bp, Artificial Sequence
  oligonucleotide
gggaacaagaagtggagaatg

SEQ ID NO: 738 - DNA, 26 bp, Artificial Sequence
  oligonucleotide
tgttcaagacagaagggcctgactca

SEQ ID NO: 739 - DNA, 19 bp, Artificial Sequence
  oligonucleotide
caagacagaagggcctgac

SEQ ID NO: 740 - DNA, 20 bp, Artificial Sequence
  oligonucleotide
cacacctattgcaagcaagg

SEQ ID NO: 741 - DNA, 30 bp, Artificial Sequence
  oligonucleotide
tcagactgacattctccacttcttgttccc

SEQ ID NO: 742 - DNA, 19 bp, Artificial Sequence
  oligonucleotide
cagccacctgaagtccaaa

SEQ ID NO: 743 - DNA, 22 bp, Artificial Sequence
  oligonucleotide
gtggagaatgtcagtctgagtc

SEQ ID NO: 744 - DNA, 24 bp, Artificial Sequence
  oligonucleotide
ttatggcgggaggtagactgaccc

SEQ ID NO: 745 - DNA, 21 bp, Artificial Sequence
  oligonucleotide
gccagaagcacaagctatttc

SEQ ID NO: 746 - DNA, 19 bp, Artificial Sequence
  oligonucleotide
tgctttgcacatgctgttt

SEQ ID NO: 747 - DNA, 22 bp, Artificial Sequence
  oligonucleotide
accagaccatgtgcaagtggca

SEQ ID NO: 748 - DNA, 20 bp, Artificial Sequence
  oligonucleotide
cctttcataccagaccatgt

SEQ ID NO: 749 - DNA, 21 bp, Artificial Sequence
  oligonucleotide
cgctgaatcaaggattctttc

SEQ ID NO: 750 - DNA, 22 bp, Artificial Sequence
  oligonucleotide
tgcaaagcattgccgctttcca

SEQ ID NO: 751 - DNA, 20 bp, Artificial Sequence
  oligonucleotide
ctatttcacccagacacctg

SEQ ID NO: 752 - DNA, 19 bp, Artificial Sequence
  oligonucleotide
aaggattctttcggctgtg

SEQ ID NO: 753 - DNA, 22 bp, Artificial Sequence
  oligonucleotide
accagaccatgtgcaagtggca

SEQ ID NO: 754 - DNA, 21 bp, Artificial Sequence
  oligonucleotide
tttgagtgcaaagaaagtctg

SEQ ID NO: 755 - DNA, 20 bp, Artificial Sequence
  oligonucleotide
acatggtctggtatgaaagg

SEQ ID NO: 756 - DNA, 22 bp, Artificial Sequence
  oligonucleotide
agacctgctgccagaagcacaa

SEQ ID NO: 757 - DNA, 19 bp, Artificial Sequence
  oligonucleotide
gacaggatgttgacaatgc

SEQ ID NO: 758 - DNA, 19 bp, Artificial Sequence
  oligonucleotide
cctcttcgtggagtttctt

SEQ ID NO: 759 - DNA, 23 bp, Artificial Sequence
  oligonucleotide
tgcgttcaaggtcaagacgtgcc

SEQ ID NO: 760 - DNA, 22 bp, Artificial Sequence
  oligonucleotide
caatctttcagacaggatgttg

SEQ ID NO: 761 - DNA, 19 bp, Artificial Sequence
  oligonucleotide
gctcctggatttcctcttc

SEQ ID NO: 762 - DNA, 23 bp, Artificial Sequence
  oligonucleotide
acaatgcgtctctggcacgtctt

SEQ ID NO: 763 - DNA, 19 bp, Artificial Sequence
  oligonucleotide
cctccgggagaaattgcag

SEQ ID NO: 764 - DNA, 19 bp, Artificial Sequence
  oligonucleotide
gtcaagacgtgccagagac

SEQ ID NO: 765 - DNA, 26 bp, Artificial Sequence
  oligonucleotide
agatgcttcagagagaggaagccgaa

SEQ ID NO: 766 - DNA, 19 bp, Artificial Sequence
  oligonucleotide
ggaggagatgcttcagaga

SEQ ID NO: 767 - DNA, 19 bp, Artificial Sequence
  oligonucleotide
attccactttgcgttcaag

SEQ ID NO: 768 - DNA, 27 bp, Artificial Sequence
  oligonucleotide
tctttcagacaggatgttgacaatgcg

SEQ ID NO: 769 - DNA, 22 bp, Artificial Sequence
  oligonucleotide
tgtaacacctacaccattatct

SEQ ID NO: 770 - DNA, 21 bp, Artificial Sequence
  oligonucleotide
tcgcaatgaaacaatcagtag

SEQ ID NO: 771 - DNA, 24 bp, Artificial Sequence
  oligonucleotide
accattacagggagctgggacact

SEQ ID NO: 772 - DNA, 20 bp, Artificial Sequence
  oligonucleotide
actccagaccagtttatgac

SEQ ID NO: 773 - DNA, 19 bp, Artificial Sequence
  oligonucleotide
ccaacggttgtttctttcc

SEQ ID NO: 774 - DNA, 24 bp, Artificial Sequence
  oligonucleotide
agctgatgagacaaggaacctgca

SEQ ID NO: 775 - DNA, 19 bp, Artificial Sequence
  oligonucleotide
gcatttggtgaacaaggag

SEQ ID NO: 776 - DNA, 21 bp, Artificial Sequence
  oligonucleotide
ccaatcttcatgtccacattc

SEQ ID NO: 777 - DNA, 24 bp, Artificial Sequence
  oligonucleotide
agctgatgagacaaggaacctgca

SEQ ID NO: 778 - DNA, 19 bp, Artificial Sequence
  oligonucleotide
tgacagctgatgagacaag

SEQ ID NO: 779 - DNA, 20 bp, Artificial Sequence
  oligonucleotide
agctccctgtaatggttatg

SEQ ID NO: 780 - DNA, 28 bp, Artificial Sequence
  oligonucleotide
cctgcagaatgtggacatgaagattggg

SEQ ID NO: 781 - DNA, 22 bp, Artificial Sequence
  oligonucleotide
tcactgagttcatggaaaactg

SEQ ID NO: 782 - DNA, 21 bp, Artificial Sequence
  oligonucleotide
gttcatctcggacaggtactt

SEQ ID NO: 783 - DNA, 23 bp, Artificial Sequence
  oligonucleotide
ccttcctccggctcaacgatggg

SEQ ID NO: 784 - DNA, 19 bp, Artificial Sequence
  oligonucleotide
ctgttcgccactatgagaa

SEQ ID NO: 785 - DNA, 20 bp, Artificial Sequence
  oligonucleotide
ggatagcactgagacttgag

SEQ ID NO: 786 - DNA, 24 bp, Artificial Sequence
  oligonucleotide
aaactgaagagctcttccaagccc

SEQ ID NO: 787 - DNA, 22 bp, Artificial Sequence
  oligonucleotide
tgtaagagaaattaaggatggc

SEQ ID NO: 788 - DNA, 20 bp, Artificial Sequence
  oligonucleotide
ctgagacttgaggaaacaga

SEQ ID NO: 789 - DNA, 23 bp, Artificial Sequence
  oligonucleotide
tgatgtggttgctgttcgccact

SEQ ID NO: 790 - DNA, 21 bp, Artificial Sequence
  oligonucleotide
tttctgtttcctcaagtctca

SEQ ID NO: 791 - DNA, 22 bp, Artificial Sequence
  oligonucleotide
acaccaaggataaccttctaat

SEQ ID NO: 792 - DNA, 29 bp, Artificial Sequence
  oligonucleotide
tgatcagccatgttgtaataggatagcac

SEQ ID NO: 793 - DNA, 20 bp, Artificial Sequence
  oligonucleotide
gccactatgagaaggcataa

SEQ ID NO: 794 - DNA, 19 bp, Artificial Sequence
  oligonucleotide
cctccacaccaaggataac

SEQ ID NO: 795 - DNA, 29 bp, Artificial Sequence
  oligonucleotide
tctgtttcctcaagtctcagtgctatcct

SEQ ID NO: 796 - DNA, 19 bp, Artificial Sequence
  oligonucleotide
gtctacctctctccttggt

SEQ ID NO: 797 - DNA, 21 bp, Artificial Sequence
  oligonucleotide
caagtgctgagggtttattat

SEQ ID NO: 798 - DNA, 24 bp, Artificial Sequence
  oligonucleotide
tggaacagcgctactgatcaccaa

SEQ ID NO: 799 - DNA, 16 bp, Artificial Sequence
  oligonucleotide
agagctcccgccactc

SEQ ID NO: 800 - DNA, 20 bp, Artificial Sequence
  oligonucleotide
aggtgtctcacggtcatctc

SEQ ID NO: 801 - DNA, 24 bp, Artificial Sequence
  oligonucleotide
aggatgtccgccttctccagcttc

SEQ ID NO: 802 - DNA, 19 bp, Artificial Sequence
  oligonucleotide
tgggtgcctccactatgat

SEQ ID NO: 803 - DNA, 20 bp, Artificial Sequence
  oligonucleotide
cttccacgtgactgagagtt

SEQ ID NO: 804 - DNA, 22 bp, Artificial Sequence
  oligonucleotide
tctgtgtgggtggatgcgtgtg

SEQ ID NO: 805 - DNA, 21 bp, Artificial Sequence
  oligonucleotide
gcctccactatgatccttaaa

SEQ ID NO: 806 - DNA, 21 bp, Artificial Sequence
  oligonucleotide
cgtgactgagagttcaatttc

SEQ ID NO: 807 - DNA, 24 bp, Artificial Sequence
  oligonucleotide
atgcgtgtgggcacgactttgtac

SEQ ID NO: 808 - DNA, 19 bp, Artificial Sequence
  oligonucleotide
tttgagaagcagggatctg

SEQ ID NO: 809 - DNA, 21 bp, Artificial Sequence
  oligonucleotide
ccaaactccgatagtccatag

SEQ ID NO: 810 - DNA, 22 bp, Artificial Sequence
  oligonucleotide
ttactttgacgcgcacgccctt

SEQ ID NO: 811 - DNA, 19 bp, Artificial Sequence
  oligonucleotide
gaccgtggatcacctgaaa

SEQ ID NO: 812 - DNA, 21 bp, Artificial Sequence
  oligonucleotide
ccaaactccgatagtccatag

SEQ ID NO: 813 - DNA, 22 bp, Artificial Sequence
  oligonucleotide
ttactttgacgcgcacgccctt

SEQ ID NO: 814 - DNA, 19 bp, Artificial Sequence
  oligonucleotide
gcccttgctatggactatc

SEQ ID NO: 815 - DNA, 21 bp, Artificial Sequence
  oligonucleotide
tctagtccttcaatgatgctc

SEQ ID NO: 816 - DNA, 24 bp, Artificial Sequence
  oligonucleotide
cctggcagaagttgcgcgttatct

SEQ ID NO: 817 - DNA, 19 bp, Artificial Sequence
  oligonucleotide
caggagggaaaggttactt

SEQ ID NO: 818 - DNA, 22 bp, Artificial Sequence
  oligonucleotide
ccttcaatgatgctcagataac

SEQ ID NO: 819 - DNA, 24 bp, Artificial Sequence
  oligonucleotide
acgcccttgctatggactatcgga

SEQ ID NO: 820 - DNA, 22 bp, Artificial Sequence
  oligonucleotide
atgacagtggatcatttgaaga

SEQ ID NO: 821 - DNA, 20 bp, Artificial Sequence
  oligonucleotide
cggaatcctatgctcatgaa

SEQ ID NO: 822 - DNA, 24 bp, Artificial Sequence
  oligonucleotide
actttgacgcacacgctcttgcca

SEQ ID NO: 823 - DNA, 19 bp, Artificial Sequence
  oligonucleotide
aagatgcttcaggcaacag

SEQ ID NO: 824 - DNA, 21 bp, Artificial Sequence
  oligonucleotide
caacttctgttaggcactctc

SEQ ID NO: 825 - DNA, 23 bp, Artificial Sequence
  oligonucleotide
aaggctactttgacgcacacgct

SEQ ID NO: 826 - DNA, 19 bp, Artificial Sequence
  oligonucleotide
gacttgtgccaactgcttt

SEQ ID NO: 827 - DNA, 20 bp, Artificial Sequence
  oligonucleotide
gcgtcaaagtagcctttacc

SEQ ID NO: 828 - DNA, 28 bp, Artificial Sequence
  oligonucleotide
tcatttgaagatgcttcaggcaacaggg

SEQ ID NO: 829 - DNA, 21 bp, Artificial Sequence
  oligonucleotide
acaaggatctgcaaagttaga

SEQ ID NO: 830 - DNA, 19 bp, Artificial Sequence
  oligonucleotide
atgaagtccatggcaagag

SEQ ID NO: 831 - DNA, 26 bp, Artificial Sequence
  oligonucleotide
cgtgtgcgtcaaagtagcctttaccc

SEQ ID NO: 832 - DNA, 18 bp, Artificial Sequence
  oligonucleotide
tgcttagacgctggattt

SEQ ID NO: 833 - DNA, 19 bp, Artificial Sequence
  oligonucleotide
tcgtagtcgaggtcatagt

SEQ ID NO: 834 - DNA, 26 bp, Artificial Sequence
  oligonucleotide
ccctcaacgttagcttcaccaacagg

SEQ ID NO: 835 - DNA, 18 bp, Artificial Sequence
  oligonucleotide
tctctgaaaggctctcct

SEQ ID NO: 836 - DNA, 19 bp, Artificial Sequence
  oligonucleotide
tcctgttggtgaagctaac

SEQ ID NO: 837 - DNA, 24 bp, Artificial Sequence
  oligonucleotide
tgcagctgcttagacgctggattt

SEQ ID NO: 838 - DNA, 17 bp, Artificial Sequence
  oligonucleotide
gacccgcttctctgaaa

SEQ ID NO: 839 - DNA, 19 bp, Artificial Sequence
  oligonucleotide
aggtcatagttcctgttgg

SEQ ID NO: 840 - DNA, 24 bp, Artificial Sequence
  oligonucleotide
tgcagctgcttagacgctggattt

SEQ ID NO: 841 - DNA, 19 bp, Artificial Sequence
  oligonucleotide
ttcgggtagtggaaaacca

SEQ ID NO: 842 - DNA, 21 bp, Artificial Sequence
  oligonucleotide
catagttcctgttggtgaagc

SEQ ID NO: 843 - DNA, 22 bp, Artificial Sequence
  oligonucleotide
ctcccgcgacgatgcccctcaa

SEQ ID NO: 844 - DNA, 19 bp, Artificial Sequence
  oligonucleotide
ctgttcgccactatgagaa

SEQ ID NO: 845 - DNA, 20 bp, Artificial Sequence
  oligonucleotide
ggatagcactgagacttgag

SEQ ID NO: 846 - DNA, 24 bp, Artificial Sequence
  oligonucleotide
aaactgaagagctcttccaagccc

SEQ ID NO: 847 - DNA, 22 bp, Artificial Sequence
  oligonucleotide
tgtaagagaaattaaggatggc

SEQ ID NO: 848 - DNA, 20 bp, Artificial Sequence
  oligonucleotide
ctgagacttgaggaaacaga

SEQ ID NO: 849 - DNA, 23 bp, Artificial Sequence
  oligonucleotide
tgatgtggttgctgttcgccact

SEQ ID NO: 850 - DNA, 21 bp, Artificial Sequence
  oligonucleotide
tttctgtttcctcaagtctca

SEQ ID NO: 851 - DNA, 22 bp, Artificial Sequence
  oligonucleotide
acaccaaggataaccttctaat

SEQ ID NO: 852 - DNA, 29 bp, Artificial Sequence
  oligonucleotide
tgatcagccatgttgtaataggatagcac

SEQ ID NO: 853 - DNA, 20 bp, Artificial Sequence
  oligonucleotide
gccactatgagaaggcataa

SEQ ID NO: 854 - DNA, 19 bp, Artificial Sequence
  oligonucleotide
cctccacaccaaggataac

SEQ ID NO: 855 - DNA, 29 bp, Artificial Sequence
  oligonucleotide
tctgtttcctcaagtctcagtgctatcct

SEQ ID NO: 856 - DNA, 22 bp, Artificial Sequence
  oligonucleotide
gttgctggtgttctaaactatt

SEQ ID NO: 857 - DNA, 19 bp, Artificial Sequence
  oligonucleotide
cccataaccacattgacca

SEQ ID NO: 858 - DNA, 22 bp, Artificial Sequence
  oligonucleotide
tttgtgggtggagtttgtgcgc

SEQ ID NO: 859 - DNA, 19 bp, Artificial Sequence
  oligonucleotide
tgtgtgtacatttgtgggt

SEQ ID NO: 860 - DNA, 22 bp, Artificial Sequence
  oligonucleotide
caagaaatggtagactcaagtt

SEQ ID NO: 861 - DNA, 25 bp, Artificial Sequence
  oligonucleotide
actgcgtggtcaatgtggttatggg

SEQ ID NO: 862 - DNA, 19 bp, Artificial Sequence
  oligonucleotide
catttgtgggtggagtttg

SEQ ID NO: 863 - DNA, 20 bp, Artificial Sequence
  oligonucleotide
gcaaccaagaaatggtagac

SEQ ID NO: 864 - DNA, 25 bp, Artificial Sequence
  oligonucleotide
actgcgtggtcaatgtggttatggg

SEQ ID NO: 865 - DNA, 19 bp, Artificial Sequence
  oligonucleotide
cgctgttgctggtgttcta

SEQ ID NO: 866 - DNA, 19 bp, Artificial Sequence
  oligonucleotide
cattgaccacgcagtgttt

SEQ ID NO: 867 - DNA, 22 bp, Artificial Sequence
  oligonucleotide
tttgtgggtggagtttgtgcgc

SEQ ID NO: 868 - DNA, 18 bp, Artificial Sequence
  oligonucleotide
acagttcagcgactgcag

SEQ ID NO: 869 - DNA, 19 bp, Artificial Sequence
  oligonucleotide
aacgaggcgtcttcaaatg

SEQ ID NO: 870 - DNA, 24 bp, Artificial Sequence
  oligonucleotide
tcagcagaggtcagatgcagaagc

SEQ ID NO: 871 - DNA, 19 bp, Artificial Sequence
  oligonucleotide
gctgatcaacggctacatc

SEQ ID NO: 872 - DNA, 19 bp, Artificial Sequence
  oligonucleotide
gcgtcttcaaatggcttct

SEQ ID NO: 873 - DNA, 22 bp, Artificial Sequence
  oligonucleotide
tgcagtcgctgaactgtgaggc

SEQ ID NO: 874 - DNA, 20 bp, Artificial Sequence
  oligonucleotide
gcagaggtcagatgcagaag

SEQ ID NO: 875 - DNA, 19 bp, Artificial Sequence
  oligonucleotide
gcggaggatgatgtggatg

SEQ ID NO: 876 - DNA, 22 bp, Artificial Sequence
  oligonucleotide
ttgaagacgcctcgtttgcgct

SEQ ID NO: 877 - DNA, 19 bp, Artificial Sequence
  oligonucleotide
agaggaggactttgagtct

SEQ ID NO: 878 - DNA, 19 bp, Artificial Sequence
  oligonucleotide
caaatggcttctgcatctg

SEQ ID NO: 879 - DNA, 22 bp, Artificial Sequence
  oligonucleotide
tgcagtcgctgaactgtgaggc

SEQ ID NO: 880 - DNA, 19 bp, Artificial Sequence
  oligonucleotide
cctgttcgtcttctgtacc

SEQ ID NO: 881 - DNA, 20 bp, Artificial Sequence
  oligonucleotide
gattccatctcctccatctg

SEQ ID NO: 882 - DNA, 22 bp, Artificial Sequence
  oligonucleotide
acgtgctgagcgttactggcag

SEQ ID NO: 883 - DNA, 19 bp, Artificial Sequence
  oligonucleotide
catcgtgtccatcgtcatc

SEQ ID NO: 884 - DNA, 18 bp, Artificial Sequence
  oligonucleotide
agtaacgctcagcacgtc

SEQ ID NO: 885 - DNA, 24 bp, Artificial Sequence
  oligonucleotide
tggtggccctgttcgtcttctgta

SEQ ID NO: 886 - DNA, 19 bp, Artificial Sequence
  oligonucleotide
catcgtgtccatcgtcatc

SEQ ID NO: 887 - DNA, 20 bp, Artificial Sequence
  oligonucleotide
ctcggatctgagattccatc

SEQ ID NO: 888 - DNA, 22 bp, Artificial Sequence
  oligonucleotide
acgtgctgagcgttactggcag

SEQ ID NO: 889 - DNA, 19 bp, Artificial Sequence
  oligonucleotide
cacaatccaggtagggaac

SEQ ID NO: 890 - DNA, 20 bp, Artificial Sequence
  oligonucleotide
cttggtacagaagacgaaca

SEQ ID NO: 891 - DNA, 22 bp, Artificial Sequence
  oligonucleotide
aaccagaccatcgccacactgc

SEQ ID NO: 892 - DNA, 19 bp, Artificial Sequence
  oligonucleotide
cctgttcgtcttctgtacc

SEQ ID NO: 893 - DNA, 20 bp, Artificial Sequence
  oligonucleotide
gattccatctcctccatctg

SEQ ID NO: 894 - DNA, 22 bp, Artificial Sequence
  oligonucleotide
acgtgctgagcgttactggcag

SEQ ID NO: 895 - DNA, 19 bp, Artificial Sequence
  oligonucleotide
cacaatccaggtagggaac

SEQ ID NO: 896 - DNA, 20 bp, Artificial Sequence
  oligonucleotide
cttggtacagaagacgaaca

SEQ ID NO: 897 - DNA, 22 bp, Artificial Sequence
  oligonucleotide
aaccagaccatcgccacactgc

SEQ ID NO: 898 - DNA, 17 bp, Artificial Sequence
  oligonucleotide
gaccagtacccgcactt

SEQ ID NO: 899 - DNA, 17 bp, Artificial Sequence
  oligonucleotide
cgcttctcgctctcgtt

SEQ ID NO: 900 - DNA, 22 bp, Artificial Sequence
  oligonucleotide
cgctgggcaagctctggagact

SEQ ID NO: 901 - DNA, 19 bp, Artificial Sequence
  oligonucleotide
catcgtgtccatcgtcatc

SEQ ID NO: 902 - DNA, 20 bp, Artificial Sequence
  oligonucleotide
ctcggatctgagattccatc

SEQ ID NO: 903 - DNA, 22 bp, Artificial Sequence
  oligonucleotide
acgtgctgagcgttactggcag

SEQ ID NO: 904 - DNA, 25 bp, Artificial Sequence
  oligonucleotide
ggtgaggatttggataaatctgatg

SEQ ID NO: 905 - DNA, 25 bp, Artificial Sequence
  oligonucleotide
acatgggccgaggcaagataagcaa

SEQ ID NO: 906 - DNA, 22 bp, Artificial Sequence
  oligonucleotide
tcaaccacaaggtccaagaaac

SEQ ID NO: 907 - DNA, 19 bp, Artificial Sequence
  oligonucleotide
gcagagattggtgaggatt

SEQ ID NO: 908 - DNA, 22 bp, Artificial Sequence
  oligonucleotide
tgggccgaggcaagataagcaa

SEQ ID NO: 909 - DNA, 18 bp, Artificial Sequence
  oligonucleotide
tccaactcaaccacaagg

SEQ ID NO: 910 - DNA, 19 bp, Artificial Sequence
  oligonucleotide
gaggcaagataagcaagga

SEQ ID NO: 911 - DNA, 27 bp, Artificial Sequence
  oligonucleotide
tcttggaccttgtggttgagttggaga

SEQ ID NO: 912 - DNA, 21 bp, Artificial Sequence
  oligonucleotide
gtggatgttctttaggcattt

SEQ ID NO: 913 - DNA, 18 bp, Artificial Sequence
  oligonucleotide
atggcagagattggtgag

SEQ ID NO: 914 - DNA, 22 bp, Artificial Sequence
  oligonucleotide
tgggccgaggcaagataagcaa

SEQ ID NO: 915 - DNA, 18 bp, Artificial Sequence
  oligonucleotide
accacaaggtccaagaaa

SEQ ID NO: 916 - DNA, 20 bp, Artificial Sequence
  oligonucleotide
ggaccactttccctacttta

SEQ ID NO: 917 - DNA, 22 bp, Artificial Sequence
  oligonucleotide
atccgccacaacctttccctgc

SEQ ID NO: 918 - DNA, 17 bp, Artificial Sequence
  oligonucleotide
gtctcccggacaaacat

SEQ ID NO: 919 - DNA, 19 bp, Artificial Sequence
  oligonucleotide
tggattgaggaccactttc

SEQ ID NO: 920 - DNA, 22 bp, Artificial Sequence
  oligonucleotide
atccgccacaacctttccctgc

SEQ ID NO: 921 - DNA, 18 bp, Artificial Sequence
  oligonucleotide
gaaggagaccttgccatt

SEQ ID NO: 922 - DNA, 18 bp, Artificial Sequence
  oligonucleotide
caacagcactgagaggaa

SEQ ID NO: 923 - DNA, 22 bp, Artificial Sequence
  oligonucleotide
acattgccaagccaggctggaa

SEQ ID NO: 924 - DNA, 17 bp, Artificial Sequence
  oligonucleotide
atgtcgtgcagggaaag

SEQ ID NO: 925 - DNA, 19 bp, Artificial Sequence
  oligonucleotide
gtgtgaactcttcgtctca

SEQ ID NO: 926 - DNA, 24 bp, Artificial Sequence
  oligonucleotide
acgagaggaggaacaggagtgaca

SEQ ID NO: 927 - DNA, 18 bp, Artificial Sequence
  oligonucleotide
ctgtccgtgcttcatagt

SEQ ID NO: 928 - DNA, 18 bp, Artificial Sequence
  oligonucleotide
aaggactcaccgtctttg

SEQ ID NO: 929 - DNA, 24 bp, Artificial Sequence
  oligonucleotide
acgagaggaggaacaggagtgaca

SEQ ID NO: 930 - DNA, 20 bp, Artificial Sequence
  oligonucleotide
gtccgtgcttcatagtcata

SEQ ID NO: 931 - DNA, 19 bp, Artificial Sequence
  oligonucleotide
ctttggaggtgtgaactct

SEQ ID NO: 932 - DNA, 24 bp, Artificial Sequence
  oligonucleotide
acgagaggaggaacaggagtgaca

SEQ ID NO: 933 - DNA, 18 bp, Artificial Sequence
  oligonucleotide
atcttctgtccgtgcttc

SEQ ID NO: 934 - DNA, 20 bp, Artificial Sequence
  oligonucleotide
cgtctttggaggtgtgaact

SEQ ID NO: 935 - DNA, 31 bp, Artificial Sequence
  oligonucleotide
cgagaggaggaacaggagtgacactctttgt

SEQ ID NO: 936 - DNA, 23 bp, Artificial Sequence
  oligonucleotide
tccgtgcttcatagtcataaagg

SEQ ID NO: 937 - DNA, 18 bp, Artificial Sequence
  oligonucleotide
ccttcaaagcccagtaca

SEQ ID NO: 938 - DNA, 22 bp, Artificial Sequence
  oligonucleotide
acgtttgaagggtgccggaagt

SEQ ID NO: 939 - DNA, 18 bp, Artificial Sequence
  oligonucleotide
tttcgaggcgtgagtatg

SEQ ID NO: 940 - DNA, 21 bp, Artificial Sequence
  oligonucleotide
cagtacatgctggtggttcac

SEQ ID NO: 941 - DNA, 24 bp, Artificial Sequence
  oligonucleotide
actggcgagaagccacacaagtgc

SEQ ID NO: 942 - DNA, 21 bp, Artificial Sequence
  oligonucleotide
ttcgaggcgtgagtatgactt

SEQ ID NO: 943 - DNA, 18 bp, Artificial Sequence
  oligonucleotide
caaagcccagtacatgct

SEQ ID NO: 944 - DNA, 22 bp, Artificial Sequence
  oligonucleotide
acgtttgaagggtgccggaagt

SEQ ID NO: 945 - DNA, 18 bp, Artificial Sequence
  oligonucleotide
gtacatgctggtggttca

SEQ ID NO: 946 - DNA, 23 bp, Artificial Sequence
  oligonucleotide
cacactggcgagaagccacacaa

SEQ ID NO: 947 - DNA, 19 bp, Artificial Sequence
  oligonucleotide
gaggcgtgagtatgacttc

SEQ ID NO: 948 - DNA, 17 bp, Artificial Sequence
  oligonucleotide
ggtgcgtcttcaggttt

SEQ ID NO: 949 - DNA, 20 bp, Artificial Sequence
  oligonucleotide
gaggaccagcatctaactac

SEQ ID NO: 950 - DNA, 26 bp, Artificial Sequence
  oligonucleotide
tcagcagaaagcacaaacacaactgc

SEQ ID NO: 951 - DNA, 20 bp, Artificial Sequence
  oligonucleotide
actcacaacctcctgaatac

SEQ ID NO: 952 - DNA, 22 bp, Artificial Sequence
  oligonucleotide
cagcagaaagcacaaacacaac

SEQ ID NO: 953 - DNA, 23 bp, Artificial Sequence
  oligonucleotide
attcaggaggttgtgagtgggct

SEQ ID NO: 954 - DNA, 19 bp, Artificial Sequence
  oligonucleotide
cactatgcagggcaccaac

SEQ ID NO: 955 - DNA, 19 bp, Artificial Sequence
  oligonucleotide
cttggaccagatggaagaa

SEQ ID NO: 956 - DNA, 26 bp, Artificial Sequence
  oligonucleotide
tcagcagaaagcacaaacacaactgc

SEQ ID NO: 957 - DNA, 17 bp, Artificial Sequence
  oligonucleotide
ccactcacaacctcctg

SEQ ID NO: 958 - DNA, 18 bp, Artificial Sequence
  oligonucleotide
tcagaggaccagcatcta

SEQ ID NO: 959 - DNA, 29 bp, Artificial Sequence
  oligonucleotide
tggaagagatcagcagaaagcacaaacac

SEQ ID NO: 960 - DNA, 20 bp, Artificial Sequence
  oligonucleotide
tcctgaatacagaagcagtt

SEQ ID NO: 961 - DNA, 17 bp, Artificial Sequence
  oligonucleotide
aaggccctcagtacctc

SEQ ID NO: 962 - DNA, 24 bp, Artificial Sequence
  oligonucleotide
tgaatcgctcagggtgtcctctcc

SEQ ID NO: 963 - DNA, 19 bp, Artificial Sequence
  oligonucleotide
gtgaccacgtcgatttctt

SEQ ID NO: 964 - DNA, 17 bp, Artificial Sequence
  oligonucleotide
ccacaaggccctcagta

SEQ ID NO: 965 - DNA, 24 bp, Artificial Sequence
  oligonucleotide
tgaatcgctcagggtgtcctctcc

SEQ ID NO: 966 - DNA, 21 bp, Artificial Sequence
  oligonucleotide
cacgtcgatttcttcctcttc

SEQ ID NO: 967 - DNA, 17 bp, Artificial Sequence
  oligonucleotide
gacaccctgagcgattc

SEQ ID NO: 968 - DNA, 29 bp, Artificial Sequence
  oligonucleotide
tgaagatgatgaagaggaagatgaagagg

SEQ ID NO: 969 - DNA, 19 bp, Artificial Sequence
  oligonucleotide
gaatgtggtgacagccttg

SEQ ID NO: 970 - DNA, 17 bp, Artificial Sequence
  oligonucleotide
gacaccctgagcgattc

SEQ ID NO: 971 - DNA, 30 bp, Artificial Sequence
  oligonucleotide
agaggaagatgaagaggaagaaatcgacgt

SEQ ID NO: 972 - DNA, 17 bp, Artificial Sequence
  oligonucleotide
gcttctccacagtgacc

SEQ ID NO: 973 - DNA, 17 bp, Artificial Sequence
  oligonucleotide
cccttctacgacagcag

SEQ ID NO: 974 - DNA, 22 bp, Artificial Sequence
  oligonucleotide
agggccttcagtactccctgca

SEQ ID NO: 975 - DNA, 17 bp, Artificial Sequence
  oligonucleotide
gggacttggagcttgag

SEQ ID NO: 976 - DNA, 17 bp, Artificial Sequence
  oligonucleotide
gtggcagattccaccca

SEQ ID NO: 977 - DNA, 24 bp, Artificial Sequence
  oligonucleotide
tctgcccatgcctctccttctgaa

SEQ ID NO: 978 - DNA, 19 bp, Artificial Sequence
  oligonucleotide
cgtagagttcagccctctc

SEQ ID NO: 979 - DNA, 17 bp, Artificial Sequence
  oligonucleotide
agcgacaacccgtacac

SEQ ID NO: 980 - DNA, 22 bp, Artificial Sequence
  oligonucleotide
agggccttcagtactccctgca

SEQ ID NO: 981 - DNA, 18 bp, Artificial Sequence
  oligonucleotide
cattgtccggtgactcgt

SEQ ID NO: 982 - DNA, 17 bp, Artificial Sequence
  oligonucleotide
tacgacagcagcgacaa

SEQ ID NO: 983 - DNA, 22 bp, Artificial Sequence
  oligonucleotide
agggccttcagtactccctgca

SEQ ID NO: 984 - DNA, 19 bp, Artificial Sequence
  oligonucleotide
ttggagcttgagtcctgag

SEQ ID NO: 985 - DNA, 21 bp, Artificial Sequence
  oligonucleotide
cttcttcatggccgcgttaat

SEQ ID NO: 986 - DNA, 24 bp, Artificial Sequence
  oligonucleotide
tccaggcagcggtagtagtggtgt

SEQ ID NO: 987 - DNA, 21 bp, Artificial Sequence
  oligonucleotide
aatgagcagaaccatggcaaa

SEQ ID NO: 988 - DNA, 17 bp, Artificial Sequence
  oligonucleotide
acgtccatcagcaatgt

SEQ ID NO: 989 - DNA, 23 bp, Artificial Sequence
  oligonucleotide
ccgcgttaatcccaattcccgct

SEQ ID NO: 990 - DNA, 19 bp, Artificial Sequence
  oligonucleotide
ttgaacaccactactaccg

SEQ ID NO: 991 - DNA, 17 bp, Artificial Sequence
  oligonucleotide
ttcatggccgcgttaat

SEQ ID NO: 992 - DNA, 22 bp, Artificial Sequence
  oligonucleotide
tccaggcagcggtagtagtggt

SEQ ID NO: 993 - DNA, 17 bp, Artificial Sequence
  oligonucleotide
agcagaaccatggcaaa

SEQ ID NO: 994 - DNA, 18 bp, Artificial Sequence
  oligonucleotide
cgcgttaatcccaattcc

SEQ ID NO: 995 - DNA, 22 bp, Artificial Sequence
  oligonucleotide
tccaggcagcggtagtagtggt

SEQ ID NO: 996 - DNA, 19 bp, Artificial Sequence
  oligonucleotide
gtcctcgcgtcgatataaa

SEQ ID NO: 997 - DNA, 20 bp, Artificial Sequence
  oligonucleotide
ctgctacaagtcaggagttc

SEQ ID NO: 998 - DNA, 29 bp, Artificial Sequence
  oligonucleotide
tggaatgattgagcggatgattgagaagc

SEQ ID NO: 999 - DNA, 17 bp, Artificial Sequence
  oligonucleotide
gatcacgcacggaaaca

SEQ ID NO: 1000 - DNA, 20 bp, Artificial Sequence
  oligonucleotide
aaatctgctacaagtcagga

SEQ ID NO: 1001 - DNA, 23 bp, Artificial Sequence
  oligonucleotide
aagctgtttccgtgcgtgatcct

SEQ ID NO: 1002 - DNA, 17 bp, Artificial Sequence
  oligonucleotide
tcccagaagcagtcgag

SEQ ID NO: 1003 - DNA, 20 bp, Artificial Sequence
  oligonucleotide
tgattgagcggatgattgag

SEQ ID NO: 1004 - DNA, 23 bp, Artificial Sequence
  oligonucleotide
aagctgtttccgtgcgtgatcct

SEQ ID NO: 1005 - DNA, 17 bp, Artificial Sequence
  oligonucleotide
ctccctcccagaagcag

SEQ ID NO: 1006 - DNA, 20 bp, Artificial Sequence
  oligonucleotide
tgagcggatgattgagaagc

SEQ ID NO: 1007 - DNA, 23 bp, Artificial Sequence
  oligonucleotide
tgtttccgtgcgtgatcctcacc

SEQ ID NO: 1008 - DNA, 17 bp, Artificial Sequence
  oligonucleotide
ttggctccctcccagaa

SEQ ID NO: 1009 - DNA, 18 bp, Artificial Sequence
  oligonucleotide
ggcaggagaggttcaaat

SEQ ID NO: 1010 - DNA, 28 bp, Artificial Sequence
  oligonucleotide
tcaacctgaatgatgtggcatctctgga

SEQ ID NO: 1011 - DNA, 17 bp, Artificial Sequence
  oligonucleotide
agccactgcttggtatg

SEQ ID NO: 1012 - DNA, 20 bp, Artificial Sequence
  oligonucleotide
gcattgcatcaacctactat

SEQ ID NO: 1013 - DNA, 22 bp, Artificial Sequence
  oligonucleotide
acataccaagcagtggctggcc

SEQ ID NO: 1014 - DNA, 18 bp, Artificial Sequence
  oligonucleotide
attctccttcagggcatc

SEQ ID NO: 1015 - DNA, 17 bp, Artificial Sequence
  oligonucleotide
ctgggcaggagaggttc

SEQ ID NO: 1016 - DNA, 28 bp, Artificial Sequence
  oligonucleotide
agaggagctcaagccatcatcattgtct

SEQ ID NO: 1017 - DNA, 20 bp, Artificial Sequence
  oligonucleotide
ccagagatgccacatcattc

SEQ ID NO: 1018 - DNA, 20 bp, Artificial Sequence
  oligonucleotide
gggcaggagaggttcaaatg

SEQ ID NO: 1019 - DNA, 30 bp, Artificial Sequence
  oligonucleotide
tcttcaacctgaatgatgtggcatctctgg

SEQ ID NO: 1020 - DNA, 20 bp, Artificial Sequence
  oligonucleotide
cagccactgcttggtatgtt

SEQ ID NO: 1021 - DNA, 17 bp, Artificial Sequence
  oligonucleotide
ccagttgcagccttctc

SEQ ID NO: 1022 - DNA, 22 bp, Artificial Sequence
  oligonucleotide
agccaaacgccgaccaaggaaa

SEQ ID NO: 1023 - DNA, 18 bp, Artificial Sequence
  oligonucleotide
ggtatggcacaggtgatg

SEQ ID NO: 1024 - DNA, 17 bp, Artificial Sequence
  oligonucleotide
agccttctcagccaaac

SEQ ID NO: 1025 - DNA, 25 bp, Artificial Sequence
  oligonucleotide
taggcatcacctgtgccataccagt

SEQ ID NO: 1026 - DNA, 20 bp, Artificial Sequence
  oligonucleotide
cctcagaacttccagaatca

SEQ ID NO: 1027 - DNA, 17 bp, Artificial Sequence
  oligonucleotide
gagggcttggttgtcag

SEQ ID NO: 1028 - DNA, 22 bp, Artificial Sequence
  oligonucleotide
agccaaacgccgaccaaggaaa

SEQ ID NO: 1029 - DNA, 19 bp, Artificial Sequence
  oligonucleotide
tcactgcaattctcatggt

SEQ ID NO: 1030 - DNA, 22 bp, Artificial Sequence
  oligonucleotide
agccaaacgccgaccaaggaaa

SEQ ID NO: 1031 - DNA, 20 bp, Artificial Sequence
  oligonucleotide
agcaaatcactgcaattctc

SEQ ID NO: 1032 - DNA, 17 bp, Artificial Sequence
  oligonucleotide
ggaccagactcgtctca

SEQ ID NO: 1033 - DNA, 18 bp, Artificial Sequence
  oligonucleotide
tcggatgaggaggtcatt

SEQ ID NO: 1034 - DNA, 26 bp, Artificial Sequence
  oligonucleotide
actggccaagtctctcatcaagtcct

SEQ ID NO: 1035 - DNA, 17 bp, Artificial Sequence
  oligonucleotide
tttggcatcggaagcat

SEQ ID NO: 1036 - DNA, 18 bp, Artificial Sequence
  oligonucleotide
attgcactggccaagtct

SEQ ID NO: 1037 - DNA, 24 bp, Artificial Sequence
  oligonucleotide
tcctggaagaagctcctggatgct

SEQ ID NO: 1038 - DNA, 17 bp, Artificial Sequence
  oligonucleotide
tccctggctttggcatc

SEQ ID NO: 1039 - DNA, 17 bp, Artificial Sequence
  oligonucleotide
agctcggatgaggaggt

SEQ ID NO: 1040 - DNA, 26 bp, Artificial Sequence
  oligonucleotide
actggccaagtctctcatcaagtcct

SEQ ID NO: 1041 - DNA, 17 bp, Artificial Sequence
  oligonucleotide
tggctttggcatcggaa

SEQ ID NO: 1042 - DNA, 19 bp, Artificial Sequence
  oligonucleotide
ctggtccacagagtattcc

SEQ ID NO: 1043 - DNA, 30 bp, Artificial Sequence
  oligonucleotide
accacagagtatttctcagtcacagatctc

SEQ ID NO: 1044 - DNA, 21 bp, Artificial Sequence
  oligonucleotide
agatagctcagttcttgagac

SEQ ID NO: 1045 - DNA, 21 bp, Artificial Sequence
  oligonucleotide
cagagtattccagcagttagt

SEQ ID NO: 1046 - DNA, 30 bp, Artificial Sequence; identical to SEQ ID NO: 1043
  oligonucleotide

SEQ ID NO: 1047 - DNA, 22 bp, Artificial Sequence
  oligonucleotide
tcatggtaagatagctcagttc

SEQ ID NO: 1048 - DNA, 19 bp, Artificial Sequence
  oligonucleotide
cacagagtattccagcagt

SEQ ID NO: 1049 - DNA, 30 bp, Artificial Sequence; identical to SEQ ID NO: 1043
  oligonucleotide

SEQ ID NO: 1050 - DNA, 21 bp, Artificial Sequence
  oligonucleotide
accttcatggtaagatagctc

SEQ ID NO: 1051 - DNA, 20 bp, Artificial Sequence
  oligonucleotide
ctcagtcacagatctcacaa

SEQ ID NO: 1052 - DNA, 30 bp, Artificial Sequence
  oligonucleotide
agaactgagctatcttaccatgaaggttgt

SEQ ID NO: 1053 - DNA, 24 bp, Artificial Sequence
  oligonucleotide
acctaaatagtagttacagtcctc

SEQ ID NO: 1054 - DNA, 17 bp, Artificial Sequence
  oligonucleotide
caccgacctcccgttag

SEQ ID NO: 1055 - DNA, 22 bp, Artificial Sequence
  oligonucleotide
accctgaggtccttcacagcct

SEQ ID NO: 1056 - DNA, 20 bp, Artificial Sequence
  oligonucleotide
gttctgagccttgagttgtg

SEQ ID NO: 1057 - DNA, 18 bp, Artificial Sequence
  oligonucleotide
cagcctgcagacacaact

SEQ ID NO: 1058 - DNA, 21 bp, Artificial Sequence
  oligonucleotide
gctttgcagatgctgaattcg

SEQ ID NO: 1059 - DNA, 23 bp, Artificial Sequence
  oligonucleotide
agcaactcttccacaaggtggcc

SEQ ID NO: 1060 - DNA, 17 bp, Artificial Sequence
  oligonucleotide
gaggtccttcacagcct

SEQ ID NO: 1061 - DNA, 25 bp, Artificial Sequence
  oligonucleotide
acaactcaaggctcagaacagcagg

SEQ ID NO: 1062 - DNA, 19 bp, Artificial Sequence
  oligonucleotide
caccttgtggaagagttgc

SEQ ID NO: 1063 - DNA, 18 bp, Artificial Sequence
  oligonucleotide
gaccctgaggtccttcac

SEQ ID NO: 1064 - DNA, 23 bp, Artificial Sequence
  oligonucleotide
agccttgagttgtgtctgcaggc

SEQ ID NO: 1065 - DNA, 18 bp, Artificial Sequence
  oligonucleotide
tgtggaagagttgctgga

SEQ ID NO: 1066 - DNA, 19 bp, Artificial Sequence
  oligonucleotide
ctgtgggcggattagggct

SEQ ID NO: 1067 - DNA, 19 bp, Artificial Sequence
  oligonucleotide
gagactctcagggtcgaaa

SEQ ID NO: 1068 - DNA, 24 bp, Artificial Sequence
  oligonucleotide
atttctaccactccaaacgccggc

SEQ ID NO: 1069 - DNA, 19 bp, Artificial Sequence
  oligonucleotide
gagactctcagggtcgaaa

SEQ ID NO: 1070 - DNA, 23 bp, Artificial Sequence
  oligonucleotide
aatctgtcatgctggtctgccgc

SEQ ID NO: 1071 - DNA, 17 bp, Artificial Sequence
  oligonucleotide
ttcctgtgggcggatta

SEQ ID NO: 1072 - DNA, 17 bp, Artificial Sequence
  oligonucleotide
aggtggacctggagact

SEQ ID NO: 1073 - DNA, 23 bp, Artificial Sequence
  oligonucleotide
aatctgtcatgctggtctgccgc

SEQ ID NO: 1074 - DNA, 20 bp, Artificial Sequence
  oligonucleotide
ggcttcctcttggagaagat

SEQ ID NO: 1075 - DNA, 20 bp, Artificial Sequence
  oligonucleotide
ggacctgtcactgtcttgta

SEQ ID NO: 1076 - DNA, 20 bp, Artificial Sequence
  oligonucleotide
aaacggcggcagaccagcat

SEQ ID NO: 1077 - DNA, 21 bp, Artificial Sequence
  oligonucleotide
gcgtttggagtggtagaaatc

SEQ ID NO: 1078 - DNA, 19 bp, Artificial Sequence
  oligonucleotide
gaagctgacctggaagaga

SEQ ID NO: 1079 - DNA, 24 bp, Artificial Sequence
  oligonucleotide
agtttgagctttctggctgcacca

SEQ ID NO: 1080 - DNA, 22 bp, Artificial Sequence
  oligonucleotide
ccacagaatttagctcggtatg

SEQ ID NO: 1081 - DNA, 19 bp, Artificial Sequence
  oligonucleotide
gcaggctagagaagcagag

SEQ ID NO: 1082 - DNA, 22 bp, Artificial Sequence
  oligonucleotide
ttccaggtcagcttcgcaaggc

SEQ ID NO: 1083 - DNA, 19 bp, Artificial Sequence
  oligonucleotide
gggagtacggatgcacttt

SEQ ID NO: 1084 - DNA, 20 bp, Artificial Sequence
  oligonucleotide
gctgacctggaagagaacat

SEQ ID NO: 1085 - DNA, 26 bp, Artificial Sequence
  oligonucleotide
tcaagtttgagctttctggctgcacc

SEQ ID NO: 1086 - DNA, 20 bp, Artificial Sequence
  oligonucleotide
gctcggtatgtcttcatgct

SEQ ID NO: 1087 - DNA, 26 bp, Artificial Sequence
  oligonucleotide
cctatcaagtttgagctttctggctg

SEQ ID NO: 1088 - DNA, 20 bp, Artificial Sequence
  oligonucleotide
gaagctgacctggaagagaa

SEQ ID NO: 1089 - DNA, 22 bp, Artificial Sequence
  oligonucleotide
ccacagaatttagctcggtatg

SEQ ID NO: 1090 - DNA, 20 bp, Artificial Sequence
  oligonucleotide
gcgagaacgacatcaacatc

SEQ ID NO: 1091 - DNA, 22 bp, Artificial Sequence
  oligonucleotide
agctcctgctcttggagaccga

SEQ ID NO: 1092 - DNA, 17 bp, Artificial Sequence
  oligonucleotide
tggattcgtcaccagca

SEQ ID NO: 1093 - DNA, 29 bp, Artificial Sequence
  oligonucleotide
aaggatcctgccttaagtcaacttatttg

SEQ ID NO: 1094 - DNA, 20 bp, Artificial Sequence
  oligonucleotide
gatccatgtagcgactttcc

SEQ ID NO: 1095 - DNA, 22 bp, Artificial Sequence
  oligonucleotide
acgaatccacattcatctcaat

SEQ ID NO: 1096 - DNA, 17 bp, Artificial Sequence
  oligonucleotide
cggagctcctgctcttg

SEQ ID NO: 1097 - DNA, 22 bp, Artificial Sequence
  oligonucleotide
tgtggattcgtcaccagcacgc

SEQ ID NO: 1098 - DNA, 22 bp, Artificial Sequence
  oligonucleotide
aggatccttccattgagatgaa

SEQ ID NO: 1099 - DNA, 18 bp, Artificial Sequence
  oligonucleotide
gctcctgctcttggagac

SEQ ID NO: 1100 - DNA, 22 bp, Artificial Sequence
  oligonucleotide
tgtggattcgtcaccagcacgc

SEQ ID NO: 1101 - DNA, 20 bp, Artificial Sequence
  oligonucleotide
gcaggatccttccattgaga

SEQ ID NO: 1102 - DNA, 20 bp, Artificial Sequence
  oligonucleotide
gtcggccaagttgatgaatg

SEQ ID NO: 1103 - DNA, 22 bp, Artificial Sequence
  oligonucleotide
acagcgtggtcctctgcctctt

SEQ ID NO: 1104 - DNA, 21 bp, Artificial Sequence
  oligonucleotide
gatgagcgtgaagtggatttg

SEQ ID NO: 1105 - DNA, 18 bp, Artificial Sequence
  oligonucleotide
cgagtcggccaagttgat

SEQ ID NO: 1106 - DNA, 22 bp, Artificial Sequence
  oligonucleotide
acagcgtggtcctctgcctctt

SEQ ID NO: 1107 - DNA, 19 bp, Artificial Sequence
  oligonucleotide
actggatgagcgtgaagtg

SEQ ID NO: 1108 - DNA, 17 bp, Artificial Sequence
  oligonucleotide
tgtacgagtcggccaag

SEQ ID NO: 1109 - DNA, 22 bp, Artificial Sequence
  oligonucleotide
acagcgtggtcctctgcctctt

SEQ ID NO: 1110 - DNA, 18 bp, Artificial Sequence
  oligonucleotide
gatttgcagggcgatgtc

SEQ ID NO: 1111 - DNA, 17 bp, Artificial Sequence
  oligonucleotide
caggatcgcctcacagt

SEQ ID NO: 1112 - DNA, 22 bp, Artificial Sequence
  oligonucleotide
acagcgtggtcctctgcctctt

SEQ ID NO: 1113 - DNA, 19 bp, Artificial Sequence
  oligonucleotide
ctcctcctcctcgtcaatg

SEQ ID NO: 1114 - DNA, 17 bp, Artificial Sequence
  oligonucleotide
cctcaacggcgagatca

SEQ ID NO: 1115 - DNA, 23 bp, Artificial Sequence
  oligonucleotide
tcgcatcttgtgtcgctgaagcg

SEQ ID NO: 1116 - DNA, 18 bp, Artificial Sequence
  oligonucleotide
acccacagagcacgtaat

SEQ ID NO: 1117 - DNA, 19 bp, Artificial Sequence
  oligonucleotide
gaatccgaagttggaaccc

SEQ ID NO: 1118 - DNA, 22 bp, Artificial Sequence
  oligonucleotide
agcaccctcaacggcgagatca

SEQ ID NO: 1119 - DNA, 18 bp, Artificial Sequence
  oligonucleotide
gcttcagcgacacaagat

SEQ ID NO: 1120 - DNA, 20 bp, Artificial Sequence
  oligonucleotide
ttggagctgaactcggaatc

SEQ ID NO: 1121 - DNA, 22 bp, Artificial Sequence
  oligonucleotide
agcaccctcaacggcgagatca

SEQ ID NO: 1122 - DNA, 18 bp, Artificial Sequence
  oligonucleotide
agcgacacaagatgcgat

SEQ ID NO: 1123 - DNA, 17 bp, Artificial Sequence
  oligonucleotide
cgctcagcaccctcaac

SEQ ID NO: 1124 - DNA, 23 bp, Artificial Sequence
  oligonucleotide
tcgcatcttgtgtcgctgaagcg

SEQ ID NO: 1125 - DNA, 20 bp, Artificial Sequence
  oligonucleotide
agcacgtaattcctcttgcc

SEQ ID NO: 1126 - DNA, 19 bp, Artificial Sequence
  oligonucleotide
agtacccgtatgggacaaa

SEQ ID NO: 1127 - DNA, 20 bp, Artificial Sequence
  oligonucleotide
ccagtttgctatggtgaaca

SEQ ID NO: 1128 - DNA, 29 bp, Artificial Sequence
  oligonucleotide
tgcaaggtcaagatggttcattatggctg

SEQ ID NO: 1129 - DNA, 19 bp, Artificial Sequence
  oligonucleotide
gggaccacaacctggattc

SEQ ID NO: 1130 - DNA, 22 bp, Artificial Sequence
  oligonucleotide
agctctacagctcccaggtgtg

SEQ ID NO: 1131 - DNA, 17 bp, Artificial Sequence
  oligonucleotide
gctcgcagccttgtcag

SEQ ID NO: 1132 - DNA, 17 bp, Artificial Sequence
  oligonucleotide
gggcactgggagctcta

SEQ ID NO: 1133 - DNA, 22 bp, Artificial Sequence
  oligonucleotide
acctactgtcctacctgcggca

SEQ ID NO: 1134 - DNA, 17 bp, Artificial Sequence
  oligonucleotide
gggctccagggtcttca

SEQ ID NO: 1135 - DNA, 19 bp, Artificial Sequence
  oligonucleotide
ggacaaattcccagctgac

SEQ ID NO: 1136 - DNA, 22 bp, Artificial Sequence
  oligonucleotide
tcccaggtgtgctgacaaggct

SEQ ID NO: 1137 - DNA, 18 bp, Artificial Sequence
  oligonucleotide
ccgcaggtaggacagtag

SEQ ID NO: 1138 - DNA, 20 bp, Artificial Sequence
  oligonucleotide
ctaccacgacgactcataca

SEQ ID NO: 1139 - DNA, 23 bp, Artificial Sequence
  oligonucleotide
tggtggcctctctctacacgact

SEQ ID NO: 1140 - DNA, 19 bp, Artificial Sequence
  oligonucleotide
gctcggtttcaggagtttg

SEQ ID NO: 1141 - DNA, 19 bp, Artificial Sequence
  oligonucleotide
atggaacagcccttctacc

SEQ ID NO: 1142 - DNA, 25 bp, Artificial Sequence
  oligonucleotide
tcatacacagctacgggatacggcc

SEQ ID NO: 1143 - DNA, 22 bp, Artificial Sequence
  oligonucleotide
tcaggagtttgtagtcgtgtag

SEQ ID NO: 1144 - DNA, 17 bp, Artificial Sequence
  oligonucleotide
cccggatgtgcactaaa

SEQ ID NO: 1145 - DNA, 21 bp, Artificial Sequence
  oligonucleotide
gctcggtttcaggagtttgta

SEQ ID NO: 1146 - DNA, 23 bp, Artificial Sequence
  oligonucleotide
tcatacacagctacgggatacgg

SEQ ID NO: 1147 - DNA, 20 bp, Artificial Sequence
  oligonucleotide
cgacgactcatacacagcta

SEQ ID NO: 1148 - DNA, 23 bp, Artificial Sequence
  oligonucleotide
aaactcctgaaaccgagcctggc

SEQ ID NO: 1149 - DNA, 19 bp, Artificial Sequence
  oligonucleotide
ctttgagactccggtaggg

SEQ ID NO: 1150 - DNA, 21 bp, Artificial Sequence
  oligonucleotide
gtggccaactacaacttcttc

SEQ ID NO: 1151 - DNA, 22 bp, Artificial Sequence
  oligonucleotide
cgcaagcccaagtgggacaaga

SEQ ID NO: 1152 - DNA, 19 bp, Artificial Sequence
  oligonucleotide
ggcatcatccactgtctct

SEQ ID NO: 1153 - DNA, 20 bp, Artificial Sequence
  oligonucleotide
tgggacaagaaccagatcac

SEQ ID NO: 1154 - DNA, 24 bp, Artificial Sequence
  oligonucleotide
acccagagacagtggatgatgcct

SEQ ID NO: 1155 - DNA, 19 bp, Artificial Sequence
  oligonucleotide
gtcacatcgctccagactt

SEQ ID NO: 1156 - DNA, 22 bp, Artificial Sequence
  oligonucleotide
ggacaagaaccagatcacatac

SEQ ID NO: 1157 - DNA, 24 bp, Artificial Sequence
  oligonucleotide
tgggtccagatcaggtgtgtagcc

SEQ ID NO: 1158 - DNA, 18 bp, Artificial Sequence
  oligonucleotide
cacgagcaaaggcatcat

SEQ ID NO: 1159 - DNA, 27 bp, Artificial Sequence
  oligonucleotide
cacatacaggatcattggctacacacc

SEQ ID NO: 1160 - DNA, 19 bp, Artificial Sequence
  oligonucleotide
gtcacatcgctccagactt

SEQ ID NO: 1161 - DNA, 20 bp, Artificial Sequence
  oligonucleotide
aagtgggacaagaaccagat

SEQ ID NO: 1162 - DNA, 19 bp, Artificial Sequence
  oligonucleotide
ggagacctgagaaccaatc

SEQ ID NO: 1163 - DNA, 19 bp, Artificial Sequence
  oligonucleotide
gactctccacgcatctctg

SEQ ID NO: 1164 - DNA, 23 bp, Artificial Sequence
  oligonucleotide
aggcagctggcagaggaatacct

SEQ ID NO: 1165 - DNA, 17 bp, Artificial Sequence
  oligonucleotide
tccacccttgtgctctt

SEQ ID NO: 1166 - DNA, 23 bp, Artificial Sequence
  oligonucleotide
aaccaatctcaccgacaggcagc

SEQ ID NO: 1167 - DNA, 18 bp, Artificial Sequence
  oligonucleotide
actctccacgcatctctg

SEQ ID NO: 1168 - DNA, 20 bp, Artificial Sequence
  oligonucleotide
agaaccaatctcaccgacag

SEQ ID NO: 1169 - DNA, 23 bp, Artificial Sequence
  oligonucleotide
tggttacactcgggtggcagaga

SEQ ID NO: 1170 - DNA, 21 bp, Artificial Sequence
  oligonucleotide
ccagagatttcgactctccac

SEQ ID NO: 1171 - DNA, 20 bp, Artificial Sequence
  oligonucleotide
tggagacctgagaaccaatc

SEQ ID NO: 1172 - DNA, 23 bp, Artificial Sequence
  oligonucleotide
aggcagctggcagaggaatacct

SEQ ID NO: 1173 - DNA, 19 bp, Artificial Sequence
  oligonucleotide
acccgagtgtaaccatagc

SEQ ID NO: 1174 - DNA, 20 bp, Artificial Sequence
  oligonucleotide
cattcccgaggagctttatg

SEQ ID NO: 1175 - DNA, 24 bp, Artificial Sequence
  oligonucleotide
tgaccactcgatccgctcctttga

SEQ ID NO: 1176 - DNA, 20 bp, Artificial Sequence
  oligonucleotide
gtcatgttcaggtccaactc

SEQ ID NO: 1177 - DNA, 20 bp, Artificial Sequence
  oligonucleotide
cccgaggagctttatgagat

SEQ ID NO: 1178 - DNA, 24 bp, Artificial Sequence
  oligonucleotide
tgaccactcgatccgctcctttga

SEQ ID NO: 1179 - DNA, 18 bp, Artificial Sequence
  oligonucleotide
gggtcatgttcaggtcca

SEQ ID NO: 1180 - DNA, 18 bp, Artificial Sequence
  oligonucleotide
tcgatccgctcctttgat

SEQ ID NO: 1181 - DNA, 23 bp, Artificial Sequence
  oligonucleotide
tgaacatgacccgctcccactct

SEQ ID NO: 1182 - DNA, 17 bp, Artificial Sequence
  oligonucleotide
ccaggctccttcttcca

SEQ ID NO: 1183 - DNA, 20 bp, Artificial Sequence
  oligonucleotide
atccgctcctttgatgatct

SEQ ID NO: 1184 - DNA, 23 bp, Artificial Sequence
  oligonucleotide
tgaacatgacccgctcccactct

SEQ ID NO: 1185 - DNA, 17 bp, Artificial Sequence
  oligonucleotide
ccttcttccacgagcca

SEQ ID NO: 1186 - DNA, 18 bp, Artificial Sequence
  oligonucleotide
tgtctctgtgcccatgat

SEQ ID NO: 1187 - DNA, 20 bp, Artificial Sequence
  oligonucleotide
cagttccaggatgtcgtagt

SEQ ID NO: 1188 - DNA, 19 bp, Artificial Sequence
  oligonucleotide
cgcctcttccacaaatcag

SEQ ID NO: 1189 - DNA, 22 bp, Artificial Sequence
  oligonucleotide
atgggcacagagacagtgctgc

SEQ ID NO: 1190 - DNA, 21 bp, Artificial Sequence
  oligonucleotide
tccaggatgtcgtagtaatgg

SEQ ID NO: 1191 - DNA, 18 bp, Artificial Sequence
  oligonucleotide
tggctcagaccaacaagt

SEQ ID NO: 1192 - DNA, 24 bp, Artificial Sequence
  oligonucleotide
actacgacatcctggaactgccct

SEQ ID NO: 1193 - DNA, 20 bp, Artificial Sequence
  oligonucleotide
cagcaatgaacatgctgagg

SEQ ID NO: 1194 - DNA, 20 bp, Artificial Sequence
  oligonucleotide
gtcgtagtaatggccatcgg

SEQ ID NO: 1195 - DNA, 20 bp, Artificial Sequence
  oligonucleotide
ccacaaatcagacggcagca

SEQ ID NO: 1196 - DNA, 25 bp, Artificial Sequence
  oligonucleotide
cccatgatggctcagaccaacaagt

SEQ ID NO: 1197 - DNA, 22 bp, Artificial Sequence
  oligonucleotide
actgagttcaccacgcccgatg

SEQ ID NO: 1198 - DNA, 19 bp, Artificial Sequence
  oligonucleotide
ggagcctgagcttatgaat

SEQ ID NO: 1199 - DNA, 23 bp, Artificial Sequence
  oligonucleotide
gaagtgaaagtcagatggtttag

SEQ ID NO: 1200 - DNA, 22 bp, Artificial Sequence
  oligonucleotide
ttggtggaggagaagggttggc

SEQ ID NO: 1201 - DNA, 20 bp, Artificial Sequence
  oligonucleotide
tatgaatgccaacccttctc

SEQ ID NO: 1202 - DNA, 19 bp, Artificial Sequence
  oligonucleotide
ccctttccgatcactttca

SEQ ID NO: 1203 - DNA, 22 bp, Artificial Sequence
  oligonucleotide
aatcaaccttggcccgtcgtcc

SEQ ID NO: 1204 - DNA, 20 bp, Artificial Sequence
  oligonucleotide
caggagcctgagcttatgaa

SEQ ID NO: 1205 - DNA, 22 bp, Artificial Sequence
  oligonucleotide
gatggtttagcatgaggattgg

SEQ ID NO: 1206 - DNA, 23 bp, Artificial Sequence
  oligonucleotide
tcagcaaatcaaccttggcccgt

SEQ ID NO: 1207 - DNA, 20 bp, Artificial Sequence
  oligonucleotide
cttgaagatctcccaacctc

SEQ ID NO: 1208 - DNA, 21 bp, Artificial Sequence
  oligonucleotide
caaggttgatttgctgagaag

SEQ ID NO: 1209 - DNA, 22 bp, Artificial Sequence
  oligonucleotide
ttggtggaggagaagggttggc

SEQ ID NO: 1210 - DNA, 23 bp, Artificial Sequence
  oligonucleotide
gcatgagaagtggaaagagaaat

SEQ ID NO: 1211 - DNA, 27 bp, Artificial Sequence
  oligonucleotide
ccaagacagatgcaccatcaggaatgg

SEQ ID NO: 1212 - DNA, 20 bp, Artificial Sequence
  oligonucleotide
tggtcaccttcctgctttat

SEQ ID NO: 1213 - DNA, 29 bp, Artificial Sequence
  oligonucleotide
acagatgcaccatcaggaatggaattaca

SEQ ID NO: 1214 - DNA, 19 bp, Artificial Sequence
  oligonucleotide
ctgagaaacatggtcacct

SEQ ID NO: 1215 - DNA, 23 bp, Artificial Sequence
  oligonucleotide
catgagaagtggaaagagaaatc

SEQ ID NO: 1216 - DNA, 23 bp, Artificial Sequence
  oligonucleotide
ggagaagtttagcatgagaagtg

SEQ ID NO: 1217 - DNA, 27 bp, Artificial Sequence
  oligonucleotide
ccaagacagatgcaccatcaggaatgg

SEQ ID NO: 1218 - DNA, 20 bp, Artificial Sequence
  oligonucleotide
tctgagaaacatggtcacct

SEQ ID NO: 1219 - DNA, 23 bp, Artificial Sequence
  oligonucleotide
gtttagcatgagaagtggaaaga

SEQ ID NO: 1220 - DNA, 27 bp, Artificial Sequence
  oligonucleotide
ccaagacagatgcaccatcaggaatgg

SEQ ID NO: 1221 - DNA, 20 bp, Artificial Sequence
  oligonucleotide
gaaacatggtcaccttcctg

SEQ ID NO: 1222 - DNA, 21 bp, Artificial Sequence
  oligonucleotide
acaaatggagctcatcctagt

SEQ ID NO: 1223 - DNA, 22 bp, Artificial Sequence
  oligonucleotide
tcaggtggctggtcggaaagga

SEQ ID NO: 1224 - DNA, 20 bp, Artificial Sequence
  oligonucleotide
gggcatagatcacatgagga

SEQ ID NO: 1225 - DNA, 24 bp, Artificial Sequence
  oligonucleotide
caaatggagctcatcctagtaaat

SEQ ID NO: 1226 - DNA, 22 bp, Artificial Sequence
  oligonucleotide
tcaggtggctggtcggaaagga

SEQ ID NO: 1227 - DNA, 19 bp, Artificial Sequence
  oligonucleotide
agagacgggcatagatcac

SEQ ID NO: 1228 - DNA, 23 bp, Artificial Sequence
  oligonucleotide
ggagctcatcctagtaaatgtgt

SEQ ID NO: 1229 - DNA, 22 bp, Artificial Sequence
  oligonucleotide
tcaggtggctggtcggaaagga

SEQ ID NO: 1230 - DNA, 18 bp, Artificial Sequence
  oligonucleotide
tccagagacgggcataga

SEQ ID NO: 1231 - DNA, 24 bp, Artificial Sequence
  oligonucleotide
gctcatcctagtaaatgtgttacc

SEQ ID NO: 1232 - DNA, 22 bp, Artificial Sequence
  oligonucleotide
tttccgaccagccacctgaagc

SEQ ID NO: 1233 - DNA, 23 bp, Artificial Sequence
  oligonucleotide
ggcatagatcacatgaggaaatc

SEQ ID NO: 1234 - DNA, 17 bp, Artificial Sequence
  oligonucleotide
agatgctgtgccttcct

SEQ ID NO: 1235 - DNA, 27 bp, Artificial Sequence
  oligonucleotide
agattcccaacttcttctggagcctgg

SEQ ID NO: 1236 - DNA, 17 bp, Artificial Sequence
  oligonucleotide
accagtgtgaccgatcc

SEQ ID NO: 1237 - DNA, 18 bp, Artificial Sequence
  oligonucleotide
ccttcctccgctgaaaca

SEQ ID NO: 1238 - DNA, 22 bp, Artificial Sequence
  oligonucleotide
acactggtgcgtggtggcatac

SEQ ID NO: 1239 - DNA, 18 bp, Artificial Sequence
  oligonucleotide
tctcgtcttctcctccca

SEQ ID NO: 1240 - DNA, 17 bp, Artificial Sequence
  oligonucleotide
gtccagatgctgtgcct

SEQ ID NO: 1241 - DNA, 24 bp, Artificial Sequence
  oligonucleotide
tcccaacttcttctggagcctggg

SEQ ID NO: 1242 - DNA, 18 bp, Artificial Sequence
  oligonucleotide
ctcccagtatgccaccac

SEQ ID NO: 1243 - DNA, 18 bp, Artificial Sequence
  oligonucleotide
accacgcaccagtgtgac

SEQ ID NO: 1244 - DNA, 19 bp, Artificial Sequence
  oligonucleotide
tgccttcctccgctgaaac

SEQ ID NO: 1245 - DNA, 24 bp, Artificial Sequence
  oligonucleotide
tcccaacttcttctggagcctggg

SEQ ID NO: 1246 - DNA, 17 bp, Artificial Sequence
  oligonucleotide
cccacactggcgagaag

SEQ ID NO: 1247 - DNA, 20 bp, Artificial Sequence
  oligonucleotide
ttcgctgaccgctccaacct

SEQ ID NO: 1248 - DNA, 20 bp, Artificial Sequence
  oligonucleotide
ttgacatctgagtgggtctg

SEQ ID NO: 1249 - DNA, 17 bp, Artificial Sequence
  oligonucleotide
catgtccggacccacac

SEQ ID NO: 1250 - DNA, 22 bp, Artificial Sequence
  oligonucleotide
tggcgagaagcccttctcctgt

SEQ ID NO: 1251 - DNA, 20 bp, Artificial Sequence
  oligonucleotide
ggcactggtacttcttgaca

SEQ ID NO: 1252 - DNA, 17 bp, Artificial Sequence
  oligonucleotide
ttctctaggccctggct

SEQ ID NO: 1253 - DNA, 22 bp, Artificial Sequence
  oligonucleotide
tacaaggccatgtccggaccca

SEQ ID NO: 1254 - DNA, 23 bp, Artificial Sequence
  oligonucleotide
ggtacttcttgacatctgagtgg

SEQ ID NO: 1255 - DNA, 17 bp, Artificial Sequence
  oligonucleotide
gccctggctgctacaag

SEQ ID NO: 1256 - DNA, 22 bp, Artificial Sequence
  oligonucleotide
actggcgagaagcccttctcct

SEQ ID NO: 1257 - DNA, 18 bp, Artificial Sequence
  oligonucleotide
ctgagtgggtctggaggt

SEQ ID NO: 1258 - DNA, 20 bp, Artificial Sequence
  oligonucleotide
cccagagagacaccagagaa

SEQ ID NO: 1259 - DNA, 19 bp, Artificial Sequence
  oligonucleotide
gctatcagccacagcaaca

SEQ ID NO: 1260 - DNA, 20 bp, Artificial Sequence
  oligonucleotide
cctggcttctggcatcctgt

SEQ ID NO: 1261 - DNA, 17 bp, Artificial Sequence
  oligonucleotide
catcgccgcagatccag

SEQ ID NO: 1262 - DNA, 24 bp, Artificial Sequence
  oligonucleotide
cccagagagacaccagagaaccca

SEQ ID NO: 1263 - DNA, 18 bp, Artificial Sequence
  oligonucleotide
tcagccacagcaacaaca

SEQ ID NO: 1264 - DNA, 19 bp, Artificial Sequence
  oligonucleotide
agagacaccagagaaccca

SEQ ID NO: 1265 - DNA, 24 bp, Artificial Sequence
  oligonucleotide
tctggcatcctgttgttgctgtgg

SEQ ID NO: 1266 - DNA, 17 bp, Artificial Sequence
  oligonucleotide
gtgggacacaggtgcag

SEQ ID NO: 1267 - DNA, 19 bp, Artificial Sequence
  oligonucleotide
ccagagagacaccagagaa

SEQ ID NO: 1268 - DNA, 24 bp, Artificial Sequence
  oligonucleotide
tctggcatcctgttgttgctgtgg

SEQ ID NO: 1269 - DNA, 19 bp, Artificial Sequence
  oligonucleotide
gaggtcggaattgcagaag

SEQ ID NO: 1270 - DNA, 19 bp, Artificial Sequence
  oligonucleotide
gaggagggcagaatcatca

SEQ ID NO: 1271 - DNA, 28 bp, Artificial Sequence
  oligonucleotide
tgcgctgatagacatccatgaacttcac

SEQ ID NO: 1272 - DNA, 20 bp, Artificial Sequence
  oligonucleotide
tctcgattggatggcagtag

SEQ ID NO: 1273 - DNA, 19 bp, Artificial Sequence
  oligonucleotide
gggcagaatcatcacgaag

SEQ ID NO: 1274 - DNA, 20 bp, Artificial Sequence
  oligonucleotide
gtctcgattggatggcagta

SEQ ID NO: 1275 - DNA, 26 bp, Artificial Sequence
  oligonucleotide
agttcatggatgtctatcagcgcagc

SEQ ID NO: 1276 - DNA, 17 bp, Artificial Sequence
  oligonucleotide
acccatggcagaaggag

SEQ ID NO: 1277 - DNA, 24 bp, Artificial Sequence
  oligonucleotide
agggcagaatcatcacgaagtggt

SEQ ID NO: 1278 - DNA, 19 bp, Artificial Sequence
  oligonucleotide
cagtagctgcgctgataga

SEQ ID NO: 1279 - DNA, 18 bp, Artificial Sequence
  oligonucleotide
gaaggaggagggcagaat

SEQ ID NO: 1280 - DNA, 22 bp, Artificial Sequence
  oligonucleotide
cagcgcagctactgccatccaa

SEQ ID NO: 1281 - DNA, 20 bp, Artificial Sequence
  oligonucleotide
tactcctggaagatgtccac

SEQ ID NO: 1282 - DNA, 18 bp, Artificial Sequence
  oligonucleotide
ccaaccgcgagaagatga

SEQ ID NO: 1283 - DNA, 23 bp, Artificial Sequence
  oligonucleotide
ccatgtacgttgctatccaggct

SEQ ID NO: 1284 - DNA, 20 bp, Artificial Sequence
  oligonucleotide
ccagaggcgtacagggatag

SEQ ID NO: 1285 - DNA, 23 bp, Artificial Sequence
  oligonucleotide
cccagatcatgtttgagaccttc

SEQ ID NO: 1286 - DNA, 22 bp, Artificial Sequence
  oligonucleotide
acgttgctatccaggctgtgct

SEQ ID NO: 1287 - DNA, 19 bp, Artificial Sequence
  oligonucleotide
gtccatcacgatgccagtg

SEQ ID NO: 1288 - DNA, 19 bp, Artificial Sequence
  oligonucleotide
ccaaccgcgagaagatgac

SEQ ID NO: 1289 - DNA, 29 bp, Artificial Sequence
  oligonucleotide
ccagatcatgtttgagaccttcaacaccc

SEQ ID NO: 1290 - DNA, 20 bp, Artificial Sequence
  oligonucleotide
ggatagcacagcctggatag

SEQ ID NO: 1291 - DNA, 21 bp, Artificial Sequence
  oligonucleotide
gatgacccagatcatgtttga

SEQ ID NO: 1292 - DNA, 25 bp, Artificial Sequence
  oligonucleotide
ccatgtacgttgctatccaggctgt

SEQ ID NO: 1293 - DNA, 19 bp, Artificial Sequence
  oligonucleotide
cagaggcgtacagggatag

SEQ ID NO: 1294 - DNA, 22 bp, Artificial Sequence
  oligonucleotide
atgagacagatgctaatggatg

SEQ ID NO: 1295 - DNA, 22 bp, Artificial Sequence
  oligonucleotide
tttagcagcatctgcaacccgc

SEQ ID NO: 1296 - DNA, 20 bp, Artificial Sequence
  oligonucleotide
ttgcttgcacgtagatgtta

SEQ ID NO: 1297 - DNA, 22 bp, Artificial Sequence
  oligonucleotide
caaactcatgagacagatgcta

SEQ ID NO: 1298 - DNA, 22 bp, Artificial Sequence
  oligonucleotide
tttagcagcatctgcaacccgc

SEQ ID NO: 1299 - DNA, 22 bp, Artificial Sequence
  oligonucleotide
gctcattagttcatcacagact

SEQ ID NO: 1300 - DNA, 19 bp, Artificial Sequence
  oligonucleotide
cagccacatcatccctgtg

SEQ ID NO: 1301 - DNA, 27 bp, Artificial Sequence
  oligonucleotide
agcagacataacatctacgtgcaagca

SEQ ID NO: 1302 - DNA, 20 bp, Artificial Sequence
  oligonucleotide
ggcaccgtagggtaattgat

SEQ ID NO: 1303 - DNA, 17 bp, Artificial Sequence
  oligonucleotide
agccacatcatccctgt

SEQ ID NO: 1304 - DNA, 22 bp, Artificial Sequence
  oligonucleotide
tttagcagcatctgcaacccgc

SEQ ID NO: 1305 - DNA, 22 bp, Artificial Sequence
  oligonucleotide
cgtagatgttatgtctgctcat

SEQ ID NO: 1306 - DNA, 22 bp, Artificial Sequence
  oligonucleotide
gtcacagcccaagatagttaag

SEQ ID NO: 1307 - DNA, 24 bp, Artificial Sequence
  oligonucleotide
tcatggaggtttgaagatgccgca

SEQ ID NO: 1308 - DNA, 19 bp, Artificial Sequence
  oligonucleotide
gcaagcaagcagaatttgg

SEQ ID NO: 1309 - DNA, 19 bp, Artificial Sequence
  oligonucleotide
cgtgtgaaccatgtgactt

SEQ ID NO: 1310 - DNA, 28 bp, Artificial Sequence
  oligonucleotide
cacagcccaagatagttaagtgggatcg

SEQ ID NO: 1311 - DNA, 19 bp, Artificial Sequence
  oligonucleotide
cctccatgatgctgcttac

SEQ ID NO: 1312 - DNA, 19 bp, Artificial Sequence
  oligonucleotide
ctttgtcacagcccaagat

SEQ ID NO: 1313 - DNA, 24 bp, Artificial Sequence
  oligonucleotide
tgggatcgagacatgtaagcagca

SEQ ID NO: 1314 - DNA, 22 bp, Artificial Sequence
  oligonucleotide
tggaattcatccaatccaaatg

SEQ ID NO: 1315 - DNA, 19 bp, Artificial Sequence
  oligonucleotide
gtatgcctgccgtgtgaac

SEQ ID NO: 1316 - DNA, 26 bp, Artificial Sequence
  oligonucleotide
aagtgggatcgagacatgtaagcagc

SEQ ID NO: 1317 - DNA, 22 bp, Artificial Sequence
  oligonucleotide
ggcatcttcaaacctccatgat

SEQ ID NO: 1318 - DNA, 19 bp, Artificial Sequence
  oligonucleotide
caaagcagtggacaagaag

SEQ ID NO: 1319 - DNA, 22 bp, Artificial Sequence
  oligonucleotide
tgggcagacttggtgaccttgc

SEQ ID NO: 1320 - DNA, 20 bp, Artificial Sequence
  oligonucleotide
gtggcaggtattagggataa

SEQ ID NO: 1321 - DNA, 21 bp, Artificial Sequence
  oligonucleotide
ctttgctgttcgtgatatgag

SEQ ID NO: 1322 - DNA, 22 bp, Artificial Sequence
  oligonucleotide
tgcggtgggtgtcatcaaagca

SEQ ID NO: 1323 - DNA, 21 bp, Artificial Sequence
  oligonucleotide
catttagccttctgagctttc

SEQ ID NO: 1324 - DNA, 21 bp, Artificial Sequence
  oligonucleotide
gaaagctcagaaggctaaatg

SEQ ID NO: 1325 - DNA, 26 bp, Artificial Sequence
  oligonucleotide
tcagtggtggaagaacggtctcagaa

SEQ ID NO: 1326 - DNA, 19 bp, Artificial Sequence
  oligonucleotide
tggccaattgaaacaaaca

SEQ ID NO: 1327 - DNA, 21 bp, Artificial Sequence
  oligonucleotide
gttcgtgatatgagacagaca

SEQ ID NO: 1328 - DNA, 22 bp, Artificial Sequence
  oligonucleotide
tgcggtgggtgtcatcaaagca

SEQ ID NO: 1329 - DNA, 25 bp, Artificial Sequence
  oligonucleotide
gggataatattcatttagccttctg

SEQ ID NO: 1330 - DNA, 21 bp, Artificial Sequence
  oligonucleotide
caagtacatcatccgagacaa

SEQ ID NO: 1331 - DNA, 23 bp, Artificial Sequence
  oligonucleotide
tcgcattgacttgcgtttccacc

SEQ ID NO: 1332 - DNA, 19 bp, Artificial Sequence
  oligonucleotide
gtgccgttccaccttatag

SEQ ID NO: 1333 - DNA, 19 bp, Artificial Sequence
  oligonucleotide
atggtgatcgcattgactt

SEQ ID NO: 1334 - DNA, 22 bp, Artificial Sequence
  oligonucleotide
accggctataaggtggaacggc

SEQ ID NO: 1335 - DNA, 19 bp, Artificial Sequence
  oligonucleotide
ctgccggttgaagataaca

SEQ ID NO: 1336 - DNA, 19 bp, Artificial Sequence
  oligonucleotide
tcgcattgacttgcgtttc

SEQ ID NO: 1337 - DNA, 22 bp, Artificial Sequence
  oligonucleotide
ccaagcccagtgaccttcacct

SEQ ID NO: 1338 - DNA, 19 bp, Artificial Sequence
  oligonucleotide
catcacacatgtgccgttc

SEQ ID NO: 1339 - DNA, 19 bp, Artificial Sequence
  oligonucleotide
cattgacttgcgtttccac

SEQ ID NO: 1340 - DNA, 25 bp, Artificial Sequence
  oligonucleotide
ttcacctgcagaccggctataaggt

SEQ ID NO: 1341 - DNA, 22 bp, Artificial Sequence
  oligonucleotide
taacaatgtccccatcacacat

SEQ ID NO: 1342 - DNA, 22 bp, Artificial Sequence
  oligonucleotide
gcttgtcttcaatgaaatcctc

SEQ ID NO: 1343 - DNA, 23 bp, Artificial Sequence
  oligonucleotide
tccaccatgagttggtaggcagc

SEQ ID NO: 1344 - DNA, 21 bp, Artificial Sequence
  oligonucleotide
ctgttcaagactgccaaattc

SEQ ID NO: 1345 - DNA, 19 bp, Artificial Sequence
  oligonucleotide
ccaactcatggtggatgtg

SEQ ID NO: 1346 - DNA, 22 bp, Artificial Sequence
  oligonucleotide
aatccgttctgccaaagccagc

SEQ ID NO: 1347 - DNA, 22 bp, Artificial Sequence
  oligonucleotide
catacatctgtagtgccaatga

SEQ ID NO: 1348 - DNA, 22 bp, Artificial Sequence
  oligonucleotide
gccagcttgtcttcaatgaaat

SEQ ID NO: 1349 - DNA, 24 bp, Artificial Sequence
  oligonucleotide
atccaccatgagttggtaggcagc

SEQ ID NO: 1350 - DNA, 21 bp, Artificial Sequence
  oligonucleotide
caaagccagcttctgttcaag

SEQ ID NO: 1351 - DNA, 22 bp, Artificial Sequence
  oligonucleotide
catggtggatgtgtttggtaat

SEQ ID NO: 1352 - DNA, 24 bp, Artificial Sequence
  oligonucleotide
ttggcagtcttgaacagaagctgg

SEQ ID NO: 1353 - DNA, 19 bp, Artificial Sequence
  oligonucleotide
cgaatccgttctgccaaag

SEQ ID NO: 1354 - DNA, 20 bp, Artificial Sequence
  oligonucleotide
caaccctgaagtgcttgata

SEQ ID NO: 1355 - DNA, 22 bp, Artificial Sequence
  oligonucleotide
tgcattctcgcttcctggaggg

SEQ ID NO: 1356 - DNA, 22 bp, Artificial Sequence
  oligonucleotide
gtttgtacccgttgatgataga

SEQ ID NO: 1357 - DNA, 21 bp, Artificial Sequence
  oligonucleotide
cacagaggaaactctgcattc

SEQ ID NO: 1358 - DNA, 23 bp, Artificial Sequence
  oligonucleotide
agggtgtccgcaatgttgccagt

SEQ ID NO: 1359 - DNA, 19 bp, Artificial Sequence
  oligonucleotide
gatgcaacagttgggtagc

SEQ ID NO: 1360 - DNA, 19 bp, Artificial Sequence
  oligonucleotide
gacaatggcagcatctaca

SEQ ID NO: 1361 - DNA, 22 bp, Artificial Sequence
  oligonucleotide
tgcattctcgcttcctggaggg

SEQ ID NO: 1362 - DNA, 19 bp, Artificial Sequence
  oligonucleotide
ccaatctgcagacagacac

SEQ ID NO: 1363 - DNA, 20 bp, Artificial Sequence
  oligonucleotide
cagcatctacaaccctgaag

SEQ ID NO: 1364 - DNA, 22 bp, Artificial Sequence
  oligonucleotide
tgcattctcgcttcctggaggg

SEQ ID NO: 1365 - DNA, 19 bp, Artificial Sequence
  oligonucleotide
gacagacactggcaacatt

SEQ ID NO: 1366 - DNA, 19 bp, Artificial Sequence
  oligonucleotide
aagggattcaggaagacga

SEQ ID NO: 1367 - DNA, 24 bp, Artificial Sequence
  oligonucleotide
aatggctctcatgtacccttgcct

SEQ ID NO: 1368 - DNA, 21 bp, Artificial Sequence
  oligonucleotide
ttctcacaacaccaccattta

SEQ ID NO: 1369 - DNA, 22 bp, Artificial Sequence
  oligonucleotide
ccctattctaaagggattcagg

SEQ ID NO: 1370 - DNA, 24 bp, Artificial Sequence
  oligonucleotide
aatggctctcatgtacccttgcct

SEQ ID NO: 1371 - DNA, 21 bp, Artificial Sequence
  oligonucleotide
actcaacatccatcttctcac

SEQ ID NO: 1372 - DNA, 22 bp, Artificial Sequence
  oligonucleotide
gattcaggaagacgacgtaatg

SEQ ID NO: 1373 - DNA, 22 bp, Artificial Sequence
  oligonucleotide
ctctcatgtacccttgcctccc

SEQ ID NO: 1374 - DNA, 22 bp, Artificial Sequence
  oligonucleotide
accaccatttaaaggtaccaaa

SEQ ID NO: 1375 - DNA, 20 bp, Artificial Sequence
  oligonucleotide
cgtaatggctctcatgtacc

SEQ ID NO: 1376 - DNA, 29 bp, Artificial Sequence
  oligonucleotide
tggtacctttaaatggtggtgttgtgaga

SEQ ID NO: 1377 - DNA, 19 bp, Artificial Sequence
  oligonucleotide
cctgcaactcaacatccat

SEQ ID NO: 1378 - DNA, 23 bp, Artificial Sequence
  oligonucleotide
gaacagagaccagaaagagtaaa

SEQ ID NO: 1379 - DNA, 24 bp, Artificial Sequence
  oligonucleotide
tgtgcttgatttgttctgcagccc

SEQ ID NO: 1380 - DNA, 20 bp, Artificial Sequence
  oligonucleotide
tcctcacggtagtccaatag

SEQ ID NO: 1381 - DNA, 22 bp, Artificial Sequence
  oligonucleotide
gggaaacttgaagaacagagac

SEQ ID NO: 1382 - DNA, 24 bp, Artificial Sequence
  oligonucleotide
tgtgcttgatttgttctgcagccc

SEQ ID NO: 1383 - DNA, 20 bp, Artificial Sequence
  oligonucleotide
caaccatgccatctggattc

SEQ ID NO: 1384 - DNA, 22 bp, Artificial Sequence
  oligonucleotide
caagcacatccttgctaatttc

SEQ ID NO: 1385 - DNA, 25 bp, Artificial Sequence
  oligonucleotide
tgaatccagatggcatggttgctct

SEQ ID NO: 1386 - DNA, 19 bp, Artificial Sequence
  oligonucleotide
cacaccatcctcacggtag

SEQ ID NO: 1387 - DNA, 23 bp, Artificial Sequence
  oligonucleotide
gcctacaagaagtacatcaaaga

SEQ ID NO: 1388 - DNA, 27 bp, Artificial Sequence
  oligonucleotide
agggaaacttgaagaacagagaccaga

SEQ ID NO: 1389 - DNA, 20 bp, Artificial Sequence
  oligonucleotide
gcaaggatgtgcttgatttg

SEQ ID NO: 1390 - DNA, 22 bp, Artificial Sequence
  oligonucleotide
tgactccttcaacaccttcttc

SEQ ID NO: 1391 - DNA, 24 bp, Artificial Sequence
  oligonucleotide
ccgggctgtgtttgtagacttgga

SEQ ID NO: 1392 - DNA, 20 bp, Artificial Sequence
  oligonucleotide
ccagtgcgaacttcatcaat

SEQ ID NO: 1393 - DNA, 21 bp, Artificial Sequence
  oligonucleotide
gactccttcaacaccttcttc

SEQ ID NO: 1394 - DNA, 24 bp, Artificial Sequence
  oligonucleotide
ccgggctgtgtttgtagacttgga

SEQ ID NO: 1395 - DNA, 20 bp, Artificial Sequence
  oligonucleotide
ccagtgcgaacttcatcaat

SEQ ID NO: 1396 - DNA, 20 bp, Artificial Sequence
  oligonucleotide
ggaggagatgactccttcaa

SEQ ID NO: 1397 - DNA, 22 bp, Artificial Sequence
  oligonucleotide
cttcttcagtgagacgggcgct

SEQ ID NO: 1398 - DNA, 20 bp, Artificial Sequence
  oligonucleotide
catcaatgactgtgggttcc

SEQ ID NO: 1399 - DNA, 19 bp, Artificial Sequence
  oligonucleotide
cagatgccaagtgacaaga

SEQ ID NO: 1400 - DNA, 22 bp, Artificial Sequence
  oligonucleotide
cttcttcagtgagacgggcgct

SEQ ID NO: 1401 - DNA, 20 bp, Artificial Sequence
  oligonucleotide
gggttccaagtctacaaaca

SEQ ID NO: 1402 - DNA, 19 bp, Artificial Sequence
  oligonucleotide
gagcacacagaatctgcta

SEQ ID NO: 1403 - DNA, 19 bp, Artificial Sequence
  oligonucleotide
aggttgtccagagctttac

SEQ ID NO: 1404 - DNA, 22 bp, Artificial Sequence
  oligonucleotide
cggattcgtggctgctgctgaa

SEQ ID NO: 1405 - DNA, 19 bp, Artificial Sequence
  oligonucleotide
acacagaatctgctactcc

SEQ ID NO: 1406 - DNA, 24 bp, Artificial Sequence
  oligonucleotide
cagttcagcagcagccacgaatcc

SEQ ID NO: 1407 - DNA, 18 bp, Artificial Sequence
  oligonucleotide
agctcatctgcctcattc

SEQ ID NO: 1408 - DNA, 17 bp, Artificial Sequence
  oligonucleotide
aggcctggaacggattc

SEQ ID NO: 1409 - DNA, 25 bp, Artificial Sequence
  oligonucleotide
agctccgtaaagctctggacaacct

SEQ ID NO: 1410 - DNA, 24 bp, Artificial Sequence
  oligonucleotide
gtctttcatgatcatttgtcttgc

SEQ ID NO: 1411 - DNA, 20 bp, Artificial Sequence
  oligonucleotide
actcctgctgactgataatg

SEQ ID NO: 1412 - DNA, 24 bp, Artificial Sequence
  oligonucleotide
ctcattcctgggcagttcagcagc

SEQ ID NO: 1413 - DNA, 19 bp, Artificial Sequence
  oligonucleotide
aggttgtccagagctttac

SEQ ID NO: 1414 - DNA, 18 bp, Artificial Sequence
  oligonucleotide
cagaacctacgcacctac

SEQ ID NO: 1415 - DNA, 25 bp, Artificial Sequence
  oligonucleotide
ccgttcagtccatcccatttctggc

SEQ ID NO: 1416 - DNA, 19 bp, Artificial Sequence
  oligonucleotide
tgaccacatcgagctttag

SEQ ID NO: 1417 - DNA, 20 bp, Artificial Sequence
  oligonucleotide
cgtgatgtctttcacacaac

SEQ ID NO: 1418 - DNA, 25 bp, Artificial Sequence
  oligonucleotide
ccgttcagtccatcccatttctggc

SEQ ID NO: 1419 - DNA, 19 bp, Artificial Sequence
  oligonucleotide
ttagccagtcacacttctg

SEQ ID NO: 1420 - DNA, 18 bp, Artificial Sequence
  oligonucleotide
acctacgtgaggagctta

SEQ ID NO: 1421 - DNA, 25 bp, Artificial Sequence
  oligonucleotide
ccgttcagtccatcccatttctggc

SEQ ID NO: 1422 - DNA, 18 bp, Artificial Sequence
  oligonucleotide
gctatacagctgtgacca

SEQ ID NO: 1423 - DNA, 17 bp, Artificial Sequence
  oligonucleotide
cgtccttgctccgtgat

SEQ ID NO: 1424 - DNA, 25 bp, Artificial Sequence
  oligonucleotide
aaccagaacctacgcacctacgtga

SEQ ID NO: 1425 - DNA, 18 bp, Artificial Sequence
  oligonucleotide
aactgtccgttcagtcca

SEQ ID NO: 1426 - DNA, 20 bp, Artificial Sequence
  oligonucleotide
catcttgctggttctgattg

SEQ ID NO: 1427 - DNA, 24 bp, Artificial Sequence
  oligonucleotide
ttcctgcatcagcaccaaccaagg

SEQ ID NO: 1428 - DNA, 21 bp, Artificial Sequence
  oligonucleotide
caaggattgtaggtggatagt

SEQ ID NO: 1429 - DNA, 18 bp, Artificial Sequence
  oligonucleotide
tctgattggagtgcaagg

SEQ ID NO: 1430 - DNA, 24 bp, Artificial Sequence
  oligonucleotide
ttcctgcatcagcaccaaccaagg

SEQ ID NO: 1431 - DNA, 21 bp, Artificial Sequence
  oligonucleotide
aggtctttcaaggattgtagg

SEQ ID NO: 1432 - DNA, 19 bp, Artificial Sequence
  oligonucleotide
agggactatccacctacaa

SEQ ID NO: 1433 - DNA, 21 bp, Artificial Sequence
  oligonucleotide
gacatgtttgaactccattct

SEQ ID NO: 1434 - DNA, 22 bp, Artificial Sequence
  oligonucleotide
cccaagcccttcctgcgagaaa

SEQ ID NO: 1435 - DNA, 22 bp, Artificial Sequence
  oligonucleotide
gaaagaccttaaacaatttgcc

SEQ ID NO: 1436 - DNA, 21 bp, Artificial Sequence
  oligonucleotide
ccaagcccttcctgcgagaaa

SEQ ID NO: 1437 - DNA, 19 bp, Artificial Sequence
  oligonucleotide
tcagttccttcacatctgc

SEQ ID NO: 1438 - DNA, 20 bp, Artificial Sequence
  oligonucleotide
gttcagaagctacaagacct

SEQ ID NO: 1439 - DNA, 28 bp, Artificial Sequence
  oligonucleotide
tctgcagaatctcttcagcctgtatccc

SEQ ID NO: 1440 - DNA, 22 bp, Artificial Sequence
  oligonucleotide
agtcatagactccttggatttc

SEQ ID NO: 1441 - DNA, 20 bp, Artificial Sequence
  oligonucleotide
ggcgggaatttattcgaaac

SEQ ID NO: 1442 - DNA, 30 bp, Artificial Sequence
  oligonucleotide
tgtagcttctgaacaaagagacgatagccc

SEQ ID NO: 1443 - DNA, 22 bp, Artificial Sequence
  oligonucleotide
ccttggatttcaagtatgtctg

SEQ ID NO: 1444 - DNA, 20 bp, Artificial Sequence
  oligonucleotide
agctacaagacctgaagaaa

SEQ ID NO: 1445 - DNA, 26 bp, Artificial Sequence
  oligonucleotide
aagtactatgaggaaccgaggaaggg

SEQ ID NO: 1446 - DNA, 21 bp, Artificial Sequence
  oligonucleotide
agaatctcttcagcctgtatc

SEQ ID NO: 1447 - DNA, 18 bp, Artificial Sequence
  oligonucleotide
actatgaggaaccgagga

SEQ ID NO: 1448 - DNA, 21 bp, Artificial Sequence
  oligonucleotide
ggagaattgcatcagtcatag

SEQ ID NO: 1449 - DNA, 28 bp, Artificial Sequence
  oligonucleotide
atacaggctgaagagattctgcagacat

SEQ ID NO: 1450 - DNA, 17 bp, Artificial Sequence
  oligonucleotide
gaggtgaagggctggat

SEQ ID NO: 1451 - DNA, 20 bp, Artificial Sequence
  oligonucleotide
tccaaccctccagccactca

SEQ ID NO: 1452 - DNA, 17 bp, Artificial Sequence
  oligonucleotide
gttgctgtggcgatgtt

SEQ ID NO: 1453 - DNA, 17 bp, Artificial Sequence
  oligonucleotide
gcagatgctctgtgctg

SEQ ID NO: 1454 - DNA, 22 bp, Artificial Sequence
  oligonucleotide
actgtgcatccagcccttcacc

SEQ ID NO: 1455 - DNA, 17 bp, Artificial Sequence
  oligonucleotide
ggcgatgttgctgagtg

SEQ ID NO: 1456 - DNA, 17 bp, Artificial Sequence
  oligonucleotide
ctggatgcacagtggga

SEQ ID NO: 1457 - DNA, 22 bp, Artificial Sequence
  oligonucleotide
tccagccactcagcaacatcgc

SEQ ID NO: 1458 - DNA, 17 bp, Artificial Sequence
  oligonucleotide
ggttgctggttgctgtg

SEQ ID NO: 1459 - DNA, 17 bp, Artificial Sequence
  oligonucleotide
caaccctccagccactc

SEQ ID NO: 1460 - DNA, 22 bp, Artificial Sequence
  oligonucleotide
acagcaaccagcaaccagacgg

SEQ ID NO: 1461 - DNA, 17 bp, Artificial Sequence
  oligonucleotide
ccgtccgcttgtgtttg

SEQ ID NO: 1462 - DNA, 19 bp, Artificial Sequence
  oligonucleotide
ctagcctcaagtgatcctc

SEQ ID NO: 1463 - DNA, 24 bp, Artificial Sequence
  oligonucleotide
atcgtcggcgcatgcttgtaatcc

SEQ ID NO: 1464 - DNA, 20 bp, Artificial Sequence
  oligonucleotide
gggagagtgatagacaaagt

SEQ ID NO: 1465 - DNA, 19 bp, Artificial Sequence
  oligonucleotide
ctagagtgcagtggctatt

SEQ ID NO: 1466 - DNA, 17 bp, Artificial Sequence
  oligonucleotide
ggcgcatgcttgtaatc

SEQ ID NO: 1467 - DNA, 22 bp, Artificial Sequence
  oligonucleotide
tgcagcctccaactcctagcct

SEQ ID NO: 1468 - DNA, 21 bp, Artificial Sequence
  oligonucleotide
cctcccaagtaggattacaag

SEQ ID NO: 1469 - DNA, 24 bp, Artificial Sequence
  oligonucleotide
ccgacgatgcccagaatccagaac

SEQ ID NO: 1470 - DNA, 22 bp, Artificial Sequence
  oligonucleotide
ctgggtgaagtttattctgttt

SEQ ID NO: 1471 - DNA, 22 bp, Artificial Sequence
  oligonucleotide
cactatattgtccaggctagag

SEQ ID NO: 1472 - DNA, 24 bp, Artificial Sequence
  oligonucleotide
agtacactgcagcctccaactcct

SEQ ID NO: 1473 - DNA, 19 bp, Artificial Sequence
  oligonucleotide
gttgagacaggaggatcac

SEQ ID NO: 1474 - DNA, 17 bp, Artificial Sequence
  oligonucleotide
gagtgcagctgcctgaa

SEQ ID NO: 1475 - DNA, 18 bp, Artificial Sequence
  oligonucleotide
ggctgctctccaaggaat

SEQ ID NO: 1476 - DNA, 23 bp, Artificial Sequence
  oligonucleotide
aattctcagctgttcggcagggc

SEQ ID NO: 1477 - DNA, 22 bp, Artificial Sequence
  oligonucleotide
agaggaagatttctgaagagtg

SEQ ID NO: 1478 - DNA, 24 bp, Artificial Sequence
  oligonucleotide
tgccgaacagctgagaattgcact

SEQ ID NO: 1479 - DNA, 24 bp, Artificial Sequence
  oligonucleotide
ctccaaggaattcttattgttctc

SEQ ID NO: 1480 - DNA, 22 bp, Artificial Sequence
  oligonucleotide
aaccatgagtgagaacaataag

SEQ ID NO: 1481 - DNA, 24 bp, Artificial Sequence
  oligonucleotide
tggagagcagcctacggcaactaa

SEQ ID NO: 1482 - DNA, 20 bp, Artificial Sequence
  oligonucleotide
cacgattctgaaactcagtc

SEQ ID NO: 1483 - DNA, 17 bp, Artificial Sequence
  oligonucleotide
gccgaacagctgagaat

SEQ ID NO: 1484 - DNA, 24 bp, Artificial Sequence
  oligonucleotide
tggagagcagcctacggcaactaa

SEQ ID NO: 1485 - DNA, 21 bp, Artificial Sequence
  oligonucleotide
catcaagttccaggtgaaatg

SEQ ID NO: 1486 - DNA, 20 bp, Artificial Sequence
  oligonucleotide
ttcatagcattcgcctactc

SEQ ID NO: 1487 - DNA, 24 bp, Artificial Sequence
  oligonucleotide
tagggacaggaagatggttggcga

SEQ ID NO: 1488 - DNA, 18 bp, Artificial Sequence
  oligonucleotide
agatgttcaggcacttgg

SEQ ID NO: 1489 - DNA, 19 bp, Artificial Sequence
  oligonucleotide
acaccctcttcttgaactg

SEQ ID NO: 1490 - DNA, 25 bp, Artificial Sequence
  oligonucleotide
agcattcgcctactccgtgaagtct

SEQ ID NO: 1491 - DNA, 17 bp, Artificial Sequence
  oligonucleotide
gccaaccatcttcctgt

SEQ ID NO: 1492 - DNA, 20 bp, Artificial Sequence
  oligonucleotide
cttcttgaactggtgctgtc

SEQ ID NO: 1493 - DNA, 24 bp, Artificial Sequence
  oligonucleotide
tagggacaggaagatggttggcga

SEQ ID NO: 1494 - DNA, 17 bp, Artificial Sequence
  oligonucleotide
agggcccagatgttcag

SEQ ID NO: 1495 - DNA, 18 bp, Artificial Sequence
  oligonucleotide
cctgttcaacaccctctt

SEQ ID NO: 1496 - DNA, 18 bp, Artificial Sequence
  oligonucleotide
ctgtccctagacttcacg

SEQ ID NO: 1497 - DNA, 26 bp, Artificial Sequence
  oligonucleotide
tctgggcttcatagcattcgcctact

SEQ ID NO: 1498 - DNA, 18 bp, Artificial Sequence
  oligonucleotide
taagagcaaggccaagag

SEQ ID NO: 1499 - DNA, 24 bp, Artificial Sequence
  oligonucleotide
tgatggactcagcagctccactct

SEQ ID NO: 1500 - DNA, 19 bp, Artificial Sequence
  oligonucleotide
gtagcctggaactgtgtag

SEQ ID NO: 1501 - DNA, 18 bp, Artificial Sequence
  oligonucleotide
ccacctctcaccaagaac

SEQ ID NO: 1502 - DNA, 18 bp, Artificial Sequence
  oligonucleotide
ggtatcagggctggaatc

SEQ ID NO: 1503 - DNA, 23 bp, Artificial Sequence
  oligonucleotide
agtcgaagtccagccgagatgct

SEQ ID NO: 1504 - DNA, 17 bp, Artificial Sequence
  oligonucleotide
atgcttccacctctcac

SEQ ID NO: 1505 - DNA, 24 bp, Artificial Sequence
  oligonucleotide
aagtcgaagtccagccgagatgct

SEQ ID NO: 1506 - DNA, 18 bp, Artificial Sequence
  oligonucleotide
cccacatgacttcctctt

SEQ ID NO: 1507 - DNA, 18 bp, Artificial Sequence
  oligonucleotide
aaagaccagagcaggaac

SEQ ID NO: 1508 - DNA, 25 bp, Artificial Sequence
  oligonucleotide
tgcttccacctctcaccaagaacca

SEQ ID NO: 1509 - DNA, 18 bp, Artificial Sequence
  oligonucleotide
gctggacttcgactttct

SEQ ID NO: 1510 - DNA, 18 bp, Artificial Sequence
  oligonucleotide
taccatctacctgggctt

SEQ ID NO: 1511 - DNA, 17 bp, Artificial Sequence
  oligonucleotide
cagggttccagcttcac

SEQ ID NO: 1512 - DNA, 22 bp, Artificial Sequence
  oligonucleotide
ccaaggagaagagcctggtcct

SEQ ID NO: 1513 - DNA, 17 bp, Artificial Sequence
  oligonucleotide
aaggagaagagcctggt

SEQ ID NO: 1514 - DNA, 22 bp, Artificial Sequence
  oligonucleotide
agcgtgagggtgtgtcttccct

SEQ ID NO: 1515 - DNA, 17 bp, Artificial Sequence
  oligonucleotide
ctgaggctgctgctatc

SEQ ID NO: 1516 - DNA, 17 bp, Artificial Sequence
  oligonucleotide
gaggcccaaggagaaga

SEQ ID NO: 1517 - DNA, 22 bp, Artificial Sequence
  oligonucleotide
acagccagggttccagcttcac

SEQ ID NO: 1518 - DNA, 17 bp, Artificial Sequence
  oligonucleotide
tgctgctatccagggaa

SEQ ID NO: 1519 - DNA, 19 bp, Artificial Sequence
  oligonucleotide
cccacgctataccatctac

SEQ ID NO: 1520 - DNA, 24 bp, Artificial Sequence
  oligonucleotide
ttcaccaggaccaggctcttctcc

SEQ ID NO: 1521 - DNA, 19 bp, Artificial Sequence
  oligonucleotide
ctatccagggaagacacac

SEQ ID NO: 1522 - DNA, 19 bp, Artificial Sequence
  oligonucleotide
gagccctacaaggtgtatc

SEQ ID NO: 1523 - DNA, 25 bp, Artificial Sequence
  oligonucleotide
ccagccagggactcagaaagtacca

SEQ ID NO: 1524 - DNA, 18 bp, Artificial Sequence
  oligonucleotide
atcctcttcctccttcct

SEQ ID NO: 1525 - DNA, 17 bp, Artificial Sequence
  oligonucleotide
cgcatggatgttgctga

SEQ ID NO: 1526 - DNA, 20 bp, Artificial Sequence
  oligonucleotide
tgatggtactttctgagtcc

SEQ ID NO: 1527 - DNA, 23 bp, Artificial Sequence
  oligonucleotide
cagttgctgccaccaggaatcgt

SEQ ID NO: 1528 - DNA, 18 bp, Artificial Sequence
  oligonucleotide
caccaggaatcgtctctg

SEQ ID NO: 1529 - DNA, 25 bp, Artificial Sequence
  oligonucleotide
ccagccagggactcagaaagtacca

SEQ ID NO: 1530 - DNA, 18 bp, Artificial Sequence
  oligonucleotide
gactgagtgtgcagttct

SEQ ID NO: 1531 - DNA, 17 bp, Artificial Sequence
  oligonucleotide
atgttgctgagccctac

SEQ ID NO: 1532 - DNA, 24 bp, Artificial Sequence
  oligonucleotide
atcagttgctgccaccaggaatcg

SEQ ID NO: 1533 - DNA, 20 bp, Artificial Sequence
  oligonucleotide
cgctttgatggtactttctg

SEQ ID NO: 1534 - DNA, 17 bp, Artificial Sequence
  oligonucleotide
aatgcgacgaacctctg

SEQ ID NO: 1535 - DNA, 24 bp, Artificial Sequence
  oligonucleotide
ctgctgcggcccttgttattcctc

SEQ ID NO: 1536 - DNA, 17 bp, Artificial Sequence
  oligonucleotide
tcacttgctgcttcagg

SEQ ID NO: 1537 - DNA, 17 bp, Artificial Sequence
  oligonucleotide
tgggacctgacggtgaa

SEQ ID NO: 1538 - DNA, 19 bp, Artificial Sequence
  oligonucleotide
cgatcttctgggtgatctg

SEQ ID NO: 1539 - DNA, 22 bp, Artificial Sequence
  oligonucleotide
ttccaggtgtccctgagcagct

SEQ ID NO: 1540 - DNA, 19 bp, Artificial Sequence
  oligonucleotide
gcatcctggtgaggaataa

SEQ ID NO: 1541 - DNA, 17 bp, Artificial Sequence
  oligonucleotide
agccagaacaggtcgtc

SEQ ID NO: 1542 - DNA, 22 bp, Artificial Sequence
  oligonucleotide
acctgaagcagcaagtgagcgg

SEQ ID NO: 1543 - DNA, 19 bp, Artificial Sequence
  oligonucleotide
cagatcacccagaagatcg

SEQ ID NO: 1544 - DNA, 20 bp, Artificial Sequence
  oligonucleotide
ttccagcagcgtctggctgt

SEQ ID NO: 1545 - DNA, 17 bp, Artificial Sequence
  oligonucleotide
ttcgtcgcatttgtcca

SEQ ID NO: 1546 - DNA, 18 bp, Artificial Sequence
  oligonucleotide
tgactgtgtctgctagtg

SEQ ID NO: 1547 - DNA, 22 bp, Artificial Sequence
  oligonucleotide
acatttggccacagcctgagca

SEQ ID NO: 1548 - DNA, 18 bp, Artificial Sequence
  oligonucleotide
caacattgacgccttctg

SEQ ID NO: 1549 - DNA, 17 bp, Artificial Sequence
  oligonucleotide
tccgaccaggacaacta

SEQ ID NO: 1550 - DNA, 22 bp, Artificial Sequence
  oligonucleotide
acatttggccacagcctgagca

SEQ ID NO: 1551 - DNA, 17 bp, Artificial Sequence
  oligonucleotide
agccacaccaacattga

SEQ ID NO: 1552 - DNA, 18 bp, Artificial Sequence
  oligonucleotide
gacaactactgcgtgact

SEQ ID NO: 1553 - DNA, 22 bp, Artificial Sequence
  oligonucleotide
tcacgagattcccaatgccggc

SEQ ID NO: 1554 - DNA, 17 bp, Artificial Sequence
  oligonucleotide
aacaggtcttgctcagg

SEQ ID NO: 1555 - DNA, 19 bp, Artificial Sequence
  oligonucleotide
gcaatctgtactgcctgaa

SEQ ID NO: 1556 - DNA, 23 bp, Artificial Sequence
  oligonucleotide
tccgaccaggacaactactgcgt

SEQ ID NO: 1557 - DNA, 17 bp, Artificial Sequence
  oligonucleotide
tggccaaatgtcacgag

SEQ ID NO: 1558 - DNA, 17 bp, Artificial Sequence
  oligonucleotide
cctgtgtgtgtgtccaa

SEQ ID NO: 1559 - DNA, 17 bp, Artificial Sequence
  oligonucleotide
ccaggtcagcgtcagat

SEQ ID NO: 1560 - DNA, 22 bp, Artificial Sequence
  oligonucleotide
aaagggtggctcctcaggcaag

SEQ ID NO: 1561 - DNA, 19 bp, Artificial Sequence
  oligonucleotide
ccatgccattgacatcatc

SEQ ID NO: 1562 - DNA, 25 bp, Artificial Sequence
  oligonucleotide
tcctaccctgtgtgtgtgtccaagg

SEQ ID NO: 1563 - DNA, 17 bp, Artificial Sequence
  oligonucleotide
gaggagccaccctttac

SEQ ID NO: 1564 - DNA, 19 bp, Artificial Sequence
  oligonucleotide
tgacatcatctgtgggttc

SEQ ID NO: 1565 - DNA, 23 bp, Artificial Sequence
  oligonucleotide
aggtggtaaagggtggctcctca

SEQ ID NO: 1566 - DNA, 18 bp, Artificial Sequence
  oligonucleotide
ggaagacaaccaggtcag

SEQ ID NO: 1567 - DNA, 19 bp, Artificial Sequence
  oligonucleotide
tgtgtccaaggtggtaaag

SEQ ID NO: 1568 - DNA, 25 bp, Artificial Sequence
  oligonucleotide
cgatctgacgctgacctggttgtct

SEQ ID NO: 1569 - DNA, 18 bp, Artificial Sequence
  oligonucleotide
aagtggtgagaggactga

SEQ ID NO: 1570 - DNA, 20 bp, Artificial Sequence
  oligonucleotide
ccaggatggttcttagactc

SEQ ID NO: 1571 - DNA, 22 bp, Artificial Sequence
  oligonucleotide
tgcaggtgaaggtggcgttgtc

SEQ ID NO: 1572 - DNA, 18 bp, Artificial Sequence
  oligonucleotide
tccgatgtgttggagaag

SEQ ID NO: 1573 - DNA, 17 bp, Artificial Sequence
  oligonucleotide
cctgagcagtggagaag

SEQ ID NO: 1574 - DNA, 22 bp, Artificial Sequence
  oligonucleotide
tcgtctgggcggtgctacaact

SEQ ID NO: 1575 - DNA, 20 bp, Artificial Sequence
  oligonucleotide
ggagtctaagaaccatcctg

SEQ ID NO: 1576 - DNA, 17 bp, Artificial Sequence
  oligonucleotide
tccaggcatgcagatcc

SEQ ID NO: 1577 - DNA, 22 bp, Artificial Sequence
  oligonucleotide
tcgtctgggcggtgctacaact

SEQ ID NO: 1578 - DNA, 17 bp, Artificial Sequence
  oligonucleotide
gttccagggcctgtctg

SEQ ID NO: 1579 - DNA, 17 bp, Artificial Sequence
  oligonucleotide
tctgggcggtgctacaa

SEQ ID NO: 1580 - DNA, 23 bp, Artificial Sequence
  oligonucleotide
tggcggccaggatggttcttaga

SEQ ID NO: 1581 - DNA, 19 bp, Artificial Sequence
  oligonucleotide
tggagaagctgcaggtgaa

SEQ ID NO: 1582 - DNA, 20 bp, Artificial Sequence
  oligonucleotide
ctacagatgaacccaaggat

SEQ ID NO: 1583 - DNA, 22 bp, Artificial Sequence
  oligonucleotide
tggatgccgacacagccaacaa

SEQ ID NO: 1584 - DNA, 19 bp, Artificial Sequence
  oligonucleotide
tgaggtcgtcagaaatgag

SEQ ID NO: 1585 - DNA, 17 bp, Artificial Sequence
  oligonucleotide
gagcccaagctgaagaa

SEQ ID NO: 1586 - DNA, 22 bp, Artificial Sequence
  oligonucleotide
ttggatgccgacacagccaaca

SEQ ID NO: 1587 - DNA, 20 bp, Artificial Sequence
  oligonucleotide
gtcagaaatgaggaggaagt

SEQ ID NO: 1588 - DNA, 18 bp, Artificial Sequence
  oligonucleotide
gctggtttcccacatcaa

SEQ ID NO: 1589 - DNA, 24 bp, Artificial Sequence
  oligonucleotide
acagatgaacccaaggatgcggaa

SEQ ID NO: 1590 - DNA, 19 bp, Artificial Sequence
  oligonucleotide
cggcatccaaggtcatatc

SEQ ID NO: 1591 - DNA, 18 bp, Artificial Sequence
  oligonucleotide
gctagagaggctggtttc

SEQ ID NO: 1592 - DNA, 24 bp, Artificial Sequence
  oligonucleotide
acagatgaacccaaggatgcggaa

SEQ ID NO: 1593 - DNA, 20 bp, Artificial Sequence
  oligonucleotide
aggtcatatccacttggaac

SEQ ID NO: 1594 - DNA, 20 bp, Artificial Sequence
  oligonucleotide
cacaggattccagctctaaa

SEQ ID NO: 1595 - DNA, 24 bp, Artificial Sequence
  oligonucleotide
atagctgactgctgaccaccctcc

SEQ ID NO: 1596 - DNA, 19 bp, Artificial Sequence
  oligonucleotide
gacaccgatgatggatgaa

SEQ ID NO: 1597 - DNA, 17 bp, Artificial Sequence
  oligonucleotide
aaagacggcacctcaat

SEQ ID NO: 1598 - DNA, 23 bp, Artificial Sequence
  oligonucleotide
tccatcatcggtgtccacgacct

SEQ ID NO: 1599 - DNA, 18 bp, Artificial Sequence
  oligonucleotide
aggcattctcaggttctg

SEQ ID NO: 1600 - DNA, 19 bp, Artificial Sequence
  oligonucleotide
aaagtccctccttctcaag

SEQ ID NO: 1601 - DNA, 24 bp, Artificial Sequence
  oligonucleotide
atagctgactgctgaccaccctcc

SEQ ID NO: 1602 - DNA, 20 bp, Artificial Sequence
  oligonucleotide
gatgatggatgaagcatgtc

SEQ ID NO: 1603 - DNA, 17 bp, Artificial Sequence
  oligonucleotide
caggcaagcttgtgcca

SEQ ID NO: 1604 - DNA, 22 bp, Artificial Sequence
  oligonucleotide
acggcacctcaattgcaggcaa

SEQ ID NO: 1605 - DNA, 17 bp, Artificial Sequence
  oligonucleotide
agggtggtcagcagtca

SEQ ID NO: 1606 - DNA, 18 bp, Artificial Sequence
  oligonucleotide
ctggcacagtggttagaa

SEQ ID NO: 1607 - DNA, 22 bp, Artificial Sequence
  oligonucleotide
aagcaagactgggagcacgctg

SEQ ID NO: 1608 - DNA, 19 bp, Artificial Sequence
  oligonucleotide
gtgacaggaggtcatgaaa

SEQ ID NO: 1609 - DNA, 20 bp, Artificial Sequence
  oligonucleotide
caggttcaccagctttatga

SEQ ID NO: 1610 - DNA, 22 bp, Artificial Sequence
  oligonucleotide
aaagcaagactgggagcacgct

SEQ ID NO: 1611 - DNA, 17 bp, Artificial Sequence
  oligonucleotide
aaacggatggtggcaaa

SEQ ID NO: 1612 - DNA, 19 bp, Artificial Sequence
  oligonucleotide
aatcagacagtacctggca

SEQ ID NO: 1613 - DNA, 22 bp, Artificial Sequence
  oligonucleotide
aaagcaagactgggagcacgct

SEQ ID NO: 1614 - DNA, 17 bp, Artificial Sequence
  oligonucleotide
cagctgtgacaggaggt

SEQ ID NO: 1615 - DNA, 21 bp, Artificial Sequence
  oligonucleotide
accagctttatgatgacagtt

SEQ ID NO: 1616 - DNA, 19 bp, Artificial Sequence
  oligonucleotide
gatggtggcaaatgaaaca

SEQ ID NO: 1617 - DNA, 22 bp, Artificial Sequence
  oligonucleotide
aagcaagactgggagcacgctg

SEQ ID NO: 1618 - DNA, 19 bp, Artificial Sequence
  oligonucleotide
gatgcaaacagccagttac

SEQ ID NO: 1619 - DNA, 24 bp, Artificial Sequence
  oligonucleotide
tgtacgaaagccctgagcggtact

SEQ ID NO: 1620 - DNA, 18 bp, Artificial Sequence
  oligonucleotide
ggtgatgagaagcactga

SEQ ID NO: 1621 - DNA, 18 bp, Artificial Sequence
  oligonucleotide
cggaaaccgtgtgtactt

SEQ ID NO: 1622 - DNA, 22 bp, Artificial Sequence
  oligonucleotide
accagtgccctggatgcaaaca

SEQ ID NO: 1623 - DNA, 18 bp, Artificial Sequence
  oligonucleotide
ctcagggctttcgtacag

SEQ ID NO: 1624 - DNA, 17 bp, Artificial Sequence
  oligonucleotide
tgccctgctgcagaatc

SEQ ID NO: 1625 - DNA, 23 bp, Artificial Sequence
  oligonucleotide
agacttcttccaaatacctgtgg

SEQ ID NO: 1626 - DNA, 22 bp, Artificial Sequence
  oligonucleotide
atgagcaccgctacctgcacag

SEQ ID NO: 1627 - DNA, 17 bp, Artificial Sequence
  oligonucleotide
gtgccctggatgcaaac

SEQ ID NO: 1628 - DNA, 22 bp, Artificial Sequence
  oligonucleotide
agggctttcgtacaggagctgc

SEQ ID NO: 1629 - DNA, 17 bp, Artificial Sequence
  oligonucleotide
cactgagcgggagtacc

SEQ ID NO: 1630 - DNA, 18 bp, Artificial Sequence
  oligonucleotide
agcgagagtcttgtgatg

SEQ ID NO: 1631 - DNA, 22 bp, Artificial Sequence
  oligonucleotide
tttgctgtgattgcgcacgtgg

SEQ ID NO: 1632 - DNA, 20 bp, Artificial Sequence
  oligonucleotide
ccggatgtagacacagtaat

SEQ ID NO: 1633 - DNA, 18 bp, Artificial Sequence
  oligonucleotide
agccagatccttccaatg

SEQ ID NO: 1634 - DNA, 17 bp, Artificial Sequence
  oligonucleotide
ttcccacgtgcgcaatc

SEQ ID NO: 1635 - DNA, 24 bp, Artificial Sequence
  oligonucleotide
tgtgatgctgaggagcagtctgga

SEQ ID NO: 1636 - DNA, 18 bp, Artificial Sequence
  oligonucleotide
ccttccaatgaagcgaga

SEQ ID NO: 1637 - DNA, 24 bp, Artificial Sequence
  oligonucleotide
tgtgatgctgaggagcagtctgga

SEQ ID NO: 1638 - DNA, 19 bp, Artificial Sequence
  oligonucleotide
cagtaatgaccggagtctg

SEQ ID NO: 1639 - DNA, 18 bp, Artificial Sequence
  oligonucleotide
tttcagccagatccttcc

SEQ ID NO: 1640 - DNA, 22 bp, Artificial Sequence
  oligonucleotide
tgggaatggcagactccggtca

SEQ ID NO: 1641 - DNA, 17 bp, Artificial Sequence
  oligonucleotide
tccatccacagcattcc

SEQ ID NO: 1642 - DNA, 24 bp, Artificial Sequence
  oligonucleotide
ctagtcaatgatcttgtgttagac

SEQ ID NO: 1643 - DNA, 22 bp, Artificial Sequence
  oligonucleotide
ggattggaattacacaagtcaa

SEQ ID NO: 1644 - DNA, 28 bp, Artificial Sequence
  oligonucleotide
agggaagaggagagagaaagagcaactg

SEQ ID NO: 1645 - DNA, 19 bp, Artificial Sequence
  oligonucleotide
cctggagaagaccattcag

SEQ ID NO: 1646 - DNA, 24 bp, Artificial Sequence
  oligonucleotide
atgctgccgtggaaatgggcttta

SEQ ID NO: 1647 - DNA, 20 bp, Artificial Sequence
  oligonucleotide
ctctccagttgctaggattt

SEQ ID NO: 1648 - DNA, 19 bp, Artificial Sequence
  oligonucleotide
ctgctatccacatcagaca

SEQ ID NO: 1649 - DNA, 28 bp, Artificial Sequence
  oligonucleotide
acctggagaagaccattcagaagatgca

SEQ ID NO: 1650 - DNA, 18 bp, Artificial Sequence
  oligonucleotide
cacggcagcattaatcac

SEQ ID NO: 1651 - DNA, 22 bp, Artificial Sequence
  oligonucleotide
gacttactcaatgcagaagatg

SEQ ID NO: 1652 - DNA, 28 bp, Artificial Sequence
  oligonucleotide
agggaagaggagagagaaagagcaactg

SEQ ID NO: 1653 - DNA, 21 bp, Artificial Sequence
  oligonucleotide
ccaggattggaattacacaag

SEQ ID NO: 1654 - DNA, 22 bp, Artificial Sequence
  oligonucleotide
gtgacatcaatgctatcatctt

SEQ ID NO: 1655 - DNA, 22 bp, Artificial Sequence
  oligonucleotide
aggagacgcacaatatatttca

SEQ ID NO: 1656 - DNA, 23 bp, Artificial Sequence
  oligonucleotide
agttgtctgtgtgcgcaaatcca

SEQ ID NO: 1657 - DNA, 20 bp, Artificial Sequence
  oligonucleotide
cagaccgtattcttcatcct

SEQ ID NO: 1658 - DNA, 24 bp, Artificial Sequence
  oligonucleotide
ttattgtgggcttcacacggcagc

SEQ ID NO: 1659 - DNA, 17 bp, Artificial Sequence
  oligonucleotide
gatttgcgcacacagac

SEQ ID NO: 1660 - DNA, 22 bp, Artificial Sequence
  oligonucleotide
tgctatcatctttcacacaaag

SEQ ID NO: 1661 - DNA, 24 bp, Artificial Sequence
  oligonucleotide
agttgtctgtgtgcgcaaatccaa

SEQ ID NO: 1662 - DNA, 19 bp, Artificial Sequence
  oligonucleotide
tgtccaattccattccaga

SEQ ID NO: 1663 - DNA, 18 bp, Artificial Sequence
  oligonucleotide
tattgtgggcttcacacg

SEQ ID NO: 1664 - DNA, 24 bp, Artificial Sequence
  oligonucleotide
tggccaatgaaggctgtgacatca

SEQ ID NO: 1665 - DNA, 22 bp, Artificial Sequence
  oligonucleotide
cgcacaatatatttcacccaag

SEQ ID NO: 1666 - DNA, 21 bp, Artificial Sequence
  oligonucleotide
ccacagaatttcatagctgac

SEQ ID NO: 1667 - DNA, 21 bp, Artificial Sequence
  oligonucleotide
gcttggttaggaagatgacac

SEQ ID NO: 1668 - DNA, 23 bp, Artificial Sequence
  oligonucleotide
actttgagacgagcagccagtgc

SEQ ID NO: 1669 - DNA, 19 bp, Artificial Sequence
  oligonucleotide
gactactttgagacgagca

SEQ ID NO: 1670 - DNA, 24 bp, Artificial Sequence
  oligonucleotide
agcccggtgtcatcttcctaacca

SEQ ID NO: 1671 - DNA, 18 bp, Artificial Sequence
  oligonucleotide
ccaggtcgctgacatatt

SEQ ID NO: 1672 - DNA, 17 bp, Artificial Sequence
  oligonucleotide
cggcagattccacagaa

SEQ ID NO: 1673 - DNA, 26 bp, Artificial Sequence
  oligonucleotide
tgactactttgagacgagcagccagt

SEQ ID NO: 1674 - DNA, 17 bp, Artificial Sequence
  oligonucleotide
gcttcgcttggttagga

SEQ ID NO: 1675 - DNA, 23 bp, Artificial Sequence
  oligonucleotide
gcagattccacagaatttcatag

SEQ ID NO: 1676 - DNA, 24 bp, Artificial Sequence
  oligonucleotide
agcccggtgtcatcttcctaacca

SEQ ID NO: 1677 - DNA, 17 bp, Artificial Sequence
  oligonucleotide
gacccactcctcactgg

SEQ ID NO: 1678 - DNA, 19 bp, Artificial Sequence
  oligonucleotide
tcgcaactttgtggtagat

SEQ ID NO: 1679 - DNA, 19 bp, Artificial Sequence
  oligonucleotide
gattcactgggatcagcac

SEQ ID NO: 1680 - DNA, 23 bp, Artificial Sequence
  oligonucleotide
tcccagccagctgtggtattcca

SEQ ID NO: 1681 - DNA, 18 bp, Artificial Sequence
  oligonucleotide
tgtcctgtctctcctcat

SEQ ID NO: 1682 - DNA, 25 bp, Artificial Sequence
  oligonucleotide
tagtagctgccttctgctctccagc

SEQ ID NO: 1683 - DNA, 17 bp, Artificial Sequence
  oligonucleotide
cctcgcggtgtaagaaa

SEQ ID NO: 1684 - DNA, 17 bp, Artificial Sequence
  oligonucleotide
agcttcctcgcaacttt

SEQ ID NO: 1685 - DNA, 26 bp, Artificial Sequence
  oligonucleotide
cagccagctgtggtattccaaaccaa

SEQ ID NO: 1686 - DNA, 18 bp, Artificial Sequence
  oligonucleotide
acagacttgcttgcttct

SEQ ID NO: 1687 - DNA, 18 bp, Artificial Sequence
  oligonucleotide
gctagtagctgccttctg

SEQ ID NO: 1688 - DNA, 25 bp, Artificial Sequence
  oligonucleotide
accacaaagttgcgaggaagcttcc

SEQ ID NO: 1689 - DNA, 20 bp, Artificial Sequence
  oligonucleotide
gctgctggtctcatagtaat

SEQ ID NO: 1690 - DNA, 20 bp, Artificial Sequence
  oligonucleotide
ctgtcatcctcattgctact

SEQ ID NO: 1691 - DNA, 20 bp, Artificial Sequence
  oligonucleotide
gccactggtgtagaaatact

SEQ ID NO: 1692 - DNA, 22 bp, Artificial Sequence
  oligonucleotide
tcggacaccacaccctgctgct

SEQ ID NO: 1693 - DNA, 17 bp, Artificial Sequence
  oligonucleotide
attgctactgccctctg

SEQ ID NO: 1694 - DNA, 20 bp, Artificial Sequence
  oligonucleotide
gccactggtgtagaaatact

SEQ ID NO: 1695 - DNA, 22 bp, Artificial Sequence
  oligonucleotide
tcggacaccacaccctgctgct

SEQ ID NO: 1696 - DNA, 18 bp, Artificial Sequence
  oligonucleotide
ctcgctgtcatcctcatt

SEQ ID NO: 1697 - DNA, 24 bp, Artificial Sequence
  oligonucleotide
acaccctgctgctttgcctacatt

SEQ ID NO: 1698 - DNA, 18 bp, Artificial Sequence
  oligonucleotide
cttgccactggtgtagaa

SEQ ID NO: 1699 - DNA, 17 bp, Artificial Sequence
  oligonucleotide
tctgcgctcctgcatct

SEQ ID NO: 1700 - DNA, 25 bp, Artificial Sequence
  oligonucleotide
ccatattcctcggacaccacaccct

SEQ ID NO: 1701 - DNA, 17 bp, Artificial Sequence
  oligonucleotide
agtgggcgggcaatgta

SEQ ID NO: 1702 - DNA, 18 bp, Artificial Sequence
  oligonucleotide
catcgcccatggttaaga

SEQ ID NO: 1703 - DNA, 24 bp, Artificial Sequence
  oligonucleotide
tggcaaatccaactgaccagaagg

SEQ ID NO: 1704 - DNA, 18 bp, Artificial Sequence
  oligonucleotide
caggaacagccaccaata

SEQ ID NO: 1705 - DNA, 19 bp, Artificial Sequence
  oligonucleotide
aatggcaaatccaactgac

SEQ ID NO: 1706 - DNA, 25 bp, Artificial Sequence
  oligonucleotide
ccttcaggaacagccaccaataagc

SEQ ID NO: 1707 - DNA, 20 bp, Artificial Sequence
  oligonucleotide
ctgtgtctctctttcctctt

SEQ ID NO: 1708 - DNA, 19 bp, Artificial Sequence
  oligonucleotide
ctcaagaatgggcagaaag

SEQ ID NO: 1709 - DNA, 23 bp, Artificial Sequence
  oligonucleotide
ccgcatcgcccatggttaagaaa

SEQ ID NO: 1710 - DNA, 20 bp, Artificial Sequence
  oligonucleotide
cttctggtcagttggatttg

SEQ ID NO: 1711 - DNA, 19 bp, Artificial Sequence
  oligonucleotide
gcagaaagcttgtctcaac

SEQ ID NO: 1712 - DNA, 23 bp, Artificial Sequence
  oligonucleotide
ccgcatcgcccatggttaagaaa

SEQ ID NO: 1713 - DNA, 18 bp, Artificial Sequence
  oligonucleotide
gcttcctccttccttctg

SEQ ID NO: 1714 - DNA, 19 bp, Artificial Sequence
  oligonucleotide
gctgacgtgtgcagtaata

SEQ ID NO: 1715 - DNA, 18 bp, Artificial Sequence
  oligonucleotide
ctggcttcgtcagaatca

SEQ ID NO: 1716 - DNA, 22 bp, Artificial Sequence
  oligonucleotide
accagagccaggagacactgca

SEQ ID NO: 1717 - DNA, 20 bp, Artificial Sequence
  oligonucleotide
ctgcagacagtgaccatcta

SEQ ID NO: 1718 - DNA, 24 bp, Artificial Sequence
  oligonucleotide
aagggaccgaggtgacagtgaagt

SEQ ID NO: 1719 - DNA, 17 bp, Artificial Sequence
  oligonucleotide
gcgtcaccttggctcta

SEQ ID NO: 1720 - DNA, 17 bp, Artificial Sequence
  oligonucleotide
aggagacactgcagaca

SEQ ID NO: 1721 - DNA, 24 bp, Artificial Sequence
  oligonucleotide
cgccggaaagctgtagatggtcac

SEQ ID NO: 1722 - DNA, 18 bp, Artificial Sequence
  oligonucleotide
ttctgagacctctggctt

SEQ ID NO: 1723 - DNA, 17 bp, Artificial Sequence
  oligonucleotide
gaaccagagccaggaga

SEQ ID NO: 1724 - DNA, 24 bp, Artificial Sequence
  oligonucleotide
cgcccaacgtgattctgacgaagc

SEQ ID NO: 1725 - DNA, 17 bp, Artificial Sequence
  oligonucleotide
tcggtcccttctgagac

SEQ ID NO: 1726 - DNA, 19 bp, Artificial Sequence
  oligonucleotide
ccttccaaagatggctgaa

SEQ ID NO: 1727 - DNA, 24 bp, Artificial Sequence
  oligonucleotide
tcaatgaggagacttgcctggtga

SEQ ID NO: 1728 - DNA, 22 bp, Artificial Sequence
  oligonucleotide
tgttcctcactactctcaaatc

SEQ ID NO: 1729 - DNA, 19 bp, Artificial Sequence
  oligonucleotide
gatggatgcttccaatctg

SEQ ID NO: 1730 - DNA, 24 bp, Artificial Sequence
  oligonucleotide
tcaatgaggagacttgcctggtga

SEQ ID NO: 1731 - DNA, 20 bp, Artificial Sequence
  oligonucleotide
aaatctgttctggaggtact

SEQ ID NO: 1732 - DNA, 22 bp, Artificial Sequence
  oligonucleotide
cttccaatctggattcaatgag

SEQ ID NO: 1733 - DNA, 25 bp, Artificial Sequence
  oligonucleotide
tgagagtagtgaggaacaagccaga

SEQ ID NO: 1734 - DNA, 18 bp, Artificial Sequence
  oligonucleotide
tgtactcatctgcacagc

SEQ ID NO: 1735 - DNA, 17 bp, Artificial Sequence
  oligonucleotide
cagcaaagaggcactgg

SEQ ID NO: 1736 - DNA, 25 bp, Artificial Sequence
  oligonucleotide
acaacctgaaccttccaaagatggc

SEQ ID NO: 1737 - DNA, 20 bp, Artificial Sequence
  oligonucleotide
tgaatccagattggaagcat

SEQ ID NO: 1738 - DNA, 18 bp, Artificial Sequence
  oligonucleotide
ccacctctcaccaagaac

SEQ ID NO: 1739 - DNA, 18 bp, Artificial Sequence
  oligonucleotide
ggtatcagggctggaatc

SEQ ID NO: 1740 - DNA, 23 bp, Artificial Sequence
  oligonucleotide
agtcgaagtccagccgagatgct

SEQ ID NO: 1741 - DNA, 18 bp, Artificial Sequence
  oligonucleotide
taagagcaaggccaagag

SEQ ID NO: 1742 - DNA, 24 bp, Artificial Sequence
  oligonucleotide
tgatggactcagcagctccactct

SEQ ID NO: 1743 - DNA, 19 bp, Artificial Sequence
  oligonucleotide
gtagcctggaactgtgtag

SEQ ID NO: 1744 - DNA, 18 bp, Artificial Sequence
  oligonucleotide
aaagaccagagcaggaac

SEQ ID NO: 1745 - DNA, 25 bp, Artificial Sequence
  oligonucleotide
tgcttccacctctcaccaagaacca

SEQ ID NO: 1746 - DNA, 18 bp, Artificial Sequence
  oligonucleotide
gctggacttcgactttct

SEQ ID NO: 1747 - DNA, 17 bp, Artificial Sequence
  oligonucleotide
atgcttccacctctcac

SEQ ID NO: 1748 - DNA, 24 bp, Artificial Sequence
  oligonucleotide
aagtcgaagtccagccgagatgct

SEQ ID NO: 1749 - DNA, 18 bp, Artificial Sequence
  oligonucleotide
cccacatgacttcctctt

SEQ ID NO: 1750 - DNA, 19 bp, Artificial Sequence
  oligonucleotide
ggagacctgagaaccaatc

SEQ ID NO: 1751 - DNA, 19 bp, Artificial Sequence
  oligonucleotide
gactctccacgcatctctg

SEQ ID NO: 1752 - DNA, 23 bp, Artificial Sequence
  oligonucleotide
aggcagctggcagaggaatacct

SEQ ID NO: 1753 - DNA, 20 bp, Artificial Sequence
  oligonucleotide
tggagacctgagaaccaatc

SEQ ID NO: 1754 - DNA, 19 bp, Artificial Sequence
  oligonucleotide
acccgagtgtaaccatagc

SEQ ID NO: 1755 - DNA, 23 bp, Artificial Sequence
  oligonucleotide
aggcagctggcagaggaatacct

SEQ ID NO: 1756 - DNA, 17 bp, Artificial Sequence
  oligonucleotide
tccacccttgtgctctt

SEQ ID NO: 1757 - DNA, 18 bp, Artificial Sequence
  oligonucleotide
actctccacgcatctctg

SEQ ID NO: 1758 - DNA, 23 bp, Artificial Sequence
  oligonucleotide
aaccaatctcaccgacaggcagc

SEQ ID NO: 1759 - DNA, 20 bp, Artificial Sequence
  oligonucleotide
agaaccaatctcaccgacag

SEQ ID NO: 1760 - DNA, 21 bp, Artificial Sequence
  oligonucleotide
ccagagatttcgactctccac

SEQ ID NO: 1761 - DNA, 23 bp, Artificial Sequence
  oligonucleotide
tggttacactcgggtggcagaga

SEQ ID NO: 1762 - DNA, 18 bp, Artificial Sequence
  oligonucleotide
aatggatggcaggccttt

SEQ ID NO: 1763 - DNA, 20 bp, Artificial Sequence
  oligonucleotide
cgctcaatcttcatcttgtg

SEQ ID NO: 1764 - DNA, 22 bp, Artificial Sequence
  oligonucleotide
tgccattgtgttccggacaccc

SEQ ID NO: 1765 - DNA, 19 bp, Artificial Sequence
  oligonucleotide
tcccacagatgtgcataaa

SEQ ID NO: 1766 - DNA, 24 bp, Artificial Sequence
  oligonucleotide
tatgccattgtgttccggacaccc

SEQ ID NO: 1767 - DNA, 17 bp, Artificial Sequence
  oligonucleotide
ttacaggccgctcaatc

SEQ ID NO: 1768 - DNA, 20 bp, Artificial Sequence
  oligonucleotide
cggttctatgaggatgatga

SEQ ID NO: 1769 - DNA, 25 bp, Artificial Sequence
  oligonucleotide
tgcacatctgtgggagagaagtccc

SEQ ID NO: 1770 - DNA, 18 bp, Artificial Sequence
  oligonucleotide
cggaacacaatggcatac

SEQ ID NO: 1771 - DNA, 20 bp, Artificial Sequence
  oligonucleotide
aggatgatgagaatggatgg

SEQ ID NO: 1772 - DNA, 26 bp, Artificial Sequence
  oligonucleotide
cacaagatgaagattgagcggcctgt

SEQ ID NO: 1773 - DNA, 20 bp, Artificial Sequence
  oligonucleotide
gtttcagttgcagaaacact

SEQ ID NO: 1774 - DNA, 20 bp, Artificial Sequence
  oligonucleotide
tgtgttgacatccagatcac

SEQ ID NO: 1775 - DNA, 20 bp, Artificial Sequence
  oligonucleotide
taggagaggttagagaaggc

SEQ ID NO: 1776 - DNA, 28 bp, Artificial Sequence
  oligonucleotide
ccaccaacttacaatgctgactatggct

SEQ ID NO: 1777 - DNA, 20 bp, Artificial Sequence
  oligonucleotide
ttgacagtccaccaacttac

SEQ ID NO: 1778 - DNA, 21 bp, Artificial Sequence
  oligonucleotide
ggaggaagggctctagtataa

SEQ ID NO: 1779 - DNA, 26 bp, Artificial Sequence
  oligonucleotide
aagctgggaagccttctctaacctct

SEQ ID NO: 1780 - DNA, 22 bp, Artificial Sequence
  oligonucleotide
gtgaataacattcccttccttc

SEQ ID NO: 1781 - DNA, 21 bp, Artificial Sequence
  oligonucleotide
tagccatagtcagcattgtaa

SEQ ID NO: 1782 - DNA, 29 bp, Artificial Sequence
  oligonucleotide
ccagatcacatttgattgacagtccacca

SEQ ID NO: 1783 - DNA, 22 bp, Artificial Sequence
  oligonucleotide
ccaacttacaatgctgactatg

SEQ ID NO: 1784 - DNA, 19 bp, Artificial Sequence
  oligonucleotide
caatcatcaggcacaggag

SEQ ID NO: 1785 - DNA, 26 bp, Artificial Sequence
  oligonucleotide
aagctgggaagccttctctaacctct

SEQ ID NO: 1786 - DNA, 18 bp, Artificial Sequence
  oligonucleotide
tgcactttggagtgatcg

SEQ ID NO: 1787 - DNA, 21 bp, Artificial Sequence
  oligonucleotide
ggttcgagaagatgatctgac

SEQ ID NO: 1788 - DNA, 26 bp, Artificial Sequence
  oligonucleotide
agggacctctctctaatcagccctct

SEQ ID NO: 1789 - DNA, 17 bp, Artificial Sequence
  oligonucleotide
agcctcttctccttcct

SEQ ID NO: 1790 - DNA, 24 bp, Artificial Sequence
  oligonucleotide
ttctgcctgctgcactttggagtg

SEQ ID NO: 1791 - DNA, 19 bp, Artificial Sequence
  oligonucleotide
agagggctgattagagaga

SEQ ID NO: 1792 - DNA, 20 bp, Artificial Sequence
  oligonucleotide
tcagatcatcttctcgaacc

SEQ ID NO: 1793 - DNA, 24 bp, Artificial Sequence
  oligonucleotide
agcccatgttgtagcaaaccctca

SEQ ID NO: 1794 - DNA, 19 bp, Artificial Sequence
  oligonucleotide
ggttatctctcagctccac

SEQ ID NO: 1795 - DNA, 17 bp, Artificial Sequence
  oligonucleotide
ctgcctgctgcactttg

SEQ ID NO: 1796 - DNA, 26 bp, Artificial Sequence
  oligonucleotide
agggacctctctctaatcagccctct

SEQ ID NO: 1797 - DNA, 18 bp, Artificial Sequence
  oligonucleotide
ggctacaggcttgtcact

SEQ ID NO: 1798 - DNA, 18 bp, Artificial Sequence
  oligonucleotide
agaggagctagtgaagga

SEQ ID NO: 1799 - DNA, 21 bp, Artificial Sequence
  oligonucleotide
ctgtgacatttgagtcctttc

SEQ ID NO: 1800 - DNA, 22 bp, Artificial Sequence
  oligonucleotide
tcccaccaccttctccctcctt

SEQ ID NO: 1801 - DNA, 18 bp, Artificial Sequence
  oligonucleotide
agaggagctagtgaagga

SEQ ID NO: 1802 - DNA, 22 bp, Artificial Sequence
  oligonucleotide
tcccaccaccttctccctcctt

SEQ ID NO: 1803 - DNA, 20 bp, Artificial Sequence
  oligonucleotide
gagatgctgtgacatttgag

SEQ ID NO: 1804 - DNA, 19 bp, Artificial Sequence
  oligonucleotide
gctagtgaaggacaacgag

SEQ ID NO: 1805 - DNA, 22 bp, Artificial Sequence
  oligonucleotide
tcccaccaccttctccctcctt

SEQ ID NO: 1806 - DNA, 17 bp, Artificial Sequence
  oligonucleotide
ctgtgggagatgctgtg

SEQ ID NO: 1807 - DNA, 17 bp, Artificial Sequence
  oligonucleotide
ccaccttctccctcctt

SEQ ID NO: 1808 - DNA, 25 bp, Artificial Sequence
  oligonucleotide
aaatgtcacagcatctcccacagcc

SEQ ID NO: 1809 - DNA, 17 bp, Artificial Sequence
  oligonucleotide
gtgctggcttgtcactg

SEQ ID NO: 1810 - DNA, 20 bp, Artificial Sequence
  oligonucleotide
ctggaaagagaacccatctg

SEQ ID NO: 1811 - DNA, 17 bp, Artificial Sequence
  oligonucleotide
ctcacggttgttctggt

SEQ ID NO: 1812 - DNA, 20 bp, Artificial Sequence
  oligonucleotide
agtatgatgcgctgctgccg

SEQ ID NO: 1813 - DNA, 17 bp, Artificial Sequence
  oligonucleotide
aacccatctgtcgctct

SEQ ID NO: 1814 - DNA, 17 bp, Artificial Sequence
  oligonucleotide
ctcacggttgttctggt

SEQ ID NO: 1815 - DNA, 20 bp, Artificial Sequence
  oligonucleotide
agtatgatgcgctgctgccg

SEQ ID NO: 1816 - DNA, 17 bp, Artificial Sequence
  oligonucleotide
ctgagcttggagcagag

SEQ ID NO: 1817 - DNA, 24 bp, Artificial Sequence
  oligonucleotide
caggaaagtgccatcgaaggaggc

SEQ ID NO: 1818 - DNA, 19 bp, Artificial Sequence
  oligonucleotide
tggtgacattggtgatctt

SEQ ID NO: 1819 - DNA, 17 bp, Artificial Sequence
  oligonucleotide
ccctggccacctctatc

SEQ ID NO: 1820 - DNA, 23 bp, Artificial Sequence
  oligonucleotide
atcctgagcttggagcagagggt

SEQ ID NO: 1821 - DNA, 17 bp, Artificial Sequence
  oligonucleotide
cagggcctggtctttct

SEQ ID NO: 1822 - DNA, 19 bp, Artificial Sequence
  oligonucleotide
ctggaagaagcagaaagga

SEQ ID NO: 1823 - DNA, 28 bp, Artificial Sequence
  oligonucleotide
ctgagagtgtcaaagaaggagacactgt

SEQ ID NO: 1824 - DNA, 21 bp, Artificial Sequence
  oligonucleotide
ttccacatgtacaagagatga

SEQ ID NO: 1825 - DNA, 20 bp, Artificial Sequence
  oligonucleotide
tttccttctgagagtgtcaa

SEQ ID NO: 1826 - DNA, 29 bp, Artificial Sequence
  oligonucleotide
acactgtcatcatctcttgtacatgtgga

SEQ ID NO: 1827 - DNA, 20 bp, Artificial Sequence
  oligonucleotide
tagtactgtgtctcctgtct

SEQ ID NO: 1828 - DNA, 19 bp, Artificial Sequence
  oligonucleotide
tgaaggaattaaccaggct

SEQ ID NO: 1829 - DNA, 24 bp, Artificial Sequence
  oligonucleotide
ttccacttcctttctgcttcttcc

SEQ ID NO: 1830 - DNA, 20 bp, Artificial Sequence
  oligonucleotide
ctttgacactctcagaagga

SEQ ID NO: 1831 - DNA, 20 bp, Artificial Sequence
  oligonucleotide
gcagaaaggaagtggaatta

SEQ ID NO: 1832 - DNA, 27 bp, Artificial Sequence
  oligonucleotide
tccttctgagagtgtcaaagaaggaga

SEQ ID NO: 1833 - DNA, 21 bp, Artificial Sequence
  oligonucleotide
gtacaagagatgatgacagtg

(19) United States
(12) Patent Application Publication
(10) Pub. No.: US 2023/0416820 A1
(43) Pub. Date: Dec. 28, 2023

(54) PCR METHOD AND KIT FOR DETERMINING PATHWAY ACTIVITY

(71) Applicant: InnoSIGN B.V., Eindhoven (NL)

(72) Inventors: Eveline Catharina Anna Clasina DEN BIEZEN, Eindhoven (NL); Dianne Arnoldina Margaretha Wilhelmina VAN STRIJP, Eindhoven (NL); Anne Godefrida Catharina VAN BRUSSEL, Eindhoven (NL); Janneke WROBEL, Eindhoven (NL); Laurentius Henricus Franciscus Maria HOLTZER, Eindhoven (NL)

(73) Assignee: InnoSIGN B.V., Eindhoven (NL)

(21) Appl. No.: 18/042,546

(22) PCT Filed: Aug. 25, 2021
(86) PCT No.: PCT/EP2021/073469

§ 371 Date: Feb. 22, 2023

(30) Foreign Application Priority Data
Aug. 28, 2020 (EP) 20193288.6

(51) Int. Cl. C12Q 1/6876

(52) U.S. Cl. CPC C12Q 1/6876; C12Q 1/686; C12Q 2600/16; C12Q 2600/158

(57) ABSTRACT

The present invention relates to assemblies of primers and probes for determining the cellular signalling pathway of the AR pathway and optionally the activities of the pathways ER, PI3K-FOXO, MAPK-AP1, Notch, HH, TGFbeta, JAK-STAT1/2 and NFkB. Kit or use of the set of 3 or more primers and probes to determine the expression levels of 3 or more genes of the AR cellular signalling pathway and optionally from a cellular signalling pathway selected from the ER, PI3K-FOXO, MAPK-AP1, Notch, HH, TGFbeta, WNT, PR, NFkB, JAK-STAT1/2 and JAK-STAT3 pathways wherein the expression levels of three or more of the reference genes selected from: ACTB, ALAS1, B2M, EEF1A1, POLR2A, PUM1, RPLPO, TBP, TPT1 and TUBA1B may also be determined.

### FIELD OF THE INVENTION

The subject matter described herein relates to cellular signaling pathway analysis based on gene expression data. More specifically the subject matter relates to primers, probes and kits which are tailored for establishing the gene expression levels in a sample for the purpose of determining cellular signaling pathway activity. The subject further relates to PCR based methods to determine the expression levels of target genes which can be used for this purpose.

### BACKGROUND OF THE INVENTION

Determining the activity of cellular signaling pathways in a sample is an emerging technology with many applications in diagnosis and prognostics, as well as biotechnological applications. It was found by the inventors that using a mathematical model, the cellular signaling pathway activity can be determined based on the expression levels of target genes of the cellular signaling pathway, as e.g. described in WO2013011479A2, WO2014102668A2, WO2015101635A1, WO2016062891A1, WO2017029215A1, WO2019068585A1, WO2019068562A1, WO2019068543A1 and WO2019120658A1 (each incorporated in its entirety by reference).

For many applications it would be desirable to determine the pathway activities of multiple cellular signaling pathways simultaneously. This means that many expression levels need to be determined in a sample at the same time. Namely, for each pathway preferably at least three target genes as well as at least three reference genes for normalization purposes. This simultaneous determination of expression levels could for example by done using Affymetrix Microarray technology, however this technology has the disadvantage that it is time consuming, expensive and cannot be performed in every lab due to the required equipment and specialized personnel.

The present invention aims, among other, to solve the above problems by the methods and products as defined in the appended claims.

### SUMMARY OF THE INVENTION

In an aspect, the invention relates to an assembly of primers and probe for determining the activity of the AR cellular signaling pathway, and optionally one or more additional cellular signaling pathway, wherein the assembly of primers and probes comprises three or more sets of primers or probes for determining the expression level of three or more target genes of the AR cellular signaling pathway, wherein said three or more sets of primers and probes are selected from Table 2 of the description,
wherein each primer and/or probe individually has the listed nucleotide sequence identified by the corresponding SEQ ID NO or has a sequence that differs at 1, 2 or 3 positions, wherein said 1, 2 or 3 differences are individually selected from a single base substitution, a single base deletion or a single base addition.

In an aspect the invention relates to a kit of parts for determining the expression levels for a plurality of genes, the kit comprising primers and probes for the amplification and detection of the expression levels of the plurality of genes, wherein the kit comprises an assembly of primers and probes as defined in the first aspect of the invention, wherein the kit further comprises primers and probes for the amplification and detection of three or more of the reference genes selected from ACTB, ALAS1, B2M, EEF1A1 POLR2A, PUM1, RPLP0, TBP, TPT1 and TUBA1B, preferably wherein said three or more sets of primers and probes are selected from Table 8 of the description, and wherein each primer and/or probe individually has the listed nucleotide sequence identified by the corresponding SEQ ID NO or has a sequence that differs at 1, 2 or 3 positions, wherein said 1, 2 or 3 differences are individually selected from a single base substitution, a single base deletion or a single base addition.

In an aspect the invention relates to the use of the assembly of primers and probes as defined in first aspect or the kit as defined in the second aspect of the invention for determining the AR cellular signaling pathway activity, and optionally the cellular signaling pathway activity of one or more cellular signaling pathways selected from the group consisting of: HH, ER, TGFbeta, PI3K-FOXO, Notch, MAPK-AP1, JAK-STAT1/2 and NFkB.

In an aspect the invention relates to the use of a set of three or more primers and probes to determine the expression levels of three or more target genes of a cellular signaling pathway, wherein the set of primers and probe combinations are as defined in the first aspect of the invention, and
wherein the three or more target genes for the AR cellular signaling pathway are selected from the group consisting of: ABCC4, AR, CREB3L4, DHCR24, ELL2, FKBP5, GUCY1A3, KLK2, KLK3, LRIG1, NDRG1, NKX3.1 (also known as NKX3_1), PLAU, PMEPA1, PPAP2A, PRKACB_2, SGK1, and TMPRSS2;
wherein the three or more target genes for the ER cellular signaling pathway are selected from the group consisting of: AP1B1, CA12, CDH26, CELSR2, CTSD, ERBB2, ESR1, GREB1, HSPB1, IGFBP4, MYC, NRIP1, PDZK1, PGR, RARA, SGK3, SOD1, TFF1, WISP2, and XBP1;
the three or more target genes for the PI3K-FOXO cellular signaling pathway are selected from the group consisting of AGRP, BCL2L11, BCL6, BNIP3, BTG1, CAT, CAV1, CCND1, CCNG2, CDKN1A, CDKN1B, ESR1, FBXO32, FOXO3, GADD45A, INSR, MXI1, SOD2, TNFSF10;
wherein the three or more target genes for the MAPK-AP1 cellular signaling pathway are selected from the group consisting of BCL2L11, CCND1, DDIT3, EGFR, ENPP2, EZR, GLRX, MMP1, MMP3, MMP9, PLAU, PLAUR, PTGS2, SERPINE1, TIMP1, TP53, VEGFD, and VIM;
wherein the three or more target genes for the Notch cellular signaling pathway are selected from the group consisting of CD44, EPHB3, FABP7, HES1, HES4, HES5, HEY1, HEY2, MYC, NOX1, NRARP, PIN1, PLXND1, and SOX9;
wherein the three or more target genes for the HH cellular signaling pathway are selected from the group consisting of CFLAR, FOXM1, FYN, GLI1, HHIP, MYCN, NKX2-2, PTCH1, PTCH2, RAB34, SPP1, TCEA2, and TSC22D1;
wherein the three or more target genes for the TGFbeta cellular signaling pathway are selected from the group consisting of ANGPTL4, CDKN1A, CTGF, GADD45A, GADD45B, ID1, IL11, JUNB, MMP2, MMP9, PDGFB, SERPINE1, SGK1, SKIL, SMAD4, SMAD7, SNAI1, TIMP1, and VEGFA;
wherein the three or more target genes for the WNT cellular signaling pathway are selected from the group consisting of CEMIP, AXIN2, CD44, RNF43, MYC, TBX3, TDGF1, SOX9, ASCL2, CXCL8, SP5, ZNRF3, EPHB2, LGR5, EPHB3, KLF6, CCND1, DEFA6, and FZD7;
wherein the three or more target genes for the PR cellular signaling pathway are selected from the group consisting of AGRP, BCL2L11, BCL6, BNIP3, BTG1, CAT, CAV1, CCND1, CCND2, CCNG2, CDKN1A, CDKN1B, ESR1, FASLG, FBXO32, GADD45A, INSR, MXI1, NOS3, PCK1, POMC, PPARGC1A, PRDX3, RBL2, SOD2 and TNFSF10;
wherein the three or more target genes for the NFkB cellular signaling pathway are selected from the group consisting of BIRC3, CCL3, CCL4, CCL5, CCL20, CXCL2, ICAM1, IL6, IRF1, MMP9, NFKB2, PTGS2, TNF, TNIP1, TRAF1, and VCAM1;
wherein the three or more target genes for the JAK-STAT1/2 cellular signaling pathway are selected from the group consisting of APOL1, BID, CXCL9, GBP1, GNAZ, IFI6, IFIT2, IFITM1, IRF1, IRF7, IRF9, ISG15, LY6E, OAS1, PDCD1, RFPL3, SSTR3, STAT1, TAP1 and USP18;
wherein the three or more target genes for the JAK-STAT3 cellular signaling pathway are selected from the group consisting of AKT1, BCL2, BCL2L1, BIRC5, CCND1, CD274, CDKNIA, CRP, FGF2, FOS, FSCN1, FSCN2, FSCN3, HIFIA, HSP90AA1, HSP90AB1, HSP90B1, HSPA1A, HSPA1B, ICAM1, IFNG, IL10, JunB, MCL1, MMP1, MMP3, MMP9, MUC1, MYC, NOS2, POU2F1, PTGS2, SAA1, STAT1, TIMP1, TNFRSF1B, TWIST1, VIM and ZEB1.

In an aspect the invention relates to a method for designing primers and probes for the detection of the expression levels of target genes of a cellular signaling pathway suitable for determining the activity of the AR cellular signaling pathway and optionally one or more additional cellular signaling pathways, the method comprising:
designing for a target gene of the AR cellular signaling pathway and optionally one or more additional cellular signaling pathway a forward primer and a reverse primer such that:
the forward and reverse primer have a GC content between 35% and 69%, preferably between 35% and 65%;
the forward and reverse primer have a melting temperature between 50 and 71 degrees Celsius, preferably between 58 and 64 degrees Celsius;
the forward and reverse primer have a length between 16 and 25 nucleotides, preferably between 17 and 24 nucleotides;
wherein the amplification product, when using the forward and reverse primers in a PCR amplification reaction, has a size between 60 and 240 base pairs, preferably between 65 and 150 base pairs, and preferably wherein the amplicon product is intron spanning;
designing the probe such that:
the probe used for detection of an amplification product comprises a binding part which is complementary to a part of the amplification product, the binding part further having the following characteristics:
the binding part of the probe has a GC content between 35% and 69%, preferably between 40% and 60%;
the binding part of the probe has a melting temperature between 56 and 72 degrees Celsius, preferably between 64 and 72 degrees Celsius;
the binding part of the probe has a length between 17 and 31 nucleotides, preferably between 18 and 30 nucleotides;
the binding part of the probe does not have a G at the 5′ part.

In a sixth aspect the invention relates to a method of determining the AR cellular signaling pathway activity and optionally one or more additional cellular signaling pathway activity or activities, by simultaneously determining the expression level of six or more genes in a sample, the method comprising simultaneously amplifying six or more gene products using a polymerase chain reaction to generate a plurality of amplification products, followed by the detection of the plurality of amplification products using a plurality of probes,
wherein the polymerase chain reaction uses, for each amplification product, a forward and a reverse primer which have the following characteristics:
the forward and reverse primer have a GC content between 35% and 69%, preferably between 35% and 65%;
the forward and reverse primer have a melting temperature between 50 and 71 degrees Celsius, preferably between 58 and 64 degrees Celsius;
the forward and reverse primer have a length between 16 and 25 nucleotides, preferably between 17 and 24 nucleotides;
wherein the amplification products have a size between 60 and 240 base pairs, preferably between 65 and 150 base pairs, and preferably wherein the amplicon product is intron spanning,
wherein each of the probes used for detection of an amplification product comprises a binding part which is complementary to a part of the amplification product, the binding part further having the following characteristics:
the binding part of the probe has a GC content between 35% and 69%, preferably between 40% and 60%;
the binding part of the probe has a melting temperature between 56 and 72 degrees Celsius, preferably between 64 and 72 degrees Celsius;
the binding part of the probe has a length between 17 and 31 nucleotides, preferably between 18 and 30 nucleotides;
the binding part of the probe does not have a G at the 5′ part, wherein the expression levels are used in a method for determining the AR cellular signaling pathway and optionally one or more cellular signaling pathway activities selected from the group consisting of: WNT, HH, ER, PR, PR, TGFbeta, NFkB, STAT1/2, STAT3, PI3K-FOXO, Notch, MAPK-AP1, and
wherein the primers and probes amplify and detect of the expression levels of three or more of the reference genes selected from: ACTB, ALAS1, B2M, EEF1A1 POLR2A, PUM1, RPLP0, TBP, TPT1 and TUBA1B, and
wherein the primers and probes further amplify and detect the expression levels of three or more target genes for the AR cellular signaling pathway and optionally one or more cellular signaling pathways selected from the group consisting of: ER, PI3K-FOXO, MAPK-AP1, HH, Notch, TGFbeta, WNT, PR, NFkB, JAK-STAT1/2, JAK-STAT3,
wherein the three or more target genes for the AR cellular signaling pathway are selected from the group consisting of: ABCC4, AR, CREB3L4, DHCR24, ELL2, FKBP5, GUCY1A3, KLK2, KLK3, LRIG1, NDRG1, NKX3.1 (also known as NKX3_1), PLAU, PMEPA1, PPAP2A, PRKACB_2, SGK1, and TMPRSS2;
wherein the three or more target genes for the ER cellular signaling pathway are selected from the group consisting of: AP1B1, CA12, CDH26, CELSR2, CTSD, ERBB2, ESR1, GREB1, HSPB1, IGFBP4, MYC, NRIP1, PDZK1, PGR, RARA, SGK3, SOD1, TFF1, WISP2, and XBP1;
the three or more target genes for the PI3K-FOXO cellular signaling pathway are selected from the group consisting of: AGRP, BCL2L11, BCL6, BNIP3, BTG1, CAT, CAV1, CCND1, CCNG2, CDKN1A, CDKN1B, ESR1, FBXO32, FOXO3, GADD45A, INSR, MXI1, SOD2, TNFSF10;
wherein the three or more target genes for the MAPK-AP1 cellular signaling pathway are selected from the group consisting of: BCL2L11, CCND1, DDIT3, EGFR, ENPP2, EZR, GLRX, MMP1, MMP3, MMP9, PLAU, PLAUR, PTGS2, SERPINE1, TIMP1, TP53, VEGFD, and VIM;
wherein the three or more target genes for the Notch cellular signaling pathway are selected from the group consisting of: CD44, EPHB3, FABP7, HES1, HES4, HES5, HEY1, HEY2, MYC, NOX1, NRARP, PIN1, PLXND1, and SOX9;
wherein the three or more target genes for the HH cellular signaling pathway are selected from the group consisting of: CFLAR, FOXM1, FYN, GLI1, HHIP, MYCN, NKX2-2, PTCH1, PTCH2, RAB34, SPP1, TCEA2, and TSC22D1;
wherein the three or more target genes for the TGFbeta cellular signaling pathway are selected from the group consisting of: ANGPTL4, CDKN1A, CTGF, GADD45A, GADD45B, ID1, IL11, JUNB, MMP2, MMP9, PDGFB, SERPINE1, SGK1, SKIL, SMAD4, SMAD7, SNAI1, TIMP1, and VEGFA;
wherein the three or more target genes for the WNT cellular signaling pathway are selected from the group consisting of: CEMIP, AXIN2, CD44, RNF43, MYC, TBX3, TDGF1, SOX9, ASCL2, CXCL8, SP5, ZNRF3, EPHB2, LGR5, EPHB3, KLF6, CCND1, DEFA6, and FZD7;
wherein the three or more target genes for the PR cellular signaling pathway are selected from the group consisting of: AGRP, BCL2L11, BCL6, BNIP3, BTG1, CAT, CAV1, CCND1, CCND2, CCNG2, CDKN1A, CDKN1B, ESR1, FASLG, FBXO32, GADD45A, INSR, MXI1, NOS3, PCK1, POMC, PPARGC1A, PRDX3, RBL2, SOD2 and TNFSF10;
wherein the three or more target genes for the NFkB cellular signaling pathway are selected from the group consisting of: BIRC3, CCL3, CCL4, CCL5, CCL20, CXCL2, ICAM1, IL6, IRF1, MMP9, NFKB2, PTGS2, TNF, TNIP1, TRAF1, and VCAM1;
wherein the three or more target genes for the JAK-STAT1/2 cellular signaling pathway are selected from the group consisting of: APOL1, BID, CXCL9, GBP1, GNAZ, IFI6, IFIT2, IFITM1, IRF1, IRF7, IRF9, ISG15, LY6E, OAS1, PDCD1, RFPL3, SSTR3, STAT1, TAP1 and USP18;
wherein the three or more target genes for the JAK-STAT3 cellular signaling pathway are selected from the group consisting of: AKT1, BCL2, BCL2L1, BIRC5, CCND1, CD274, CDKNIA, CRP, FGF2, FOS, FSCN1, FSCN2, FSCN3, HIFIA, HSP90AA1, HSP90AB1, HSP90B1, HSPA1A, HSPA1B, ICAM1, IFNG, IL10, JunB, MCL1, MMP1, MMP3, MMP9, MUC1, MYC, NOS2, POU2F1, PTGS2, SAA1, STAT1, TIMP1, TNFRSF1B, TWIST1, VIM and ZEB1, wherein the primers and probes are able to amplify and detect the respective genes under the following reaction conditions:
50 mM monovalent salt;
400 nM forward primer
400 nM reverse primer
3.0 mM divalent salt, preferably the divalent salt being Mg²⁺;
100 nM probe; and
0.8 mM dNTP.

### DETAILED DESCRIPTION OF THE INVENTION

An alternative to e.g. a microarray based technology would be to use PCR based technology like qPCR, however the problem with such technology is that the used primers and probes often require different conditions for optimal performance, meaning many different reactions need to be performed. The present invention aims to overcome the above problems, among others, by narrowly defining the reaction conditions combined with the selection criteria for the primers and probes, and further by providing sets of primers and probes suitable for the designed reaction conditions which can be used to determine the expression levels of the different target genes which can be used to determining cellular signaling pathway activity. This allows the primers and probes to be used under the same reaction conditions (e.g. in the same multi-well plate or even as a multiplex).

Therefore, in a first aspect the present invention relates to a method for simultaneously determining the expression level of six or more genes in a sample, the method comprising simultaneously amplifying six or more gene products using a polymerase chain reaction to generate a plurality of amplification products, followed by the detection of the plurality of amplification products using a plurality of probes,
wherein the polymerase chain reaction uses, for each amplification product, a forward and a reverse primer which have the following characteristics:
the forward and reverse primer have a GC content between 35% and 69%, preferably between 35% and 65%;
the forward and reverse primer have a melting temperature between 50 and 71 degrees Celsius, preferably between 58 and 64 degrees Celsius;
the forward and reverse primer have a length between 16 and 25 nucleotides, preferably between 17 and 24 nucleotides;
wherein the amplification products have a size between 60 and 240 base pairs, preferably between 65 and 150 base pairs, and preferably wherein the amplicon product is intron spanning,
wherein each of the probes used for detection of an amplification product comprises a binding part which is complementary to a part of the amplification product, the binding part further having the following characteristics:
the binding part of the probe has a GC content between 35% and 69%, preferably between 40% and 60%;
the binding part of the probe has a melting temperature between 56 and 72 degrees Celsius, preferably between 64 and 72 degrees Celsius;
the binding part of the probe has a length between 17 and 31 nucleotides, preferably between 18 and 30 nucleotides;
the binding part of the probe does not have a G at the 5′ part,
wherein the expression levels are suitable for use in a method for determining one or more cellular signaling pathway activities selected from the group consisting of: WNT, HH, AR, ER, PR, PR, TGFbeta, NFkB, STAT1/2, STAT3, PI3K-FOXO, Notch, MAPK-AP1, and
wherein the primers and probes amplify and detect of the expression levels of three or more of the reference genes selected from: ACTB, ALAS1, B2M, EEF1A1 POLR2A, PUM1, RPLP0, TBP, TPT1 and TUBA1B, and
wherein the primers and probes further amplify and detect the expression levels of three or more target genes for one or more cellular signaling pathways selected from the group consisting of: AR, ER, PI3K-FOXO, MAPK-AP1, HH, Notch, TGFbeta, WNT, PR, NFkB, JAK-STAT1/2, JAK-STAT3,
wherein the three or more target genes for the ER cellular signaling pathway are selected from the group consisting of: AP1B1, CA12, CDH26, CELSR2, CTSD, ERBB2, ESR1, GREB1, HSPB1, IGFBP4, MYC, NRIP1, PDZK1, PGR, RARA, SGK3, SOD1, TFF1, WISP2, and XBP1;
the three or more target genes for the AR cellular signaling pathway are selected from the group consisting of: ABCC4, AR, CREB3L4, DHCR24, ELL2, FKBP5, GUCY1A3, KLK2, KLK3, LRIG1, NDRG1, NKX3.1 (also known as NKX3_1), PLAU, PMEPA1, PPAP2A, PRKACB_2, SGK1, and TMPRSS2;
the three or more target genes for the PI3K-FOXO cellular signaling pathway are selected from the group consisting of: AGRP, BCL2L11, BCL6, BNIP3, BTG1, CAT, CAV1, CCND1, CCNG2, CDKN1A, CDKN1B, ESR1, FBXO32, FOXO3, GADD45A, INSR, MXI1, SOD2, TNFSF10;
wherein the three or more target genes for the MAPK-AP1 cellular signaling pathway are selected from the group consisting of: BCL2L11, CCND1, DDIT3, EGFR, ENPP2, EZR, GLRX, MMP1, MMP3, MMP9, PLAU, PLAUR, PTGS2, SERPINE1, TIMP1, TP53, VEGFD, and VIM;
wherein the three or more target genes for the Notch cellular signaling pathway are selected from the group consisting of: CD44, EPHB3, FABP7, HES1, HES4, HES5, HEY1, HEY2, MYC, NOX1, NRARP, PIN1, PLXND1, and SOX9;
wherein the three or more target genes for the HH cellular signaling pathway are selected from the group consisting of: CFLAR, FOXM1, FYN, GLI1, HHIP, MYCN, NKX2-2, PTCH1, PTCH2, RAB34, SPP1, TCEA2, and TSC22D1;
wherein the three or more target genes for the TGFbeta cellular signaling pathway are selected from the group consisting of: ANGPTL4, CDKN1A, CTGF, GADD45A, GADD45B, ID1, IL11, JUNB, MMP2, MMP9, PDGFB, SERPINE1, SGK1, SKIL, SMAD4, SMAD7, SNAI1, TIMP1, and VEGFA;
wherein the three or more target genes for the WNT cellular signaling pathway are selected from the group consisting of: CEMIP, AXIN2, CD44, RNF43, MYC, TBX3, TDGF1, SOX9, ASCL2, CXCL8, SP5, ZNRF3, EPHB2, LGR5, EPHB3, KLF6, CCND1, DEFA6, and FZD7;
wherein the three or more target genes for the PR cellular signaling pathway are selected from the group consisting of: AGRP, BCL2L11, BCL6, BNIP3, BTG1, CAT, CAV1, CCND1, CCND2, CCNG2, CDKN1A, CDKN1B, ESR1, FASLG, FBXO32, GADD45A, INSR, MXI1, NOS3, PCK1, POMC, PPARGC1A, PRDX3, RBL2, SOD2 and TNFSF10;
wherein the three or more target genes for the NFkB cellular signaling pathway are selected from the group consisting of: BIRC3, CCL3, CCL4, CCL5, CCL20, CXCL2, ICAM1, IL6, IRF1, MMP9, NFKB2, PTGS2, TNF, TNIP1, TRAF1, and VCAM1;
wherein the three or more target genes for the JAK-STAT1/2 cellular signaling pathway are selected from the group consisting of: APOL1, BID, CXCL9, GBP1, GNAZ, IFI6, IFIT2, IFITM1, IRF1, IRF7, IRF9, ISG15, LY6E, OAS1, PDCD1, RFPL3, SSTR3, STAT1, TAP1 and USP18;
wherein the three or more target genes for the JAK-STAT3 cellular signaling pathway are selected from the group consisting of: AKT1, BCL2, BCL2L1, BIRC5, CCND1, CD274, CDKNIA, CRP, FGF2, FOS, FSCN1, FSCN2, FSCN3, HIFIA, HSP90AA1, HSP90AB1, HSP90B1, HSPA1A, HSPA1B, ICAM1, IFNG, IL10, JunB, MCL1, MMP1, MMP3, MMP9, MUC1, MYC, NOS2, POU2F1, PTGS2, SAA1, STAT1, TIMP1, TNFRSF1B, TWIST1, VIM and ZEB1.

One of the advantages of the above method is that it allows the detection of target genes for multiple cellular signaling pathways, and can be performed in a single reaction. This allows the very quick (e.g. within 2-3 hours) detection of expression levels in target genes and subsequent determination of cellular signaling pathway activities. This is very advantageous, for example in critical care, where cellular signaling pathway analysis may be used for quick diagnosis of a patient. In such cases it is essential that the method can be performed fast and without specialized equipment or personnel, as is the case with the disclosed method. The present method allows the detection of the expression levels of target genes in a sample for multiple cellular signaling pathways in a single reaction.

A further advantage of the method as disclosed herein is that it allows reliable determination of expression levels even in samples in which this is typically difficult to do so such as Formalin-Fixed Paraffin-Embedded (FFPE) tissue.

The present method requires a simple qPCR device to run, preferably a thermal cycler with fluorescence readout such as, but not limited to, the Idylla platform. For example a premade plate or container can be used comprising the required reagents and primers and probes, on which the sample is deposited. The PCR protocol does not need to be amended depending on the pathway(s) that are to be analyzed as it is standardized. During amplification cycling, probe intensity can be measured and pathway activity can be determined following conclusion of the protocol, based on the determined expression levels of the target genes. This final step uses a mathematical method to relate the input numerical values representing the expression levels of three or more target genes to a pathway activity. This step is almost instantaneously and can either be performed locally e.g. on a computer or a phone or on a remote server.

Although the method describes target genes for each cellular signaling pathway, it is envisioned that due to the selection criteria for the primers and probes, the method can be applied to alternative target genes for the mentioned cellular signaling pathways that are not listed here. Alternatively, the expression levels of the target genes for cellular signaling pathways not listed herein may be determined using primers and probes constructed using the above criteria combined with the above method.

PCR reactions are typically performed in a 96 well format, however it will be obvious to the skilled artisan that the method is not limited to this format. It is envisioned that the method may also be performed for example, but not limited to, in a single reaction tube (e.g. when multiplexing), “PCR strips” such as 8 well strips or 12 well strips, 384 well plates or 1536 well plates, or any other format, depending on the available volume of sample and the amount of target genes that need to be analyzed.

The method of the invention determines the expression level of six or more genes in a sample, as it is envisioned that preferably for each pathway the expression level of at least three target genes is analyzed, combined with the expression level of at least three reference genes. It is therefore understood that for each additional pathway that is to be analyzed (i.e. for which the activity is to be determined) the expression levels of an additional three or more target genes should be determined in the method. Therefore in order to determine the activity of a single cellular signaling pathway, the expression levels of at least six target genes should be determined, in order to determine the activity of two cellular signaling pathways, the expression levels of at least nine target genes should be determined, in order to determine the activity of three cellular signaling pathways, the expression levels of at least twelve target genes should be determined, etcetera. The method of the invention may be used to determine the activity of one or more, e.g. one, two, three, four, five, six, seven, eight, nine, ten, eleven, twelve, thirteen or more activities of cellular signaling pathways.

For each cellular signaling pathway individually, the expression levels of three or more target genes are to be determined in the method of the invention, e.g. the expression levels of three, four, five, six, seven, eight, nine, ten, eleven, twelve, thirteen or more target genes. Similarly, the expression levels of three or more reference genes are to be determined in the method of the invention, e.g. the expression levels of three, four, five, six, seven, eight, nine, ten, eleven, twelve, thirteen or more reference genes.

When used herein, “simultaneously determining the expression levels” should be interpreted as in a single PCR run, meaning the genes may be amplified and detected in individual wells or containers in the PCR device (e.g. in the separate wells of a 96 well plate, or in separate reaction tubes) or they may also be pooled and amplified in a single reaction (multiplexed), or partially pooled.

The term “sample” as used herein refers to any medium containing nucleotides, preferably RNA, such as but not limited to a medium containing cells, tissue, body fluids, culture medium, or any medium derive from these after further processing steps such as lysis, fixation or isolation of nucleotides, preferably RNA.

It is understood that in order to amplify RNA using PCR technology, first a reverse transcriptase step must be performed. In such a step a reverse transcriptase enzyme is used to generate a complementary DNA form an RNA template. The reverse transcriptase enzyme typically employs a primer sequence which is reverse complementary to a part of the RNA. Generally oligo dT primers may be used for such reaction, or target specific primers. For the method disclosed herein, preferably the reverse primers are used both for the reverse transcriptase reaction and the amplification reaction.

Amplification using DNA primers (polymerase chain reaction) is a technology well known to the skilled person. PCR methods rely on thermal cycling. Thermal cycling exposes reactants to repeated cycles of heating and cooling to permit different temperature dependent reactions, such as DNA melting and enzyme driven DNA replication. PCR employs two main reagents, the forward and reverse primers, which are short single strand DNA fragments known as oligonucleotides that are a complementary sequence to the target DNA region, and a DNA polymerase.

In method disclosed herein the primers and probes amplify and detect of the expression levels of three or more of the reference genes selected from: ACTB, ALAS1, B2M, EEF1A1 POLR2A, PUM1, RPLP0, TBP, TPT1 and TUBA1B, and wherein the primers and probes further amplify and detect the expression levels of three or more target genes for one or more cellular signaling pathways selected from the group consisting of: AR, ER, PI3K-FOXO, MAPK-AP1, HH, Notch, TGFbeta, WNT, PR, NFkB, JAK-STAT1/2 and JAK-STAT3. Therefore the method may include primers and probes for the amplification and detection of three or more target genes for one, two, three, four, five, six seven, eight, nine, ten, eleven, twelve or more cellular signaling pathways. In a preferred embodiment the one or more, e.g. one, two, three, four, five, six, seven, eight, nine, ten, eleven, twelve or more, cellular signaling pathways comprise one or more, e.g. one, two, three, four, five, six or seven, cellular signaling pathways selected from the group consisting of the AR, ER, PI3K-FOXO, MAPK-AP1, HH, Notch and TGFbeta cellular signaling pathways. In a more preferred embodiment the one or more, e.g. one, two, three, four, five, six seven, eight, nine, ten, eleven, twelve or more, cellular signaling pathways comprise one or more, e.g. one, two, three or four, cellular signaling pathways selected from the group consisting of the AR, ER, PI3K-FOXO and MAPK-AP1 cellular signaling pathways, and may optionally further comprise one or more, e.g. one two or three cellular signaling pathway selected from the group consisting of HH, Notch and TGFbeta.

In a further preferred embodiment the one or more, e.g. one, two, three, four, five six or seven or more cellular signaling pathways are selected from JAK-STAT1/2, NFkB, TGFbeta, PI3K-FOXO, MAPK-AP1, Notch and AR. In a further preferred embodiment the one or more, e.g. one, two, three, four, five six, seven, eight, nine, ten, eleven or twelve or more cellular signaling pathway comprise one or more, e.g. one, two, three, four, five six or seven or more cellular signaling pathways selected from JAK-STAT1/2, NFkB, TGFbeta, PI3K-FOXO, MAPK-AP1, Notch and AR. These embodiments are particularly useful as the JAK-STAT1/2, NFkB, TGFbeta, PI3K-FOXO, MAPK-AP1, Notch and AR pathways all play a role in immunity, thus a product allowing to determine part or all of these seven signaling pathway activities is a useful tool in e.g. diagnostics of immune related diseases and disorders, such as infection, autoimmune diseases, but also cancer.

Suitable probes types for qPCR are known to the skilled person. For example, the probes may be fluorophore based, such as TaqMan probes. Fluorophore based probes generally work by including a quencher. TaqMan probes consist of a fluorophore covalently attached to the 5′-end of the oligonucleotide probe and a quencher at the 3′-end. The principle relies on the 5′-3′ exonuclease activity of Taq polymerase to cleave a dual-labeled probe during hybridization to the complementary target sequence and fluorophore-based detection. When bound to the probe the fluorophore is quenched, but once cleaved from the probe the distance with the quencher is increased resulting in fluorescence of the fluorophore when excited at the right wavelength.

In a preferred embodiment of the method of the first aspect of the invention, the primers and probes are able to amplify and detect the respective genes under the following reaction conditions:
50 mM monovalent salt;
400 nM forward primer
400 nM reverse primer
3.0 mM divalent salt, preferably the divalent salt being Mg²⁺;
100 nM probe; and
0.8 mM dNTP.

Therefore, the primers and probes are preferably able to detect the expression level of a single target gen in a medium comprising 50 mM monovalent salt, 3.0 mM divalent salt, preferably the divalent salt being Mg²⁺ and 0.8 mM dNTP, when the primers are present in a concentration of 400 nM for each forward and reverse primer, and wherein the probe is present in a concentration of 100 nM. It is understood that the primers and probes may work outside these parameters, but that the parameters for designing the primers and probe combinations are designed are such that they are suitable for these reaction conditions.

Preferably the divalent salt is MgSO₄ or MgCl₂.

In further preferred embodiment of the method of the first aspect of the invention, the primers and probes are able to amplify and detect the respective genes under the following reaction conditions:
a RT reaction at 500 Celsius for 30 minutes in order to synthesize cDNA, followed by a 5 minute denaturation step at 950 Celsius, followed by 45 cycli of a 15 second denaturation step at 950 Celsius and a 30 second Elongation step at 60° Celsius.

In an embodiment of the method of the first aspect of the invention, the method is used to determine one or more cellular signaling pathway activity or activities. Preferably the one or more cellular signaling Pathway activities are selected from the group consisting of AR, ER, PI3K-FOXO, MAPK-AP1, HH, Notch, TGFbeta, WNT, PR, NFkB, JAK-STAT1/2 and JAK-STAT3, even more preferably from the group consisting of AR, ER, PI3K-FOXO, MAPK-AP1, HH, Notch, TGFbeta, JAK-STAT1/2 and NFkB. An especially advantageous aspect of the invention is the ability to determine multiple pathway activities in a sample in a single reaction, therefore preferably two or more, e.g. three, four, five, six or seven or more cellular signaling pathway activities are determined using the method of the invention. Particularly preferred is using the method to determining four or more cellular signaling pathway activities, wherein the four or more cellular signaling pathway activities comprise the ER, AR, PI3K-FOXO and AP1-MAPK cellular signaling pathways. Further particularly preferred is using the method to determining seven or more cellular signaling pathway activities, wherein the seven or more cellular signaling pathway activities comprise the ER, AR, PI3K-FOXO, AP1-MAPK, Notch, HH and TGFbeta cellular signaling pathways.

In a further preferred embodiment the one or more, e.g. one, two, three, four, five six or seven or more cellular signaling pathways are selected from JAK-STAT1/2, NFkB, TGFbeta, PI3K-FOXO, MAPK-AP1, Notch and AR. In a further preferred embodiment the one or more, e.g. one, two, three, four, five six, seven, eight, nine, ten, eleven or twelve or more cellular signaling pathway comprise one or more, e.g. one, two, three, four, five six or seven or more cellular signaling pathways selected from JAK-STAT1/2, NFkB, TGFbeta, PI3K-FOXO, MAPK-AP1, Notch and AR. These embodiments are particularly useful as the JAK-STAT1/2, NFkB, TGFbeta, PI3K-FOXO, MAPK-AP1, Notch and AR pathways all play a role in immunity, thus a product allowing to determine part or all of these seven signaling pathway activities is a useful tool in e.g. diagnostics of immune related diseases and disorders, such as infection, autoimmune diseases, but also cancer.

It is considered that there are multiple practical applications where the determination of multiple cellular signaling pathway activities may be extremely useful. Although determining pathway activity has been described in the literature by alternative means, known methods are generally not very quantitative and/or easy and/or fast to perform. It is therefore postulated that the presently described solution wherein in a single qPCR reaction multiple cellular signaling pathways activities can be inferred offers extremely useful applications in research and diagnostics, as often multiple cellular signaling pathways are relevant for e.g. making a clinical decision based on pathway activities in a patient sample. The invention provides an easy to perform assay where a quantitative assessment of the activities of e.g. 7 cellular signaling pathways can be determined on a difficult to process sample such as FFPE (Formalin-fixed paraffin embedded) tissue in a matter of 2-3 hours in a single reaction.

When used herein, the terms AP1-MAPK, MAPK-AP1 and MAPK are used interchangeably and refer to the MAPK signaling pathway controlled by the AP1 transcription factor complex. When used herein the terms PI3K-FOXO, FOXO-PI3K and PI3K are used interchangeably and refer to the PI3K signaling pathway, which activity may be determined by taking the inverse of the determined by FOXO pathway activity. When used herein, the terms HH and Hedgehog are used interchangeably and refer to the Hedgehog cellular signaling pathway.

In a preferred embodiment the primers and probes for the ER, AR, PI3K-FOXO, AP1-MAPK, Notch, HH, TGFbeta, JAK-STAT1/2 and NFkB cellular signaling pathways and the reference genes are selected from Tables 1 to 8 and 10 and 11 as described herein below.

In a second aspect the invention relates to an assembly of primers and probe for determining the activity of the ER cellular signaling pathway, wherein the assembly of primers and probes comprises at least three sets of primers or probes for determining the expression level of three or more target genes of the ER cellular signaling pathway, wherein said three or more sets of primers and probes are selected from Table 1 below, wherein each primer and/or probe individually is identical to the corresponding sequence in Table 1 or differs at 1, 2 or 3 positions, wherein said 1, 2 or 3 differences are selected from a single base substitution, a single base deletion or a single base addition.

Tables 1 to 8, 10 and 11 disclose different sets of primers and probes for the different target genes of the referred cellular signaling pathways. The lists are sorted per gene, each gene may have multiple sets of primers and probes, each set of primers and probes consisting of a forward primer, a reverse primer and a probe. Each set is indicated as an “assay”, therefore when used herein, a set of primers and probe refers to a forward primer, a reverse primer and a probe with the same assay name as indicated in one of tables 1 to 8, 10 and 11. The primers and probes are indicated with their respective SEQ ID NO and name (assay), the name being generated according to the following format: [GENE NAME]_[NUMBER], wherein primers and probe with the same name (thus same gene name and number) are part of a single set.

Therefore, the invention further relates to an assembly of primers and probes for determining the activity of the ER cellular signaling pathway, wherein the set of primers and probes comprises at least three sets of primers or probes for determining the expression level of three or more target genes of the ER cellular signaling pathway, wherein said sets of primers and probes are selected from Table 1 wherein the forward primer, the reverse primer and the probe have the same assay name; and
wherein each primer and/or probe individually has the listed nucleotide sequence identified by the corresponding SEQ ID NO or has a sequence that differs at 1, 2 or 3 positions, wherein said 1, 2 or 3 differences are individually selected from a single base substitution, a single base deletion or a single base addition.

Preferably the assembly further comprises three or more sets of primers and probes for determining the expression levels of three or more reference genes, wherein said sets of primers and probes are selected from Table 8 wherein the forward primer, the reverse primer and the probe have the same assay name; and
wherein each primer and/or probe individually has the listed nucleotide sequence identified by the corresponding SEQ ID NO or has a sequence that differs at 1, 2 or 3 positions, wherein said 1, 2 or 3 differences are individually selected from a single base substitution, a single base deletion or a single base addition.

The assembly may further comprise one or more, e.g. one, two, three, four, five, six, seven, eight or more, additional assemblies of primers and probes as defined herein.

It is recognized that the PCR reaction is tolerant to a few, e.g. 1, 2 or 3, mismatches in the primer or probe sequences and still allows amplification and detection of the target gene expression level. Therefore, up to 3 mismatches may be allowed in the sequences of the primers and probes each. A mismatch or difference at a position of a sequence when used herein refers to a single base substitution, a single base deletion or a single base insertion with respect to the reference sequence (e.g. the respective SEQ ID NO). It is recognized that the PCR reaction is general more tolerant to a substitution as compared to a deletion or insertion in the primers or probes, therefore preferably the mismatch or difference at a position of a sequence is a single base substitution, although a deletion or insertion ay be desirable to account for genetic variation in the target DNA of the sample.

It is understood that avoiding mismatches is preferable for optimal amplification and detection of the target gene. Therefore preferably each primer and/or probe individually is identical to the corresponding SEQ ID NO or differs at 1 or 2 positions, more preferably each primer and/or probe individually is identical to the corresponding SEQ ID NO or differs at 1 position, most preferably each primer and/or probe individually is identical to the corresponding SEQ ID NO.

When used herein, a set of primers and probe refers to set consisting of a forward primer and a reverse primer for amplifying a genomic sequence and a probe for detecting the genomic sequence, where the genomic sequence is a target gene suited for determining the activity of a cellular signaling pathway. The forward and reverse primer and corresponding probe belonging to a single “set” are defined in the “assays” listed in Tables 1 to 8, and thus the primers and probe have an identical assay name when belonging to the same “set”.

When used herein, an assembly of primers and probes refers to multiple (e.g. at least two) sets of primers and probes as defined herein.

In a third aspect the invention further relates to an assembly of primers and probes for determining the activity of the AR cellular signaling pathway, wherein the set of primers and probes comprises at least three sets of primers or probes for determining the expression level of three or more target genes of the AR cellular signaling pathway, wherein said sets of primers and probes are selected from Table 2 wherein the forward primer, the reverse primer and the probe have the same assay name; and
wherein each primer and/or probe individually has the listed nucleotide sequence identified by the corresponding SEQ ID NO or has a sequence that differs at 1, 2 or 3 positions, wherein said 1, 2 or 3 differences are individually selected from a single base substitution, a single base deletion or a single base addition.

Preferably the assembly further comprises three or more sets of primers and probes for determining the expression levels of three or more reference genes, wherein said sets of primers and probes are selected from Table 8 wherein the forward primer, the reverse primer and the probe have the same assay name; and
wherein each primer and/or probe individually has the listed nucleotide sequence identified by the corresponding SEQ ID NO or has a sequence that differs at 1, 2 or 3 positions, wherein said 1, 2 or 3 differences are individually selected from a single base substitution, a single base deletion or a single base addition.

It is recognized that the PCR reaction is tolerant to a few, e.g. 1, 2 or 3, mismatches in the primer or probe sequences and still allows amplification and detection of the target gene expression level. Therefore, up to 3 mismatches may be allowed in the sequences of the primers and probes each. A mismatch or difference at a position of a sequence when used herein refers to a single base substitution, a single base deletion or a single base insertion with respect to the reference sequence (e.g. the respective SEQ ID NO). It is recognized that the PCR reaction is general more tolerant to a substitution as compared to a deletion or insertion in the primers or probes, therefore preferably the mismatch or difference at a position of a sequence is a single base substitution, although a deletion or insertion ay be desirable to account for genetic variation in the target DNA of the sample.

It is understood that avoiding mismatches is preferable for optimal amplification and detection of the target gene. Therefore preferably each primer and/or probe individually is identical to the corresponding SEQ ID NO or differs at 1 or 2 positions, more preferably each primer and/or probe individually is identical to the corresponding SEQ ID NO or differs at 1 position, most preferably each primer and/or probe individually is identical to the corresponding SEQ ID NO.

The assembly may further comprise one or more, e.g. one, two, three, four, five, six, seven, eight or more, additional assemblies of primers and probes as defined herein.

In a fourth aspect the invention further relates to an assembly of primers and probes for determining the activity of the PI3K-FOXO cellular signaling pathway, wherein the set of primers and probes comprises at least three sets of primers or probes for determining the expression level of three or more target genes of the PI3K-FOXO cellular signaling pathway, wherein said sets of primers and probes are selected from Table 3 wherein the forward primer, the reverse primer and the probe have the same assay name; and
wherein each primer and/or probe individually has the listed nucleotide sequence identified by the corresponding SEQ ID NO or has a sequence that differs at 1, 2 or 3 positions, wherein said 1, 2 or 3 differences are individually selected from a single base substitution, a single base deletion or a single base addition.

Preferably the assembly further comprises three or more sets of primers and probes for determining the expression levels of three or more reference genes, wherein said sets of primers and probes are selected from Table 8 wherein the forward primer, the reverse primer and the probe have the same assay name; and
wherein each primer and/or probe individually has the listed nucleotide sequence identified by the corresponding SEQ ID NO or has a sequence that differs at 1, 2 or 3 positions, wherein said 1, 2 or 3 differences are individually selected from a single base substitution, a single base deletion or a single base addition.

It is recognized that the PCR reaction is tolerant to a few, e.g. 1, 2 or 3, mismatches in the primer or probe sequences and still allows amplification and detection of the target gene expression level. Therefore, up to 3 mismatches may be allowed in the sequences of the primers and probes each. A mismatch or difference at a position of a sequence when used herein refers to a single base substitution, a single base deletion or a single base insertion with respect to the reference sequence (e.g. the respective SEQ ID NO). It is recognized that the PCR reaction is general more tolerant to a substitution as compared to a deletion or insertion in the primers or probes, therefore preferably the mismatch or difference at a position of a sequence is a single base substitution, although a deletion or insertion ay be desirable to account for genetic variation in the target DNA of the sample.

It is understood that avoiding mismatches is preferable for optimal amplification and detection of the target gene. Therefore preferably each primer and/or probe individually is identical to the corresponding SEQ ID NO or differs at 1 or 2 positions, more preferably each primer and/or probe individually is identical to the corresponding SEQ ID NO or differs at 1 position, most preferably each primer and/or probe individually is identical to the corresponding SEQ ID NO.

The assembly may further comprise one or more, e.g. one, two, three, four, five, six, seven, eight or more, additional assemblies of primers and probes as defined herein.

In a fifth aspect the invention further relates to an assembly of primers and probes for determining the activity of the AP1-MAPK cellular signaling pathway, wherein the set of primers and probes comprises at least three sets of primers or probes for determining the expression level of three or more target genes of the AP1-MAPK cellular signaling pathway, wherein said sets of primers and probes are selected from Table 4 wherein the forward primer, the reverse primer and the probe have the same assay name; and
wherein each primer and/or probe individually has the listed nucleotide sequence identified by the corresponding SEQ ID NO or has a sequence that differs at 1, 2 or 3 positions, wherein said 1, 2 or 3 differences are individually selected from a single base substitution, a single base deletion or a single base addition.

Preferably the assembly further comprises three or more sets of primers and probes for determining the expression levels of three or more reference genes, wherein said sets of primers and probes are selected from Table 8 wherein the forward primer, the reverse primer and the probe have the same assay name; and
wherein each primer and/or probe individually has the listed nucleotide sequence identified by the corresponding SEQ ID NO or has a sequence that differs at 1, 2 or 3 positions, wherein said 1, 2 or 3 differences are individually selected from a single base substitution, a single base deletion or a single base addition.

It is recognized that the PCR reaction is tolerant to a few, e.g. 1, 2 or 3, mismatches in the primer or probe sequences and still allows amplification and detection of the target gene expression level. Therefore, up to 3 mismatches may be allowed in the sequences of the primers and probes each. A mismatch or difference at a position of a sequence when used herein refers to a single base substitution, a single base deletion or a single base insertion with respect to the reference sequence (e.g. the respective SEQ ID NO). It is recognized that the PCR reaction is general more tolerant to a substitution as compared to a deletion or insertion in the primers or probes, therefore preferably the mismatch or difference at a position of a sequence is a single base substitution, although a deletion or insertion ay be desirable to account for genetic variation in the target DNA of the sample.

It is understood that avoiding mismatches is preferable for optimal amplification and detection of the target gene. Therefore preferably each primer and/or probe individually is identical to the corresponding SEQ ID NO or differs at 1 or 2 positions, more preferably each primer and/or probe individually is identical to the corresponding SEQ ID NO or differs at 1 position, most preferably each primer and/or probe individually is identical to the corresponding SEQ ID NO.

The assembly may further comprise one or more, e.g. one, two, three, four, five, six, seven, eight or more, additional assemblies of primers and probes as defined herein.

In a sixth aspect the invention further relates to an assembly of primers and probes for determining the activity of the Notch cellular signaling pathway, wherein the set of primers and probes comprises at least three sets of primers or probes for determining the expression level of three or more target genes of the Notch cellular signaling pathway, wherein said sets of primers and probes are selected from Table 5 wherein the forward primer, the reverse primer and the probe have the same assay name; and
wherein each primer and/or probe individually has the listed nucleotide sequence identified by the corresponding SEQ ID NO or has a sequence that differs at 1, 2 or 3 positions, wherein said 1, 2 or 3 differences are individually selected from a single base substitution, a single base deletion or a single base addition.

Preferably the assembly further comprises three or more sets of primers and probes for determining the expression levels of three or more reference genes, wherein said sets of primers and probes are selected from Table 8 wherein the forward primer, the reverse primer and the probe have the same assay name; and
wherein each primer and/or probe individually has the listed nucleotide sequence identified by the corresponding SEQ ID NO or has a sequence that differs at 1, 2 or 3 positions, wherein said 1, 2 or 3 differences are individually selected from a single base substitution, a single base deletion or a single base addition.

It is recognized that the PCR reaction is tolerant to a few, e.g. 1, 2 or 3, mismatches in the primer or probe sequences and still allows amplification and detection of the target gene expression level. Therefore, up to 3 mismatches may be allowed in the sequences of the primers and probes each. A mismatch or difference at a position of a sequence when used herein refers to a single base substitution, a single base deletion or a single base insertion with respect to the reference sequence (e.g. the respective SEQ ID NO). It is recognized that the PCR reaction is general more tolerant to a substitution as compared to a deletion or insertion in the primers or probes, therefore preferably the mismatch or difference at a position of a sequence is a single base substitution, although a deletion or insertion ay be desirable to account for genetic variation in the target DNA of the sample.

It is understood that avoiding mismatches is preferable for optimal amplification and detection of the target gene. Therefore preferably each primer and/or probe individually is identical to the corresponding SEQ ID NO or differs at 1 or 2 positions, more preferably each primer and/or probe individually is identical to the corresponding SEQ ID NO or differs at 1 position, most preferably each primer and/or probe individually is identical to the corresponding SEQ ID NO.

The assembly may further comprise one or more, e.g. one, two, three, four, five, six, seven, eight or more, additional assemblies of primers and probes as defined herein.

In a seventh aspect the invention further relates to an assembly of primers and probes for determining the activity of the HH cellular signaling pathway, wherein the set of primers and probes comprises at least three sets of primers or probes for determining the expression level of three or more target genes of the HH cellular signaling pathway, wherein said sets of primers and probes are selected from Table 6 wherein the forward primer, the reverse primer and the probe have the same assay name; and
wherein each primer and/or probe individually has the listed nucleotide sequence identified by the corresponding SEQ ID NO or has a sequence that differs at 1, 2 or 3 positions, wherein said 1, 2 or 3 differences are individually selected from a single base substitution, a single base deletion or a single base addition.

Preferably the assembly further comprises three or more sets of primers and probes for determining the expression levels of three or more reference genes, wherein said sets of primers and probes are selected from Table 8 wherein the forward primer, the reverse primer and the probe have the same assay name; and
wherein each primer and/or probe individually has the listed nucleotide sequence identified by the corresponding SEQ ID NO or has a sequence that differs at 1, 2 or 3 positions, wherein said 1, 2 or 3 differences are individually selected from a single base substitution, a single base deletion or a single base addition.

It is recognized that the PCR reaction is tolerant to a few, e.g. 1, 2 or 3, mismatches in the primer or probe sequences and still allows amplification and detection of the target gene expression level. Therefore, up to 3 mismatches may be allowed in the sequences of the primers and probes each. A mismatch or difference at a position of a sequence when used herein refers to a single base substitution, a single base deletion or a single base insertion with respect to the reference sequence (e.g. the respective SEQ ID NO). It is recognized that the PCR reaction is general more tolerant to a substitution as compared to a deletion or insertion in the primers or probes, therefore preferably the mismatch or difference at a position of a sequence is a single base substitution, although a deletion or insertion ay be desirable to account for genetic variation in the target DNA of the sample.

It is understood that avoiding mismatches is preferable for optimal amplification and detection of the target gene. Therefore preferably each primer and/or probe individually is identical to the corresponding SEQ ID NO or differs at 1 or 2 positions, more preferably each primer and/or probe individually is identical to the corresponding SEQ ID NO or differs at 1 position, most preferably each primer and/or probe individually is identical to the corresponding SEQ ID NO.

The assembly may further comprise one or more, e.g. one, two, three, four, five, six, seven, eight or more, additional assemblies of primers and probes as defined herein.

In a eight aspect the invention further relates to an assembly of primers and probes for determining the activity of the TGFbeta cellular signaling pathway, wherein the set of primers and probes comprises at least three sets of primers or probes for determining the expression level of three or more target genes of the TGFbeta cellular signaling pathway, wherein said sets of primers and probes are selected from Table 7 wherein the forward primer, the reverse primer and the probe have the same assay name; and
wherein each primer and/or probe individually has the listed nucleotide sequence identified by the corresponding SEQ ID NO or has a sequence that differs at 1, 2 or 3 positions, wherein said 1, 2 or 3 differences are individually selected from a single base substitution, a single base deletion or a single base addition.

Preferably the assembly further comprises three or more sets of primers and probes for determining the expression levels of three or more reference genes, wherein said sets of primers and probes are selected from Table 8 wherein the forward primer, the reverse primer and the probe have the same assay name; and
wherein each primer and/or probe individually has the listed nucleotide sequence identified by the corresponding SEQ ID NO or has a sequence that differs at 1, 2 or 3 positions, wherein said 1, 2 or 3 differences are individually selected from a single base substitution, a single base deletion or a single base addition.

It is recognized that the PCR reaction is tolerant to a few, e.g. 1, 2 or 3, mismatches in the primer or probe sequences and still allows amplification and detection of the target gene expression level. Therefore, up to 3 mismatches may be allowed in the sequences of the primers and probes each. A mismatch or difference at a position of a sequence when used herein refers to a single base substitution, a single base deletion or a single base insertion with respect to the reference sequence (e.g. the respective SEQ ID NO). It is recognized that the PCR reaction is general more tolerant to a substitution as compared to a deletion or insertion in the primers or probes, therefore preferably the mismatch or difference at a position of a sequence is a single base substitution, although a deletion or insertion ay be desirable to account for genetic variation in the target DNA of the sample.

It is understood that avoiding mismatches is preferable for optimal amplification and detection of the target gene. Therefore preferably each primer and/or probe individually is identical to the corresponding SEQ ID NO or differs at 1 or 2 positions, more preferably each primer and/or probe individually is identical to the corresponding SEQ ID NO or differs at 1 position, most preferably each primer and/or probe individually is identical to the corresponding SEQ ID NO.

In a ninth aspect the invention further relates to an assembly of primers and probes according to any one of the preceding claims further comprising primers and probes for determining the activity of the JAK-STAT1/2 cellular signaling pathway, wherein the assembly of primers and probes comprises three or more sets of primers or probes for determining the expression level of three or more target genes of the JAK-STAT1/2 cellular signaling pathway, wherein said three or more sets of primers and probes are selected from Table 10 of the description,
wherein each primer and/or probe individually has the listed nucleotide sequence identified by the corresponding SEQ ID NO or has a sequence that differs at 1, 2 or 3 positions, wherein said 1, 2 or 3 differences are individually selected from a single base substitution, a single base deletion or a single base addition.

Preferably the assembly further comprises three or more sets of primers and probes for determining the expression levels of three or more reference genes, wherein said sets of primers and probes are selected from Table 10 wherein the forward primer, the reverse primer and the probe have the same assay name; and
wherein each primer and/or probe individually has the listed nucleotide sequence identified by the corresponding SEQ ID NO or has a sequence that differs at 1, 2 or 3 positions, wherein said 1, 2 or 3 differences are individually selected from a single base substitution, a single base deletion or a single base addition.

It is recognized that the PCR reaction is tolerant to a few, e.g. 1, 2 or 3, mismatches in the primer or probe sequences and still allows amplification and detection of the target gene expression level. Therefore, up to 3 mismatches may be allowed in the sequences of the primers and probes each. A mismatch or difference at a position of a sequence when used herein refers to a single base substitution, a single base deletion or a single base insertion with respect to the reference sequence (e.g. the respective SEQ ID NO). It is recognized that the PCR reaction is general more tolerant to a substitution as compared to a deletion or insertion in the primers or probes, therefore preferably the mismatch or difference at a position of a sequence is a single base substitution, although a deletion or insertion ay be desirable to account for genetic variation in the target DNA of the sample.

It is understood that avoiding mismatches is preferable for optimal amplification and detection of the target gene. Therefore preferably each primer and/or probe individually is identical to the corresponding SEQ ID NO or differs at 1 or 2 positions, more preferably each primer and/or probe individually is identical to the corresponding SEQ ID NO or differs at 1 position, most preferably each primer and/or probe individually is identical to the corresponding SEQ ID NO.

The assembly may further comprise one or more, e.g. one, two, three, four, five, six, seven, eight or more, additional assemblies of primers and probes as defined herein.

In a tenth aspect the invention relates to an assembly of primers and probes according to any one of the preceding claims further comprising primers and probes for determining the activity of the NFkB cellular signaling pathway, wherein the assembly of primers and probes comprises three or more sets of primers or probes for determining the expression level of three or more target genes of the NFkB cellular signaling pathway, wherein said three or more sets of primers and probes are selected from Table 11 of the description,
wherein each primer and/or probe individually has the listed nucleotide sequence identified by the corresponding SEQ ID NO or has a sequence that differs at 1, 2 or 3 positions, wherein said 1, 2 or 3 differences are individually selected from a single base substitution, a single base deletion or a single base addition.

Preferably the assembly further comprises three or more sets of primers and probes for determining the expression levels of three or more reference genes, wherein said sets of primers and probes are selected from Table 11 wherein the forward primer, the reverse primer and the probe have the same assay name; and
wherein each primer and/or probe individually has the listed nucleotide sequence identified by the corresponding SEQ ID NO or has a sequence that differs at 1, 2 or 3 positions, wherein said 1, 2 or 3 differences are individually selected from a single base substitution, a single base deletion or a single base addition.

It is recognized that the PCR reaction is tolerant to a few, e.g. 1, 2 or 3, mismatches in the primer or probe sequences and still allows amplification and detection of the target gene expression level. Therefore, up to 3 mismatches may be allowed in the sequences of the primers and probes each. A mismatch or difference at a position of a sequence when used herein refers to a single base substitution, a single base deletion or a single base insertion with respect to the reference sequence (e.g. the respective SEQ ID NO). It is recognized that the PCR reaction is general more tolerant to a substitution as compared to a deletion or insertion in the primers or probes, therefore preferably the mismatch or difference at a position of a sequence is a single base substitution, although a deletion or insertion ay be desirable to account for genetic variation in the target DNA of the sample.

It is understood that avoiding mismatches is preferable for optimal amplification and detection of the target gene. Therefore preferably each primer and/or probe individually is identical to the corresponding SEQ ID NO or differs at 1 or 2 positions, more preferably each primer and/or probe individually is identical to the corresponding SEQ ID NO or differs at 1 position, most preferably each primer and/or probe individually is identical to the corresponding SEQ ID NO.

The assembly may further comprise one or more, e.g. one, two, three, four, five, six, seven, eight or more, additional assemblies of primers and probes as defined herein.

In a particularly preferred embodiment, the invention relates to an assembly of primers and probe for determining the activity of the AR cellular signaling pathway, and optionally one or more additional cellular signaling pathway, wherein the assembly of primers and probes comprises three or more sets of primers or probes for determining the expression level of three or more target genes of the AR cellular signaling pathway, wherein said three or more sets of primers and probes are selected from Table 2 of the description,
wherein each primer and/or probe individually has the listed nucleotide sequence identified by the corresponding SEQ ID NO or has a sequence that differs at 1, 2 or 3 positions, wherein said 1, 2 or 3 differences are individually selected from a single base substitution, a single base deletion or a single base addition,
wherein said invention further comprises one or more, such as one, two, three, four, five, six, seven or eight or more, additional assemblies as defined herein, e.g. an assembly of primers and probes for determining the ER, PI3K-FOXO, MAPK-AP1, Notch, HH, TGFbeta, JAK-STAT1/2 and/or NFkB pathway activity. However it is envisioned that other combinations of assemblies may also be made in accordance with the invention, e.g. combinations of assemblies not including an assembly of primers and probes for determining the AR pathway activity.

In an embodiment of the assembly according the second to the tenth aspect of the invention, all of the primers and probes in the three or more sets of primers and probes in the assembly are identical to the corresponding sequences according to Tables 1 to 7, 10 and 11 or the sequences represented by the indicated SEQ ID Nos.

In a eleventh aspect the invention relates to a kit of parts for determining the expression levels for a plurality of genes, the kit comprising primers and probes for the amplification and detection of the expression levels of the plurality of genes, wherein the kit comprises primers and probes are as defined in the first aspect of the invention or an assembly of primers and probes as defined in any one of the second to the tenth aspect of the invention, wherein the kit further comprises primers and probes for the amplification and detection of three or more of the reference genes selected from ACTB, ALAS1, B2M, EEF1A1 POLR2A, PUM1, RPLP0, TBP, TPT1 and TUBA1B. Preferably said three or more sets of primers and probes are selected from Table 8 of the description, and wherein each primer and/or probe individually has the listed nucleotide sequence identified by the corresponding SEQ ID NO or has a sequence that differs at 1, 2 or 3 positions, wherein said 1, 2 or 3 differences are individually selected from a single base substitution, a single base deletion or a single base addition.

Preferably the kit is suitable for determining the activity of a cellular signaling pathway. Therefore the kit preferably comprises at least three sets of primers which are suitable for the amplification and detection of the expression levels of at least three target genes of a cellular signaling pathway as described in the first to the eight aspect of the invention. Preferably the cellular signaling pathway is selected from the group consisting of ER, AR, PI3K-FOXO, MAPK-AP1, WNT, HH, PR, TGFbeta, NFkB, STAT1/2, STAT3, and Notch. More preferably the kit comprises three or more sets of primers and probes, e.g. three, four, five, six, seven, eight, nine, ten, eleven or twelve or more, as described herein each for the detection of one or more, e.g. one, two, three, four, five, six, seven, eight, nine, ten, eleven or twelve, pathway activities selected from the group consisting of ER, AR, PI3K-FOXO, MAPK-AP1, WNT, HH, PR, TGFbeta, NFkB, STAT1/2, STAT3, and Notch, more preferably selected from the group consisting of ER, AR, PI3K-FOXO, MAPK-AP1, Notch, HH and TGFbeta. Preferably said one or more, e.g. one, two, three, four, five, six, seven, eight, nine, ten, eleven or twelve, pathway activities selected from the group consisting of ER, AR, PI3K-FOXO, MAPK-AP1, WNT, HH, PR, TGFbeta, NFkB, STAT1/2, STAT3, and Notch comprise one or more, e.g. one, two, three, four, five, six or seven, eight or nine pathways selected from the group consisting of ER, AR, PI3K-FOXO, MAPK-AP1, Notch, HH, TGFbeta, JAK-STAT1/2 and NFkB. Preferably said one or more, e.g. one, two, three, four, five, six, seven, eight, nine, ten, eleven or twelve, pathway activities selected from the group consisting of ER, AR, PI3K-FOXO, MAPK-AP1, WNT, HH, PR, TGFbeta, NFkB, STAT1/2, STAT3, and Notch comprise one or more, e.g. one, two, three or four, pathways selected from the group consisting of ER, AR, PI3K-FOXO and MAPK-AP1, or comprises one or more, e.g. one, two, three, four, five, six or seven pathways selected from the groups consisting of ER, AR, PI3K-FOXO, MAPK-AP1, Notch, HH, TGFbeta, or comprises one or more, e.g. one, two, three, four, five, six or seven pathways selected from the groups consisting of JAK-STAT 1/2, NFkB, TGFbeta, PI3K-FOXO, MAPK-AP1, Notch en AR.

Optionally the primers and probes for the amplification and detection of the reference genes are selected from Table 8 below, although it is understood that other sets of primers and probes may be used, provided the primers and probes are suitable at the reaction conditions described herein above.

The kit may optionally further comprise one or more of a polymerase enzyme, a reverse transcriptase enzyme, a suitable buffer and a container for performing the PCR reaction. Suitable containers may be reaction tubes, PCR strips such as 8 well or 12 well strips or multiwell plates, also known as microwell plates, microtiter plates or microplates, such as 6, 12, 24, 48, 96, 384, or 1536 well plates, or any other container which can be used in a thermal cycler, preferably a thermal cycler with fluorescence readout capability.

In an twelfth aspect the invention relates to the use of the primers and probes as defined in the first aspect or the assembly of primers and probes as defined in the second to the tenth aspect or the kit as defined in eleventh aspect for determining the cellular signaling pathway activity for one or more cellular signaling pathways selected from the group consisting of: ER, AR, PI3K-FOXO, MAPK-AP1, WNT, HH, PR, TGFbeta, NFkB, STAT1/2, STAT3, and Notch.

In a thirteenth aspect the invention relates to the use of a set of three or more primers and probes to determine the expression levels of three or more target genes of a cellular signaling pathway, wherein the primers and probe combinations are as defined in the second to the eleventh aspect, and
wherein the three or more target genes for the AR cellular signaling pathway are selected from the group consisting of: ABCC4, AR, CREB3L4, DHCR24, ELL2, FKBP5, GUCY1A3, KLK2, KLK3, LRIG1, NDRG1, NKX3.1 (also known as NKX3_1), PLAU, PMEPA1, PPAP2A, PRKACB_2, SGK1, and TMPRSS2;
wherein the three or more target genes for the ER cellular signaling pathway are selected from the group consisting of: AP1B1, CA12, CDH26, CELSR2, CTSD, ERBB2, ESR1, GREB1, HSPB1, IGFBP4, MYC, NRIP1, PDZK1, PGR, RARA, SGK3, SOD1, TFF1, WISP2, and XBP1;
the three or more target genes for the PI3K-FOXO cellular signaling pathway are selected from the group consisting of: AGRP, BCL2L11, BCL6, BNIP3, BTG1, CAT, CAV1, CCND1, CCNG2, CDKN1A, CDKN1B, ESR1, FBXO32, FOXO3, GADD45A, INSR, MXI1, SOD2, TNFSF10;
wherein the three or more target genes for the MAPK-AP1 cellular signaling pathway are selected from the group consisting of: BCL2L11, CCND1, DDIT3, EGFR, ENPP2, EZR, GLRX, MMP1, MMP3, MMP9, PLAU, PLAUR, PTGS2, SERPINE1, TIMP1, TP53, VEGFD, and VIM;
wherein the three or more target genes for the Notch cellular signaling pathway are selected from the group consisting of: CD44, EPHB3, FABP7, HES1, HES4, HES5, HEY1, HEY2, MYC, NOX1, NRARP, PIN1, PLXND1, and SOX9;
wherein the three or more target genes for the HH cellular signaling pathway are selected from the group consisting of: CFLAR, FOXM1, FYN, GLI1, HHIP, MYCN, NKX2-2, PTCH1, PTCH2, RAB34, SPP1, TCEA2, and TSC22D1;
wherein the three or more target genes for the TGFbeta cellular signaling pathway are selected from the group consisting of: ANGPTL4, CDKN1A, CTGF, GADD45A, GADD45B, ID1, IL11, JUNB, MMP2, MMP9, PDGFB, SERPINE1, SGK1, SKIL, SMAD4, SMAD7, SNAI1, TIMP1, and VEGFA;
wherein the three or more target genes for the WNT cellular signaling pathway are selected from the group consisting of: CEMIP, AXIN2, CD44, RNF43, MYC, TBX3, TDGF1, SOX9, ASCL2, CXCL8, SP5, ZNRF3, EPHB2, LGR5, EPHB3, KLF6, CCND1, DEFA6, and FZD7;
wherein the three or more target genes for the PR cellular signaling pathway are selected from the group consisting of: AGRP, BCL2L11, BCL6, BNIP3, BTG1, CAT, CAV1, CCND1, CCND2, CCNG2, CDKN1A, CDKN1B, ESR1, FASLG, FBXO32, GADD45A, INSR, MXI1, NOS3, PCK1, POMC, PPARGC1A, PRDX3, RBL2, SOD2 and TNFSF10;
wherein the three or more target genes for the NFkB cellular signaling pathway are selected from the group consisting of: BIRC3, CCL3, CCL4, CCL5, CCL20, CXCL2, ICAM1, IL6, IRF1, MMP9, NFKB2, PTGS2, TNF, TNIP1, TRAF1, and VCAM1;
wherein the three or more target genes for the JAK-STAT1/2 cellular signaling pathway are selected from the group consisting of: APOL1, BID, CXCL9, GBP1, GNAZ, IFI6, IFIT2, IFITM1, IRF1, IRF7, IRF9, ISG15, LY6E, OAS1, PDCD1, RFPL3, SSTR3, STAT1, TAP1 and USP18;
wherein the three or more target genes for the JAK-STAT3 cellular signaling pathway are selected from the group consisting of: AKT1, BCL2, BCL2L1, BIRC5, CCND1, CD274, CDKNIA, CRP, FGF2, FOS, FSCN1, FSCN2, FSCN3, HIFIA, HSP90AA1, HSP90AB1, HSP90B1, HSPA1A, HSPA1B, ICAM1, IFNG, IL10, JunB, MCL1, MMP1, MMP3, MMP9, MUC1, MYC, NOS2, POU2F1, PTGS2, SAA1, STAT1, TIMP1, TNFRSF1B, TWIST1, VIM and ZEB1.

In a fourteenth aspect the invention relates to a method for designing primers and probes for the detection of the expression levels of target genes of a cellular signaling pathway suitable for determining the activity of said cellular signaling pathway, said cellular pathway preferably being one or more, for example on, two, three, four, five six, seven, eight or nine, cellular signaling pathways selected from the groups consisting of ER, AR, PI3K-FOXO, MAPK-AP1, HH, Notch, TGFBeta, JAK-STAT1/2 and NFkB, the method comprising:
designing for a target gene of a cellular signaling pathway a forward primer and a reverse primer such that:
the forward and reverse primer have a GC content between 35% and 69%, preferably between 35% and 65%;
the forward and reverse primer have a melting temperature between 50 and 71 degrees Celsius, preferably between 58 and 64 degrees Celsius;
the forward and reverse primer have a length between 16 and 25 nucleotides, preferably between 17 and 24 nucleotides;
wherein the amplification product, when using the forward and reverse primers in a PCR amplification reaction, has a size between 60 and 240 base pairs, preferably between 65 and 150 base pairs, and preferably wherein the amplicon product is intron spanning;
designing the probe such that:
the probe used for detection of an amplification product comprises a binding part which is complementary to a part of the amplification product, the binding part further having the following characteristics:
the binding part of the probe has a GC content between 35% and 69%, preferably between 40% and 60%;
the binding part of the probe has a melting temperature between 56 and 72 degrees Celsius, preferably between 64 and 72 degrees Celsius;
the binding part of the probe has a length between 17 and 31 nucleotides, preferably between 18 and 30 nucleotides;
the binding part of the probe does not have a G at the 5′ part.

Primers designed according t this method are useful as they can be used in an assay for determining one or more cellular signaling pathway activities. Preferably the primers and probes are able to detect the expression level of a target gene under the following reaction conditions:
50 mM monovalent salt;
400 nM forward primer
400 nM reverse primer
3.0 mM divalent salt, preferably the divalent salt being Mg²⁺;
100 nM probe; and
0.8 mM dNTP
Preferably the divalent salt is MgSO₄ or MgCl₂.

Preferably the primers and probes are able to amplify and detect the respective genes under the following reaction conditions:
a RT reaction at 500 Celsius for 30 minutes in order to synthesize cDNA, followed by a 5 minute denaturation step at 950 Celsius, followed by 44 cycli of a 15 second denaturation step at 950 Celsius and a 30 second Elongation step at 60° Celsius.

In a fifteenth embodiment the invention relates to a method of determining the a cellular signaling pathway activity selected from ER, AR, PI3K-FOXO, MAPK-AP1, HH, Notch, TGFBeta, JAK-STAT1/2 and NFkB and optionally one or more additional cellular signaling pathway activity or activities, preferably said one or more additional cellular signaling pathway activities are also selected from ER, AR, PI3K-FOXO, MAPK-AP1, HH, Notch, TGFBeta, JAK-STAT1/2 and NFkB, by simultaneously determining the expression level of six or more genes in a sample, the method comprising simultaneously amplifying six or more gene products using a polymerase chain reaction to generate a plurality of amplification products, followed by the detection of the plurality of amplification products using a plurality of probes,
wherein the polymerase chain reaction uses, for each amplification product, a forward and a reverse primer which have the following characteristics:
the forward and reverse primer have a GC content between 35% and 69%, preferably between 35% and 65%;
the forward and reverse primer have a melting temperature between 50 and 71 degrees Celsius, preferably between 58 and 64 degrees Celsius;
the forward and reverse primer have a length between 16 and 25 nucleotides, preferably between 17 and 24 nucleotides;
wherein the amplification products have a size between 60 and 240 base pairs, preferably between 65 and 150 base pairs, and preferably wherein the amplicon product is intron spanning,
wherein each of the probes used for detection of an amplification product comprises a binding part which is complementary to a part of the amplification product, the binding part further having the following characteristics:
the binding part of the probe has a GC content between 35% and 69%, preferably between 40% and 60%;
the binding part of the probe has a melting temperature between 56 and 72 degrees Celsius, preferably between 64 and 72 degrees Celsius;
the binding part of the probe has a length between 17 and 31 nucleotides, preferably between 18 and 30 nucleotides;
the binding part of the probe does not have a G at the 5′ part,
wherein the expression levels are used in a method for determining the AR cellular signaling pathway and optionally one or more cellular signaling pathway activities selected from the group consisting of: WNT, HH, ER, PR, PR, TGFbeta, NFkB, STAT1/2, STAT3, PI3K-FOXO, Notch, MAPK-AP1, and
wherein the primers and probes amplify and detect of the expression levels of three or more of the reference genes selected from: ACTB, ALAS1, B2M, EEF1A1 POLR2A, PUM1, RPLP0, TBP, TPT1 and TUBA1B, and
wherein the primers and probes further amplify and detect the expression levels of three or more target genes for the AR cellular signaling pathway and optionally one or more cellular signaling pathways selected from the group consisting of: ER, PI3K-FOXO, MAPK-AP1, HH, Notch, TGFbeta, WNT, PR, NFkB, JAK-STAT1/2, JAK-STAT3,
wherein the three or more target genes for the AR cellular signaling pathway are selected from the group consisting of: ABCC4, AR, CREB3L4, DHCR24, ELL2, FKBP5, GUCY1A3, KLK2, KLK3, LRIG1, NDRG1, NKX3.1 (also known as NKX3_1), PLAU, PMEPA1, PPAP2A, PRKACB_2, SGK1, and TMPRSS2;
wherein the three or more target genes for the ER cellular signaling pathway are selected from the group consisting of: AP1B1, CA12, CDH26, CELSR2, CTSD, ERBB2, ESR1, GREB1, HSPB1, IGFBP4, MYC, NRIP1, PDZK1, PGR, RARA, SGK3, SOD1, TFF1, WISP2, and XBP1;
the three or more target genes for the PI3K-FOXO cellular signaling pathway are selected from the group consisting of AGRP, BCL2L11, BCL6, BNIP3, BTG1, CAT, CAV1, CCND1, CCNG2, CDKN1A, CDKN1B, ESR1, FBXO32, FOXO3, GADD45A, INSR, MXI1, SOD2, TNFSF10;
wherein the three or more target genes for the MAPK-AP1 cellular signaling pathway are selected from the group consisting of BCL2L11, CCND1, DDIT3, EGFR, ENPP2, EZR, GLRX, MMP1, MMP3, MMP9, PLAU, PLAUR, PTGS2, SERPINE1, TIMP1, TP53, VEGFD, and VIM;
wherein the three or more target genes for the Notch cellular signaling pathway are selected from the group consisting of CD44, EPHB3, FABP7, HES1, HES4, HES5, HEY1, HEY2, MYC, NOX1, NRARP, PIN1, PLXND1, and SOX9;
wherein the three or more target genes for the HH cellular signaling pathway are selected from the group consisting of CFLAR, FOXM1, FYN, GLI1, HHIP, MYCN, NKX2-2, PTCH1, PTCH2, RAB34, SPP1, TCEA2, and TSC22D1;
wherein the three or more target genes for the TGFbeta cellular signaling pathway are selected from the group consisting of ANGPTL4, CDKN1A, CTGF, GADD45A, GADD45B, ID1, IL11, JUNB, MMP2, MMP9, PDGFB, SERPINE1, SGK1, SKIL, SMAD4, SMAD7, SNAI1, TIMP1, and VEGFA;
wherein the three or more target genes for the WNT cellular signaling pathway are selected from the group consisting of CEMIP, AXIN2, CD44, RNF43, MYC, TBX3, TDGF1, SOX9, ASCL2, CXCL8, SP5, ZNRF3, EPHB2, LGR5, EPHB3, KLF6, CCND1, DEFA6, and FZD7;
wherein the three or more target genes for the PR cellular signaling pathway are selected from the group consisting of AGRP, BCL2L11, BCL6, BNIP3, BTG1, CAT, CAV1, CCND1, CCND2, CCNG2, CDKN1A, CDKN1B, ESR1, FASLG, FBXO32, GADD45A, INSR, MXI1, NOS3, PCK1, POMC, PPARGC1A, PRDX3, RBL2, SOD2 and TNFSF10;
wherein the three or more target genes for the NFkB cellular signaling pathway are selected from the group consisting of BIRC3, CCL3, CCL4, CCL5, CCL20, CXCL2, ICAM1, IL6, IRF1, MMP9, NFKB2, PTGS2, TNF, TNIP1, TRAF1, and VCAM1;
wherein the three or more target genes for the JAK-STAT1/2 cellular signaling pathway are selected from the group consisting of APOL1, BID, CXCL9, GBP1, GNAZ, IFI6, IFIT2, IFITM1, IRF1, IRF7, IRF9, ISG15, LY6E, OAS1, PDCD1, RFPL3, SSTR3, STAT1, TAP1 and USP18;
wherein the three or more target genes for the JAK-STAT3 cellular signaling pathway are selected from the group consisting of AKT1, BCL2, BCL2L1, BIRC5, CCND1, CD274, CDKNIA, CRP, FGF2, FOS, FSCN1, FSCN2, FSCN3, HIFIA, HSP90AA1, HSP90AB1, HSP90B1, HSPA1A, HSPA1B, ICAM1, IFNG, IL10, JunB, MCL1, MMP1, MMP3, MMP9, MUC1, MYC, NOS2, POU2F1, PTGS2, SAA1, STAT1, TIMP1, TNFRSF1B, TWIST1, VIM and ZEB1, wherein the primers and probes are able to amplify and detect the respective genes under the following reaction conditions:
50 mM monovalent salt;
400 nM forward primer
400 nM reverse primer
3.0 mM divalent salt, preferably the divalent salt being Mg²⁺;
100 nM probe; and
0.8 mM dNTP.

This application describes several preferred embodiments. Modifications and alterations may occur to others upon reading and understanding the preceding detailed description. It is intended that the application is construed as including all such modifications and alterations insofar as they come within the scope of the appended claims or the equivalents thereof.

Other variations to the disclosed embodiments can be understood and effected by those skilled in the art in practicing the claimed invention, from a study of the drawings, the disclosure, and the appended claims.

It shall be understood that the assemblies of primers and probes, the kit, the different uses, the method for designing primers and probes, and the method for determining AR activity have similar and/or identical preferred embodiments, in particular, as defined in the dependent claims.

In the claims, the word “comprising” does not exclude other elements or steps, and the indefinite article “a” or “an” does not exclude a plurality.

A single unit or device may fulfill the functions of several items recited in the claims. The mere fact that certain measures are recited in mutually different dependent claims does not indicate that a combination of these measures cannot be used to advantage.

It shall be understood that a preferred embodiment of the present invention can also be any combination of the dependent claims or above embodiments with the respective independent claim.

These and other aspects of the invention will be apparent from and elucidated with reference to the embodiments described hereinafter.

### BRIEF DESCRIPTION OF THE DRAWINGS

FIG. 1 depicts probe fluorescence of the ABCC4_2 assay (top panel) using different concentrations of target RNA. The vertical axis depicts relative fluorescence units (RFU), the horizontal axis depicts the cycle number of the qPCR protocol. The bottom panel depicts the melting curves for the same conditions. The vertical axis depicts the change of relative fluorescence units overtime (−d(RFU)/dT), the horizontal axis depicts the temperature in degrees Celsius.

FIG. 2 depicts probe fluorescence of the ABCC4_2 assay (top panel) using different amounts of starting material (cell line derived RNA). The vertical axis depicts relative fluorescence units (RFU), the horizontal axis depicts the cycle number of the qPCR protocol. The bottom panel depicts the melting curves for the same conditions. The vertical axis depicts the change of relative fluorescence units overtime (−d(RFU)/dT), the horizontal axis depicts the temperature in degrees Celsius.

FIG. 3 depicts probe fluorescence of the ABCC4_2 assay (top panel) using different amounts of starting material (PPFE sample derived RNA). The vertical axis depicts relative fluorescence units (RFU), the horizontal axis depicts the cycle number of the qPCR protocol. The bottom panel depicts the melting curves for the same conditions. The vertical axis depicts the change of relative fluorescence units over time (−d(RFU)/dT), the horizontal axis depicts the temperature in degrees Celsius.

FIG. 4 depicts probe fluorescence of the GREB1_2 assay (top panel) using different concentrations of target RNA. The vertical axis depicts relative fluorescence units (RFU), the horizontal axis depicts the cycle number of the qPCR protocol. The bottom panel depicts the melting curves for the same conditions. The vertical axis depicts the change of relative fluorescence units overtime (−d(RFU)/dT), the horizontal axis depicts the temperature in degrees Celsius.

FIG. 5 depicts probe fluorescence of the GREB1_2 assay (top panel) using different amounts of starting material (cell line derived RNA). The vertical axis depicts relative fluorescence units (RFU), the horizontal axis depicts the cycle number of the qPCR protocol. The bottom panel depicts the melting curves for the same conditions. The vertical axis depicts the change of relative fluorescence units overtime (−d(RFU)/dT), the horizontal axis depicts the temperature in degrees Celsius.

FIG. 6 depicts probe fluorescence of the GREB1_2 assay (top panel) using different amounts of starting material (PPFE sample derived RNA). The vertical axis depicts relative fluorescence units (RFU), the horizontal axis depicts the cycle number of the qPCR protocol. The bottom panel depicts the melting curves for the same conditions. The vertical axis depicts the change of relative fluorescence units over time (−d(RFU)/dT), the horizontal axis depicts the temperature in degrees Celsius.

FIG. 7 depicts probe fluorescence of the GADD45A_2 assay (top panel) using different concentrations of target RNA. The vertical axis depicts relative fluorescence units (RFU), the horizontal axis depicts the cycle number of the qPCR protocol. The bottom panel depicts the melting curves for the same conditions. The vertical axis depicts the change of relative fluorescence units overtime (−d(RFU)/dT), the horizontal axis depicts the temperature in degrees Celsius.

FIG. 8 depicts probe fluorescence of the GADD45A_2 assay (top panel) using different amounts of starting material (cell line derived RNA). The vertical axis depicts relative fluorescence units (RFU), the horizontal axis depicts the cycle number of the qPCR protocol. The bottom panel depicts the melting curves for the same conditions. The vertical axis depicts the change of relative fluorescence units overtime (−d(RFU)/dT), the horizontal axis depicts the temperature in degrees Celsius.

FIG. 9 depicts probe fluorescence of the GADD45A_2 assay (top panel) using different amounts of starting material (PPFE sample derived RNA). The vertical axis depicts relative fluorescence units (RFU), the horizontal axis depicts the cycle number of the qPCR protocol. The bottom panel depicts the melting curves for the same conditions. The vertical axis depicts the change of relative fluorescence units over time (−d(RFU)/dT), the horizontal axis depicts the temperature in degrees Celsius.

### EXAMPLES

### Example 1 Design of Forward and Reverse Primers and Probes

Primers and probes for target genes of the following cellular signaling pathways were designed as described herein:
ER, AR, PI3K-FOXO, AP1-MAPK, Notch, HH and TGFbeta.

The designed primers and probes are listed below in Tables 1 to 7. Further, the same principle was used to design primers and probes to detect the expression levels of several reference genes, which are listed below in Table 8. All primers and probes were validated using the following conditions:

### Medium Conditions

50 mM monovalent salt;
400 nM forward primer
400 nM reverse primer
3.0 mM MgCl₂;
100 nM probe; and
0.8 mM dNTP

### Reaction Reaction Conditions:

a RT reaction at 500 Celsius for 30 minutes (using the reverse primer) in order to synthesize cDNA, followed by a 5 minute denaturation step at 950 Celsius, followed by 45 cycli of a 15 second denaturation step at 95° Celsius and a 30 second Elongation step at 60° Celsius.

All sets of primers and probes were found to amplify and detect the expression levels of the desired target genes at the indicated reaction conditions.

### Example 2—Validation of Primers and Probes

All primers and probes were validated under the following conditions:

A RT reaction at 50° Celsius for 30 minutes (using the reverse primer) in order to synthesize cDNA, followed by a 5 minute denaturation step at 95° Celsius, followed by 45 cycli of a 15 second denaturation step at 95° Celsius and a 30 second Elongation step at 60° Celsius, the reaction was performed on a qPCR device with fluorescence readout.

Melt curves were determined in the range of 60° C. to 95° C.

### Example 3—Representative Examples of Primer/Probe Pairs

Some exemplary data sets for validated genes are provided below, although it is noted that all genes described below were validated.

Additional information is provided for the following assays: ABCC4_2, GREB1_2 and GADD45A_2, which comprise primers and probes for the amplification and detection of the respective genes ABCC4, GREB1 and GADD45A. Assay ABCC4_2 results in an amplification product of 82 nucleotides spanning an 11594 nucleotide intron. Assay GREB1_2 results in an amplification product of 105 nucleotides spanning an 9696 nucleotide intron. Assay GADD45A_2 results in an amplification product of 82 nucleotides spanning an 1037 nucleotide intron.

Some general information on the assays is listed in Table 9 below.

In order to validate the assays, they are tested on a Bio-Rad CFX96 Touch Real-Time PCR Detection System. The assays were tested on in vitro RNA, cell culture isolate RNA and RNA isolated from a formalin fixed paraffin embedded (FFPE) sample, to test their suitability. For all tests the probes were tested on decreasing amounts of material (RNA) in order to test their efficiency.

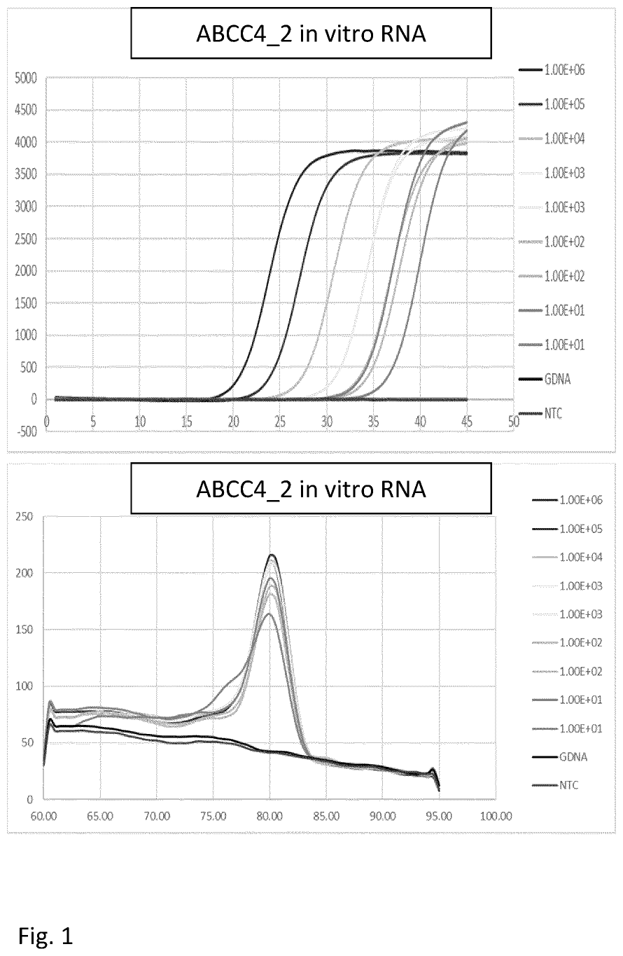

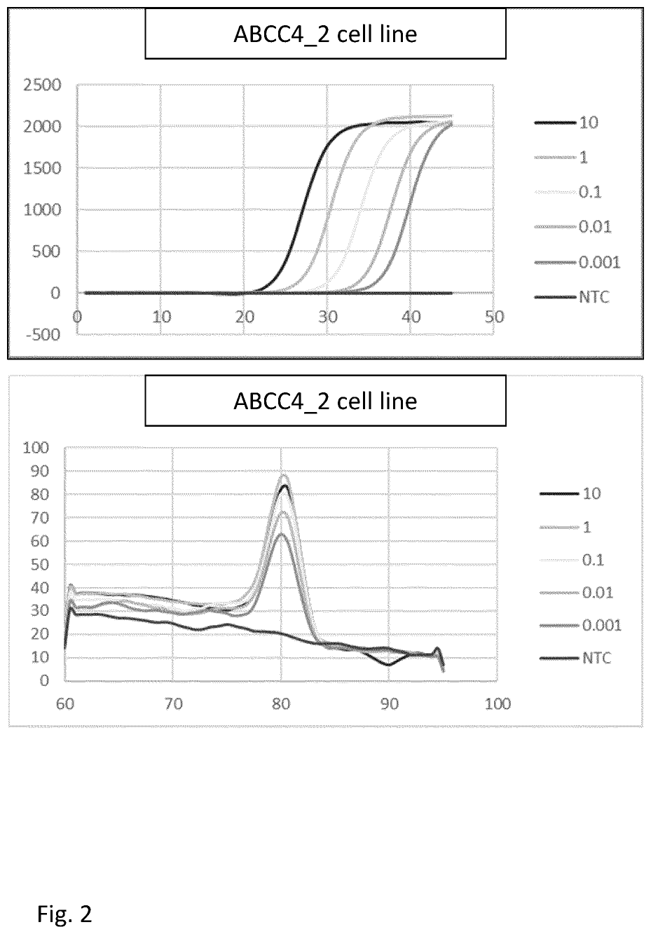

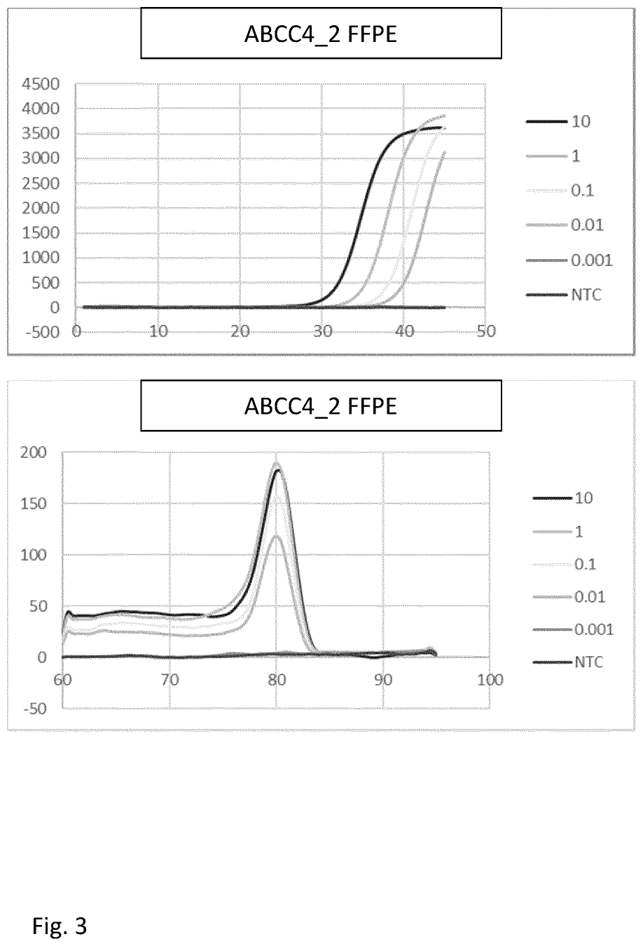

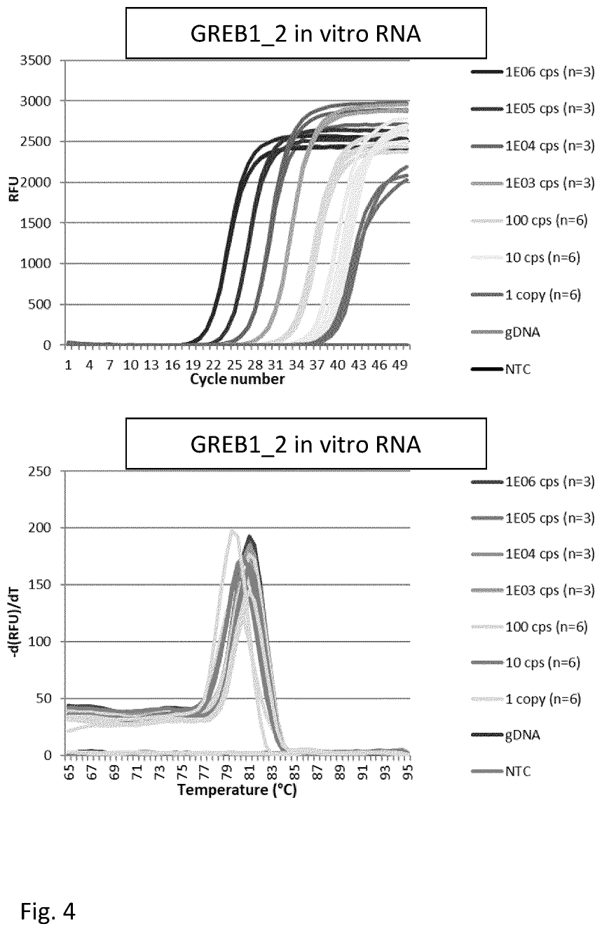

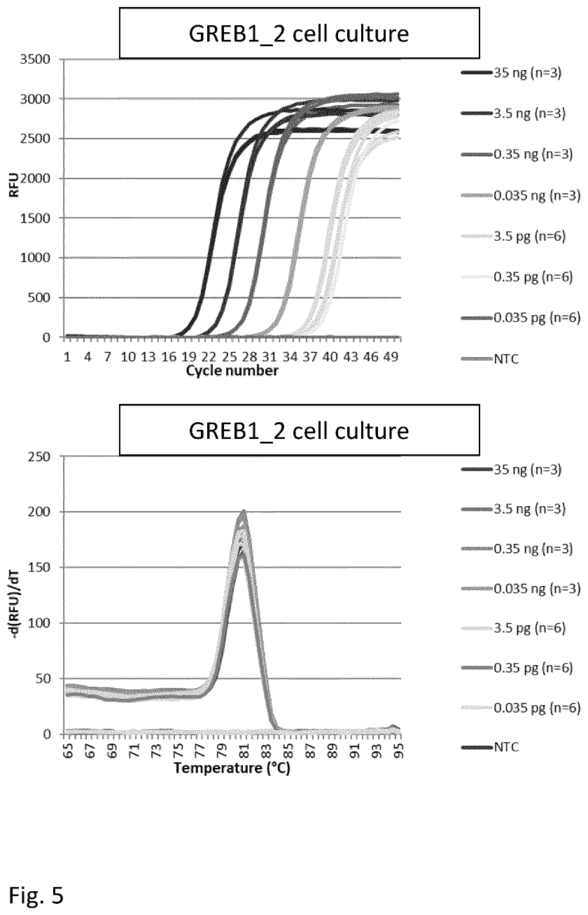

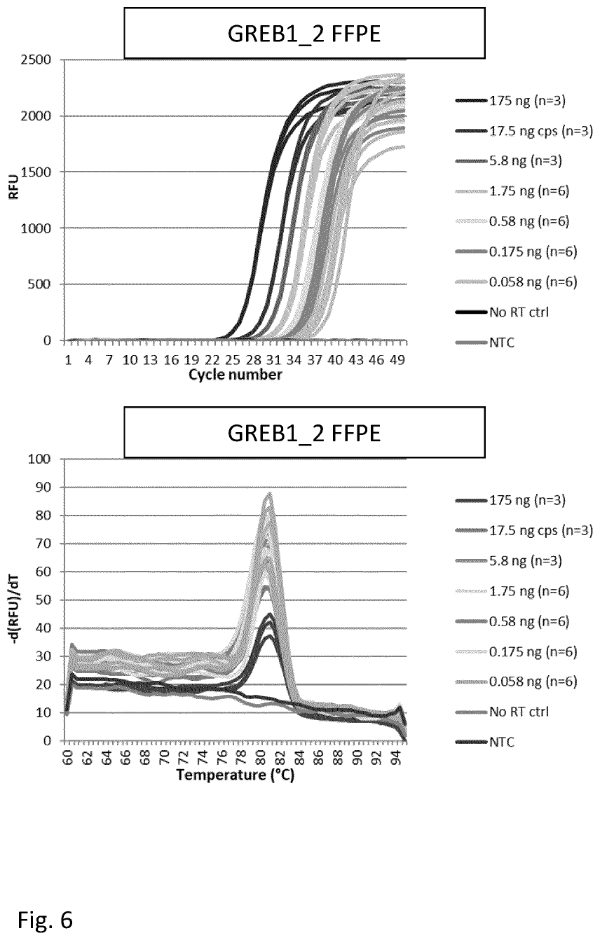

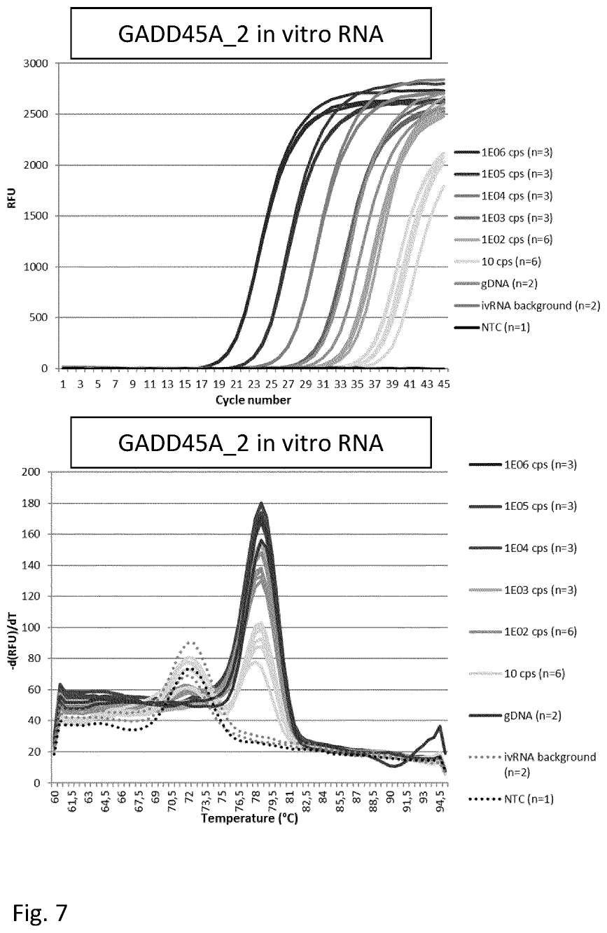

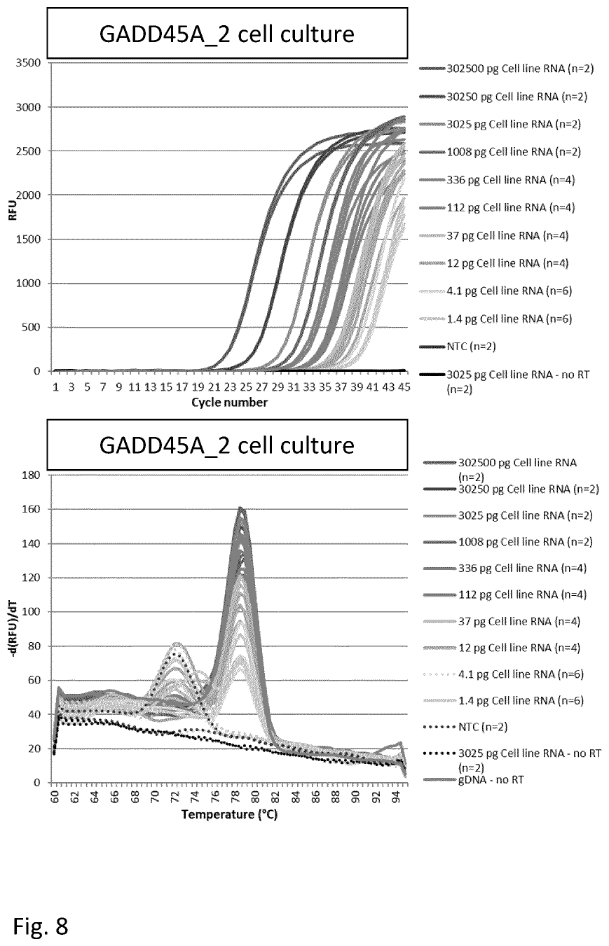

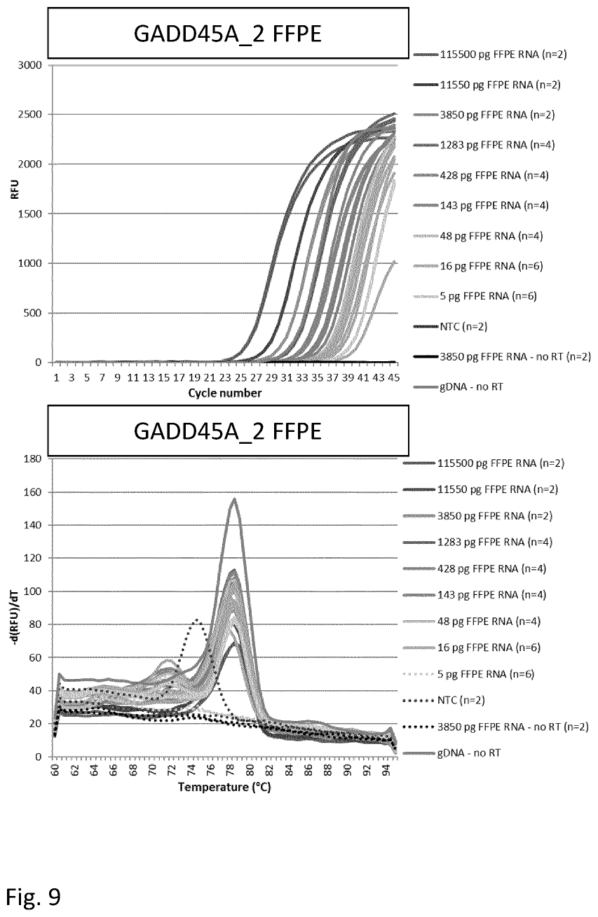

Real-time probe fluorescence curves were generated for all tests performed, as well temperature melt curves over a range of 60 to 95 degrees Celsius. These data are depicting in FIGS. 1 to 9 for the three exemplary genes ABCC4, GREB1 and GADD45A. In each figure, the top panel depicts the probe fluoresce intensity and the bottom panel the melt curves.

Most probes and conditions resulted in a probe efficiency of over 90%, indicating the probes are suitable for quantitative measurements of target gene expression levels.

TABLE 1
Sets of primers and probes for determining the ER cellular signaling pathway
activity.

| SEQ | | | | | |
|---|---|---|---|---|---|
| ID | | | | | |
| NO | Pathway | Gene | Assay | Oligo | Sequence |
| 1 | ER | AP1B1 | AP1B1_1 | Forward primer | AGGCTGTGCGTGCTATT |
| 2 | ER | AP1B1 | AP1B1_1 | Probe | CCATCAAGGTGGAGCAATCTGCGG |
| 3 | ER | AP1B1 | AP1B1_1 | Reverse primer | TTGACCTTGGTCTGGATGAG |
| 4 | ER | AP1B1 | AP1B1_2 | Forward primer | CATCAAGGTGGAGCAATCTG |
| 5 | ER | AP1B1 | AP1B1_2 | Probe | TGCTCGACCTCATCCAGACCAAGG |
| 6 | ER | AP1B1 | AP1B1_2 | Reverse Primer | CTCCTGGACCACATAGTTGA |
| 7 | ER | AP1B1 | AP1B1_3 | Forward Primer | GTGTTGGCAGAGCTGAAAG |
| 8 | ER | AP1B1 | AP1B1_3 | Probe | ACTTTGTACGGAAGGCTGTGCGTG |
| 9 | ER | AP1B1 | AP1B1_3 | Reverse Primer | CAGATTGCTCCACCTTGATG |
| 10 | ER | CA12 | CA12_1 | Forward Primer | GCATTCTTGGCATCTGTATT |
| 11 | ER | CA12 | CA12_1 | Probe | TGGTGGTGGTGTCCATTTGGCTTT |
| 12 | ER | CA12 | CA12_1 | Reverse Primer | GTGGCTGGCTTGTAAATG |
| 13 | ER | CA12 | CA12_2 | Probe | TGGTGGTGGTGTCCATTTGGCTTT |
| 14 | ER | CA12 | CA12_2 | Forward Primer | GGCATTCTTGGCATCTGTATT |
| 15 | ER | CA12 | CA12_2 | Reverse Primer | GCTTGTAAATGACTCCCTTGTT |
| 16 | ER | CA12 | CA12_3 | Forward Primer | TATTGTGGTGGTGGTGTC |
| 17 | ER | CA12 | CA12_3 | Probe | AAGGGAGTCATTTACAAGCCAGCCAC |
| 18 | ER | CA12 | CA12_3 | Reverse Primer | GCCTCAGTCTCCATCTTG |
| 19 | ER | CA12 | CA12_4 | Forward Primer | TGGCATTCTTGGCATCT |
| 20 | ER | CA12 | CA12_4 | Probe | TGGTGGTGGTGTCCATTTGGCTTT |
| 21 | ER | CA12 | CA12_4 | Reverse Primer | GACTCCCTTGTTATCACCTT |
| 22 | ER | CDH26 | CDH26_1 | Forward Primer | CACAATGCACAGACAACTC |
| 23 | ER | CDH26 | CDH26_1 | Probe | TGCCAATGTGCTGGAAGATGACCC |
| 24 | ER | CDH26 | CDH26_1 | Reverse Primer | TGTAGACGTGAGGTAGGT |
| 25 | ER | CDH26 | CDH26_2 | Forward Primer | CTTACCCAGATGCCACAA |
| 26 | ER | CDH26 | CDH26_2 | Probe | CGGTGGAAGGAAGGATGGCAGAGA |
| 27 | ER | CDH26 | CDH26_2 | Reverse Primer | GGTCATCTTCCAGCACAT |
| 28 | ER | CDH26 | CDH26_3 | Forward Primer | GGATGGCAGAGACATTGA |
| 29 | ER | CDH26 | CDH26_3 | Probe | TGCCAATGTGCTGGAAGATGACCC |
| 30 | ER | CDH26 | CDH26_3 | Reverse Primer | CCTTCCTCGCTGTAGAC |
| 31 | ER | CELSR2 | CELSR2_1 | Forward Primer | TGCTCTGACCACCAAGT |
| 32 | ER | CELSR2 | CELSR2_1 | Probe | ACCCTGACCTCGTCCTACAACTGC |
| 33 | ER | CELSR2 | CELSR2_1 | Reverse Primer | TCTCCGTAGGGCTGGTA |
| 34 | ER | CELSR2 | CELSR2_2 | Reverse primer | TCCGTAGGGCTGGTACA |
| 35 | ER | CELSR2 | CELSR2_2 | Forward primer | GGTCCGGAAAGCACTCAA |
| 36 | ER | CELSR2 | CELSR2_2 | Probe | TCCTACAACTGCCCCAGCCCCTA |
| 37 | ER | CELSR2 | CELSR2_3 | Forward Primer | CCTGACCCTGCTCTGAC |
| 38 | ER | CELSR2 | CELSR2_3 | Probe | ACCCTGACCTCGTCCTACAACTGC |
| 39 | ER | CELSR2 | CELSR2_3 | Reverse Primer | GTACAGCCGCCCATCTG |
| 40 | ER | CELSR2 | CELSR2_4 | Forward Primer | TCTGACCACCAAGTCCAC |
| 41 | ER | CELSR2 | CELSR2_4 | Probe | CCTGACCTCGTCCTACAACTGCCC |
| 42 | ER | CELSR2 | CELSR2_4 | Reverse Primer | TGGTGCTGTGCAGAGAG |
| 43 | ER | CTSD | CTSD_1 | Forward Primer | CCTCGTTTGACATCCACTA |
| 44 | ER | CTSD | CTSD_1 | Probe | ACACAGTGTCCTGGCTCAGGTACC |
| 45 | ER | CTSD | CTSD_1 | Reverse Primer | TGCCTCTCCACTTTGAC |
| 46 | ER | CTSD | CTSD_2 | Forward Primer | GGTACCTCGTTTGACATCC |
| 47 | ER | CTSD | CTSD_2 | Probe | ACACAGTGTCCTGGCTCAGGTACC |
| 48 | ER | CTSD | CTSD_2 | Reverse Primer | AAAGACCTGCCTCTCCA |
| 49 | ER | CTSD | CTSD_3 | PCR primer | GTACCTCGTTTGACATCCACTAT |
| | | | | fwd | |
| 50 | ER | CTSD | CTSD_3 | probe | CGTCGTCAGCCTCTGCCCTG |
| 51 | ER | CTSD | CTSD_3 | PCR primer | ACCTGCCTCTCCACTTTGAC |
| | | | | rev | |
| 52 | ER | CTSD | CTSD_4 | Probe | ACACTGTGTCGGTGCCCTGCCAG |
| 53 | ER | CTSD | CTSD_4 | Reverse primer | AAGACCTGCCTCTCCACTTT |
| 54 | ER | CTSD | CTSD_4 | Forward primer | GAATGGTACCTCGTTTGACATCC |
| 55 | ER | ERBB2 | ERBB2_1 | Forward Primer | GTCACCTACAACACAGACA |
| 56 | ER | ERBB2 | ERBB2_1 | Probe | CGTTTGAGTCCATGCCCAATCCCG |
| 57 | ER | ERBB2 | ERBB2_1 | Reverse Primer | CACGTCCGTAGAAAGGTAG |
| 58 | ER | ERBB2 | ERBB2_2 | Forward Primer | TGAGTCCATGCCCAATC |
| 59 | ER | ERBB2 | ERBB2_2 | Probe | ACACAGCTGGCGCCGAATGTATAC |
| 60 | ER | ERBB2 | ERBB2_2 | Reverse Primer | GTCCGTAGAAAGGTAGTTGT |
| 61 | ER | ERBB2 | ERBB2_3 | Forward primer | CGAGGGCCGGTATACATT |
| 62 | ER | ERBB2 | ERBB2_3 | Reverse primer | CACGTCCGTAGAAAGGTAGTT |
| 63 | ER | ERBB2 | ERBB2_3 | Probe | AGCTGTGTGACTGCCTGTCCCTA |
| 64 | ER | ERBB2 | ERBB2_4 | Forward Primer | GAGGGCCGGTATACATTC |
| 65 | ER | ERBB2 | ERBB2_4 | Probe | TTTCTACGGACGTGGGATCCTGCA |
| 66 | ER | ERBB2 | ERBB2_4 | Reverse Primer | CTGTCACCTCTTGGTTGT |
| 67 | ER | ESR1 | ESR1_1 | Forward Primer | CTTCGATGATGGGCTTACT |
| 68 | ER | ESR1 | ESR1_1 | Probe | CATGTGAACCAGCTCCCTGTCTGC |
| 69 | ER | ESR1 | ESR1_1 | Reverse Primer | GGAGGGTCAAATCCACAA |
| 70 | ER | ESR1 | ESR1_2 | Probe | CAACTGGGCGAAGAGGGTGCCA |
| 71 | ER | ESR1 | ESR1_2 | Forward primer | AGCTTCGATGATGGGCTTAC |
| 72 | ER | ESR1 | ESR1_2 | Reverse primer | CCTGATCATGGAGGGTCAAA |
| 73 | ER | ESR1 | ESR1_3 | Forward Primer | GGAGCTGGTTCACATGAT |
| 74 | ER | ESR1 | ESR1_3 | Probe | AGGGTCAAATCCACAAAGCCTGGC |
| 75 | ER | ESR1 | ESR1_3 | Reverse Primer | CTAGCCAGGCACATTCTA |
| 76 | ER | ESR1 | ESR1_4 | Forward Primer | GATGGGCTTACTGACCAA |
| 77 | ER | ESR1 | ESR1_4 | Probe | CATGTGAACCAGCTCCCTGTCTGC |
| 78 | ER | ESR1 | ESR1_4 | Reverse Primer | CTGATCATGGAGGGTCAAA |
| 79 | ER | GREB1 | GREB1_1 | Forward Primer | GAGGTTCTTGCCAGATGA |
| 80 | ER | GREB1 | GREB1_1 | Probe | TGTGTTGGCTGTGGAAAGAAAGGCT |
| 81 | ER | GREB1 | GREB1_1 | Reverse Primer | TTGGAGAATTCCGTGAAGTA |
| 82 | ER | GREB1 | GREB1_2 | Probe | TCTCTGGGAATTGTGTTGGCTGTGGA |
| 83 | ER | GREB1 | GREB1_2 | Reverse primer | GGAGAATTCCGTGAAGTAACAG |
| 84 | ER | GREB1 | GREB1_2 | Forward primer | AAGAGGTTCTTGCCAGATGA |
| 85 | ER | GREB1 | GREB1_3 | Forward Primer | GATGACAATGGCCACAATG |
| 86 | ER | GREB1 | GREB1_3 | Probe | TGTGTTGGCTGTGGAAAGAAAGGCT |
| 87 | ER | GREB1 | GREB1_3 | Reverse Primer | CTTCTTGGGTTGAGTGGT |
| 88 | ER | GREB1 | GREB1_4 | Forward Primer | CCGTTGACAAGAGGTTCT |
| 89 | ER | GREB1 | GREB1_4 | Probe | CCAGAGAAACCAAGAAGAGCATTGTGGC |
| 90 | ER | GREB1 | GREB1_4 | Reverse Primer | CCACAGCCAACACAATTC |
| 91 | ER | HSPB1 | HSPB1_1 | Reverse primer | GGTCAGTGTGCCCTCAG |
| 92 | ER | HSPB1 | HSPB1_1 | Forward primer | GGACGAGCATGGCTACAT |
| 93 | ER | HSPB1 | HSPB1_1 | Probe | ACCCAAGTTTCCTCCTCCCTGTCC |
| 94 | ER | HSPB1 | HSPB1_2 | Forward Primer | CTTCACGCGGAAATACAC |
| 95 | ER | HSPB1 | HSPB1_2 | Probe | ACCCAAGTTTCCTCCTCCCTGTCC |
| 96 | ER | HSPB1 | HSPB1_2 | Reverse Primer | GATGGTGATCTCGTTGGA |
| 97 | ER | HSPB1 | HSPB1_3 | Forward Primer | AGCATGGCTACATCTCC |
| 98 | ER | HSPB1 | HSPB1_3 | Probe | TGCTTCACGCGGAAATACACGCTG |
| 99 | ER | HSPB1 | HSPB1_3 | Reverse Primer | GAGGAGGAAACTTGGGT |
| 100 | ER | HSPB1 | HSPB1_4 | Forward Primer | GACGAGCATGGCTACAT |
| 101 | ER | HSPB1 | HSPB1_4 | Probe | TGCTTCACGCGGAAATACACGCTG |
| 102 | ER | HSPB1 | HSPB1_4 | Reverse Primer | ACAGGGAGGAGGAAACT |
| 103 | ER | IGFBP4 | IGFBP4_1 | Forward Primer | ACGAGGACCTCTACATCATC |
| 104 | ER | IGFBP4 | IGFBP4_1 | Probe | AAGCAGTGTCACCCAGCTCTGGAT |
| 105 | ER | IGFBP4 | IGFBP4_1 | Reverse Primer | CCACACACCAGCACTTG |
| 106 | ER | IGFBP4 | IGFBP4_2 | Forward Primer | CCAACTGCGACCGCAAC |
| 107 | ER | IGFBP4 | IGFBP4_2 | Reverse primer | GTCTTCCGGTCCACACAC |
| 108 | ER | IGFBP4 | IGFBP4_2 | Probe | CAAGCAGTGTCACCCAGCTCTGGA |
| 109 | ER | IGFBP4 | IGFBP4_3 | Forward Primer | CTGGCCGCTTCACAGAG |
| 110 | ER | IGFBP4 | IGFBP4_3 | Probe | TGATGTAGAGGTCCTCGTGGGTGC |
| 111 | ER | IGFBP4 | IGFBP4_3 | Reverse Primer | CAGAGCTGGGTGACACTG |
| 112 | ER | IGFBP4 | IGFBP4_4 | Forward Primer | GCAACGGCAACTTCCAC |
| 113 | ER | IGFBP4 | IGFBP4_4 | Probe | AAGCAGTGTCACCCAGCTCTGGAT |
| 114 | ER | IGFBP4 | IGFBP4_4 | Reverse Primer | GTCTTCCGGTCCACACA |
| 115 | ER | MYC | MYC_1 | Forward Primer | TGCTTAGACGCTGGATTT |
| 116 | ER | MYC | MYC_1 | Probe | CCCTCAACGTTAGCTTCACCAACAGG |
| 117 | ER | MYC | MYC_1 | Reverse Primer | TCGTAGTCGAGGTCATAGT |
| 118 | ER | MYC | MYC_2 | Forward Primer | TCTCTGAAAGGCTCTCCT |
| 119 | ER | MYC | MYC_2 | Probe | TGCAGCTGCTTAGACGCTGGATTT |
| 120 | ER | MYC | MYC_2 | Reverse Primer | TCCTGTTGGTGAAGCTAAC |
| 121 | ER | MYC | MYC_3 | Forward Primer | GACCCGCTTCTCTGAAA |
| 122 | ER | MYC | MYC_3 | Probe | TGCAGCTGCTTAGACGCTGGATTT |
| 123 | ER | MYC | MYC_3 | Reverse Primer | AGGTCATAGTTCCTGTTGG |
| 124 | ER | NRIP1 | NRIP1_1 | Forward Primer | CCGGATGACATCAGAGCTA |
| 125 | ER | NRIP1 | NRIP1_1 | Probe | TCTCAGAAAGCAGAGGCTCAGAGCTT |
| 126 | ER | NRIP1 | NRIP1_1 | Reverse Primer | AATGCAAATATCAGTGTTCGTC |
| 127 | ER | NRIP1 | NRIP1_2 | Forward Primer | CTCAGAGCTTGGAGACAGAC |
| 128 | ER | NRIP1 | NRIP1_2 | Probe | AGGATTCTATCTGCTTACTGCTACAGACCT |
| 129 | ER | NRIP1 | NRIP1_2 | Reverse Primer | GCAAGGAGGAGGAGAAGAAT |
| 130 | ER | NRIP1 | NRIP1_3 | Probe | AAGCAGAGGCTCAGAGCTTGGAGA |
| 131 | ER | NRIP1 | NRIP1_3 | Forward primer | CAACAGCCTTCTCAATTTTCT |
| 132 | ER | NRIP1 | NRIP1_3 | Reverse primer | CCCATTAAATGCAAATATCAGTG |
| 133 | ER | NRIP1 | NRIP1_4 | Forward Primer | TCAGAGCTTGGAGACAGA |
| 134 | ER | NRIP1 | NRIP1_4 | Probe | AAGGATTCTATCTGCTTACTGCTACAGA |
| 135 | ER | NRIP1 | NRIP1_4 | Reverse Primer | GAGAAGAATTCCTTAACACATAGG |
| 136 | ER | PDZK1 | PDZK1_2 | Reverse primer | TGCTCAACATGACGCTTGTC |
| 137 | ER | PDZK1 | PDZK1_2 | Forward primer | GCCATGAGGAAGTGGTTGAAA |
| 138 | ER | PDZK1 | PDZK1_2 | Probe | AAGCCGTGTCATGTTCCTGCTGGT |
| 139 | ER | PGR | PGR_1 | Forward Primer | GAGTTCCTCTGTATGAAAGTATTG |
| 140 | ER | PGR | PGR_1 | Probe | TGGAAGGGCTACGAAGTCAAACCCA |
| 141 | ER | PGR | PGR_1 | Reverse Primer | ATGTAGCTTGACCTCATCTC |
| 142 | ER | PGR | PGR_2 | Forward primer | TGGCAGATCCCACAGGAGTT |
| 143 | ER | PGR | PGR_2 | Probe | AGCTTCAAGTTAGCCAAGAAGAGTTCCTCT |
| 144 | ER | PGR | PGR_2 | Reverse primer | AGCCCTTCCAAAGGAATTGTATTA |
| 145 | ER | PGR | PGR_3 | Forward Primer | AGTTAGCCAAGAAGAGTTCC |
| 146 | ER | PGR | PGR_3 | Probe | TGGAAGGGCTACGAAGTCAAACCCA |
| 147 | ER | PGR | PGR_3 | Reverse Primer | AGCTCTCTAATGTAGCTTGAC |
| 148 | ER | PGR | PGR_4 | Forward Primer | CCAAGAAGAGTTCCTCTGTAT |
| 149 | ER | PGR | PGR_4 | Probe | TGGAAGGGCTACGAAGTCAAACCCA |
| 150 | ER | PGR | PGR_4 | Reverse Primer | CTTGACCTCATCTCCTCAAA |
| 151 | ER | RARA | RARA_1 | Forward Primer | CAGATCACCCTCCTCAA |
| 152 | ER | RARA | RARA_1 | Probe | CCTGGACATCCTGATCCTGCGGA |
| 153 | ER | RARA | RARA_1 | Reverse Primer | CGAGAAGGTCATGGTGT |
| 154 | ER | RARA | RARA_2 | Forward Primer | TTCACCACCCTCACCAT |
| 155 | ER | RARA | RARA_2 | Probe | AGCCTTGAGGAGGGTGATCTGGTC |
| 156 | ER | RARA | RARA_2 | Reverse Primer | AGAAGGTCATGGTGTCCTG |
| 157 | ER | RARA | RARA_3 | Reverse primer | GTGTACCGCGTGCAGAT |
| 158 | ER | RARA | RARA_3 | Forward primer | CATTAAGACTGTGGAGTTCGC |
| 159 | ER | RARA | RARA_3 | Probe | ACCAGATCACCCTCCTCAAGGCT |
| 160 | ER | RARA | RARA_4 | Forward Primer | CTGCCTGCCTGGACATC |
| 161 | ER | RARA | RARA_4 | Probe | CGAGAAGGTCATGGTGTCCTGCTC |
| 162 | ER | RARA | RARA_4 | Reverse Primer | CATCTGGGTCCGGTTCAG |
| 163 | ER | SGK3 | SGK3_1 | Forward Primer | GCTATGGCCCTGAAGATT |
| 164 | ER | SGK3 | SGK3_1 | Probe | ACAAAGACGAGCAGGACTAAACGA |
| 165 | ER | SGK3 | SGK3_1 | Reverse Primer | ACCTAACTAGGTTCTGAATGAA |
| 166 | ER | SGK3 | SGK3_2 | Forward Primer | AAACAGTTTCCTGCTATGG |
| 167 | ER | SGK3 | SGK3_2 | Probe | CCCTGAAGATTCCTGCCAAGAGAA |
| 168 | ER | SGK3 | SGK3_2 | Reverse Primer | TAGTCCTGCTCGTCTTTG |
| 169 | ER | SGK3 | SGK3_3 | Forward Primer | AGATTCCTGCCAAGAGAATA |
| 170 | ER | SGK3 | SGK3_3 | Probe | ACAAAGACGAGCAGGACTAAACGA |
| 171 | ER | SGK3 | SGK3_3 | Reverse Primer | GGATACCTAACTAGGTTCTGA |
| 172 | ER | SGK3 | SGK3_4 | Probe | ACAAAGACGAGCAGGACTAAACGA |
| 173 | ER | SGK3 | SGK3_4 | Reverse Primer | TGGATACCTAACTAGGTTCTGAATG |
| 174 | ER | SGK3 | SGK3_4 | Forward Primer | CTGCCAAGAGAATATTTGGTGATAA |
| 175 | ER | SOD1 | SOD1_1 | Forward Primer | TGCAGGTCCTCACTTTAATC |
| 176 | ER | SOD1 | SOD1_1 | Probe | AAACACGGTGGGCCAAAGGATGAA |
| 177 | ER | SOD1 | SOD1_1 | Reverse Primer | CTTTGTCAGCAGTCACATTG |
| 178 | ER | SOD1 | SOD1_2 | Forward Primer | GGTCCTCACTTTAATCCTCTATC |
| 179 | ER | SOD1 | SOD1_2 | Probe | AAACACGGTGGGCCAAAGGATGAA |
| 180 | ER | SOD1 | SOD1_2 | Reverse Primer | ACCATCTTTGTCAGCAGTC |
| 181 | ER | SOD1 | SOD1_3 | Forward Primer | GTGCAGGTCCTCACTTT |
| 182 | ER | SOD1 | SOD1_3 | Probe | AAACACGGTGGGCCAAAGGATGAA |
| 183 | ER | SOD1 | SOD1_3 | Reverse Primer | GCCCAAGTCTCCAACAT |
| 184 | ER | TTF1 | TTF1_1 | Forward Primer | CCTCCCAGTGTGCAAATA |
| 185 | ER | TTF1 | TTF1_1 | Probe | ACGTCCCTCCAGAAGAGGAGTGTG |
| 186 | ER | TTF1 | TTF1_1 | Reverse Primer | CCCTGCAGAAGTGTCTAAA |
| 187 | ER | TTF1 | TTF1_2 | Forward Primer | CCCTGGTGCTTCTATCCTAA |
| 188 | ER | TTF1 | TTF1_2 | Reverse Primer | ATCCCTGCAGAAGTGTCTAA |
| 189 | ER | TTF1 | TTF1_2 | Probe | ACCATCGACGTCCCTCCAGAA |
| 190 | ER | TTF1 | TTF1_3 | Forward Primer | GGTGCTTCTATCCTAATACCA |
| 191 | ER | TTF1 | TTF1_3 | Probe | AGACACTTCTGCAGGGATCTGCCT |
| 192 | ER | TTF1 | TTF1_3 | Reverse Primer | TCTGGGACTAATCACCGT |
| 193 | ER | TTF1 | TTF1_4 | Forward Primer | CCTGGTGCTTCTATCCTAAT |
| 194 | ER | TTF1 | TTF1_4 | Probe | ACGTCCCTCCAGAAGAGGAGTGTG |
| 195 | ER | TTF1 | TTF1_4 | Reverse Primer | GATCCCTGCAGAAGTGT |
| 196 | ER | WISP2 | WISP2_1 | Forward Primer | GGACATGAGAGGCACAC |
| 197 | ER | WISP2 | WISP2_1 | Probe | TCTCCCTCCTCTGCCTCCTCTCAA |
| 198 | ER | WISP2 | WISP2_1 | Reverse Primer | GGCAGGTACATGGTGTC |
| 199 | ER | WISP2 | WISP2_2 | Forward Primer | TCCTCTGCCTCCTCTCAAA |
| 200 | ER | WISP2 | WISP2_2 | Probe | CAGCTGTGCCCGACACCATGTAC |
| 201 | ER | WISP2 | WISP2_2 | Reverse Primer | CAGCCATCCAGCACCAG |
| 202 | ER | WISP2 | WISP2_3 | Forward Primer | ACACCGAAGACCCACCT |
| 203 | ER | WISP2 | WISP2_3 | Probe | TCTCCCTCCTCTGCCTCCTCTCAA |
| 204 | ER | WISP2 | WISP2_3 | Reverse Primer | CATGGTGTCGGGCACAG |
| 205 | ER | WISP2 | WISP2_4 | Reverse primer | CATGGTGTCGGGCACAG |
| 206 | ER | WISP2 | WISP2_4 | Forward primer | GAGAGGCACACCGAAGAC |
| 207 | ER | WISP2 | WISP2_4 | Probe | TTCTCCCTCCTCTGCCTCCT |
| 208 | ER | XBP1 | XBP1_1 | Forward Primer | TGGATTCTGGCGGTATTG |
| 209 | ER | XBP1 | XBP1_1 | Probe | TTGGGCATTCTGGACAACTTGGACC |
| 210 | ER | XBP1 | XBP1_1 | Reverse Primer | GGAAGGGCATTTGAAGAAC |
| 211 | ER | XBP1 | XBP1_2 | Reverse primer | CATGACTGGGTCCAAGTTGTC |
| 212 | ER | XBP1 | XBP1_2 | Forward primer | GGATTCTGGCGGTATTGACT |
| 213 | ER | XBP1 | XBP1_2 | Probe | TCAGAGTCTGATATCCTGTTGGGCATTCTG |
| 214 | ER | XBP1 | XBP1_3 | Forward Primer | GCGGTATTGACTCTTCAGAT |
| 215 | ER | XBP1 | XBP1_3 | Probe | TCTGATATCCTGTTGGGCATTCTGGACA |
| 216 | ER | XBP1 | XBP1_3 | Reverse Primer | GAACATGACTGGGTCCAA |
| 217 | ER | XBP1 | XBP1_4 | Forward Primer | CTGGCGGTATTGACTCTT |
| 218 | ER | XBP1 | XBP1_4 | Probe | TTGGGCATTCTGGACAACTTGGACC |
| 219 | ER | XBP1 | XBP1_4 | Reverse Primer | GGCATTTGAAGAACATGACT |

TABLE 2
Sets of primers and probes for determining the AR cellular signaling
pathway activity.

| SEQ | | | | | |
|---|---|---|---|---|---|
| ID | | | | | |
| NO | Pathway | Gene | Assay | Oligo | Sequence |
| 220 | AR | ABCC4 | ABCC4_1 | Forward Primer | CCATTGAGAGGGTGTCAGA |
| 221 | AR | ABCC4 | ABCC4_1 | Reverse Primer | TGGATTCTTCGGATGCTGACGATTGC |
| 222 | AR | ABCC4 | ABCC4_1 | Probe | CGCTGTGATATCTCATCAAGTA |
| 223 | AR | ABCC4 | ABCC4_2 | Forward primer | GAGAGGGTGTCAGAGGCAAT |
| 224 | AR | ABCC4 | ABCC4_2 | Reverse primer | GCGCTGTGATATCTCATCAAGTAG |
| 225 | AR | ABCC4 | ABCC4_2 | Probe | CGTCAGCATCCGAAGAATCCAGACCT |
| 226 | AR | ABCC4 | ABCC4_3 | Forward Primer | GTGTCAGAGGCAATCGTC |
| 227 | AR | ABCC4 | ABCC4_3 | Reverse Primer | CGTCAGCTGCCGTCAGAT |
| 228 | AR | ABCC4 | ABCC4_3 | Probe | CAGACCTTTTTGCTACTTGATGAGATATCACAGC |
| 229 | AR | ABCC4 | ABCC4_4 | Forward Primer | GCCATTGAGAGGGTGTCAG |
| 230 | AR | ABCC4 | ABCC4_4 | Reverse Primer | TATCACAGCGCAACCGTC |
| 231 | AR | ABCC4 | ABCC4_4 | Probe | GCATCCGAAGAATCCAGACCTTTTTGCT |
| 232 | AR | AR | AR_1 | Forward primer | CCTGATCTGTGGAGATGAAGC |
| 233 | AR | AR | AR_1 | Reverse primer | GCGCACAGGTACTTCTGTT |
| 234 | AR | AR | AR_1 | Probe | TGGAAGCTGCAAGGTCTTCTTC |
| 235 | AR | AR | AR_2 | Forward Primer | GCTTCTGGGTGTCACTATG |
| 236 | AR | AR | AR_2 | Reverse Primer | CGCACAGGTACTTCTGTTT |
| 237 | AR | AR | AR_2 | Probe | TGTGGAAGCTGCAAGGTCTTCTTCA |
| 238 | AR | AR | AR_3 | Forward Primer | CTGATCTGTGGAGATGAAGC |
| 239 | AR | AR | AR_3 | Reverse Primer | GGTACTTCTGTTTCCCTTCAG |
| 240 | AR | AR | AR_3 | Probe | TGTGGAAGCTGCAAGGTCTTCTTCA |
| 241 | AR | AR | AR_4 | Forward Primer | GCTGAAGGGAAACAGAAGTA |
| 242 | AR | AR | AR_4 | Reverse Primer | TGCGCCAGCAGAAATGATTGCAC |
| 243 | AR | AR | AR_4 | Probe | GAAGACGACAAGATGGACAA |
| 244 | AR | CREB3L4 | CREB3L4_1 | Forward Primer | GCATTTATGGTGCCTGATTC |
| 245 | AR | CREB3L4 | CREB3L4_1 | Reverse Primer | CAGGAACAGGGTTTGACAG |
| 246 | AR | CREB3L4 | CREB3L4_1 | Probe | AGTGAGCTGCCCTTTGATGCTCA |
| 247 | AR | CREB3L4 | CREB3L4_2 | Forward Primer | TGATTCCTGCATGGTCAGT |
| 248 | AR | CREB3L4 | CREB3L4_2 | Reverse Primer | ACAACCCTGCTGCCCTGTCAAA |
| 249 | AR | CREB3L4 | CREB3L4_2 | Probe | CTCATCGGTCAGGAACAGG |
| 250 | AR | CREB3L4 | CREB3L4_3 | Forward primer | TGATGCTCATGCCCACATC |
| 251 | AR | CREB3L4 | CREB3L4_3 | Reverse Primer | GCAGACGCTTCTCCTCATC |
| 252 | AR | CREB3L4 | CREB3L4_3 | Probe | TGCTGCCCTGTCAAACCCTGTT |
| 253 | AR | CREB3L4 | CREB3L4_4 | Forward primer | CCAGAGCAGGCACCGTAG |
| 254 | AR | CREB3L4 | CREB3L4_4 | Reverse primer | AGCAGACGCTTCTCCTCATC |
| 255 | AR | CREB3L4 | CREB3L4_4 | Probe | CAACCCTGCTGCCCTGTCAAAC |
| 256 | AR | DHCR24 | DHCR24_1 | Forward primer | GAGGCAGCTGGAGAAGTTT |
| 257 | AR | DHCR24 | DHCR24_1 | Reverse primer | GCAGCTTGTGGTACAAGGAG |
| 258 | AR | DHCR24 | DHCR24_1 | Probe | TATGCCGACTGCTACATGAACCGG |
| 259 | AR | DHCR24 | DHCR24_2 | Forward Primer | TGGCTTCCAGATGCTGTAT |
| 260 | AR | DHCR24 | DHCR24_2 | Reverse Primer | TGGTACAAGGAGCCATCAA |
| 261 | AR | DHCR24 | DHCR24_2 | Probe | AACCGGGAGGAGTTCTGGGAGA |
| 262 | AR | DHCR24 | DHCR24_3 | Forward Primer | AGGCAGCTGGAGAAGTTTG |
| 263 | AR | DHCR24 | DHCR24_3 | Reverse Primer | GCAGCTTGTGGTACAAGGA |
| 264 | AR | DHCR24 | DHCR24_3 | Probe | AACCGGGAGGAGTTCTGGGAGA |
| 265 | AR | DHCR24 | DHCR24_4 | Forward Primer | AACACTTTGAAGCCAGGTC |
| 266 | AR | DHCR24 | DHCR24_4 | Reverse Primer | CAAACATCTCCCAGAACTCC |
| 267 | AR | DHCR24 | DHCR24_4 | Probe | TGCCGACTGCTACATGAACCGG |
| 268 | AR | ELL2 | ELL2_1 | Forward Primer | CAACATCACCGTACTGCAT |
| 269 | AR | ELL2 | ELL2_1 | ReversePrimer | GAGTCCTTGGAACTGGATTG |
| 270 | AR | ELL2 | ELL2_1 | Probe | TGAAGCTCACCGAGACGGCGAT |
| 271 | AR | ELL2 | ELL2_2 | Forward primer | CATGTGAAGCTCACCGAGAC |
| 272 | AR | ELL2 | ELL2_2 | Reverse primer | GGAACTGGATTGAAGGTCGAAA |
| 273 | AR | ELL2 | ELL2_2 | Probe | CTCGAGACTTACCAGAGCCACAA |
| 274 | AR | ELL2 | ELL2_3 | Forward Primer | AGAAGTTTGTCCGCAGC |
| 275 | AR | ELL2 | ELL2_3 | Reverse Primer | GAGTCCTTGGAACTGGATT |
| 276 | AR | ELL2 | ELL2_3 | Probe | TCGAGACTTACCAGAGCCACAAAATTTAATTC |
| 277 | AR | ELL2 | ELL2_4 | Forward Primer | CGTACTGCATGTGAAGCTC |
| 278 | AR | ELL2 | ELL2_4 | Reverse Primer | GAACTGGATTGAAGGTCGAAA |
| 279 | AR | ELL2 | ELL2_4 | Probe | TTTGTGGCTCTGGTAAGTCTCGAGC |
| 280 | AR | FKBP5 | FKBP5_1 | Forward Primer | AACGGAAAGGAGAGGGATA |
| 281 | AR | FKBP5 | FKBP5_1 | Reverse Primer | TTTGACTGCAGAGATGTGG |
| 282 | AR | FKBP5 | FKBP5_1 | Probe | AAATCCACCTGGAAGGCCGCT |
| 283 | AR | FKBP5 | FKBP5_2 | Forward Primer | TGAAGATGGAGGCATTATCC |
| 284 | AR | FKBP5 | FKBP5_2 | Reverse Primer | AACAGTAGAAATCCACCTGG |
| 285 | AR | FKBP5 | FKBP5_2 | Probe | GGAGAACCAAACGGAAAGGAGAGGG |
| 286 | AR | FKBP5 | FKBP5_3 | Forward primer | ACGGAAAGGAGAGGGATATTCA |
| 287 | AR | FKBP5 | FKBP5_3 | Reverse primer | GTCAAACATCCTTCCACCACAG |
| 288 | AR | FKBP5 | FKBP5_3 | Probe | ACAGTAGAAATCCACCTGGAAGGCCG |
| 289 | AR | FKBP5 | FKBP5_4 | Forward Primer | ACGGAAAGGAGAGGGATATT |
| 290 | AR | FKBP5 | FKBP5_4 | Reverse Primer | CCACAGTGAATGCATCCTT |
| 291 | AR | FKBP5 | FKBP5_4 | Probe | AAATCCACCTGGAAGGCCGCT |
| 292 | AR | GUCY1A3 | GUCY1A3_1 | Forward Primer | GGGATTACACAAAGAGAGTGATA |
| 293 | AR | GUCY1A3 | GUCY1A3_1 | Reverse Primer | TCATCTTCAGGGCCATCAGCGC |
| 294 | AR | GUCY1A3 | GUCY1A3_1 | Probe | GGGAGACATAACTTCATCAGAG |
| 295 | AR | GUCY1A3 | GUCY1A3_2 | Reverse primer | CCAGAGTGCAGTCCAATTC |
| 296 | AR | GUCY1A3 | GUCY1A3_2 | Forward primer | GGGATTACACAAAGAGAGTGATAC |
| 297 | AR | GUCY1A3 | GUCY1A3_2 | Probe | TGGCCCTGAAGATGATGGAGCTCT |
| 298 | AR | GUCY1A3 | GUCY1A3_3 | Forward Primer | GGCGATGCCTATTGTGTAG |
| 299 | AR | GUCY1A3 | GUCY1A3_3 | Reverse Primer | TCAGAGAGCTCCATCATCTT |
| 300 | AR | GUCY1A3 | GUCY1A3_3 | Probe | GGGCCATCAGCGCTATCTGAACA |
| 301 | AR | GUCY1A3 | GUCY1A3_4 | Forward Primer | GAGAGTGATACTCATGCTGTT |
| 302 | AR | GUCY1A3 | GUCY1A3_4 | Reverse Primer | CAATTCGCATCTTGATAGGTTC |
| 303 | AR | GUCY1A3 | GUCY1A3_4 | Probe | GCGCTGATGGCCCTGAAGATGA |
| 304 | AR | KLK2 | KLK2_1 | Forward Primer | TCGAACCAGAGGAGTTCTTGC |
| 305 | AR | KLK2 | KLK2_1 | Reverse Primer | AGCACACATGTCATTGGACAG |
| 306 | AR | KLK2 | KLK2_1 | Probe | CCCAGGAGTCTTCAGTGTGTGAGCC |
| 307 | AR | KLK2 | KLK2_2 | Forward Primer | AGGAGTCTTCAGTGTGTGA |
| 308 | AR | KLK2 | KLK2_2 | Reverse Primer | ACAACATGAACTCTGTCACC |
| 309 | AR | KLK2 | KLK2_2 | Probe | TGTCATTGGACAGGAGATGGAGGC |
| 310 | AR | KLK2 | KLK2_3 | Forward Primer | GTGTGTGAGCCTCCATCTC |
| 311 | AR | KLK2 | KLK2_3 | Reverse Primer | CCAGCACACAACATGAACTC |
| 312 | AR | KLK2 | KLK2_3 | Probe | CTGTCCAATGACATGTGTGCTAGAGCT |
| 313 | AR | KLK2 | KLK2_4 | Forward Primer | CATCGAACCAGAGGAGTTC |
| 314 | AR | KLK2 | KLK2_4 | Reverse Primer | GAACTCTGTCACCTTCTCAG |
| 315 | AR | KLK2 | KLK2_4 | Probe | TGTGAGCCTCCATCTCCTGTCCA |
| 316 | AR | KLK3 | KLK3_1 | Forward Primer | GAACCAGAGGAGTTCTTGAC |
| 317 | AR | KLK3 | KLK3_1 | Reverse Primer | TGAACTTGGTCACCTTCTG |
| 318 | AR | KLK3 | KLK3_1 | Probe | CCAATGACGTGTGTGCGCAAGTT |
| 319 | AR | KLK3 | KLK3_2 | Forward Primer | GTGTGGACCTCCATGTTATTT |
| 320 | AR | KLK3 | KLK3_2 | Reverse Primer | ACACAGCATGAACTTGGTC |
| 321 | AR | KLK3 | KLK3_2 | Probe | TGTGCGCAAGTTCACCCTCAGAA |
| 322 | AR | KLK3 | KLK3_3 | Forward Primer | GATGCTGTGAAGGTCATGG |
| 323 | AR | KLK3 | KLK3_3 | Reverse Primer | CACACACTGAAGTTTCTTTGG |
| 324 | AR | KLK3 | KLK3_3 | Probe | ACTCCTCTGGTTCAATGCTGCCC |
| 325 | AR | KLK3 | KLK3_4 | Forward primer | GCAGCATTGAACCAGAGGA |
| 326 | AR | KLK3 | KLK3_4 | Reverse primer | GCACACACGTCATTGGAAATAA |
| 327 | AR | KLK3 | KLK3_4 | Probe | CCCAAAGAAACTTCAGTGTGTGGACCT |
| 328 | AR | LRIG1 | LRIG1_1 | Forward Primer | GCCTATAAAGGAGCTCAACC |
| 329 | AR | LRIG1 | LRIG1_1 | Reverse Primer | CCGTGACAGACCATCAAAT |
| 330 | AR | LRIG1 | LRIG1_1 | Probe | TCGGATTGGCACCCTGGAGTTG |
| 331 | AR | LRIG1 | LRIG1_2 | Forward Primer | GAACAACATCACGGAAGTG |
| 332 | AR | LRIG1 | LRIG1_2 | Reverse Primer | CATCAAATGCTCCCAACTC |
| 333 | AR | LRIG1 | LRIG1_2 | Probe | ACACCTGCTTTCCACACGGACC |
| 334 | AR | LRIG1 | LRIG1_3 | Forward Primer | ACACGGACCGCCTATAAA |
| 335 | AR | LRIG1 | LRIG1_3 | Reverse Primer | CCATCAAATGCTCCCAACTC |
| 336 | AR | LRIG1 | LRIG1_3 | Probe | AGCTCAACCTGGCAGGCAATCG |
| 337 | AR | LRIG1 | LRIG1_4 | Forward Primer | CACACGGACCGCCTATAAA |
| 338 | AR | LRIG1 | LRIG1_4 | Reverse Primer | TTGCTCAGGCGAAGAGTTAG |
| 339 | AR | LRIG1 | LRIG1_4 | Probe | TCGGATTGGCACCCTGGAGTTG |
| 340 | AR | NDRG1 | NDRG1_1 | Forward primer | GCATTATTGGCATGGGAACAG |
| 341 | AR | NDRG1 | NDRG1_1 | Reverse primer | CCACCATCTCAGGGTTGTTTAG |
| 342 | AR | NDRG1 | NDRG1_1 | Probe | CGCCTACATCCTAACTCGATTTGCT |
| 343 | AR | NDRG1 | NDRG1_2 | Forward Primer | CATTATTGGCATGGGAACAG |
| 344 | AR | NDRG1 | NDRG1_2 | Reverse Primer | AGGGTTCACGTTGATAAGG |
| 345 | AR | NDRG1 | NDRG1_2 | Probe | AAACAACCCTGAGATGGTGGAGGG |
| 346 | AR | NDRG1 | NDRG1_3 | Forward Primer | TGAAATGCTTCCTGGAGTC |
| 347 | AR | NDRG1 | NDRG1_3 | Reverse Primer | CAGGGTTGTTTAGAGCAAATC |
| 348 | AR | NDRG1 | NDRG1_3 | Probe | TTGGCATGGGAACAGGAGCAGG |
| 349 | AR | NDRG1 | NDRG1_4 | Forward Primer | CTTCCTGGAGTCCTTCAAC |
| 350 | AR | NDRG1 | NDRG1_4 | Reverse Primer | ACCATCTCAGGGTTGTTTAG |
| 351 | AR | NDRG1 | NDRG1_4 | Probe | TTGGCATGGGAACAGGAGCAGG |
| 352 | AR | NKX3_1 | NKX3_1_1 | Reverse primer | CTTCTGCGGCTGCTTAGG |
| 353 | AR | NKX3_1 | NKX3_1_1 | Forward primer | CAGAGACCGAGCCAGAAAG |
| 354 | AR | NKX3_1 | NKX3_1_1 | Probe | AAACACTTCAGGCGCCCTTCCAA |
| 355 | AR | NKX3_1 | NKX3_1_2 | Forward Primer | CAGAGACCGAGCCAGAAAG |
| 356 | AR | NKX3_1 | NKX3_1_2 | Reverse Primer | CACCTGAGTGTGGGAGAAG |
| 357 | AR | NKX3_1 | NKX3_1_2 | Probe | AACACTTCAGGCGCCCTTCCAA |
| 358 | AR | NKX3_1 | NKX3_1_3 | Forward Primer | GAGCCAGAAAGGCACTTGG |
| 359 | AR | NKX3_1 | NKX3_1_3 | Reverse Primer | TCTCCAACTCGATCACCTGAG |
| 360 | AR | NKX3_1 | NKX3_1_3 | Probe | AACACTTCAGGCGCCCTTCCAA |
| 361 | AR | NKX3_1 | NKX3_1_4 | Forward Primer | GGTCTTATCTGTTGGACTCTG |
| 362 | AR | NKX3_1 | NKX3_1_4 | Reverse Primer | CTGAACTTCCTCTCCAACTC |
| 363 | AR | NKX3_1 | NKX3_1_4 | Probe | AACACTTCAGGCGCCCTTCCAA |
| 364 | AR | PLAU | PLAU_1 | Forward Primer | TCGAACTGTGACTGTCTAAATG |
| 365 | AR | PLAU | PLAU_1 | Reverse Primer | CTGCCCTCCGAATTTCTTT |
| 366 | AR | PLAU | PLAU_1 | Probe | AACATTCACTGGTGCAACTGCCC |
| 367 | AR | PLAU | PLAU_2 | Forward Primer | GTTCCATCGAACTGTGACT |
| 368 | AR | PLAU | PLAU_2 | Reverse Primer | CGAATTTCTTTGGGCAGTTG |
| 369 | AR | PLAU | PLAU_2 | Probe | TGGAGGAACATGTGTGTCCAACAAGT |
| 370 | AR | PLAU | PLAU_3 | Forward Primer | GTGCAACTGCCCAAAGAAAT |
| 371 | AR | PLAU | PLAU_3 | Reverse Primer | GACAGTGGCAGAGTTCCAG |
| 372 | AR | PLAU | PLAU_3 | Probe | AGGAAAGGCCAGCACTGACACC |
| 373 | AR | PLAU | PLAU_4 | Forward primer | ACTGCCCAAAGAAATTCGG |
| 374 | AR | PLAU | PLAU_4 | Reverse primer | CTGGCCTTTCCTCGGTAAA |
| 375 | AR | PLAU | PLAU_4 | Probe | CAGCACTGTGAAATAGATAAGTCAAAAACCT |
| 376 | AR | PMEPA1 | PMEPA1_1 | Forward Primer | TGGTGATGATGGTGATGGT |
| 377 | AR | PMEPA1 | PMEPA1_1 | Reverse Primer | CAGGGCATCTTCTCTCCTC |
| 378 | AR | PMEPA1 | PMEPA1_1 | Probe | ACAAGCTGTCTGCACGGTCCTT |
| 379 | AR | PMEPA1 | PMEPA1_2 | Forward primer | AGCCACTACAAGCTGTCTGC |
| 380 | AR | PMEPA1 | PMEPA1_2 | Reverse primer | TGACACTGTGCTCTCCGAG |
| 381 | AR | PMEPA1 | PMEPA1_2 | Probe | AGAGAAGATGCCCTGTCCTCAGAA |
| 382 | AR | PMEPA1 | PMEPA1_3 | Forward Primer | GTCTGCACGGTCCTTCATC |
| 383 | AR | PMEPA1 | PMEPA1_3 | Reverse Primer | GATTCCGTTGCCTGACACT |
| 384 | AR | PMEPA1 | PMEPA1_3 | Probe | AGAAGATGCCCTGTCCTCAGAAGGA |
| 385 | AR | PMEPA1 | PMEPA1_4 | Forward Primer | ATCATCGTGGTGGTGATGA |
| 386 | AR | PMEPA1 | PMEPA1_4 | Reverse Primer | CTGAGGACAGGGCATCTTC |
| 387 | AR | PMEPA1 | PMEPA1_4 | Probe | ACAAGCTGTCTGCACGGTCCTT |
| 388 | AR | PPAP2A | PPAP2A_1 | Forward Primer | CAGCGATGGTTACATTGAATAC |
| 389 | AR | PPAP2A | PPAP2A_1 | Reverse Primer | CGAAGAGTGGCCTGAATAG |
| 390 | AR | PPAP2A | PPAP2A_1 | Probe | ACAACCTGCCTTCCTTAACTCTTTCTGC |
| 391 | AR | PPAP2A | PPAP2A_2 | Forward Primer | GGCAGGTTGTCCTTCTATT |
| 392 | AR | PPAP2A | PPAP2A_2 | Reverse Primer | TCATCCTGGCTTGAAGATAAA |
| 393 | AR | PPAP2A | PPAP2A_2 | Probe | TGTACTGCATGCTGTTTGTGGCAC |
| 394 | AR | PPAP2A | PPAP2A_3 | Forward Primer | ATGTCGAGGGAATGCAGAAAG |
| 395 | AR | PPAP2A | PPAP2A_3 | Reverse Primer | CAGGTTGTCCTTCTATTCAGGCCA |
| 396 | AR | PPAP2A | PPAP2A_3 | Probe | CAGGTTGTCCTTCTATTCAGGCCA |
| 397 | AR | PPAP2A | PPAP2A_4 | Forward Primer | GAGGGAATGCAGAAAGAGTTA |
| 398 | AR | PPAP2A | PPAP2A_4 | Reverse Primer | GAAGATAAAGTGCCACAAACAG |
| 399 | AR | PPAP2A | PPAP2A_4 | Probe | AGGCAGGTTGTCCTTCTATTCAGGC |
| 400 | AR | PRKACB_2 | PRKACB_2_1 | Forward Primer | CTCTAAAGGTACTGCACATGA |
| 401 | AR | PRKACB_2 | PRKACB_2_1 | Reverse Primer | CTTTGGCTTTGGCTAGAAAC |
| 402 | AR | PRKACB_2 | PRKACB_2_1 | Probe | ACAGCCTTCATTTCTCTGAACATACTGCC |
| 403 | AR | PRKACB_2 | PRKACB_2_2 | Forward Primer | GCTAGCCGGTTATTTCATAGA |
| 404 | AR | PRKACB_2 | PRKACB_2_2 | Reverse Primer | CTTTCATTGATCTGTCCCATAAG |
| 405 | AR | PRKACB_2 | PRKACB_2_2 | Probe | TGACAGCCTTCATTTCTCTGAACATACTGCC |
| 406 | AR | PRKACB_2 | PRKACB_2_3 | Forward Primer | CCAGTATACAGGTACAACTACAG |
| 407 | AR | PRKACB_2 | PRKACB_2_3 | Reverse Primer | TAAGGCAGTATGTTCAGAGAAA |
| 408 | AR | PRKACB_2 | PRKACB_2_3 | Probe | TGCTAGCCGGTTATTTCATAGACACTCT |
| 409 | AR | PRKACB_2 | PRKACB_2_4 | Forward primer | ACTCTAAAGGTACTGCACATGATC |
| 410 | AR | PRKACB_2 | PRKACB_2_4 | Reverse primer | CTTTGGCTTTGGCTAGAAACTC |
| 411 | AR | PRKACB_2 | PRKACB_2_4 | Probe | AAAACAGCTCTGGAAAATGACAGCCTTCA |
| 412 | AR | SGK1 | SGK1_1 | Forward Primer | GGAGCCTGAGCTTATGAAT |
| 413 | AR | SGK1 | SGK1_1 | Reverse Primer | GAAGTGAAAGTCAGATGGTTTAG |
| 414 | AR | SGK1 | SGK1_1 | Probe | TTGGTGGAGGAGAAGGGTTGGC |
| 415 | AR | SGK1 | SGK1_2 | Forward Primer | TATGAATGCCAACCCTTCTC |
| 416 | AR | SGK1 | SGK1_2 | Reverse Primer | CCCTTTCCGATCACTTTCA |
| 417 | AR | SGK1 | SGK1_2 | Probe | AATCAACCTTGGCCCGTCGTCC |
| 418 | AR | SGK1 | SGK1_3 | Forward primer | CAGGAGCCTGAGCTTATGAA |
| 419 | AR | SGK1 | SGK1_3 | Reverse primer | GATGGTTTAGCATGAGGATTGG |
| 420 | AR | SGK1 | SGK1_3 | Probe | TCAGCAAATCAACCTTGGCCCGT |
| 421 | AR | SGK1 | SGK1_4 | Forward Primer | CTTGAAGATCTCCCAACCTC |
| 422 | AR | SGK1 | SGK1_4 | Reverse Primer | CAAGGTTGATTTGCTGAGAAG |
| 423 | AR | SGK1 | SGK1_4 | Probe | TTGGTGGAGGAGAAGGGTTGGC |
| 424 | AR | TMPRSS2 | TMPRSS2_1 | Forward Primer | ATGAAACTGAACACAAGTGC |
| 425 | AR | TMPRSS2 | TMPRSS2_1 | Reverse Primer | AGGCTATACAGCGTAAAGAAA |
| 426 | AR | TMPRSS2 | TMPRSS2_1 | Probe | CTGTACCACAGTGATGCCTGTTCTTCA |
| 427 | AR | TMPRSS2 | TMPRSS2_2 | Forward primer | CTGTTCTTCAAAAGCAGTGGTTT |
| 428 | AR | TMPRSS2 | TMPRSS2_2 | Reverse primer | TGGCGGCTTGAGTTCAA |
| 429 | AR | TMPRSS2 | TMPRSS2_2 | Probe | TTACGCTGTATAGCCTGCGGGGTCA |
| 430 | AR | TMPRSS2 | TMPRSS2_3 | Forward Primer | CGGATCCACCAGCTTTATG |
| 431 | AR | TMPRSS2 | TMPRSS2_3 | Reverse Primer | TTTGAAGAACAGGCATCACT |
| 432 | AR | TMPRSS2 | TMPRSS2_3 | Probe | ACACAAGTGCCGGCAATGTCGATA |
| 433 | AR | TMPRSS2 | TMPRSS2_4 | Forward Primer | CTTGAAGATCTCCCAACCTC |
| 434 | AR | TMPRSS2 | TMPRSS2_4 | Reverse Primer | CAAGGTTGATTTGCTGAGAAG |
| 435 | AR | TMPRSS2 | TMPRSS2_4 | Probe | TTGGTGGAGGAGAAGGGTTGGC |

TABLE 3
Sets of primers and probes for determining the PI3K-FOXO cellular
signaling pathway activity.

| SEQ | | | | | |
|---|---|---|---|---|---|
| ID | | | | | |
| NO | Pathway | Gene | Assay | Oligo | Sequence |
| 436 | PI3K-FOXO | AGRP | AGRP_1 | Forward | CCCACTGAAGAAGACAACTG |
| | | | | Primer | |
| 437 | PI3K-FOXO | AGRP | AGRP_1 | Reverse | TGCAGGTCTAGTACCTCTG |
| | | | | Primer | |
| 438 | PI3K-FOXO | AGRP | AGRP_1 | Probe | ATCTGTTGCAGGAGGCTCAGGC |
| 439 | PI3K-FOXO | AGRP | AGRP_2 | Forward | AACAGGCAGAAGAGGATCTG |
| | | | | Primer | |
| 440 | PI3K-FOXO | AGRP | AGRP_2 | Reverse | AGGACTCATGCAGCCTTAC |
| | | | | Primer | |
| 441 | PI3K-FOXO | AGRP | AGRP_2 | Probe | TACTAGACCTGCAGGACCGCGA |
| 442 | PI3K-FOXO | AGRP | AGRP_3 | Forward | AGAAGAGGATCTGTTGCAGGA |
| | | | | Primer | |
| 443 | PI3K-FOXO | AGRP | AGRP_3 | Reverse | CACATGGGTCACAGCAAGG |
| | | | | Primer | |
| 444 | PI3K-FOXO | AGRP | AGRP_3 | Probe | TCGCTGCGTAAGGCTGCATGA |
| 445 | PI3K-FOXO | AGRP | AGRP_4 | Forward | CCTTGGCAGAGGTACTAGA |
| | | | | Primer | |
| 446 | PI3K-FOXO | AGRP | AGRP_4 | Reverse | ATTGAAGAAGCGGCAGTAG |
| | | | | Primer | |
| 447 | PI3K-FOXO | AGRP | AGRP_4 | Probe | AGGTGCCTTGCTGTGACCCAT |
| 448 | PI3K-FOXO | BCL2L11 | BCL2L11_1 | Forward | CCTTTCTTGGCCCTTGTT |
| | | | | primer | |
| 449 | PI3K-FOXO | BCL2L11 | BCL2L11_1 | Reverse | AAGGTTGCTTTGCCATTTG |
| | | | | primer | |
| 450 | PI3K-FOXO | BCL2L11 | BCL2L11_1 | Probe | TGACTCTCGGACTGAGAAACGCAA |
| 451 | PI3K-FOXO | BCL2L11 | BCL2L11_2 | Forward | ATCGCATCATCGCGGTATT |
| | | | | Primer | |
| 452 | PI3K-FOXO | BCL2L11 | BCL2L11_2 | Reverse | GAGTCAGAGTCAGACATTTGGG |
| | | | | Primer | |
| 453 | PI3K-FOXO | BCL2L11 | BCL2L11_2 | Probe | CGCCCTTTCTTGGCCCTTGTTC |
| 454 | PI3K-FOXO | BCL6 | BCL6_1 | Forward | GGCCTGTTCTATAGCATCTT |
| | | | | Primer | |
| 455 | PI3K-FOXO | BCL6 | BCL6_1 | Reverse | GTGTACATGAAGTCCAGGAG |
| | | | | Primer | |
| 456 | PI3K-FOXO | BCL6 | BCL6_1 | Probe | TCCTGAGATCAACCCTGAGGGATTCT |
| 457 | PI3K-FOXO | BCL6 | BCL6_2 | Forward | GAGCCGTGAGCAGTTTAG |
| | | | | primer | |
| 458 | PI3K-FOXO | BCL6 | BCL6_2 | Reverse | GATCACACTAAGGTTGCATTTC |
| | | | | primer | |
| 459 | PI3K-FOXO | BCL6 | BCL6_2 | Probe | AAACGGTCCTCATGGCCTGCA |
| 460 | PI3K-FOXO | BCL6 | BCL6_3 | Forward | GTCGAGACATCTTGACTGATG |
| | | | | Primer | |
| 461 | PI3K-FOXO | BCL6 | BCL6_3 | Reverse | GCTATAGAACAGGCCACTG |
| | | | | Primer | |
| 462 | PI3K-FOXO | BCL6 | BCL6_3 | Probe | TCATTGTTGTGAGCCGTGAGCAGT |
| 463 | PI3K-FOXO | BCL6 | BCL6_4 | Forward | GTGATGTTCTTCTCAACCTTAATC |
| | | | | Primer | |
| 464 | PI3K-FOXO | BCL6 | BCL6_4 | Reverse | TTATGGGCTCTAAACTGCTC |
| | | | | Primer | |
| 465 | PI3K-FOXO | BCL6 | BCL6_4 | Probe | ACTGATGTTGTCATTGTTGTGAGCCGT |
| 466 | PI3K-FOXO | BNIP3 | BNIP3_1 | Forward | ATGAGTCTGGACGGAGTAG |
| | | | | Primer | |
| 467 | PI3K-FOXO | BNIP3 | BNIP3_1 | Reverse | CTCTCCAATGCTATGGGTATC |
| | | | | Primer | |
| 468 | PI3K-FOXO | BNIP3 | BNIP3_1 | Probe | TCGCTCGCAGACACCACAAGAT |
| 469 | PI3K-FOXO | BNIP3 | BNIP3_2 | Forward | CCCATAGCATTGGAGAGAAA |
| | | | | Primer | |
| 470 | PI3K-FOXO | BNIP3 | BNIP3_2 | Reverse | ACTTGACCAATCCCATATCC |
| | | | | Primer | |
| 471 | PI3K-FOXO | BNIP3 | BNIP3_2 | Probe | ACAGCTCACAGTCTGAGGAAGATGA |
| 472 | PI3K-FOXO | BNIP3 | BNIP3_3 | Forward | CACAAGATACCAACAGAGCTTC |
| | | | | primer | |
| 473 | PI3K-FOXO | BNIP3 | BNIP3_3 | Reverse | GCTTTCAACTTCTTTCCTTCTTTC |
| | | | | primer | |
| 474 | PI3K-FOXO | BNIP3 | BNIP3_3 | Probe | ACAGCTCACAGTCTGAGGAAGATGA |
| 475 | PI3K-FOXO | BNIP3 | BNIP3_3 | Forward | AGAGCTTCTGAAACAGATACC |
| | | | | Primer | |
| 476 | PI3K-FOXO | BNIP3 | BNIP3_3 | Reverse | GCTTTCAACTTCTTTCCTTCTT |
| | | | | Primer | |
| 477 | PI3K-FOXO | BNIP3 | BNIP3_3 | Probe | ACAGCTCACAGTCTGAGGAAGATGA |
| 478 | PI3K-FOXO | BTG1 | BTG1_1 | Forward | TCCTTCATCTCCAAGTTTCTC |
| | | | | Primer | |
| 479 | PI3K-FOXO | BTG1 | BTG1_1 | Reverse | TGGGAACCAGTGATGTTTAT |
| | | | | Primer | |
| 480 | PI3K-FOXO | BTG1 | BTG1_1 | Probe | AGCGACAGCTGCAGACCTTCAG |
| 481 | PI3K-FOXO | BTG1 | BTG1_2 | Forward | CGACAGCTGCAGACCTT |
| | | | | primer | |
| 482 | PI3K-FOXO | BTG1 | BTG1_2 | Reverse | GTTGATGCGAATACAACGG |
| | | | | primer | |
| 483 | PI3K-FOXO | BTG1 | BTG1_2 | Probe | CAGGAGCTGCTGGCAGAACATTA |
| 484 | PI3K-FOXO | BTG1 | BTG1_3 | Forward | ACATCACTGGTTCCCAGAA |
| | | | | Primer | |
| 485 | PI3K-FOXO | BTG1 | BTG1_3 | Reverse | GCCTGTCCAATCAGAGGAT |
| | | | | Primer | |
| 486 | PI3K-FOXO | BTG1 | BTG1_3 | Probe | ACAACGGTAACCCGATCCCTTGC |
| 487 | PI3K-FOXO | BTG1 | BTG1_4 | Forward | ATCGGGTTACCGTTGTATTC |
| | | | | Primer | |
| 488 | PI3K-FOXO | BTG1 | BTG1_4 | Reverse | CTGACTGCTCAGTCCAATC |
| | | | | Primer | |
| 489 | PI3K-FOXO | BTG1 | BTG1_4 | Probe | CCTCTGATTGGACAGGCAGCACA |
| 490 | PI3K-FOXO | CAT | CAT_1 | Forward | GAGAAGTGCGGAGATTCAA |
| | | | | Primer | |
| 491 | PI3K-FOXO | CAT | CAT_1 | Reverse | TTCTCACACAGACGTTTCC |
| | | | | Primer | |
| 492 | PI3K-FOXO | CAT | CAT_1 | Probe | ACGTTACTCAGGTGCGGGCATT |
| 493 | PI3K-FOXO | CAT | CAT_2 | Forward | GTGCTGAATGAGGAACAGA |
| | | | | Primer | |
| 494 | PI3K-FOXO | CAT | CAT_2 | Reverse | AGTTCTTGACCGCTTTCTT |
| | | | | Primer | |
| 495 | PI3K-FOXO | CAT | CAT_2 | Probe | ACGTCTGTGTGAGAACATTGCCGG |
| 496 | PI3K-FOXO | CAT | CAT_3 | Forward | CTCCGGAACAACAGCCTTC |
| | | | | Primer | |
| 497 | PI3K-FOXO | CAT | CAT_3 | Reverse | CATCATTGGCAGTGTTGAATCTC |
| | | | | Primer | |
| 498 | PI3K-FOXO | CAT | CAT_3 | Probe | TATTGGATGCTGTGCTCCAGGGC |
| 499 | PI3K-FOXO | CAT | CAT_4 | Forward | AACACTGCCAATGATGATAAC |
| | | | | primer | |
| 500 | PI3K-FOXO | CAT | CAT_4 | Reverse | ACAGACGTTTCCTCTGTTC |
| | | | | primer | |
| 501 | PI3K-FOXO | CAT | CAT_4 | Probe | CGGGCATTCTATGTGAACGTGCT |
| 502 | PI3K-FOXO | CAV1 | CAV1_1 | Forward | CGATGACGTGGTCAAGAT |
| | | | | primer | |
| 503 | PI3K-FOXO | CAV1 | CAV1_1 | Reverse | CTTCCAAATGCCGTCAAA |
| | | | | primer | |
| 504 | PI3K-FOXO | CAV1 | CAV1_1 | Probe | TTGCAGAACCAGAAGGGACACACA |
| 505 | PI3K-FOXO | CAV1 | CAV1_2 | Forward | CCAGAAGGGACACACAGTTT |
| | | | | Primer | |
| 506 | PI3K-FOXO | CAV1 | CAV1_2 | Reverse | AAAGAGGGCAGACAGCAAG |
| | | | | Primer | |
| 507 | PI3K-FOXO | CAV1 | CAV1_2 | Probe | AAGGCCAGCTTCACCACCTTCA |
| 508 | PI3K-FOXO | CAV1 | CAV1_3 | Forward | GCACACCAAGGAGATCGAC |
| | | | | Primer | |
| 509 | PI3K-FOXO | CAV1 | CAV1_3 | Reverse | CCCTTCTGGTTCTGCAATCA |
| | | | | Primer | |
| 510 | PI3K-FOXO | CAV1 | CAV1_3 | Probe | TGGTCAACCGCGACCCTAAACAC |
| 511 | PI3K-FOXO | CAV1 | CAV1_4 | Forward | CGATGACGTGGTCAAGATT |
| | | | | Primer | |
| 512 | PI3K-FOXO | CAV1 | CAV1_4 | Reverse | AACTGTGTGTCCCTTCTGGTTCTGC |
| | | | | Primer | |
| 634 | PI3K-FOXO | CCND1 | CCND1_1 | Forward | CCTCGGTGTCCTACTTCAAA |
| | | | | Primer | |
| 635 | PI3K-FOXO | CCND1 | CCND1_1 | Reverse | ACTTCTGTTCCTCGCAGAC |
| | | | | Primer | |
| 636 | PI3K-FOXO | CCND1 | CCND1_1 | Probe | AAGATCGTCGCCACCTGGATGC |
| 637 | PI3K-FOXO | CCND1 | CCND1_2 | Forward | CTTCAAATGTGTGCAGAAGGAG |
| | | | | Primer | |
| 638 | PI3K-FOXO | CCND1 | CCND1_2 | Reverse | GAAGCGGTCCAGGTAGTTC |
| | | | | Primer | |
| 639 | PI3K-FOXO | CCND1 | CCND1_2 | Probe | TGCGAGGAACAGAAGTGCGAGG |
| 640 | PI3K-FOXO | CCND1 | CCND1_3 | Forward | CATGCGGAAGATCGTCGC |
| | | | | Primer | |
| 641 | PI3K-FOXO | CCND1 | CCND1_3 | Reverse | GACCTCCTCCTCGCACT |
| | | | | Primer | |
| 642 | PI3K-FOXO | CCND1 | CCND1_3 | Probe | CTGGATGCTGGAGGTCTGCGAGGAA |
| 643 | PI3K-FOXO | CCND1 | CCND1_4 | Forward | GTGTCCTACTTCAAATGTGTGC |
| | | | | Primer | |
| 644 | PI3K-FOXO | CCND1 | CCND1_4 | Reverse | CCTCCTCGCACTTCTGTTC |
| | | | | Primer | |
| 645 | PI3K-FOXO | CCND1 | CCND1_4 | Probe | AAGATCGTCGCCACCTGGATGC |
| 513 | PI3K-FOXO | CAV1 | CAV1_4 | Probe | CACAGTGAAGGTGGTGAAG |
| 514 | PI3K-FOXO | CCNG2 | CCNG2_1 | Forward | ACAGGTTCTTGGCTCTTATG |
| | | | | Primer | |
| 515 | PI3K-FOXO | CCNG2 | CCNG2_1 | Reverse | TGCAGTCTTCTTCAACTATTCT |
| | | | | Primer | |
| 516 | PI3K-FOXO | CCNG2 | CCNG2_1 | Probe | CCTAAACATTTGTCTTGCATTGGAGTCTGT |
| 517 | PI3K-FOXO | CCNG2 | CCNG2_2 | Forward | TTGCTGGCTGCTAGAATAG |
| | | | | Primer | |
| 518 | PI3K-FOXO | CCNG2 | CCNG2_2 | Reverse | GTCAGAAGCAGTACATTTACAC |
| | | | | Primer | |
| 519 | PI3K-FOXO | CCNG2 | CCNG2_2 | Probe | TCCATCCACTCATGATGTGATCCGGA |
| 520 | PI3K-FOXO | CCNG2 | CCNG2_3 | Forward | ACAGGTTCTTGGCTCTTATG |
| | | | | primer | |
| 521 | PI3K-FOXO | CCNG2 | CCNG2_3 | Reverse | TGCAGTCTTCTTCAACTATTCT |
| | | | | primer | |
| 522 | PI3K-FOXO | CCNG2 | CCNG2_3 | Probe | ACATTTGTCTTGCATTGGAGTCTGT |
| 523 | PI3K-FOXO | CCNG2 | CCNG2_4 | Forward | TGGATCTTGCACTGAAACT |
| | | | | Primer | |
| 524 | PI3K-FOXO | CCNG2 | CCNG2_4 | Reverse | CTCCAATGCAAGACAAATGTT |
| | | | | Primer | |
| 525 | PI3K-FOXO | CCNG2 | CCNG2_4 | Probe | CACCTTCATAAGAGCCAAGAACCTGTCC |
| 1066 | PI3K-FOXO | CDKN1A | CDKN1A_1 | Reverse | CTGTGGGCGGATTAGGGCT |
| | | | | primer | |
| 1067 | PI3K-FOXO | CDKN1A | CDKN1A_1 | Forward | GAGACTCTCAGGGTCGAAA |
| | | | | primer | |
| 1068 | PI3K-FOXO | CDKN1A | CDKN1A_1 | Probe | ATTTCTACCACTCCAAACGCCGGC |
| 1069 | PI3K-FOXO | CDKN1A | CDKN1A_2 | Forward | GAGACTCTCAGGGTCGAAA |
| | | | | Primer | |
| 1070 | PI3K-FOXO | CDKN1A | CDKN1A_2 | Probe | AATCTGTCATGCTGGTCTGCCGC |
| 1071 | PI3K-FOXO | CDKN1A | CDKN1A_2 | Reverse | TTCCTGTGGGCGGATTA |
| | | | | Primer | |
| 1072 | PI3K-FOXO | CDKN1A | CDKN1A_3 | Forward | AGGTGGACCTGGAGACT |
| | | | | Primer | |
| 1073 | PI3K-FOXO | CDKN1A | CDKN1A_3 | Probe | AATCTGTCATGCTGGTCTGCCGC |
| 1074 | PI3K-FOXO | CDKN1A | CDKN1A_3 | Reverse | GGCTTCCTCTTGGAGAAGAT |
| | | | | Primer | |
| 1075 | PI3K-FOXO | CDKN1A | CDKN1A_4 | Forward | GGACCTGTCACTGTCTTGTA |
| | | | | Primer | |
| 1076 | PI3K-FOXO | CDKN1A | CDKN1A_4 | Probe | AAACGGCGGCAGACCAGCAT |
| 1077 | PI3K-FOXO | CDKN1A | CDKN1A_4 | Reverse | GCGTTTGGAGTGGTAGAAATC |
| | | | | Primer | |
| 526 | PI3K-FOXO | CDKN1B | CDKN1B_1 | Forward | AGAGCCAACAGAACAGAAG |
| | | | | Primer | |
| 527 | PI3K-FOXO | CDKN1B | CDKN1B_1 | Reverse | TCGAGCTGTTTACGTTTGA |
| | | | | Primer | |
| 528 | PI3K-FOXO | CDKN1B | CDKN1B_1 | Probe | AAATGCCGGTTCTGTGGAGCAGA |
| 529 | PI3K-FOXO | CDKN1B | CDKN1B_2 | Forward | CAAACGTAAACAGCTCGAATTA |
| | | | | Primer | |
| 530 | PI3K-FOXO | CDKN1B | CDKN1B_2 | Reverse | TCCATGAAGTCAGCGATATG |
| | | | | Primer | |
| 531 | PI3K-FOXO | CDKN1B | CDKN1B_2 | Probe | ACATCACTGCTTGATGAAGCAAGGAAGA |
| 532 | PI3K-FOXO | CDKN1B | CDKN1B_3 | Forward | AAGAAGCCTGGCCTCAGAA |
| | | | | Primer | |
| 533 | PI3K-FOXO | CDKN1B | CDKN1B_3 | Reverse | TCATGTATATCTTCCTTGCTTCATC |
| | | | | Primer | |
| 534 | PI3K-FOXO | CDKN1B | CDKN1B_3 | Probe | TCTTAATTCGAGCTGTTTACGTTTGACGTC |
| 535 | PI3K-FOXO | CDKN1B | CDKN1B_4 | Forward | CGGTTCTGTGGAGCAGACG |
| | | | | Primer | |
| 536 | PI3K-FOXO | CDKN1B | CDKN1B_4 | Reverse | CTTCATCAAGCAGTGATGTATCTG |
| | | | | Primer | |
| 537 | PI3K-FOXO | CDKN1B | CDKN1B_4 | Probe | CCTGGCCTCAGAAGACGTCAAAC |
| 67 | PI3K-FOXO | ESR1 | ESR1_1 | Forward | CTTCGATGATGGGCTTACT |
| | | | | Primer | |
| 68 | PI3K-FOXO | ESR1 | ESR1_1 | Probe | CATGTGAACCAGCTCCCTGTCTGC |
| 69 | PI3K-FOXO | ESR1 | ESR1_1 | Reverse | GGAGGGTCAAATCCACAA |
| | | | | Primer | |
| 70 | PI3K-FOXO | ESR1 | ESR1_2 | Probe | CAACTGGGCGAAGAGGGTGCCA |
| 71 | PI3K-FOXO | ESR1 | ESR1_2 | Forward | AGCTTCGATGATGGGCTTAC |
| | | | | primer | |
| 72 | PI3K-FOXO | ESR1 | ESR1_2 | Reverse | CCTGATCATGGAGGGTCAAA |
| | | | | primer | |
| 73 | PI3K-FOXO | ESR1 | ESR1_3 | Forward | GGAGCTGGTTCACATGAT |
| | | | | Primer | |
| 74 | PI3K-FOXO | ESR1 | ESR1_3 | Probe | AGGGTCAAATCCACAAAGCCTGGC |
| 75 | PI3K-FOXO | ESR1 | ESR1_3 | Reverse | CTAGCCAGGCACATTCTA |
| | | | | Primer | |
| 76 | PI3K-FOXO | ESR1 | ESR1_4 | Forward | GATGGGCTTACTGACCAA |
| | | | | Primer | |
| 77 | PI3K-FOXO | ESR1 | ESR1_4 | Probe | CATGTGAACCAGCTCCCTGTCTGC |
| 78 | PI3K-FOXO | ESR1 | ESR1_4 | Reverse | CTGATCATGGAGGGTCAAA |
| | | | | Primer | |
| 538 | PI3K-FOXO | FBXO32 | FBXO32_1 | Forward | GCTGCTGTGGAAGAAACT |
| | | | | primer | |
| 539 | PI3K-FOXO | FBXO32 | FBXO32_1 | Reverse | GCCCTTTGTCTGACAGAATTA |
| | | | | primer | |
| 540 | PI3K-FOXO | FBXO32 | FBXO32_1 | Probe | TGCCAGTACCACTTCTCCGAGC |
| 541 | PI3K-FOXO | FBXO32 | FBXO32_2 | Forward | ATCCGCAAACGATTAATTCTG |
| | | | | Primer | |
| 542 | PI3K-FOXO | FBXO32 | FBXO32_2 | Reverse | TCCATACTGCTCTTTCCTTG |
| | | | | Primer | |
| 543 | PI3K-FOXO | FBXO32 | FBXO32_2 | Probe | ACAAAGGGCAGCTGGATTGGAAGA |
| 544 | PI3K-FOXO | FBXO32 | FBXO32_3 | Forward | AAGAAACTCTGCCAGTACC |
| | | | | Primer | |
| 545 | PI3K-FOXO | FBXO32 | FBXO32_3 | Reverse | ACATCGGACAAGTTTGAAATAC |
| | | | | Primer | |
| 546 | PI3K-FOXO | FBXO32 | FBXO32_3 | Probe | AGCGGCAGATCCGCAAACGATTA |
| 547 | PI3K-FOXO | FBXO32 | FBXO32_4 | Forward | CTGCTGTGGAAGAAACTCT |
| | | | | Primer | |
| 548 | PI3K-FOXO | FBXO32 | FBXO32_4 | Reverse | CTTGGGTAACATCGGACAA |
| | | | | Primer | |
| 549 | PI3K-FOXO | FBXO32 | FBXO32_4 | Probe | AGCGGCAGATCCGCAAACGATTA |
| 550 | PI3K-FOXO | FOXO3_2 | FOX03_1 | Forward | GTGCCCTACTTCAAGGATAAG |
| | | | | Primer | |
| 551 | PI3K-FOXO | FOXO3_2 | FOXO3_1 | Reverse | CTTGCCAGTTCCCTCATTC |
| | | | | Primer | |
| 552 | PI3K-FOXO | FOXO3_2 | FOXO3_1 | Probe | AACTCCATCCGGCACAACCTGT |
| 553 | PI3K-FOXO | FOXO3_2 | FOXO3_2 | Forward | CAACCTGTCACTGCATAGT |
| | | | | Primer | |
| 554 | PI3K-FOXO | FOXO3_2 | FOXO3_2 | Reverse | TTGATGATCCACCAAGAGC |
| | | | | Primer | |
| 555 | PI3K-FOXO | FOXO3_2 | FOXO3_2 | Probe | TGCGGGTCCAGAATGAGGGAAC |
| 556 | PI3K-FOXO | FOXO3_2 | FOXO3_3 | Forward | ACAAACGGCTCACTCTGTC |
| | | | | Primer | |
| 557 | PI3K-FOXO | FOXO3_2 | FOXO3_3 | Reverse | TGTTGCTGTCGCCCTTATC |
| | | | | Primer | |
| 558 | PI3K-FOXO | FOXO3_2 | FOXO3_3 | Probe | TACGAGTGGATGGTGCGTTGCG |
| 559 | PI3K-FOXO | FOXO3_2 | FOXO3_4 | Forward | CGTGCCCTACTTCAAGGATAA |
| | | | | Primer | |
| 560 | PI3K-FOXO | FOXO3_2 | FOXO3_4 | Reverse | TCATTCTGGACCCGCATG |
| | | | | Primer | |
| 561 | PI3K-FOXO | FOXO3_2 | FOXO3_4 | Probe | CGGCTGGAAGAACTCCATCCGGCA |
| 562 | PI3K-FOXO | GADD45A | GADD45A_1 | Forward | GGTGACGAATCCACATTCA |
| | | | | Primer | |
| 563 | PI3K-FOXO | GADD45A | GADD45A_1 | Reverse | TCACCGTTCAGGGAGATTA |
| | | | | Primer | |
| 564 | PI3K-FOXO | GADD45A | GADD45A_1 | Probe | TGCCGGGAAAGTCGCTACATGG |
| 565 | PI3K-FOXO | GADD45A | GADD45A_2 | Forward | ACGAATCCACATTCATCTCAAT |
| | | | | primer | |
| 566 | PI3K-FOXO | GADD45A | GADD45A_2 | Reverse | GATCCATGTAGCGACTTTCC |
| | | | | primer | |
| 567 | PI3K-FOXO | GADD45A | GADD45A_2 | Probe | AAGGATCCTGCCTTAAGTCAACTTATTTG |
| 568 | PI3K-FOXO | GADD45A | GADD45A_3 | Forward | CCTGCCTTAAGTCAACTTATTT |
| | | | | Primer | |
| 569 | PI3K-FOXO | GADD45A | GADD45A_3 | Reverse | TCATTCAGATGCCATCACC |
| | | | | Primer | |
| 570 | PI3K-FOXO | GADD45A | GADD45A_3 | Probe | TGCCGGGAAAGTCGCTACATGG |
| 571 | PI3K-FOXO | GADD45A | GADD45A_4 | Forward | CACATTCATCTCAATGGAAGG |
| | | | | Primer | |
| 572 | PI3K-FOXO | GADD45A | GADD45A_4 | Reverse | CAGGGAGATTAATCACTGGAA |
| | | | | Primer | |
| 573 | PI3K-FOXO | GADD45A | GADD45A_4 | Probe | TGCCGGGAAAGTCGCTACATGG |
| 574 | PI3K-FOXO | INSR | INSR_1 | Forward | GACCCAGTATGCCATCTTT |
| | | | | Primer | |
| 575 | PI3K-FOXO | INSR | INSR_1 | Reverse | GGCATCTGTCTGGACATAAA |
| | | | | Primer | |
| 576 | PI3K-FOXO | INSR | INSR_1 | Probe | TTTCGGATGAACGCCGGACCT |
| 577 | PI3K-FOXO | INSR | INSR_2 | Forward | CTGGATCCAATCTCAGTGTC |
| | | | | Primer | |
| 578 | PI3K-FOXO | INSR | INSR_2 | Reverse | CAGGTAGTGGGTGATGTTG |
| | | | | Primer | |
| 579 | PI3K-FOXO | INSR | INSR_2 | Probe | AAGTGGAAACCACCCTCCGACC |
| 580 | PI3K-FOXO | INSR | INSR_3 | Forward | GGCCAAGAGTGACATCATTTAT |
| | | | | primer | |
| 581 | PI3K-FOXO | INSR | INSR_3 | Reverse | GGTGGTTTCCACTTCAGAATAAT |
| | | | | primer | |
| 582 | PI3K-FOXO | INSR | INSR_3 | Probe | AACCCCTCTGTGCCCCT |
| 583 | PI3K-FOXO | INSR | INSR_4 | Forward | CCATCTTTGTGAAGACCCT |
| | | | | Primer | |
| 584 | PI3K-FOXO | INSR | INSR_4 | Reverse | AATCTGGGATGATGAGTTAGAC |
| | | | | Primer | |
| 585 | PI3K-FOXO | INSR | INSR_4 | Probe | TCCAGACAGATGCCACCAACCC |
| 586 | PI3K-FOXO | MXI1 | MXI1_1 | Forward | CATCTGCGCCTTTGTTTAG |
| | | | | Primer | |
| 587 | PI3K-FOXO | MXI1 | MXI1_1 | Reverse | GTGCTTTGGCTTTGTTGAG |
| | | | | Primer | |
| 588 | PI3K-FOXO | MXI1 | MXI1_1 | Probe | CCCGGCACACAACACTTGGTTTG |
| 589 | PI3K-FOXO | MXI1 | MXI1_2 | Forward | GCACACAACACTTGGTTT |
| | | | | primer | |
| 590 | PI3K-FOXO | MXI1 | MXI1_2 | Reverse | CTGTTCTCGTTCCAAATTCTC |
| | | | | primer | |
| 591 | PI3K-FOXO | MXI1 | MXI1_2 | Probe | AGCACACATCAAGAAACTTGAAGAAGCTGA |
| 592 | PI3K-FOXO | MXI1 | MXI1_3 | Forward | CCAAAGCACACATCAAGAAA |
| | | | | Primer | |
| 593 | PI3K-FOXO | MXI1 | MXI1_3 | Reverse | TTCGTATTCGTTCCATCTCC |
| | | | | Primer | |
| 594 | PI3K-FOXO | MXI1 | MXI1_3 | Probe | AAAGCCAGCACCAGCTCGAGAA |
| 595 | PI3K-FOXO | MXI1 | MXI1_4 | Forward | TGATTCCACTAGGACCAGA |
| | | | | Primer | |
| 596 | PI3K-FOXO | MXI1 | MXI1_4 | Reverse | TCTTTCAGCTTCTTCAAGTTTC |
| | | | | Primer | |
| 597 | PI3K-FOXO | MXI1 | MXI1_4 | Probe | CCCGGCACACAACACTTGGTTTG |
| 598 | PI3K-FOXO | SOD2 | SOD2_1 | Forward | AGCGGCTTCAGCAGATC |
| | | | | primer | |
| 599 | PI3K-FOXO | SOD2 | SOD2_1 | Reverse | GCCTGGAGCCCAGATAC |
| | | | | primer | |
| 600 | PI3K-FOXO | SOD2 | SOD2_1 | Probe | ACTAGCAGCATGTTGAGCCGGG |
| 601 | PI3K-FOXO | SOD2 | SOD2_2 | Forward | GCACTAGCAGCATGTTGAG |
| | | | | Primer | |
| 602 | PI3K-FOXO | SOD2 | SOD2_2 | Reverse | CGTTGATGTGAGGTTCCAG |
| | | | | Primer | |
| 603 | PI3K-FOXO | SOD2 | SOD2_2 | Probe | TCCAGGCAGAAGCACAGCCT |
| 604 | PI3K-FOXO | SOD2 | SOD2_3 | Forward | CGACCTGCCCTACGACTAC |
| | | | | Primer | |
| 605 | PI3K-FOXO | SOD2 | SOD2_3 | Reverse | GGTGACGTTCAGGTTGTTCAC |
| | | | | Primer | |
| 606 | PI3K-FOXO | SOD2 | SOD2_3 | Probe | TGGAACCTCACATCAACGCGCA |
| 607 | PI3K-FOXO | SOD2 | SOD2_4 | Forward | CCTGGAACCTCACATCAAC |
| | | | | Primer | |
| 608 | PI3K-FOXO | SOD2 | SOD2_4 | Reverse | CCTCCTGGTACTTCTCCTC |
| | | | | Primer | |
| 609 | PI3K-FOXO | SOD2 | SOD2_4 | Probe | TCAGGTTGTTCACGTAGGCCGC |
| 610 | PI3K-FOXO | TNFSF10 | TNFSF10_1 | Forward | CTCTCTGTGTGGCTGTAAC |
| | | | | Primer | |
| 611 | PI3K-FOXO | TNFSF10 | TNFSF10_1 | Reverse | GGGCTGTTCATACTCTCTTC |
| | | | | Primer | |
| 612 | PI3K-FOXO | TNFSF10 | TNFSF10_1 | Probe | ACCAACGAGCTGAAGCAGATGCA |
| 613 | PI3K-FOXO | TNFSF10 | TNFSF10_2 | Forward | GGCTGTAACTTACGTGTACTT |
| | | | | Primer | |
| 614 | PI3K-FOXO | TNFSF10 | TNFSF10_2 | Reverse | GGGTCCCAATAACTGTCATC |
| | | | | Primer | |
| 615 | PI3K-FOXO | TNFSF10 | TNFSF10_2 | Probe | ACCAACGAGCTGAAGCAGATGCA |
| 616 | PI3K-FOXO | TNFSF10 | TNFSF10_3 | Forward | CCTGCAGTCTCTCTGTGT |
| | | | | primer | |
| 617 | PI3K-FOXO | TNFSF10 | TNFSF10_3 | Reverse | GCCACTTTTGGAGTACTTGT |
| | | | | primer | |
| 618 | PI3K-FOXO | TNFSF10 | TNFSF10_3 | Probe | TACCAACGAGCTGAAGCAGATGCA |
| 619 | PI3K-FOXO | TNFSF10 | TNFSF10_3 | Forward | GCTGATCGTGATCTTCACA |
| | | | | Primer | |
| 620 | PI3K-FOXO | TNFSF10 | TNFSF10_3 | Reverse | GAGTACTTGTCCTGCATCTG |
| | | | | Primer | |
| 621 | PI3K-FOXO | TNFSF10 | TNFSF10_3 | Probe | TGCAGTCTCTCTGTGTGGCTGT |

TABLE 4
Sets of primers and probes for determining the AP1-MAPK cellular signaling
pathway activity.

| SEQ | | | | | |
|---|---|---|---|---|---|
| ID | | | | | |
| NO | Pathway | Gene | Assay | Oligo | Sequence |
| 448 | AP1-MAPK | BCL2L11 | BCL2L11_1 | Forward | CCTTTCTTGGCCCTTGTT |
| | | | | primer | |
| 449 | AP1-MAPK | BCL2L11 | BCL2L11_1 | Reverse | AAGGTTGCTTTGCCATTTG |
| | | | | primer | |
| 450 | AP1-MAPK | BCL2L11 | BCL2L11_1 | Probe | TGACTCTCGGACTGAGAAACGCAA |
| 451 | AP1-MAPK | BCL2L11 | BCL2L11_2 | Forward | ATCGCATCATCGCGGTATT |
| | | | | Primer | |
| 452 | AP1-MAPK | BCL2L11 | BCL2L11_2 | Reverse | GAGTCAGAGTCAGACATTTGGG |
| | | | | Primer | |
| 453 | AP1-MAPK | BCL2L11 | BCL2L11_2 | Probe | CGCCCTTTCTTGGCCCTTGTTC |
| 622 | AP1-MAPK | DDIT3 | DDIT3_1 | Forward | TCCAGTACAACTTTACCTACAA |
| | | | | Primer | |
| 623 | AP1-MAPK | DDIT3 | DDIT3_1 | Reverse | AGCACATCTGCAGGATAAT |
| | | | | Primer | |
| 624 | AP1-MAPK | DDIT3 | DDIT3_1 | Probe | CAGGCATCAGACCAGCTTGCCA |
| 625 | AP1-MAPK | DDIT3 | DDIT3_2 | Forward | TGCTACATGGAGCTTGTTC |
| | | | | Primer | |
| 626 | AP1-MAPK | DDIT3 | DDIT3_2 | Reverse | CGAAGGAGAAAGGCAATGA |
| | | | | Primer | |
| 627 | AP1-MAPK | DDIT3 | DDIT3_2 | Probe | CCAACTGCAGAGATGGCAGCTGA |
| 628 | AP1-MAPK | DDIT3 | DDIT3_3 | Forward | GGCTGTATTCCAGTACAACTT |
| | | | | Primer | |
| 629 | AP1-MAPK | DDIT3 | DDIT3_3 | Reverse | CAGTTGGATCAGTCTGGAAA |
| | | | | Primer | |
| 630 | AP1-MAPK | DDIT3 | DDIT3_3 | Probe | CAGGCATCAGACCAGCTTGCCA |
| 631 | AP1-MAPK | DDIT3 | DDIT3_4 | Forward | CACCAAAGCAGCCATAAAC |
| | | | | Primer | |
| 632 | AP1-MAPK | DDIT3 | DDIT3_4 | Reverse | CTCCATGTAGCAAACAGTCTA |
| | | | | Primer | |
| 633 | AP1-MAPK | DDIT3 | DDIT3_4 | Probe | CAGGCATCAGACCAGCTTGCCA |
| 634 | AP1-MAPK | CCND1 | CCND1_1 | Forward | CCTCGGTGTCCTACTTCAAA |
| | | | | Primer | |
| 635 | AP1-MAPK | CCND1 | CCND1_1 | Reverse | ACTTCTGTTCCTCGCAGAC |
| | | | | Primer | |
| 636 | AP1-MAPK | CCND1 | CCND1_1 | Probe | AAGATCGTCGCCACCTGGATGC |
| 637 | AP1-MAPK | CCND1 | CCND1_2 | Forward | CTTCAAATGTGTGCAGAAGGAG |
| | | | | Primer | |
| 638 | AP1-MAPK | CCND1 | CCND1_2 | Reverse | GAAGCGGTCCAGGTAGTTC |
| | | | | Primer | |
| 639 | AP1-MAPK | CCND1 | CCND1_2 | Probe | TGCGAGGAACAGAAGTGCGAGG |
| 640 | AP1-MAPK | CCND1 | CCND1_3 | Forward | CATGCGGAAGATCGTCGC |
| | | | | Primer | |
| 641 | AP1-MAPK | CCND1 | CCND1_3 | Reverse | GACCTCCTCCTCGCACT |
| | | | | Primer | |
| 642 | AP1-MAPK | CCND1 | CCND1_3 | Probe | CTGGATGCTGGAGGTCTGCGAGGAA |
| 643 | AP1-MAPK | CCND1 | CCND1_4 | Forward | GTGTCCTACTTCAAATGTGTGC |
| | | | | Primer | |
| 644 | AP1-MAPK | CCND1 | CCND1_4 | Reverse | CCTCCTCGCACTTCTGTTC |
| | | | | Primer | |
| 645 | AP1-MAPK | CCND1 | CCND1_4 | Probe | AAGATCGTCGCCACCTGGATGC |
| 646 | AP1-MAPK | EGFR | EGFR_1 | Forward | TACCAGATGGATGTGAACCC |
| | | | | primer | |
| 647 | AP1-MAPK | EGFR | EGFR_1 | Reverse | CCGTGATCTGTCACCACATA |
| | | | | primer | |
| 648 | AP1-MAPK | EGFR | EGFR_1 | Probe | TGCCACCTGCGTGAAGAAGTGT |
| 649 | AP1-MAPK | ENPP2 | ENPP2_1 | Forward | GATGCATTCCTTGTAACCAATA |
| | | | | primer | |
| 650 | AP1-MAPK | ENPP2 | ENPP2_1 | Reverse | ATTTCTTCACCAATACCCTTTG |
| | | | | primer | |
| 651 | AP1-MAPK | ENPP2 | ENPP2_1 | Probe | TCCTGCTTTCAAACGGGTCTGGA |
| 652 | AP1-MAPK | EZR | EZR_1 | Forward | GAGTGAAATCAGGAACATCTC |
| | | | | primer | |
| 653 | AP1-MAPK | EZR | EZR_1 | Reverse | CTTGTTGATTCTCAGACGTG |
| | | | | primer | |
| 654 | AP1-MAPK | EZR | EZR_1 | Probe | ATCGACAAGAAGGCACCTGACTTTG |
| 655 | AP1-MAPK | GLRX | GLRX_1 | Forward | CAGTCAATTGCCCATCAAACA |
| | | | | primer | |
| 656 | AP1-MAPK | GLRX | GLRX_1 | Reverse | GTGAGCTGTTGCAAATAATCTT |
| | | | | primer | |
| 657 | AP1-MAPK | GLRX | GLRX_1 | Probe | CACAGCCACCAACCACACTAACGA |
| 658 | AP1-MAPK | MMP1 | MMP1_1 | Forward | GTGTGACAGTAAGCTAACCTTTG |
| | | | | primer | |
| 659 | AP1-MAPK | MMP1 | MMP1_1 | Reverse | GCTCAACTTCCGGGTAGAA |
| | | | | primer | |
| 660 | AP1-MAPK | MMP1 | MMP1_1 | Probe | AGACAGATTCTACATGCGCACAAATCCC |
| 661 | AP1-MAPK | MMP3 | MMP3_1 | Forward | CGATGCAGCCATTTCTGATA |
| | | | | primer | |
| 662 | AP1-MAPK | MMP3 | MMP3_1 | Reverse | GGAAAGTCTTCAGCTATTTGCT |
| | | | | primer | |
| 663 | AP1-MAPK | MMP3 | MMP3_1 | Probe | CTGGAGATTTGATGAGAAGAGAAATTCCAT |
| 664 | AP1-MAPK | MMP3 | MMP3_2 | Forward | CATCCACACCCTAGGTTTC |
| | | | | Primer | |
| 665 | AP1-MAPK | MMP3 | MMP3_2 | Reverse | CTCCAGTATTTGTCCTCTACAA |
| | | | | Primer | |
| 666 | AP1-MAPK | MMP3 | MMP3_2 | Probe | TCGATGCAGCCATTTCTGATAAGGA |
| 667 | AP1-MAPK | MMP3 | MMP3_3 | Forward | CCCTAGGTTTCCCTCCAAC |
| | | | | Primer | |
| 668 | AP1-MAPK | MMP3 | MMP3_3 | Reverse | GCTCCATGGAATTTCTCTTCTC |
| | | | | Primer | |
| 669 | AP1-MAPK | MMP3 | MMP3_3 | Probe | TCGATGCAGCCATTTCTGATAAGGA |
| 670 | AP1-MAPK | MMP3 | MMP3_4 | Forward | GTAGAGGACAAATACTGGAGAT |
| | | | | Primer | |
| 671 | AP1-MAPK | MMP3 | MMP3_4 | Reverse | GAGTCAATCCCTGGAAAGT |
| | | | | Primer | |
| 672 | AP1-MAPK | MMP3 | MMP3_4 | Probe | CCATGGAGCCAGGCTTTCCCAA |
| 673 | AP1-MAPK | MMP9 | MMP9_1_1 | Forward | TGGAGACCTGAGAACCAATC |
| | | | | Primer | |
| 674 | AP1-MAPK | MMP9 | MMP9_1 | Reverse | ACCCGAGTGTAACCATAGC |
| | | | | Primer | |
| 675 | AP1-MAPK | MMP9 | MMP9_1 | Probe | AGGCAGCTGGCAGAGGAATACCT |
| 676 | AP1-MAPK | MMP9 | MMP9_2 | Forward | GGAGACCTGAGAACCAATC |
| | | | | primer | |
| 677 | AP1-MAPK | MMP9 | MMP9_2 | Reverse | GACTCTCCACGCATCTCTG |
| | | | | primer | |
| 678 | AP1-MAPK | MMP9 | MMP9_2 | Probe | AGGCAGCTGGCAGAGGAATACCT |
| 679 | AP1-MAPK | MMP9 | MMP9_3 | Forward | AGAACCAATCTCACCGACAG |
| | | | | Primer | |
| 680 | AP1-MAPK | MMP9 | MMP9_3 | Reverse | CCAGAGATTTCGACTCTCCAC |
| | | | | Primer | |
| 681 | AP1-MAPK | MMP9 | MMP9_3 | Probe | TGGTTACACTCGGGTGGCAGAGA |
| 682 | AP1-MAPK | MMP9 | MMP9_4 | Forward | TCCACCCTTGTGCTCTT |
| | | | | Primer | |
| 683 | AP1-MAPK | MMP9 | MMP9_4 | Reverse | ACTCTCCACGCATCTCTG |
| | | | | Primer | |
| 684 | AP1-MAPK | MMP9 | MMP9_4 | Probe | AACCAATCTCACCGACAGGCAGC |
| 364 | AP1-MAPK | PLAU | PLAU_1 | Forward | TCGAACTGTGACTGTCTAAATG |
| | | | | Primer | |
| 365 | AP1-MAPK | PLAU | PLAU_1 | Reverse | CTGCCCTCCGAATTTCTTT |
| | | | | Primer | |
| 366 | AP1-MAPK | PLAU | PLAU_1 | Probe | AACATTCACTGGTGCAACTGCCC |
| 367 | AP1-MAPK | PLAU | PLAU_2 | Forward | GTTCCATCGAACTGTGACT |
| | | | | Primer | |
| 368 | AP1-MAPK | PLAU | PLAU_2 | Reverse | CGAATTTCTTTGGGCAGTTG |
| | | | | Primer | |
| 369 | AP1-MAPK | PLAU | PLAU_2 | Probe | TGGAGGAACATGTGTGTCCAACAAGT |
| 370 | AP1-MAPK | PLAU | PLAU_3 | Forward | GTGCAACTGCCCAAAGAAAT |
| | | | | Primer | |
| 371 | AP1-MAPK | PLAU | PLAU_3 | Reverse | GACAGTGGCAGAGTTCCAG |
| | | | | Primer | |
| 372 | AP1-MAPK | PLAU | PLAU_3 | Probe | AGGAAAGGCCAGCACTGACACC |
| 373 | AP1-MAPK | PLAU | PLAU_4 | Forward | ACTGCCCAAAGAAATTCGG |
| | | | | primer | |
| 374 | AP1-MAPK | PLAU | PLAU_4 | Reverse | CTGGCCTTTCCTCGGTAAA |
| | | | | primer | |
| 375 | AP1-MAPK | PLAU | PLAU_4 | Probe | CAGCACTGTGAAATAGATAAGTCAAAAACCT |
| 685 | AP1-MAPK | PLAUR | PLAUR_1 | Forward | TGTGTGGGTTAGACTTGTG |
| | | | | primer | |
| 686 | AP1-MAPK | PLAUR | PLAUR_1 | Reverse | GTAACGGCTTCGGGAATAG |
| | | | | primer | |
| 687 | AP1-MAPK | PLAUR | PLAUR_1 | Probe | AACCAGGGCAACTCTGGCCG |
| 688 | AP1-MAPK | PLAUR | PLAUR_2 | Forward | TGTGTGGGTTAGACTTGTG |
| | | | | Primer | |
| 689 | AP1-MAPK | PLAUR | PLAUR_2 | Reverse | CACAGCTCATGTCTGATGA |
| | | | | Primer | |
| 690 | AP1-MAPK | PLAUR | PLAUR_2 | Probe | CCGAAGCCGTTACCTCGAATGCA |
| 691 | AP1-MAPK | PLAUR | PLAUR_3 | Forward | CTCAGAGAAGACCAACAGG |
| | | | | Primer | |
| 692 | AP1-MAPK | PLAUR | PLAUR_3 | Reverse | CTTCGGGAATAGGTGACAG |
| | | | | Primer | |
| 693 | AP1-MAPK | PLAUR | PLAUR_3 | Probe | ACTTGTGCAACCAGGGCAACTCT |
| 694 | AP1-MAPK | PLAUR | PLAUR_4 | Forward | GAGGTTGTGTGTGGGTTAG |
| | | | | Primer | |
| 695 | AP1-MAPK | PLAUR | PLAUR_4 | Reverse | TGATGAGCCACAGGAAATG |
| | | | | Primer | |
| 696 | AP1-MAPK | PLAUR | PLAUR_4 | Probe | ACTTGTGCAACCAGGGCAACTCT |
| 697 | AP1-MAPK | PTGS2 | PTGS2_1 | Forward | TTGACAGTCCACCAACTTAC |
| | | | | Primer | |
| 698 | AP1-MAPK | PTGS2 | PTGS2_1 | Reverse | GGAGGAAGGGCTCTAGTATAA |
| | | | | Primer | |
| 699 | AP1-MAPK | PTGS2 | PTGS2_1 | Probe | AAGCTGGGAAGCCTTCTCTAACCTCT |
| 700 | AP1-MAPK | PTGS2 | PTGS2_2 | Forward | GTGAATAACATTCCCTTCCTTC |
| | | | | Primer | |
| 701 | AP1-MAPK | PTGS2 | PTGS2_2 | Reverse | TAGCCATAGTCAGCATTGTAA |
| | | | | Primer | |
| 702 | AP1-MAPK | PTGS2 | PTGS2_2 | Probe | CCAGATCACATTTGATTGACAGTCCACCA |
| 703 | AP1-MAPK | PTGS2 | PTGS2_3 | Forward | TGTGTTGACATCCAGATCAC |
| | | | | primer | |
| 704 | AP1-MAPK | PTGS2 | PTGS2_3 | Reverse | TAGGAGAGGTTAGAGAAGGC |
| | | | | primer | |
| 705 | AP1-MAPK | PTGS2 | PTGS2_3 | Probe | CCACCAACTTACAATGCTGACTATGGCT |
| 706 | AP1-MAPK | PTGS2 | PTGS2_4 | Forward | CCAACTTACAATGCTGACTATG |
| | | | | Primer | |
| 707 | AP1-MAPK | PTGS2 | PTGS2_4 | Reverse | CAATCATCAGGCACAGGAG |
| | | | | Primer | |
| 708 | AP1-MAPK | PTGS2 | PTGS2_4 | Probe | AAGCTGGGAAGCCTTCTCTAACCTCT |
| 709 | AP1-MAPK | SERPINE1 | SERPINE1_1 | Forward | TGTCTCTGTGCCCATGAT |
| | | | | Primer | |
| 710 | AP1-MAPK | SERPINE1 | SERPINE1_1 | Reverse | CAGTTCCAGGATGTCGTAGT |
| | | | | Primer | |
| 711 | AP1-MAPK | SERPINE1 | SERPINE1_1 | Probe | ACTGAGTTCACCACGCCCGATG |
| 712 | AP1-MAPK | SERPINE1 | SERPINE1_2 | Forward | CGCCTCTTCCACAAATCAG |
| | | | | Primer | |
| 713 | AP1-MAPK | SERPINE1 | SERPINE1_2 | Reverse | TCCAGGATGTCGTAGTAATGG |
| | | | | Primer | |
| 714 | AP1-MAPK | SERPINE1 | SERPINE1_2 | Probe | ATGGGCACAGAGACAGTGCTGC |
| 715 | AP1-MAPK | SERPINE1 | SERPINE1_3 | Forward | TGGCTCAGACCAACAAGT |
| | | | | Primer | |
| 716 | AP1-MAPK | SERPINE1 | SERPINE1_3 | Reverse | CAGCAATGAACATGCTGAGG |
| | | | | Primer | |
| 717 | AP1-MAPK | SERPINE1 | SERPINE1_3 | Probe | ACTACGACATCCTGGAACTGCCCT |
| 718 | AP1-MAPK | SERPINE1 | SERPINE1_4 | Forward | CCACAAATCAGACGGCAGCA |
| | | | | primer | |
| 719 | AP1-MAPK | SERPINE1 | SERPINE1_4 | Reverse | GTCGTAGTAATGGCCATCGG |
| | | | | primer | |
| 720 | AP1-MAPK | SERPINE1 | SERPINE1_4 | Probe | CCCATGATGGCTCAGACCAACAAGT |
| 721 | AP1-MAPK | TIMP1 | TIMP1_1 | Forward | CCAGAGAGACACCAGAGAA |
| | | | | Primer | |
| 722 | AP1-MAPK | TIMP1 | TIMP1_1 | Reverse | GAGGTCGGAATTGCAGAAG |
| | | | | Primer | |
| 723 | AP1-MAPK | TIMP1 | TIMP1_1 | Probe | TCTGGCATCCTGTTGTTGCTGTGG |
| 724 | AP1-MAPK | TIMP1 | TIMP1_2 | Forward | CCCAGAGAGACACCAGAGAA |
| | | | | primer | |
| 725 | AP1-MAPK | TIMP1 | TIMP1_2 | Reverse | GCTATCAGCCACAGCAACA |
| | | | | primer | |
| 726 | AP1-MAPK | TIMP1 | TIMP1_2 | Probe | CCTGGCTTCTGGCATCCTGT |
| 727 | AP1-MAPK | TIMP1 | TIMP1_3 | Forward | AGAGACACCAGAGAACCCA |
| | | | | Primer | |
| 728 | AP1-MAPK | TIMP1 | TIMP1_3 | Reverse | GTGGGACACAGGTGCAG |
| | | | | Primer | |
| 729 | AP1-MAPK | TIMP1 | TIMP1_3 | Probe | TCTGGCATCCTGTTGTTGCTGTGG |
| 730 | AP1-MAPK | TIMP1 | TIMP1_4 | Forward | CATCGCCGCAGATCCAG |
| | | | | Primer | |
| 731 | AP1-MAPK | TIMP1 | TIMP1_4 | Reverse | TCAGCCACAGCAACAACA |
| | | | | Primer | |
| 732 | AP1-MAPK | TIMP1 | TIMP1_4 | Probe | CCCAGAGAGACACCAGAGAACCCA |
| 733 | AP1-MAPK | TP53 | TP53_1 | Forward | AAACTCATGTTCAAGACAGAAG |
| | | | | Primer | |
| 734 | AP1-MAPK | TP53 | TP53_1 | Reverse | CAAGCAAGGGTTCAAAGAC |
| | | | | Primer | |
| 735 | AP1-MAPK | TP53 | TP53_1 | Probe | TGGAGAATGTCAGTCTGAGTCAGGCC |
| 736 | AP1-MAPK | TP53 | TP53_2 | Forward | GTCTACCTCCCGCCATAAA |
| | | | | Primer | |
| 737 | AP1-MAPK | TP53 | TP53_2 | Reverse | GGGAACAAGAAGTGGAGAATG |
| | | | | Primer | |
| 738 | AP1-MAPK | TP53 | TP53_2 | Probe | TGTTCAAGACAGAAGGGCCTGACTCA |
| 739 | AP1-MAPK | TP53 | TP53_3 | Forward | CAAGACAGAAGGGCCTGAC |
| | | | | Primer | |
| 740 | AP1-MAPK | TP53 | TP53_3 | Reverse | CACACCTATTGCAAGCAAGG |
| | | | | Primer | |
| 741 | AP1-MAPK | TP53 | TP53_3 | Probe | TCAGACTGACATTCTCCACTTCTTGTTCCC |
| 742 | AP1-MAPK | TP53 | TP53_4 | Forward | CAGCCACCTGAAGTCCAAA |
| | | | | Primer | |
| 743 | AP1-MAPK | TP53 | TP53_4 | Reverse | GTGGAGAATGTCAGTCTGAGTC |
| | | | | Primer | |
| 744 | AP1-MAPK | TP53 | TP53_4 | Probe | TTATGGCGGGAGGTAGACTGACCC |
| 745 | AP1-MAPK | VEGFD | VEGFD_1 | Forward | GCCAGAAGCACAAGCTATTTC |
| | | | | primer | |
| 746 | AP1-MAPK | VEGFD | VEGFD_1 | Reverse | TGCTTTGCACATGCTGTTT |
| | | | | primer | |
| 747 | AP1-MAPK | VEGFD | VEGFD_1 | Probe | ACCAGACCATGTGCAAGTGGCA |
| 748 | AP1-MAPK | VEGFD | VEGFD_2 | Forward | CCTTTCATACCAGACCATGT |
| | | | | Primer | |
| 749 | AP1-MAPK | VEGFD | VEGFD_2 | Reverse | CGCTGAATCAAGGATTCTTTC |
| | | | | Primer | |
| 750 | AP1-MAPK | VEGFD | VEGFD_2 | Probe | TGCAAAGCATTGCCGCTTTCCA |
| 751 | AP1-MAPK | VEGFD | VEGFD_3 | Forward | CTATTTCACCCAGACACCTG |
| | | | | Primer | |
| 752 | AP1-MAPK | VEGFD | VEGFD_3 | Reverse | AAGGATTCTTTCGGCTGTG |
| | | | | Primer | |
| 753 | AP1-MAPK | VEGFD | VEGFD_3 | Probe | ACCAGACCATGTGCAAGTGGCA |
| 754 | AP1-MAPK | VEGFD | VEGFD_4 | Forward | TTTGAGTGCAAAGAAAGTCTG |
| | | | | Primer | |
| 755 | AP1-MAPK | VEGFD | VEGFD_4 | Reverse | ACATGGTCTGGTATGAAAGG |
| | | | | Primer | |
| 756 | AP1-MAPK | VEGFD | VEGFD_4 | Probe | AGACCTGCTGCCAGAAGCACAA |
| 757 | AP1-MAPK | VIM | VIM_1 | Forward | GACAGGATGTTGACAATGC |
| | | | | Primer | |
| 758 | AP1-MAPK | VIM | VIM_1 | Reverse | CCTCTTCGTGGAGTTTCTT |
| | | | | Primer | |
| 759 | AP1-MAPK | VIM | VIM_1 | Probe | TGCGTTCAAGGTCAAGACGTGCC |
| 760 | AP1-MAPK | VIM | VIM_2 | Forward | CAATCTTTCAGACAGGATGTTG |
| | | | | Primer | |
| 761 | AP1-MAPK | VIM | VIM_2 | Reverse | GCTCCTGGATTTCCTCTTC |
| | | | | Primer | |
| 762 | AP1-MAPK | VIM | VIM_2 | Probe | ACAATGCGTCTCTGGCACGTCTT |
| 763 | AP1-MAPK | VIM | VIM_3 | Forward | CCTCCGGGAGAAATTGCAG |
| | | | | Primer | |
| 764 | AP1-MAPK | VIM | VIM_3 | Reverse | GTCAAGACGTGCCAGAGAC |
| | | | | Primer | |
| 765 | AP1-MAPK | VIM | VIM 3 | Probe | AGATGCTTCAGAGAGAGGAAGCCGAA |
| 766 | AP1-MAPK | VIM | VIM_4 | Forward | GGAGGAGATGCTTCAGAGA |
| | | | | primer | |
| 767 | AP1-MAPK | VIM | VIM_4 | Reverse | ATTCCACTTTGCGTTCAAG |
| | | | | primer | |
| 768 | AP1-MAPK | VIM | VIM_4 | Probe | TCTTTCAGACAGGATGTTGACAATGCG |

TABLE 5
Sets of primers and probes for determining the Notch cellular signaling
pathway activity.

| SEQ | | | | | |
|---|---|---|---|---|---|
| ID | | | | | |
| NO | Pathway | Gene | Assay | Oligo | Sequence |
| 769 | Notch | CD44 | CD44_1 | Forward | TGTAACACCTACACCATTATCT |
| | | | | Primer | |
| 770 | Notch | CD44 | CD44_1 | Reverse | TCGCAATGAAACAATCAGTAG |
| | | | | Primer | |
| 771 | Notch | CD44 | CD44_1 | Probe | ACCATTACAGGGAGCTGGGACACT |
| 772 | Notch | CD44 | CD44_2 | Forward | ACTCCAGACCAGTTTATGAC |
| | | | | Primer | |
| 773 | Notch | CD44 | CD44_2 | Reverse | CCAACGGTTGTTTCTTTCC |
| | | | | Primer | |
| 774 | Notch | CD44 | CD44_2 | Probe | AGCTGATGAGACAAGGAACCTGCA |
| 775 | Notch | CD44 | CD44_3 | Forward | GCATTTGGTGAACAAGGAG |
| | | | | Primer | |
| 776 | Notch | CD44 | CD44_3 | Reverse | CCAATCTTCATGTCCACATTC |
| | | | | Primer | |
| 777 | Notch | CD44 | CD44_3 | Probe | AGCTGATGAGACAAGGAACCTGCA |
| 778 | Notch | CD44 | CD44_4 | Forward | TGACAGCTGATGAGACAAG |
| | | | | Primer | |
| 779 | Notch | CD44 | CD44_4 | Reverse | AGCTCCCTGTAATGGTTATG |
| | | | | Primer | |
| 780 | Notch | CD44 | CD44_4 | Probe | CCTGCAGAATGTGGACATGAAGATTGGG |
| 781 | Notch | EPHB3 | EPHB3_1 | Forward | TCACTGAGTTCATGGAAAACTG |
| | | | | primer | |
| 782 | Notch | EPHB3 | EPHB3_1 | Reverse | GTTCATCTCGGACAGGTACTT |
| | | | | primer | |
| 783 | Notch | EPHB3 | EPHB3_1 | Probe | CCTTCCTCCGGCTCAACGATGGG |
| 784 | Notch | FABP7 | FABP7_1 | Forward | CTGTTCGCCACTATGAGAA |
| | | | | Primer | |
| 785 | Notch | FABP7 | FABP7_1 | Reverse | GGATAGCACTGAGACTTGAG |
| | | | | Primer | |
| 786 | Notch | FABP7 | FABP7_2 | Probe | AAACTGAAGAGCTCTTCCAAGCCC |
| 787 | Notch | FABP7 | FABP7_2 | Forward | TGTAAGAGAAATTAAGGATGGC |
| | | | | Primer | |
| 788 | Notch | FABP7 | FABP7_2 | Reverse | CTGAGACTTGAGGAAACAGA |
| | | | | Primer | |
| 789 | Notch | FABP7 | FABP7_2 | Probe | TGATGTGGTTGCTGTTCGCCACT |
| 790 | Notch | FABP7 | FABP7_3 | Forward | TTTCTGTTTCCTCAAGTCTCA |
| | | | | Primer | |
| 791 | Notch | FABP7 | FABP7_3 | Reverse | ACACCAAGGATAACCTTCTAAT |
| | | | | Primer | |
| 792 | Notch | FABP7 | FABP7_3 | Probe | TGATCAGCCATGTTGTAATAGGATAGCAC |
| 793 | Notch | FABP7 | FABP7_4 | Forward | GCCACTATGAGAAGGCATAA |
| | | | | Primer | |
| 794 | Notch | FABP7 | FABP7_4 | Reverse | CCTCCACACCAAGGATAAC |
| | | | | Primer | |
| 795 | Notch | FABP7 | FABP7_4 | Probe | TCTGTTTCCTCAAGTCTCAGTGCTATCCT |
| 796 | Notch | HES1 | HES1_1 | Forward | GTCTACCTCTCTCCTTGGT |
| | | | | primer | |
| 797 | Notch | HES1 | HES1_1 | Reverse | CAAGTGCTGAGGGTTTATTAT |
| | | | | primer | |
| 798 | Notch | HES1 | HES1_1 | Probe | TGGAACAGCGCTACTGATCACCAA |
| 799 | Notch | HES4 | HES4_1 | Forward | AGAGCTCCCGCCACTC |
| | | | | primer | |
| 800 | Notch | HES4 | HES4_1 | Reverse | AGGTGTCTCACGGTCATCTC |
| | | | | primer | |
| 801 | Notch | HES4 | HES4_1 | Probe | AGGATGTCCGCCTTCTCCAGCTTC |
| 802 | Notch | HES5 | HES5_1 | Forward | TGGGTGCCTCCACTATGAT |
| | | | | Primer | |
| 803 | Notch | HES5 | HES5_1 | Reverse | CTTCCACGTGACTGAGAGTT |
| | | | | Primer | |
| 804 | Notch | HES5 | HES5_1 | Probe | TCTGTGTGGGTGGATGCGTGTG |
| 805 | Notch | HES5 | HES5_2 | Forward | GCCTCCACTATGATCCTTAAA |
| | | | | primer | |
| 806 | Notch | HES5 | HES5_2 | Reverse | CGTGACTGAGAGTTCAATTTC |
| | | | | primer | |
| 807 | Notch | HES5 | HES5_2 | Probe | ATGCGTGTGGGCACGACTTTGTAC |
| 808 | Notch | HEY1 | HEY1_1 | Forward | TTTGAGAAGCAGGGATCTG |
| | | | | Primer | |
| 809 | Notch | HEY1 | HEY1_1 | Reverse | CCAAACTCCGATAGTCCATAG |
| | | | | Primer | |
| 810 | Notch | HEY1 | HEY1_1 | Probe | TTACTTTGACGCGCACGCCCTT |
| 811 | Notch | HEY1 | HEY1_2 | Forward | GACCGTGGATCACCTGAAA |
| | | | | primer | |
| 812 | Notch | HEY1 | HEY1_2 | Reverse | CCAAACTCCGATAGTCCATAG |
| | | | | primer | |
| 813 | Notch | HEY1 | HEY1_2 | Probe | TTACTTTGACGCGCACGCCCTT |
| 814 | Notch | HEY1 | HEY1_3 | Forward | GCCCTTGCTATGGACTATC |
| | | | | Primer | |
| 815 | Notch | HEY1 | HEY1_3 | Reverse | TCTAGTCCTTCAATGATGCTC |
| | | | | Primer | |
| 816 | Notch | HEY1 | HEY1_3 | Probe | CCTGGCAGAAGTTGCGCGTTATCT |
| 817 | Notch | HEY1 | HEY1_4 | Forward | CAGGAGGGAAAGGTTACTT |
| | | | | Primer | |
| 818 | Notch | HEY1 | HEY1_4 | Reverse | CCTTCAATGATGCTCAGATAAC |
| | | | | Primer | |
| 819 | Notch | HEY1 | HEY1_4 | Probe | ACGCCCTTGCTATGGACTATCGGA |
| 820 | Notch | HEY2 | HEY2_1 | Forward | ATGACAGTGGATCATTTGAAGA |
| | | | | primer | |
| 821 | Notch | HEY2 | HEY2_1 | Reverse | CGGAATCCTATGCTCATGAA |
| | | | | primer | |
| 822 | Notch | HEY2 | HEY2_1 | Probe | ACTTTGACGCACACGCTCTTGCCA |
| 823 | Notch | HEY2 | HEY2_2 | Forward | AAGATGCTTCAGGCAACAG |
| | | | | Primer | |
| 824 | Notch | HEY2 | HEY2_2 | Reverse | CAACTTCTGTTAGGCACTCTC |
| | | | | Primer | |
| 825 | Notch | HEY2 | HEY2_2 | Probe | AAGGCTACTTTGACGCACACGCT |
| 826 | Notch | HEY2 | HEY2_3 | Forward | GACTTGTGCCAACTGCTTT |
| | | | | Primer | |
| 827 | Notch | HEY2 | HEY2_3 | Reverse | GCGTCAAAGTAGCCTTTACC |
| | | | | Primer | |
| 828 | Notch | HEY2 | HEY2_3 | Probe | TCATTTGAAGATGCTTCAGGCAACAGGG |
| 829 | Notch | HEY2 | HEY2_4 | Forward | ACAAGGATCTGCAAAGTTAGA |
| | | | | Primer | |
| 830 | Notch | HEY2 | HEY2_4 | Reverse | ATGAAGTCCATGGCAAGAG |
| | | | | Primer | |
| 831 | Notch | HEY2 | HEY2_4 | Probe | CGTGTGCGTCAAAGTAGCCTTTACCC |
| 832 | Notch | MYC | MYC_1 | Forward | TGCTTAGACGCTGGATTT |
| | | | | Primer | |
| 833 | Notch | MYC | MYC_1 | Reverse | TCGTAGTCGAGGTCATAGT |
| | | | | Primer | |
| 834 | Notch | MYC | MYC_1 | Probe | CCCTCAACGTTAGCTTCACCAACAGG |
| 835 | Notch | MYC | MYC_2 | Forward | TCTCTGAAAGGCTCTCCT |
| | | | | Primer | |
| 836 | Notch | MYC | MYC_2 | Reverse | TCCTGTTGGTGAAGCTAAC |
| | | | | Primer | |
| 837 | Notch | MYC | MYC_2 | Probe | TGCAGCTGCTTAGACGCTGGATTT |
| 838 | Notch | MYC | MYC_3 | Forward | GACCCGCTTCTCTGAAA |
| | | | | Primer | |
| 839 | Notch | MYC | MYC_3 | Reverse | AGGTCATAGTTCCTGTTGG |
| | | | | Primer | |
| 840 | Notch | MYC | MYC_3 | Probe | TGCAGCTGCTTAGACGCTGGATTT |
| 841 | Notch | MYC | MYC_4 | Forward | TTCGGGTAGTGGAAAACCA |
| | | | | primer | |
| 842 | Notch | MYC | MYC_4 | Reverse | CATAGTTCCTGTTGGTGAAGC |
| | | | | primer | |
| 843 | Notch | MYC | MYC_4 | Probe | CTCCCGCGACGATGCCCCTCAA |
| 844 | Notch | NOX1 | NOX1_1 | Forward | CTGTTCGCCACTATGAGAA |
| | | | | Primer | |
| 845 | Notch | NOX1 | NOX1_1 | Reverse | GGATAGCACTGAGACTTGAG |
| | | | | Primer | |
| 846 | Notch | NOX1 | NOX1_1 | Probe | AAACTGAAGAGCTCTTCCAAGCCC |
| 847 | Notch | NOX1 | NOX1_2 | Forward | TGTAAGAGAAATTAAGGATGGC |
| | | | | Primer | |
| 848 | Notch | NOX1 | NOX1_2 | Reverse | CTGAGACTTGAGGAAACAGA |
| | | | | Primer | |
| 849 | Notch | NOX1 | NOX1_2 | Probe | TGATGTGGTTGCTGTTCGCCACT |
| 850 | Notch | NOX1 | NOX1_3 | Forward | TTTCTGTTTCCTCAAGTCTCA |
| | | | | Primer | |
| 851 | Notch | NOX1 | NOX1_3 | Reverse | ACACCAAGGATAACCTTCTAAT |
| | | | | Primer | |
| 852 | Notch | NOX1 | NOX1_3 | Probe | TGATCAGCCATGTTGTAATAGGATAGCAC |
| 853 | Notch | NOX1 | NOX1_4 | Forward | GCCACTATGAGAAGGCATAA |
| | | | | Primer | |
| 854 | Notch | NOX1 | NOX1_4 | Reverse | CCTCCACACCAAGGATAAC |
| | | | | Primer | |
| 855 | Notch | NOX1 | NOX1_4 | Probe | TCTGTTTCCTCAAGTCTCAGTGCTATCCT |
| 856 | Notch | NRARP | NRARP_1 | Forward | GTTGCTGGTGTTCTAAACTATT |
| | | | | Primer | |
| 857 | Notch | NRARP | NRARP_1 | Reverse | CCCATAACCACATTGACCA |
| | | | | Primer | |
| 858 | Notch | NRARP | NRARP_1 | Probe | TTTGTGGGTGGAGTTTGTGCGC |
| 859 | Notch | NRARP | NRARP_2 | Forward | TGTGTGTACATTTGTGGGT |
| | | | | Primer | |
| 860 | Notch | NRARP | NRARP_2 | Reverse | CAAGAAATGGTAGACTCAAGTT |
| | | | | Primer | |
| 861 | Notch | NRARP | NRARP_2 | Probe | ACTGCGTGGTCAATGTGGTTATGGG |
| 862 | Notch | NRARP | NRARP_3 | Forward | CATTTGTGGGTGGAGTTTG |
| | | | | primer | |
| 863 | Notch | NRARP | NRARP_3 | Reverse | GCAACCAAGAAATGGTAGAC |
| | | | | primer | |
| 864 | Notch | NRARP | NRARP_3 | Probe | ACTGCGTGGTCAATGTGGTTATGGG |
| 865 | Notch | NRARP | NRARP_4 | Forward | CGCTGTTGCTGGTGTTCTA |
| | | | | Primer | |
| 866 | Notch | NRARP | NRARP_4 | Reverse | CATTGACCACGCAGTGTTT |
| | | | | Primer | |
| 867 | Notch | NRARP | NRARP_4 | Probe | TTTGTGGGTGGAGTTTGTGCGC |
| 868 | Notch | PIN1 | PIN1_1 | Forward | ACAGTTCAGCGACTGCAG |
| | | | | primer | |
| 869 | Notch | PIN1 | PIN1_1 | Reverse | AACGAGGCGTCTTCAAATG |
| | | | | primer | |
| 870 | Notch | PIN1 | PIN1_1 | Probe | TCAGCAGAGGTCAGATGCAGAAGC |
| 871 | Notch | PIN1 | PIN1_2 | Forward | GCTGATCAACGGCTACATC |
| | | | | Primer | |
| 872 | Notch | PIN1 | PIN1_2 | Reverse | GCGTCTTCAAATGGCTTCT |
| | | | | Primer | |
| 873 | Notch | PIN1 | PIN1_2 | Probe | TGCAGTCGCTGAACTGTGAGGC |
| 874 | Notch | PIN1 | PIN1_3 | Forward | GCAGAGGTCAGATGCAGAAG |
| | | | | Primer | |
| 875 | Notch | PIN1 | PIN1_3 | Reverse | GCGGAGGATGATGTGGATG |
| | | | | Primer | |
| 876 | Notch | PIN1 | PIN1_3 | Probe | TTGAAGACGCCTCGTTTGCGCT |
| 877 | Notch | PIN1 | PIN1_4 | Forward | AGAGGAGGACTTTGAGTCT |
| | | | | Primer | |
| 878 | Notch | PIN1 | PIN1_4 | Reverse | CAAATGGCTTCTGCATCTG |
| | | | | Primer | |
| 879 | Notch | PIN1 | PIN1_4 | Probe | TGCAGTCGCTGAACTGTGAGGC |
| 880 | Notch | PLXND1 | PLXND1_1 | Forward | CCTGTTCGTCTTCTGTACC |
| | | | | Primer | |
| 881 | Notch | PLXND1 | PLXND1_1 | Reverse | GATTCCATCTCCTCCATCTG |
| | | | | Primer | |
| 882 | Notch | PLXND1 | PLXND1_1 | Probe | ACGTGCTGAGCGTTACTGGCAG |
| 883 | Notch | PLXND1 | PLXND1_2 | Forward | CATCGTGTCCATCGTCATC |
| | | | | primer | |
| 884 | Notch | PLXND1 | PLXND1_2 | Reverse | AGTAACGCTCAGCACGTC |
| | | | | primer | |
| 885 | Notch | PLXND1 | PLXND1_2 | Probe | TGGTGGCCCTGTTCGTCTTCTGTA |
| 886 | Notch | PLXND1 | PLXND1_3 | Forward | CATCGTGTCCATCGTCATC |
| | | | | Primer | |
| 887 | Notch | PLXND1 | PLXND1_3 | Reverse | CTCGGATCTGAGATTCCATC |
| | | | | Primer | |
| 888 | Notch | PLXND1 | PLXND1_3 | Probe | ACGTGCTGAGCGTTACTGGCAG |
| 889 | Notch | PLXND1 | PLXND1_4 | Forward | CACAATCCAGGTAGGGAAC |
| | | | | Primer | |
| 890 | Notch | PLXND1 | PLXND1_4 | Reverse | CTTGGTACAGAAGACGAACA |
| | | | | Primer | |
| 891 | Notch | PLXND1 | PLXND1_4 | Probe | AACCAGACCATCGCCACACTGC |
| 892 | Notch | SOX9 | SOX9_1 | Forward | CCTGTTCGTCTTCTGTACC |
| | | | | Primer | |
| 893 | Notch | SOX9 | SOX9_1 | Reverse | GATTCCATCTCCTCCATCTG |
| | | | | Primer | |
| 894 | Notch | SOX9 | SOX9_1 | Probe | ACGTGCTGAGCGTTACTGGCAG |
| 895 | Notch | SOX9 | SOX9_2 | Forward | CACAATCCAGGTAGGGAAC |
| | | | | Primer | |
| 896 | Notch | SOX9 | SOX9_2 | Reverse | CTTGGTACAGAAGACGAACA |
| | | | | Primer | |
| 897 | Notch | SOX9 | SOX9_2 | Probe | AACCAGACCATCGCCACACTGC |
| 898 | Notch | SOX9 | SOX9_3 | Forward | GACCAGTACCCGCACTT |
| | | | | primer | |
| 899 | Notch | SOX9 | SOX9_3 | Reverse | CGCTTCTCGCTCTCGTT |
| | | | | primer | |
| 900 | Notch | SOX9 | SOX9_3 | Probe | CGCTGGGCAAGCTCTGGAGACT |
| 901 | Notch | SOX9 | SOX9_4 | Forward | CATCGTGTCCATCGTCATC |
| | | | | Primer | |
| 902 | Notch | SOX9 | SOX9_4 | Reverse | CTCGGATCTGAGATTCCATC |
| | | | | Primer | |
| 903 | Notch | SOX9 | SOX9_4 | Probe | ACGTGCTGAGCGTTACTGGCAG |

TABLE 6
Sets of primers and probes for determining the HH cellular signaling
pathway activity.

| SEQ | | | | | |
|---|---|---|---|---|---|
| ID | | | | | |
| NO | Pathway | Gene | Assay | Oligo | Sequence |
| 904 | HH | CFLAR | CFLAR_1 | Forward primer | GGTGAGGATTTGGATAAATCTGATG |
| 905 | HH | CFLAR | CFLAR_1 | Probe | ACATGGGCCGAGGCAAGATAAGCAA |
| 906 | HH | CFLAR | CFLAR_1 | Reverse primer | TCAACCACAAGGTCCAAGAAAC |
| 907 | HH | CFLAR | CFLAR_2 | Forward Primer | GCAGAGATTGGTGAGGATT |
| 908 | HH | CFLAR | CFLAR_2 | Probe | TGGGCCGAGGCAAGATAAGCAA |
| 909 | HH | CFLAR | CFLAR_2 | Reverse Primer | TCCAACTCAACCACAAGG |
| 910 | HH | CFLAR | CFLAR_3 | Forward Primer | GAGGCAAGATAAGCAAGGA |
| 911 | HH | CFLAR | CFLAR_3 | Probe | TCTTGGACCTTGTGGTTGAGTTGGAGA |
| 912 | HH | CFLAR | CFLAR_3 | Reverse Primer | GTGGATGTTCTTTAGGCATTT |
| 913 | HH | CFLAR | CFLAR_4 | Forward Primer | ATGGCAGAGATTGGTGAG |
| 914 | HH | CFLAR | CFLAR_4 | Probe | TGGGCCGAGGCAAGATAAGCAA |
| 915 | HH | CFLAR | CFLAR_4 | Reverse Primer | ACCACAAGGTCCAAGAAA |
| 916 | HH | FOXM1 | FOXM1_1 | Forward Primer | GGACCACTTTCCCTACTTTA |
| 917 | HH | FOXM1 | FOXM1_1 | Probe | ATCCGCCACAACCTTTCCCTGC |
| 918 | HH | FOXM1 | FOXM1_1 | Reverse Primer | GTCTCCCGGACAAACAT |
| 919 | HH | FOXM1 | FOXM1_2 | Forward Primer | TGGATTGAGGACCACTTTC |
| 920 | HH | FOXM1 | FOXM1_2 | Probe | ATCCGCCACAACCTTTCCCTGC |
| 921 | HH | FOXM1 | FOXM1_2 | Reverse Primer | GAAGGAGACCTTGCCATT |
| 922 | HH | FOXM1 | FOXM1_3 | Forward Primer | CAACAGCACTGAGAGGAA |
| 923 | HH | FOXM1 | FOXM1_3 | Probe | ACATTGCCAAGCCAGGCTGGAA |
| 924 | HH | FOXM1 | FOXM1_3 | Reverse Primer | ATGTCGTGCAGGGAAAG |
| 925 | HH | FYN | FYN_1 | Forward Primer | GTGTGAACTCTTCGTCTCA |
| 926 | HH | FYN | FYN_1 | Probe | ACGAGAGGAGGAACAGGAGTGACA |
| 927 | HH | FYN | FYN_1 | Reverse Primer | CTGTCCGTGCTTCATAGT |
| 928 | HH | FYN | FYN_2 | Forward Primer | AAGGACTCACCGTCTTTG |
| 929 | HH | FYN | FYN_2 | Probe | ACGAGAGGAGGAACAGGAGTGACA |
| 930 | HH | FYN | FYN_2 | Reverse Primer | GTCCGTGCTTCATAGTCATA |
| 931 | HH | FYN | FYN_3 | Forward Primer | CTTTGGAGGTGTGAACTCT |
| 932 | HH | FYN | FYN_3 | Probe | ACGAGAGGAGGAACAGGAGTGACA |
| 933 | HH | FYN | FYN_3 | Reverse Primer | ATCTTCTGTCCGTGCTTC |
| 934 | HH | FYN | FYN_4 | Forward primer | CGTCTTTGGAGGTGTGAACT |
| 935 | HH | FYN | FYN_4 | Probe | CGAGAGGAGGAACAGGAGTGACACTCTTTGT |
| 936 | HH | FYN | FYN_4 | Reverse primer | TCCGTGCTTCATAGTCATAAAGG |
| 937 | HH | GLI1 | GLI1_1 | Forward Primer | CCTTCAAAGCCCAGTACA |
| 938 | HH | GLI1 | GLI1_1 | Probe | ACGTTTGAAGGGTGCCGGAAGT |
| 939 | HH | GLI1 | GLI1_1 | Reverse Primer | TTTCGAGGCGTGAGTATG |
| 940 | HH | GLI1 | GLI1_2 | Forward primer | CAGTACATGCTGGTGGTTCAC |
| 941 | HH | GLI1 | GLI1_2 | Probe | ACTGGCGAGAAGCCACACAAGTGC |
| 942 | HH | GLI1 | GLI1_2 | Reverse primer | TTCGAGGCGTGAGTATGACTT |
| 943 | HH | GLI1 | GLI1_3 | Forward Primer | CAAAGCCCAGTACATGCT |
| 944 | HH | GLI1 | GLI1_3 | Probe | ACGTTTGAAGGGTGCCGGAAGT |
| 945 | HH | GLI1 | GLI1_4 | Forward Primer | GTACATGCTGGTGGTTCA |
| 946 | HH | GLI1 | GLI1_4 | Probe | CACACTGGCGAGAAGCCACACAA |
| 947 | HH | GLI1 | GLI1_4 | Reverse Primer | GAGGCGTGAGTATGACTTC |
| 948 | HH | GLI1 | GLI1_X3 | Reverse Primer | GGTGCGTCTTCAGGTTT |
| 949 | HH | HHIP | HHIP_1 | Forward Primer | GAGGACCAGCATCTAACTAC |
| 950 | HH | HHIP | HHIP_1 | Probe | TCAGCAGAAAGCACAAACACAACTGC |
| 951 | HH | HHIP | HHIP_1 | Reverse Primer | ACTCACAACCTCCTGAATAC |
| 952 | HH | HHIP | HHIP_2 | Forward Primer | CAGCAGAAAGCACAAACACAAC |
| 953 | HH | HHIP | HHIP_2 | Probe | ATTCAGGAGGTTGTGAGTGGGCT |
| 954 | HH | HHIP | HHIP_2 | Reverse primer | CACTATGCAGGGCACCAAC |
| 955 | HH | HHIP | HHIP_3 | Forward | CTTGGACCAGATGGAAGAA |
| | | | | Primer | |
| 956 | HH | HHIP | HHIP_3 | Probe | TCAGCAGAAAGCACAAACACAACTGC |
| 957 | HH | HHIP | HHIP_3 | Reverse Primer | CCACTCACAACCTCCTG |
| 958 | HH | HHIP | HHIP_4 | Forward Primer | TCAGAGGACCAGCATCTA |
| 959 | HH | HHIP | HHIP_4 | Probe | TGGAAGAGATCAGCAGAAAGCACAAACAC |
| 960 | HH | HHIP | HHIP_4 | Reverse Primer | TCCTGAATACAGAAGCAGTT |
| 961 | HH | MYCN | MYCN_1 | Forward Primer | AAGGCCCTCAGTACCTC |
| 962 | HH | MYCN | MYCN_1 | Probe | TGAATCGCTCAGGGTGTCCTCTCC |
| 963 | HH | MYCN | MYCN_1 | Reverse Primer | GTGACCACGTCGATTTCTT |
| 964 | HH | MYCN | MYCN_2 | Forward Primer | CCACAAGGCCCTCAGTA |
| 965 | HH | MYCN | MYCN_2 | Probe | TGAATCGCTCAGGGTGTCCTCTCC |
| 966 | HH | MYCN | MYCN_2 | Reverse Primer | CACGTCGATTTCTTCCTCTTC |
| 967 | HH | MYCN | MYCN_3 | Forward primer | GACACCCTGAGCGATTC |
| 968 | HH | MYCN | MYCN_3 | Probe | TGAAGATGATGAAGAGGAAGATGAAGAGG |
| 969 | HH | MYCN | MYCN_3 | Reverse primer | GAATGTGGTGACAGCCTTG |
| 970 | HH | MYCN | MYCN_4 | Forward Primer | GACACCCTGAGCGATTC |
| 971 | HH | MYCN | MYCN_4 | Probe | AGAGGAAGATGAAGAGGAAGAAATCGACGT |
| 972 | HH | MYCN | MYCN_4 | Reverse Primer | GCTTCTCCACAGTGACC |
| 973 | HH | NKX2-2 | NKX2-2_1 | Forward Primer | CCCTTCTACGACAGCAG |
| 974 | HH | NKX2-2 | NKX2-2_1 | Probe | AGGGCCTTCAGTACTCCCTGCA |
| 975 | HH | NKX2-2 | NKX2-2_1 | Reverse Primer | GGGACTTGGAGCTTGAG |
| 976 | HH | NKX2-2 | NKX2-2_2 | Forward primer | GTGGCAGATTCCACCCA |
| 977 | HH | NKX2-2 | NKX2-2_2 | Probe | TCTGCCCATGCCTCTCCTTCTGAA |
| 978 | HH | NKX2-2 | NKX2-2_2 | Reverse primer | CGTAGAGTTCAGCCCTCTC |
| 979 | HH | NKX2-2 | NKX2-2_3 | Forward Primer | AGCGACAACCCGTACAC |
| 980 | HH | NKX2-2 | NKX2-2_3 | Probe | AGGGCCTTCAGTACTCCCTGCA |
| 981 | HH | NKX2-2 | NKX2-2_3 | Reverse Primer | CATTGTCCGGTGACTCGT |
| 982 | HH | NKX2-2 | NKX2-2_4 | Forward Primer | TACGACAGCAGCGACAA |
| 983 | HH | NKX2-2 | NKX2-2_4 | Probe | AGGGCCTTCAGTACTCCCTGCA |
| 984 | HH | NKX2-2 | NKX2-2_4 | Reverse Primer | TTGGAGCTTGAGTCCTGAG |
| 985 | HH | PTCH1 | PTCH1_1 | Forward primer | CTTCTTCATGGCCGCGTTAAT |
| 986 | HH | PTCH1 | PTCH1_1 | Probe | TCCAGGCAGCGGTAGTAGTGGTGT |
| 987 | HH | PTCH1 | PTCH1_1 | Reverse primer | AATGAGCAGAACCATGGCAAA |
| 988 | HH | PTCH1 | PTCH1_2 | Forward Primer | ACGTCCATCAGCAATGT |
| 989 | HH | PTCH1 | PTCH1_2 | Probe | CCGCGTTAATCCCAATTCCCGCT |
| 990 | HH | PTCH1 | PTCH1_2 | Reverse Primer | TTGAACACCACTACTACCG |
| 991 | HH | PTCH1 | PTCH1_3 | Forward Primer | TTCATGGCCGCGTTAAT |
| 992 | HH | PTCH1 | PTCH1_3 | Probe | TCCAGGCAGCGGTAGTAGTGGT |
| 993 | HH | PTCH1 | PTCH1_3 | Reverse Primer | AGCAGAACCATGGCAAA |
| 994 | HH | PTCH1 | PTCH1_4 | Forward Primer | CGCGTTAATCCCAATTCC |
| 995 | HH | PTCH1 | PTCH1_4 | Probe | TCCAGGCAGCGGTAGTAGTGGT |
| 996 | HH | PTCH1 | PTCH1_4 | Reverse Primer | GTCCTCGCGTCGATATAAA |
| 997 | HH | PTCH2 | PTCH2_1 | Forward Primer | CTGCTACAAGTCAGGAGTTC |
| 998 | HH | PTCH2 | PTCH2_1 | Probe | TGGAATGATTGAGCGGATGATTGAGAAGC |
| 999 | HH | PTCH2 | PTCH2_1 | Reverse Primer | GATCACGCACGGAAACA |
| 1000 | HH | PTCH2 | PTCH2_2 | Forward Primer | AAATCTGCTACAAGTCAGGA |
| 1001 | HH | PTCH2 | PTCH2_2 | Probe | AAGCTGTTTCCGTGCGTGATCCT |
| 1002 | HH | PTCH2 | PTCH2_2 | Reverse Primer | TCCCAGAAGCAGTCGAG |
| 1003 | HH | PTCH2 | PTCH2_3 | Forward Primer | TGATTGAGCGGATGATTGAG |
| 1004 | HH | PTCH2 | PTCH2_3 | Probe | AAGCTGTTTCCGTGCGTGATCCT |
| 1005 | HH | PTCH2 | PTCH2_3 | Reverse Primer | CTCCCTCCCAGAAGCAG |
| 1006 | HH | PTCH2 | PTCH2_4 | Forward Primer | TGAGCGGATGATTGAGAAGC |
| 1007 | HH | PTCH2 | PTCH2_4 | Probe | TGTTTCCGTGCGTGATCCTCACC |
| 1008 | HH | PTCH2 | PTCH2_4 | Reverse Primer | TTGGCTCCCTCCCAGAA |
| 1009 | HH | RAB34 | RAB34_1 | Forward Primer | GGCAGGAGAGGTTCAAAT |
| 1010 | HH | RAB34 | RAB34_1 | Probe | TCAACCTGAATGATGTGGCATCTCTGGA |
| 1011 | HH | RAB34 | RAB34_1 | Reverse Primer | AGCCACTGCTTGGTATG |
| 1012 | HH | RAB34 | RAB34_2 | Forward Primer | GCATTGCATCAACCTACTAT |
| 1013 | HH | RAB34 | RAB34_2 | Probe | ACATACCAAGCAGTGGCTGGCC |
| 1014 | HH | RAB34 | RAB34_2 | Reverse Primer | ATTCTCCTTCAGGGCATC |
| 1015 | HH | RAB34 | RAB34_3 | Forward Primer | CTGGGCAGGAGAGGTTC |
| 1016 | HH | RAB34 | RAB34_3 | Probe | AGAGGAGCTCAAGCCATCATCATTGTCT |
| 1017 | HH | RAB34 | RAB34_3 | Reverse Primer | CCAGAGATGCCACATCATTC |
| 1018 | HH | RAB34 | RAB34_4 | Forward primer | GGGCAGGAGAGGTTCAAATG |
| 1019 | HH | RAB34 | RAB34_4 | Probe | TCTTCAACCTGAATGATGTGGCATCTCTGG |
| 1020 | HH | RAB34 | RAB34_4 | Reverse primer | CAGCCACTGCTTGGTATGTT |
| 1021 | HH | SPP1 | SPP1_1 | Forward Primer | CCAGTTGCAGCCTTCTC |
| 1022 | HH | SPP1 | SPP1_1 | Probe | AGCCAAACGCCGACCAAGGAAA |
| 1023 | HH | SPP1 | SPP1_1 | Reverse Primer | GGTATGGCACAGGTGATG |
| 1024 | HH | SPP1 | SPP1_2 | Forward primer | AGCCTTCTCAGCCAAAC |
| 1025 | HH | SPP1 | SPP1_2 | Probe | TAGGCATCACCTGTGCCATACCAGT |
| 1026 | HH | SPP1 | SPP1_2 | Reverse primer | CCTCAGAACTTCCAGAATCA |
| 1027 | HH | SPP1 | SPP1_3 | Forward Primer | GAGGGCTTGGTTGTCAG |
| 1028 | HH | SPP1 | SPP1_3 | Probe | AGCCAAACGCCGACCAAGGAAA |
| 1029 | HH | SPP1 | SPP1_3 | Reverse Primer | TCACTGCAATTCTCATGGT |
| 1030 | HH | SPP1 | SPP1_4 | Probe | AGCCAAACGCCGACCAAGGAAA |
| 1031 | HH | SPP1 | SPP1_4 | Reverse Primer | AGCAAATCACTGCAATTCTC |
| 1032 | HH | SPP1 | SPP1_X4 | Forward Primer | GGACCAGACTCGTCTCA |
| 1033 | HH | TCEA2 | TCEA2_1 | Forward Primer | TCGGATGAGGAGGTCATT |
| 1034 | HH | TCEA2 | TCEA2_1 | Probe | ACTGGCCAAGTCTCTCATCAAGTCCT |
| 1035 | HH | TCEA2 | TCEA2_1 | Reverse Primer | TTTGGCATCGGAAGCAT |
| 1036 | HH | TCEA2 | TCEA2_2 | Forward Primer | ATTGCACTGGCCAAGTCT |
| 1037 | HH | TCEA2 | TCEA2_2 | Probe | TCCTGGAAGAAGCTCCTGGATGCT |
| 1038 | HH | TCEA2 | TCEA2_2 | Reverse Primer | TCCCTGGCTTTGGCATC |
| 1039 | HH | TCEA2 | TCEA2_3 | Forward Primer | AGCTCGGATGAGGAGGT |
| 1040 | HH | TCEA2 | TCEA2_3 | Probe | ACTGGCCAAGTCTCTCATCAAGTCCT |
| 1041 | HH | TCEA2 | TCEA2_3 | Reverse Primer | TGGCTTTGGCATCGGAA |
| 1042 | HH | TSC22D1 | TSC22D1_1 | Forward Primer | CTGGTCCACAGAGTATTCC |
| 1043 | HH | TSC22D1 | TSC22D1_1 | Probe | ACCACAGAGTATTTCTCAGTCACAGATCTC |
| 1044 | HH | TSC22D1 | TSC22D1_1 | Reverse Primer | AGATAGCTCAGTTCTTGAGAC |
| 1045 | HH | TSC22D1 | TSC22D1_2 | Forward Primer | CAGAGTATTCCAGCAGTTAGT |
| 1046 | HH | TSC22D1 | TSC22D1_2 | Probe | ACCACAGAGTATTTCTCAGTCACAGATCTC |
| 1047 | HH | TSC22D1 | TSC22D1_2 | Forward Primer | TCATGGTAAGATAGCTCAGTTC |
| 1048 | HH | TSC22D1 | TSC22D1_3 | Reverse Primer | CACAGAGTATTCCAGCAGT |
| 1049 | HH | TSC22D1 | TSC22D1_3 | Probe | ACCACAGAGTATTTCTCAGTCACAGATCTC |
| 1050 | HH | TSC22D1 | TSC22D1_3 | Forward Primer | ACCTTCATGGTAAGATAGCTC |
| 1051 | HH | TSC22D1 | TSC22D1_4 | Reverse Primer | CTCAGTCACAGATCTCACAA |
| 1052 | HH | TSC22D1 | TSC22D1_4 | Probe | AGAACTGAGCTATCTTACCATGAAGGTTGT |
| 1053 | HH | TSC22D1 | TSC22D1_4 | Reverse Primer | ACCTAAATAGTAGTTACAGTCCTC |

TABLE 7
Sets of primers and probes for determining the TGFbeta cellular signaling
pathway activity.

| SEQ | | | | | |
|---|---|---|---|---|---|
| ID | | | | | |
| NO | Pathway | Gene | Assay | Oligo | Sequence |
| 1054 | TGFb | ANGPTL4 | ANGPTL4_1 | Forward Primer | CACCGACCTCCCGTTAG |
| 1055 | TGFb | ANGPTL4 | ANGPTL4_1 | Probe | ACCCTGAGGTCCTTCACAGCCT |
| 1056 | TGFb | ANGPTL4 | ANGPTL4_1 | Reverse Primer | GTTCTGAGCCTTGAGTTGTG |
| 1057 | TGFb | ANGPTL4 | ANGPTL4_2 | Forward Primer | CAGCCTGCAGACACAACT |
| 1058 | TGFb | ANGPTL4 | ANGPTL4_2 | Reverse primer | GCTTTGCAGATGCTGAATTCG |
| 1059 | TGFb | ANGPTL4 | ANGPTL4_2 | Probe | AGCAACTCTTCCACAAGGTGGCC |
| 1060 | TGFb | ANGPTL4 | ANGPTL4_3 | Forward Primer | GAGGTCCTTCACAGCCT |
| 1061 | TGFb | ANGPTL4 | ANGPTL4_3 | Probe | ACAACTCAAGGCTCAGAACAGCAGG |
| 1062 | TGFb | ANGPTL4 | ANGPTL4_3 | Reverse Primer | CACCTTGTGGAAGAGTTGC |
| 1063 | TGFb | ANGPTL4 | ANGPTL4_4 | Forward Primer | GACCCTGAGGTCCTTCAC |
| 1064 | TGFb | ANGPTL4 | ANGPTL4_4 | Probe | AGCCTTGAGTTGTGTCTGCAGGC |
| 1065 | TGFb | ANGPTL4 | ANGPTL4_4 | Reverse Primer | TGTGGAAGAGTTGCTGGA |
| 1066 | TGFb | CDKN1A | CDKN1A_1 | Reverse primer | CTGTGGGCGGATTAGGGCT |
| 1067 | TGFb | CDKN1A | CDKN1A_1 | Forward primer | GAGACTCTCAGGGTCGAAA |
| 1068 | TGFb | CDKN1A | CDKN1A_1 | Probe | ATTTCTACCACTCCAAACGCCGGC |
| 1069 | TGFb | CDKN1A | CDKN1A_2 | Forward Primer | GAGACTCTCAGGGTCGAAA |
| 1070 | TGFb | CDKN1A | CDKN1A_2 | Probe | AATCTGTCATGCTGGTCTGCCGC |
| 1071 | TGFb | CDKN1A | CDKN1A_2 | Reverse Primer | TTCCTGTGGGCGGATTA |
| 1072 | TGFb | CDKN1A | CDKN1A_3 | Forward Primer | AGGTGGACCTGGAGACT |
| 1073 | TGFb | CDKN1A | CDKN1A_3 | Probe | AATCTGTCATGCTGGTCTGCCGC |
| 1074 | TGFb | CDKN1A | CDKN1A_3 | Reverse Primer | GGCTTCCTCTTGGAGAAGAT |
| 1075 | TGFb | CDKN1A | CDKN1A_4 | Forward Primer | GGACCTGTCACTGTCTTGTA |
| 1076 | TGFb | CDKN1A | CDKN1A_4 | Probe | AAACGGCGGCAGACCAGCAT |
| 1077 | TGFb | CDKN1A | CDKN1A_4 | Reverse Primer | GCGTTTGGAGTGGTAGAAATC |
| 1078 | TGFb | CTGF | CTGF_1 | Forward Primer | GAAGCTGACCTGGAAGAGA |
| 1079 | TGFb | CTGF | CTGF_1 | Probe | AGTTTGAGCTTTCTGGCTGCACCA |
| 1080 | TGFb | CTGF | CTGF_1 | Reverse Primer | CCACAGAATTTAGCTCGGTATG |
| 1081 | TGFb | CTGF | CTGF_2 | Forward Primer | GCAGGCTAGAGAAGCAGAG |
| 1082 | TGFb | CTGF | CTGF_2 | Probe | TTCCAGGTCAGCTTCGCAAGGC |
| 1083 | TGFb | CTGF | CTGF_2 | Reverse Primer | GGGAGTACGGATGCACTTT |
| 1084 | TGFb | CTGF | CTGF_3 | Forward Primer | GCTGACCTGGAAGAGAACAT |
| 1085 | TGFb | CTGF | CTGF_3 | Probe | TCAAGTTTGAGCTTTCTGGCTGCACC |
| 1086 | TGFb | CTGF | CTGF_3 | Reverse Primer | GCTCGGTATGTCTTCATGCT |
| 1087 | TGFb | CTGF | CTGF_4 | Probe | CCTATCAAGTTTGAGCTTTCTGGCTG |
| 1088 | TGFb | CTGF | CTGF_4 | Forward primer | GAAGCTGACCTGGAAGAGAA |
| 1089 | TGFb | CTGF | CTGF_4 | Reverse primer | CCACAGAATTTAGCTCGGTATG |
| 1090 | TGFb | GADD45A | GADD45A_1 | Forward Primer | GCGAGAACGACATCAACATC |
| 1091 | TGFb | GADD45A | GADD45A_1 | Probe | AGCTCCTGCTCTTGGAGACCGA |
| 1092 | TGFb | GADD45A | GADD45A_1 | Reverse Primer | TGGATTCGTCACCAGCA |
| 1093 | TGFb | GADD45A | GADD45A_2 | Probe | AAGGATCCTGCCTTAAGTCAACTTATTTG |
| 1094 | TGFb | GADD45A | GADD45A_2 | Reverse primer | GATCCATGTAGCGACTTTCC |
| 1095 | TGFb | GADD45A | GADD45A_2 | Forward Primer | ACGAATCCACATTCATCTCAAT |
| 1096 | TGFb | GADD45A | GADD45A_3 | Forward Primer | CGGAGCTCCTGCTCTTG |
| 1097 | TGFb | GADD45A | GADD45A_3 | Probe | TGTGGATTCGTCACCAGCACGC |
| 1098 | TGFb | GADD45A | GADD45A_3 | Reverse Primer | AGGATCCTTCCATTGAGATGAA |
| 1099 | TGFb | GADD45A | GADD45A_4 | Forward Primer | GCTCCTGCTCTTGGAGAC |
| 1100 | TGFb | GADD45A | GADD45A_4 | Probe | TGTGGATTCGTCACCAGCACGC |
| 1101 | TGFb | GADD45A | GADD45A_4 | Reverse Primer | GCAGGATCCTTCCATTGAGA |
| 1102 | TGFb | GADD45B | GADD45B_1 | Forward Primer | GTCGGCCAAGTTGATGAATG |
| 1103 | TGFb | GADD45B | GADD45B_1 | Probe | ACAGCGTGGTCCTCTGCCTCTT |
| 1104 | TGFb | GADD45B | GADD45B_1 | Reverse Primer | GATGAGCGTGAAGTGGATTTG |
| 1105 | TGFb | GADD45B | GADD45B_2 | Forward Primer | CGAGTCGGCCAAGTTGAT |
| 1106 | TGFb | GADD45B | GADD45B_2 | Probe | ACAGCGTGGTCCTCTGCCTCTT |
| 1107 | TGFb | GADD45B | GADD45B_2 | Reverse Primer | ACTGGATGAGCGTGAAGTG |
| 1108 | TGFb | GADD45B | GADD45B_3 | Forward Primer | TGTACGAGTCGGCCAAG |
| 1109 | TGFb | GADD45B | GADD45B_3 | Probe | ACAGCGTGGTCCTCTGCCTCTT |
| 1110 | TGFb | GADD45B | GADD45B_3 | Reverse Primer | GATTTGCAGGGCGATGTC |
| 1111 | TGFb | GADD45B | GADD45B_4 | Forward Primer | CAGGATCGCCTCACAGT |
| 1112 | TGFb | GADD45B | GADD45B_4 | Probe | ACAGCGTGGTCCTCTGCCTCTT |
| 1113 | TGFb | GADD45B | GADD45B_4 | Reverse Primer | CTCCTCCTCCTCGTCAATG |
| 1114 | TGFb | ID1 | ID1_1 | Forward Primer | CCTCAACGGCGAGATCA |
| 1115 | TGFb | ID1 | ID1_1 | Probe | TCGCATCTTGTGTCGCTGAAGCG |
| 1116 | TGFb | ID1 | ID1_1 | Reverse Primer | ACCCACAGAGCACGTAAT |
| 1117 | TGFb | ID1 | ID1_2 | Forward Primer | GAATCCGAAGTTGGAACCC |
| 1118 | TGFb | ID1 | ID1_2 | Probe | AGCACCCTCAACGGCGAGATCA |
| 1119 | TGFb | ID1 | ID1_2 | Reverse Primer | GCTTCAGCGACACAAGAT |
| 1120 | TGFb | ID1 | ID1_3 | Forward Primer | TTGGAGCTGAACTCGGAATC |
| 1121 | TGFb | ID1 | ID1_3 | Probe | AGCACCCTCAACGGCGAGATCA |
| 1122 | TGFb | ID1 | ID1_3 | Reverse Primer | AGCGACACAAGATGCGAT |
| 1123 | TGFb | ID1 | ID1_4 | Forward Primer | CGCTCAGCACCCTCAAC |
| 1124 | TGFb | ID1 | ID1_4 | Probe | TCGCATCTTGTGTCGCTGAAGCG |
| 1125 | TGFb | ID1 | ID1_4 | Reverse Primer | AGCACGTAATTCCTCTTGCC |
| 1126 | TGFb | IL-11 | IL-11_1 | Forward Primer | AGTACCCGTATGGGACAAA |
| 1127 | TGFb | IL-11 | IL-11_1 | Reverse primer | CCAGTTTGCTATGGTGAACA |
| 1128 | TGFb | IL-11 | IL-11_1 | Probe | TGCAAGGTCAAGATGGTTCATTATGGCTG |
| 1129 | TGFb | IL-11 | IL-11_2 | Forward Primer | GGGACCACAACCTGGATTC |
| 1130 | TGFb | IL-11 | IL-11_2 | Probe | AGCTCTACAGCTCCCAGGTGTG |
| 1131 | TGFb | IL-11 | IL-11_2 | Reverse Primer | GCTCGCAGCCTTGTCAG |
| 1132 | TGFb | IL-11 | IL-11_3 | Forward Primer | GGGCACTGGGAGCTCTA |
| 1133 | TGFb | IL-11 | IL-11_3 | Probe | ACCTACTGTCCTACCTGCGGCA |
| 1134 | TGFb | IL-11 | IL-11_3 | Reverse Primer | GGGCTCCAGGGTCTTCA |
| 1135 | TGFb | IL-11 | IL-11_4 | Forward Primer | GGACAAATTCCCAGCTGAC |
| 1136 | TGFb | IL-11 | IL-11_4 | Probe | TCCCAGGTGTGCTGACAAGGCT |
| 1137 | TGFb | IL-11 | IL-11_4 | Reverse Primer | CCGCAGGTAGGACAGTAG |
| 1138 | TGFb | JUNB | JUNB_1 | Forward Primer | CTACCACGACGACTCATACA |
| 1139 | TGFb | JUNB | JUNB_1 | Probe | TGGTGGCCTCTCTCTACACGACT |
| 1140 | TGFb | JUNB | JUNB_1 | Reverse Primer | GCTCGGTTTCAGGAGTTTG |
| 1141 | TGFb | JUNB | JUNB_2 | Forward Primer | ATGGAACAGCCCTTCTACC |
| 1142 | TGFb | JUNB | JUNB_2 | Probe | TCATACACAGCTACGGGATACGGCC |
| 1143 | TGFb | JUNB | JUNB_2 | Reverse Primer | TCAGGAGTTTGTAGTCGTGTAG |
| 1144 | TGFb | JUNB | JUNB_3 | Forward Primer | CCCGGATGTGCACTAAA |
| 1145 | TGFb | JUNB | JUNB_3 | Reverse primer | GCTCGGTTTCAGGAGTTTGTA |
| 1146 | TGFb | JUNB | JUNB_3 | Probe | TCATACACAGCTACGGGATACGG |
| 1147 | TGFb | JUNB | JUNB_4 | Forward Primer | CGACGACTCATACACAGCTA |
| 1148 | TGFb | JUNB | JUNB_4 | Probe | AAACTCCTGAAACCGAGCCTGGC |
| 1149 | TGFb | JUNB | JUNB_4 | Reverse Primer | CTTTGAGACTCCGGTAGGG |
| 1150 | TGFb | MMP2 | MMP2_1 | Forward Primer | GTGGCCAACTACAACTTCTTC |
| 1151 | TGFb | MMP2 | MMP2_1 | Probe | CGCAAGCCCAAGTGGGACAAGA |
| 1152 | TGFb | MMP2 | MMP2_1 | Reverse Primer | GGCATCATCCACTGTCTCT |
| 1153 | TGFb | MMP2 | MMP2_2 | Forward Primer | TGGGACAAGAACCAGATCAC |
| 1154 | TGFb | MMP2 | MMP2_2 | Probe | ACCCAGAGACAGTGGATGATGCCT |
| 1155 | TGFb | MMP2 | MMP2_2 | Reverse Primer | GTCACATCGCTCCAGACTT |
| 1156 | TGFb | MMP2 | MMP2_3 | Forward Primer | GGACAAGAACCAGATCACATAC |
| 1157 | TGFb | MMP2 | MMP2_3 | Probe | TGGGTCCAGATCAGGTGTGTAGCC |
| 1158 | TGFb | MMP2 | MMP2_3 | Reverse Primer | CACGAGCAAAGGCATCAT |
| 1159 | TGFb | MMP2 | MMP2_4 | Probe | CACATACAGGATCATTGGCTACACACC |
| 1160 | TGFb | MMP2 | MMP2_4 | Reverse primer | GTCACATCGCTCCAGACTT |
| 1161 | TGFb | MMP2 | MMP2_4 | Forward Primer | AAGTGGGACAAGAACCAGAT |
| 1162 | TGFb | MMP9 | MMP9_1 | Forward Primer | GGAGACCTGAGAACCAATC |
| 1163 | TGFb | MMP9 | MMP9_1 | Reverse primer | GACTCTCCACGCATCTCTG |
| 1164 | TGFb | MMP9 | MMP9_1 | Probe | AGGCAGCTGGCAGAGGAATACCT |
| 1165 | TGFb | MMP9 | MMP9_2 | Forward Primer | TCCACCCTTGTGCTCTT |
| 1166 | TGFb | MMP9 | MMP9_2 | Probe | AACCAATCTCACCGACAGGCAGC |
| 1167 | TGFb | MMP9 | MMP9_2 | Reverse Primer | ACTCTCCACGCATCTCTG |
| 1168 | TGFb | MMP9 | MMP9_3 | Forward Primer | AGAACCAATCTCACCGACAG |
| 1169 | TGFb | MMP9 | MMP9_3 | Probe | TGGTTACACTCGGGTGGCAGAGA |
| 1170 | TGFb | MMP9 | MMP9_3 | Reverse Primer | CCAGAGATTTCGACTCTCCAC |
| 1171 | TGFb | MMP9 | MMP9_4 | Forward Primer | TGGAGACCTGAGAACCAATC |
| 1172 | TGFb | MMP9 | MMP9_4 | Probe | AGGCAGCTGGCAGAGGAATACCT |
| 1173 | TGFb | MMP9 | MMP9_4 | Reverse Primer | ACCCGAGTGTAACCATAGC |
| 1174 | TGFb | PDGFB | PDGFB_1 | Forward Primer | CATTCCCGAGGAGCTTTATG |
| 1175 | TGFb | PDGFB | PDGFB_1 | Probe | TGACCACTCGATCCGCTCCTTTGA |
| 1176 | TGFb | PDGFB | PDGFB_1 | Reverse Primer | GTCATGTTCAGGTCCAACTC |
| 1177 | TGFb | PDGFB | PDGFB_2 | Forward Primer | CCCGAGGAGCTTTATGAGAT |
| 1178 | TGFb | PDGFB | PDGFB_2 | Probe | TGACCACTCGATCCGCTCCTTTGA |
| 1179 | TGFb | PDGFB | PDGFB_2 | Reverse Primer | GGGTCATGTTCAGGTCCA |
| 1180 | TGFb | PDGFB | PDGFB_3 | Forward Primer | TCGATCCGCTCCTTTGAT |
| 1181 | TGFb | PDGFB | PDGFB_3 | Probe | TGAACATGACCCGCTCCCACTCT |
| 1182 | TGFb | PDGFB | PDGFB_3 | Reverse Primer | CCAGGCTCCTTCTTCCA |
| 1183 | TGFb | PDGFB | PDGFB_4 | Forward Primer | ATCCGCTCCTTTGATGATCT |
| 1184 | TGFb | PDGFB | PDGFB_4 | Probe | TGAACATGACCCGCTCCCACTCT |
| 1185 | TGFb | PDGFB | PDGFB_4 | Reverse Primer | CCTTCTTCCACGAGCCA |
| 1186 | TGFb | SERPINE1 | SERPINE1_1 | Forward Primer | TGTCTCTGTGCCCATGAT |
| 1187 | TGFb | SERPINE1 | SERPINE1_1 | Reverse Primer | CAGTTCCAGGATGTCGTAGT |
| 1188 | TGFb | SERPINE1 | SERPINE1_2 | Forward Primer | CGCCTCTTCCACAAATCAG |
| 1189 | TGFb | SERPINE1 | SERPINE1_2 | Probe | ATGGGCACAGAGACAGTGCTGC |
| 1190 | TGFb | SERPINE1 | SERPINE1_2 | Reverse Primer | TCCAGGATGTCGTAGTAATGG |
| 1191 | TGFb | SERPINE1 | SERPINE1_3 | Forward Primer | TGGCTCAGACCAACAAGT |
| 1192 | TGFb | SERPINE1 | SERPINE1_3 | Probe | ACTACGACATCCTGGAACTGCCCT |
| 1193 | TGFb | SERPINE1 | SERPINE1_3 | Reverse Primer | CAGCAATGAACATGCTGAGG |
| 1194 | TGFb | Serpine1 | Serpine1_4 | Reverse primer | GTCGTAGTAATGGCCATCGG |
| 1195 | TGFb | Serpine1 | Serpine1_4 | Forward Primer | CCACAAATCAGACGGCAGCA |
| 1196 | TGFb | Serpine1 | Serpine1_4 | Probe | CCCATGATGGCTCAGACCAACAAGT |
| 1197 | TGFb | SERPINE1 | SERPINE1_X1 | Probe | ACTGAGTTCACCACGCCCGATG |
| 1198 | TGFb | SGK1 | SGK1_1 | Forward Primer | GGAGCCTGAGCTTATGAAT |
| 1199 | TGFb | SGK1 | SGK1_1 | Reverse Primer | GAAGTGAAAGTCAGATGGTTTAG |
| 1200 | TGFb | SGK1 | SGK1_1 | Probe | TTGGTGGAGGAGAAGGGTTGGC |
| 1201 | TGFb | SGK1 | SGK1_2 | Forward Primer | TATGAATGCCAACCCTTCTC |
| 1202 | TGFb | SGK1 | SGK1_2 | Reverse Primer | CCCTTTCCGATCACTTTCA |
| 1203 | TGFb | SGK1 | SGK1_2 | Probe | AATCAACCTTGGCCCGTCGTCC |
| 1204 | TGFb | SGK1 | SGK1_3 | Forward Primer | CAGGAGCCTGAGCTTATGAA |
| 1205 | TGFb | SGK1 | SGK1_3 | Reverse primer | GATGGTTTAGCATGAGGATTGG |
| 1206 | TGFb | SGK1 | SGK1_3 | Probe | TCAGCAAATCAACCTTGGCCCGT |
| 1207 | TGFb | SGK1 | SGK1_4 | Forward Primer | CTTGAAGATCTCCCAACCTC |
| 1208 | TGFb | SGK1 | SGK1_4 | Reverse Primer | CAAGGTTGATTTGCTGAGAAG |
| 1209 | TGFb | SGK1 | SGK1_4 | Probe | TTGGTGGAGGAGAAGGGTTGGC |
| 1210 | TGFb | SKIL | SKIL_1 | Forward Primer | GCATGAGAAGTGGAAAGAGAAAT |
| 1211 | TGFb | SKIL | SKIL_1 | Probe | CCAAGACAGATGCACCATCAGGAATGG |
| 1212 | TGFb | SKIL | SKIL_1 | Reverse Primer | TGGTCACCTTCCTGCTTTAT |
| 1213 | TGFb | SKIL | SKIL_2 | Probe | ACAGATGCACCATCAGGAATGGAATTACA |
| 1214 | TGFb | SKIL | SKIL_2 | Reverse primer | CTGAGAAACATGGTCACCT |
| 1215 | TGFb | SKIL | SKIL_2 | Forward Primer | CATGAGAAGTGGAAAGAGAAATC |
| 1216 | TGFb | SKIL | SKIL_3 | Forward Primer | GGAGAAGTTTAGCATGAGAAGTG |
| 1217 | TGFb | SKIL | SKIL_3 | Probe | CCAAGACAGATGCACCATCAGGAATGG |
| 1218 | TGFb | SKIL | SKIL_3 | Reverse Primer | TCTGAGAAACATGGTCACCT |
| 1219 | TGFb | SKIL | SKIL_4 | Forward Primer | GTTTAGCATGAGAAGTGGAAAGA |
| 1220 | TGFb | SKIL | SKIL_4 | Probe | CCAAGACAGATGCACCATCAGGAATGG |
| 1221 | TGFb | SKIL | SKIL_4 | Reverse Primer | GAAACATGGTCACCTTCCTG |
| 1222 | TGFb | SMAD4 | SMAD4_1 | Forward Primer | ACAAATGGAGCTCATCCTAGT |
| 1223 | TGFb | SMAD4 | SMAD4_1 | Probe | TCAGGTGGCTGGTCGGAAAGGA |
| 1224 | TGFb | SMAD4 | SMAD4_1 | Reverse Primer | GGGCATAGATCACATGAGGA |
| 1225 | TGFb | SMAD4 | SMAD4_2 | Forward Primer | CAAATGGAGCTCATCCTAGTAAAT |
| 1226 | TGFb | SMAD4 | SMAD4_2 | Probe | TCAGGTGGCTGGTCGGAAAGGA |
| 1227 | TGFb | SMAD4 | SMAD4_2 | Reverse Primer | AGAGACGGGCATAGATCAC |
| 1228 | TGFb | SMAD4 | SMAD4_3 | Forward Primer | GGAGCTCATCCTAGTAAATGTGT |
| 1229 | TGFb | SMAD4 | SMAD4_3 | Probe | TCAGGTGGCTGGTCGGAAAGGA |
| 1230 | TGFb | SMAD4 | SMAD4_3 | Reverse Primer | TCCAGAGACGGGCATAGA |
| 1231 | TGFb | SMAD4 | SMAD4_4 | Forward Primer | GCTCATCCTAGTAAATGTGTTACC |
| 1232 | TGFb | SMAD4 | SMAD4_4 | Probe | TTTCCGACCAGCCACCTGAAGC |
| 1233 | TGFb | SMAD4 | SMAD4_4 | Reverse Primer | GGCATAGATCACATGAGGAAATC |
| 1234 | TGFb | SMAD7 | SMAD7_1 | Forward Primer | AGATGCTGTGCCTTCCT |
| 1235 | TGFb | SMAD7 | SMAD7_1 | Probe | AGATTCCCAACTTCTTCTGGAGCCTGG |
| 1236 | TGFb | SMAD7 | SMAD7_1 | Reverse Primer | ACCAGTGTGACCGATCC |
| 1237 | TGFb | SMAD7 | SMAD7_2 | Forward Primer | CCTTCCTCCGCTGAAACA |
| 1238 | TGFb | SMAD7 | SMAD7_2 | Probe | ACACTGGTGCGTGGTGGCATAC |
| 1239 | TGFb | SMAD7 | SMAD7_2 | Reverse Primer | TCTCGTCTTCTCCTCCCA |
| 1240 | TGFb | SMAD7 | SMAD7_3 | Forward Primer | GTCCAGATGCTGTGCCT |
| 1241 | TGFb | SMAD7 | SMAD7_3 | Probe | TCCCAACTTCTTCTGGAGCCTGGG |
| 1242 | TGFb | SMAD7 | SMAD7_3 | Reverse Primer | CTCCCAGTATGCCACCAC |
| 1243 | TGFb | SMAD7 | SMAD7_4 | Reverse primer | ACCACGCACCAGTGTGAC |
| 1244 | TGFb | SMAD7 | SMAD7_4 | Forward Primer | TGCCTTCCTCCGCTGAAAC |
| 1245 | TGFb | SMAD7 | SMAD7_4 | Probe | TCCCAACTTCTTCTGGAGCCTGGG |
| 1246 | TGFb | SNAI1 | SNAI1_1 | Forward Primer | CCCACACTGGCGAGAAG |
| 1247 | TGFb | SNAI1 | SNAI1_1 | Probe | TTCGCTGACCGCTCCAACCT |
| 1248 | TGFb | SNAI1 | SNAI1_1 | Reverse Primer | TTGACATCTGAGTGGGTCTG |
| 1249 | TGFb | SNAI1 | SNAI1_2 | Forward Primer | CATGTCCGGACCCACAC |
| 1250 | TGFb | SNAI1 | SNAI1_2 | Probe | TGGCGAGAAGCCCTTCTCCTGT |
| 1251 | TGFb | SNAI1 | SNAI1_2 | Reverse Primer | GGCACTGGTACTTCTTGACA |
| 1252 | TGFb | SNAI1 | SNAI1_3 | Forward Primer | TTCTCTAGGCCCTGGCT |
| 1253 | TGFb | SNAI1 | SNAI1_3 | Probe | TACAAGGCCATGTCCGGACCCA |
| 1254 | TGFb | SNAI1 | SNAI1_3 | Reverse Primer | GGTACTTCTTGACATCTGAGTGG |
| 1255 | TGFb | SNAI1 | SNAI1_4 | Forward Primer | GCCCTGGCTGCTACAAG |
| 1256 | TGFb | SNAI1 | SNAI1_4 | Probe | ACTGGCGAGAAGCCCTTCTCCT |
| 1257 | TGFb | SNAI1 | SNAI1_4 | Reverse Primer | CTGAGTGGGTCTGGAGGT |
| 1258 | TGFb | SNAI1 | TIMP1_1 | Forward Primer | CCCAGAGAGACACCAGAGAA |
| 1259 | TGFb | SNAI1 | TIMP1_1 | Reverse Primer | GCTATCAGCCACAGCAACA |
| 1260 | TGFb | SNAI1 | TIMP1_1 | Probe | CCTGGCTTCTGGCATCCTGT |
| 1261 | TGFb | SNAI1 | TIMP1_2 | Forward Primer | CATCGCCGCAGATCCAG |
| 1262 | TGFb | SNAI1 | TIMP1_2 | Probe | CCCAGAGAGACACCAGAGAACCCA |
| 1263 | TGFb | SNAI1 | TIMP1_2 | Reverse Primer | TCAGCCACAGCAACAACA |
| 1264 | TGFb | SNAI1 | TIMP1_3 | Forward Primer | AGAGACACCAGAGAACCCA |
| 1265 | TGFb | SNAI1 | TIMP1_3 | Probe | TCTGGCATCCTGTTGTTGCTGTGG |
| 1266 | TGFb | SNAI1 | TIMP1_3 | Reverse Primer | GTGGGACACAGGTGCAG |
| 1267 | TGFb | SNAI1 | TIMP1_4 | Forward Primer | CCAGAGAGACACCAGAGAA |
| 1268 | TGFb | SNAI1 | TIMP1_4 | Probe | TCTGGCATCCTGTTGTTGCTGTGG |
| 1269 | TGFb | SNAI1 | TIMP1_4 | Reverse Primer | GAGGTCGGAATTGCAGAAG |
| 1270 | TGFb | VEGFA | VEGFA_1 | Forward Primer | GAGGAGGGCAGAATCATCA |
| 1271 | TGFb | VEGFA | VEGFA_1 | Probe | TGCGCTGATAGACATCCATGAACTTCAC |
| 1272 | TGFb | VEGFA | VEGFA_1 | Reverse Primer | TCTCGATTGGATGGCAGTAG |
| 1273 | TGFb | VEGFA | VEGFA_2 | Forward Primer | GGGCAGAATCATCACGAAG |
| 1274 | TGFb | VEGFA | VEGFA_2 | Reverse Primer | GTCTCGATTGGATGGCAGTA |
| 1275 | TGFb | VEGFA | VEGFA_2 | Probe | AGTTCATGGATGTCTATCAGCGCAGC |
| 1276 | TGFb | VEGFA | VEGFA_3 | Forward Primer | ACCCATGGCAGAAGGAG |
| 1277 | TGFb | VEGFA | VEGFA_3 | Probe | AGGGCAGAATCATCACGAAGTGGT |
| 1278 | TGFb | VEGFA | VEGFA_3 | Reverse Primer | CAGTAGCTGCGCTGATAGA |
| 1279 | TGFb | VEGFA | VEGFA_4 | Forward Primer | GAAGGAGGAGGGCAGAAT |
| 1280 | TGFb | VEGFA | VEGFA_4 | Probe | CAGCGCAGCTACTGCCATCCAA |
| 1281 | TGFb | VEGFA | VEGFA_4 | Reverse Primer | TACTCCTGGAAGATGTCCAC |

TABLE 8
Sets of primers and probes for determining the expression levels of reference genes.

| NO | | | | | |
|---|---|---|---|---|---|
| SEQ | | | | | |
| ID | Pathway | Gene | Assay | Oligo | Sequence |
| 1282 | REF | ACTB | ACTB_1 | Forward primer | CCAACCGCGAGAAGATGA |
| 1283 | REF | ACTB | ACTB_1 | Probe | CCATGTACGTTGCTATCCAGGCT |
| 1284 | REF | ACTB | ACTB_1 | Reverse primer | CCAGAGGCGTACAGGGATAG |
| 1285 | REF | ACTB | ACTB_2 | Forward primer | CCCAGATCATGTTTGAGACCTTC |
| 1286 | REF | ACTB | ACTB_2 | Probe | ACGTTGCTATCCAGGCTGTGCT |
| 1287 | REF | ACTB | ACTB_2 | Reverse primer | GTCCATCACGATGCCAGTG |
| 1288 | REF | ACTB | ACTB_3 | Forward primer | CCAACCGCGAGAAGATGAC |
| 1289 | REF | ACTB | ACTB_3 | Probe | CCAGATCATGTTTGAGACCTTCAACACCC |
| 1290 | REF | ACTB | ACTB_3 | Reverse primer | GGATAGCACAGCCTGGATAG |
| 1291 | REF | ACTB | ACTB_4 | Forward primer | GATGACCCAGATCATGTTTGA |
| 1292 | REF | ACTB | ACTB_4 | Probe | CCATGTACGTTGCTATCCAGGCTGT |
| 1293 | REF | ACTB | ACTB_4 | Reverse primer | CAGAGGCGTACAGGGATAG |
| 1294 | REF | ALAS1 | ALAS1_1 | Forward primer | ATGAGACAGATGCTAATGGATG |
| 1295 | REF | ALAS1 | ALAS1_1 | Probe | TTTAGCAGCATCTGCAACCCGC |
| 1296 | REF | ALAS1 | ALAS1_1 | Reverse primer | TTGCTTGCACGTAGATGTTA |
| 1297 | REF | ALAS1 | ALAS1_2 | Forward primer | CAAACTCATGAGACAGATGCTA |
| 1298 | REF | ALAS1 | ALAS1_2 | Probe | TTTAGCAGCATCTGCAACCCGC |
| 1299 | REF | ALAS1 | ALAS1_2 | Reverse primer | GCTCATTAGTTCATCACAGACT |
| 1300 | REF | ALAS1 | ALAS1_3 | Forward primer | CAGCCACATCATCCCTGTG |
| 1301 | REF | ALAS1 | ALAS1_3 | Probe | AGCAGACATAACATCTACGTGCAAGCA |
| 1302 | REF | ALAS1 | ALAS1_3 | Reverse primer | GGCACCGTAGGGTAATTGAT |
| 1303 | REF | ALAS1 | ALAS1_4 | Forward primer | AGCCACATCATCCCTGT |
| 1304 | REF | ALAS1 | ALAS1_4 | Probe | TTTAGCAGCATCTGCAACCCGC |
| 1305 | REF | ALAS1 | ALAS1_4 | Reverse primer | CGTAGATGTTATGTCTGCTCAT |
| 1306 | REF | B2M | B2M_1 | Forward primer | GTCACAGCCCAAGATAGTTAAG |
| 1307 | REF | B2M | B2M_1 | Probe | TCATGGAGGTTTGAAGATGCCGCA |
| 1308 | REF | B2M | B2M_1 | Reverse primer | GCAAGCAAGCAGAATTTGG |
| 1309 | REF | B2M | B2M_2 | Forward primer | CGTGTGAACCATGTGACTT |
| 1310 | REF | B2M | B2M_2 | Probe | CACAGCCCAAGATAGTTAAGTGGGATCG |
| 1311 | REF | B2M | B2M_2 | Reverse primer | CCTCCATGATGCTGCTTAC |
| 1312 | REF | B2M | B2M_3 | Forward primer | CTTTGTCACAGCCCAAGAT |
| 1313 | REF | B2M | B2M_3 | Probe | TGGGATCGAGACATGTAAGCAGCA |
| 1314 | REF | B2M | B2M_3 | Reverse primer | TGGAATTCATCCAATCCAAATG |
| 1315 | REF | B2M | B2M_4 | Forward primer | GTATGCCTGCCGTGTGAAC |
| 1316 | REF | B2M | B2M_4 | Probe | AAGTGGGATCGAGACATGTAAGCAGC |
| 1317 | REF | B2M | B2M_4 | Reverse primer | GGCATCTTCAAACCTCCATGAT |
| 1318 | REF | EEF1A1 | EEF1A1_1 | Forward primer | CAAAGCAGTGGACAAGAAG |
| 1319 | REF | EEF1A1 | EEF1A1_1 | Probe | TGGGCAGACTTGGTGACCTTGC |
| 1320 | REF | EEF1A1 | EEF1A1_1 | Reverse primer | GTGGCAGGTATTAGGGATAA |
| 1321 | REF | EEF1A1 | EEF1A1_2 | Forward primer | CTTTGCTGTTCGTGATATGAG |
| 1322 | REF | EEF1A1 | EEF1A1_2 | Probe | TGCGGTGGGTGTCATCAAAGCA |
| 1323 | REF | EEF1A1 | EEF1A1_2 | Reverse primer | CATTTAGCCTTCTGAGCTTTC |
| 1324 | REF | EEF1A1 | EEF1A1_3 | Forward primer | GAAAGCTCAGAAGGCTAAATG |
| 1325 | REF | EEF1A1 | EEF1A1_3 | Probe | TCAGTGGTGGAAGAACGGTCTCAGAA |
| 1326 | REF | EEF1A1 | EEF1A1_3 | Reverse primer | TGGCCAATTGAAACAAACA |
| 1327 | REF | EEF1A1 | EEF1A1_4 | Forward primer | GTTCGTGATATGAGACAGACA |
| 1328 | REF | EEF1A1 | EEF1A1_4 | Probe | TGCGGTGGGTGTCATCAAAGCA |
| 1329 | REF | EEF1A1 | EEF1A1_4 | Reverse primer | GGGATAATATTCATTTAGCCTTCTG |
| 1330 | REF | POLR2A | POLR2A_1 | Forward primer | CAAGTACATCATCCGAGACAA |
| 1331 | REF | POLR2A | POLR2A_1 | Probe | TCGCATTGACTTGCGTTTCCACC |
| 1332 | REF | POLR2A | POLR2A_1 | Reverse primer | GTGCCGTTCCACCTTATAG |
| 1333 | REF | POLR2A | POLR2A_2 | Forward primer | ATGGTGATCGCATTGACTT |
| 1334 | REF | POLR2A | POLR2A_2 | Probe | ACCGGCTATAAGGTGGAACGGC |
| 1335 | REF | POLR2A | POLR2A_2 | Reverse primer | CTGCCGGTTGAAGATAACA |
| 1336 | REF | POLR2A | POLR2A_3 | Forward primer | TCGCATTGACTTGCGTTTC |
| 1337 | REF | POLR2A | POLR2A_3 | Probe | CCAAGCCCAGTGACCTTCACCT |
| 1338 | REF | POLR2A | POLR2A_3 | Reverse primer | CATCACACATGTGCCGTTC |
| 1339 | REF | POLR2A | POLR2A_4 | Forward primer | CATTGACTTGCGTTTCCAC |
| 1340 | REF | POLR2A | POLR2A_4 | Probe | TTCACCTGCAGACCGGCTATAAGGT |
| 1341 | REF | POLR2A | POLR2A_4 | Reverse primer | TAACAATGTCCCCATCACACAT |
| 1342 | REF | PUM1 | PUM1_1 | Forward primer | GCTTGTCTTCAATGAAATCCTC |
| 1343 | REF | PUM1 | PUM1_1 | Probe | TCCACCATGAGTTGGTAGGCAGC |
| 1344 | REF | PUM1 | PUM1_1 | Reverse primer | CTGTTCAAGACTGCCAAATTC |
| 1345 | REF | PUM1 | PUM1_2 | Forward primer | CCAACTCATGGTGGATGTG |
| 1346 | REF | PUM1 | PUM1_2 | Probe | AATCCGTTCTGCCAAAGCCAGC |
| 1347 | REF | PUM1 | PUM1_2 | Reverse primer | CATACATCTGTAGTGCCAATGA |
| 1348 | REF | PUM1 | PUM1_3 | Forward primer | GCCAGCTTGTCTTCAATGAAAT |
| 1349 | REF | PUM1 | PUM1_3 | Probe | ATCCACCATGAGTTGGTAGGCAGC |
| 1350 | REF | PUM1 | PUM1_3 | Reverse primer | CAAAGCCAGCTTCTGTTCAAG |
| 1351 | REF | PUM1 | PUM1_4 | Forward primer | CATGGTGGATGTGTTTGGTAAT |
| 1352 | REF | PUM1 | PUM1_4 | Probe | TTGGCAGTCTTGAACAGAAGCTGG |
| 1353 | REF | PUM1 | PUM1_4 | Reverse primer | CGAATCCGTTCTGCCAAAG |
| 1354 | REF | RPLP0 | RPLP0_1 | Forward primer | CAACCCTGAAGTGCTTGATA |
| 1355 | REF | RPLP0 | RPLP0_1 | Probe | TGCATTCTCGCTTCCTGGAGGG |
| 1356 | REF | RPLP0 | RPLP0_1 | Reverse primer | GTTTGTACCCGTTGATGATAGA |
| 1357 | REF | RPLP0 | RPLP0_2 | Forward primer | CACAGAGGAAACTCTGCATTC |
| 1358 | REF | RPLP0 | RPLP0_2 | Probe | AGGGTGTCCGCAATGTTGCCAGT |
| 1359 | REF | RPLP0 | RPLP0_2 | Reverse primer | GATGCAACAGTTGGGTAGC |
| 1360 | REF | RPLP0 | RPLP0_3 | Forward primer | GACAATGGCAGCATCTACA |
| 1361 | REF | RPLP0 | RPLP0_3 | Probe | TGCATTCTCGCTTCCTGGAGGG |
| 1362 | REF | RPLP0 | RPLP0_3 | Reverse primer | CCAATCTGCAGACAGACAC |
| 1363 | REF | RPLP0 | RPLP0_4 | Forward primer | CAGCATCTACAACCCTGAAG |
| 1364 | REF | RPLP0 | RPLP0_4 | Probe | TGCATTCTCGCTTCCTGGAGGG |
| 1365 | REF | RPLP0 | RPLP0_4 | Reverse primer | GACAGACACTGGCAACATT |
| 1366 | REF | TBP | TBP_1 | Forward primer | AAGGGATTCAGGAAGACGA |
| 1367 | REF | TBP | TBP_1 | Probe | AATGGCTCTCATGTACCCTTGCCT |
| 1368 | REF | TBP | TBP_1 | Reverse primer | TTCTCACAACACCACCATTTA |
| 1369 | REF | TBP | TBP_2 | Forward primer | CCCTATTCTAAAGGGATTCAGG |
| 1370 | REF | TBP | TBP_2 | Probe | AATGGCTCTCATGTACCCTTGCCT |
| 1371 | REF | TBP | TBP_2 | Reverse primer | ACTCAACATCCATCTTCTCAC |
| 1372 | REF | TBP | TBP_3 | Forward primer | GATTCAGGAAGACGACGTAATG |
| 1373 | REF | TBP | TBP_3 | Probe | CTCTCATGTACCCTTGCCTCCC |
| 1374 | REF | TBP | TBP_3 | Reverse primer | ACCACCATTTAAAGGTACCAAA |
| 1375 | REF | TBP | TBP_4 | Forward primer | CGTAATGGCTCTCATGTACC |
| 1376 | REF | TBP | TBP_4 | Probe | TGGTACCTTTAAATGGTGGTGTTGTGAGA |
| 1377 | REF | TBP | TBP_4 | Reverse primer | CCTGCAACTCAACATCCAT |
| 1378 | REF | TPT1 | TPT1_1 | Forward primer | GAACAGAGACCAGAAAGAGTAAA |
| 1379 | REF | TPT1 | TPT1_1 | Probe | TGTGCTTGATTTGTTCTGCAGCCC |
| 1380 | REF | TPT1 | TPT1_1 | Reverse primer | TCCTCACGGTAGTCCAATAG |
| 1381 | REF | TPT1 | TPT1_2 | Forward primer | GGGAAACTTGAAGAACAGAGAC |
| 1382 | REF | TPT1 | TPT1_2 | Probe | TGTGCTTGATTTGTTCTGCAGCCC |
| 1383 | REF | TPT1 | TPT1_2 | Reverse primer | CAACCATGCCATCTGGATTC |
| 1384 | REF | TPT1 | TPT1_3 | Forward primer | CAAGCACATCCTTGCTAATTTC |
| 1385 | REF | TPT1 | TPT1_3 | Probe | TGAATCCAGATGGCATGGTTGCTCT |
| 1386 | REF | TPT1 | TPT1_3 | Reverse primer | CACACCATCCTCACGGTAG |
| 1387 | REF | TPT1 | TPT1_4 | Forward primer | GCCTACAAGAAGTACATCAAAGA |
| 1388 | REF | TPT1 | TPT1_4 | Probe | AGGGAAACTTGAAGAACAGAGACCAGA |
| 1389 | REF | TPT1 | TPT1_4 | Reverse primer | GCAAGGATGTGCTTGATTTG |
| 1390 | REF | TUBA1B | TUBA1B_1 | Forward primer | TGACTCCTTCAACACCTTCTTC |
| 1391 | REF | TUBA1B | TUBA1B_1 | Probe | CCGGGCTGTGTTTGTAGACTTGGA |
| 1392 | REF | TUBA1B | TUBA1B_1 | Reverse primer | CCAGTGCGAACTTCATCAAT |
| 1393 | REF | TUBA1B | TUBA1B_2 | Forward primer | GACTCCTTCAACACCTTCTTC |
| 1394 | REF | TUBA1B | TUBA1B_2 | Probe | CCGGGCTGTGTTTGTAGACTTGGA |
| 1395 | REF | TUBA1B | TUBA1B_2 | Reverse primer | CCAGTGCGAACTTCATCAAT |
| 1396 | REF | TUBA1B | TUBA1B_3 | Forward primer | GGAGGAGATGACTCCTTCAA |
| 1397 | REF | TUBA1B | TUBA1B_3 | Probe | CTTCTTCAGTGAGACGGGCGCT |
| 1398 | REF | TUBA1B | TUBA1B_3 | Reverse primer | CATCAATGACTGTGGGTTCC |
| 1399 | REF | TUBA1B | TUBA1B_4 | Forward primer | CAGATGCCAAGTGACAAGA |
| 1400 | REF | TUBA1B | TUBA1B_4 | Probe | CTTCTTCAGTGAGACGGGCGCT |
| 1401 | REF | TUBA1B | TUBA1B_4 | Reverse primer | GGGTTCCAAGTCTACAAACA |

TABLE 9
Validation of selected representative primers and probes

| SEQ | | | | GC | | |
|---|---|---|---|---|---|---|
| ID NO: | Assay | Oligo | Strand | content | Length | Tm* |
| 223 | ABCC4_2 | Forward | Sense | 55.0% | 20 | 64.4 |
| | | primer | | | | |
| 224 | ABCC4_2 | Reverse | Anti- | 45.8% | 24 | 63.6 |
| | | Primer | sense | | | |
| 225 | ABCC4 2 | Probe | Sense | 53.8% | 26 | 69.8 |
| 84 | GREB1 2 | Forward | Sense | 45% | 20 | 61 |
| | | primer | | | | |
| 83 | GREB1 2 | Reverse | Anti- | 46% | 22 | 69 |
| | | Primer | sense | | | |
| 82 | GREB1 2 | Probe | Sense | 50% | 26 | 61 |
| 565 | GADD45A_2 | Forward | Sense | 36% | 22 | 52.2 |
| | | primer | | | | |
| 566 | GADD45A_2 | Reverse | Anti- | 50% | 20 | 53 |
| | | Primer | sense | | | |
| 567 | GADD45A 2 | Probe | Sense | 38% | 29 | 57.1 |

TABLE 10
Sets of primers and probes for determining the JAK-STAT1/2 cellular signaling
pathway activity.

| NO | | | | | |
|---|---|---|---|---|---|
| SEQ | | | | | |
| ID | Pathway | Gene | Assay | Oligo | Sequence |
| 1402 | STAT1/2 | APOL1 | APOL1_1 | Forward primer | GAGCACACAGAATCTGCTA |
| 1403 | STAT1/2 | APOL1 | APOL1_1 | Reverse Primer | AGGTTGTCCAGAGCTTTAC |
| 1404 | STAT1/2 | APOL1 | APOL1_1 | Probe | CGGATTCGTGGCTGCTGCTGAA |
| 1405 | STAT1/2 | APOL1 | APOL1_2 | Forward primer | ACACAGAATCTGCTACTCC |
| 1406 | STAT1/2 | APOL1 | APOL1_2 | Probe | CAGTTCAGCAGCAGCCACGAATCC |
| 1407 | STAT1/2 | APOL1 | APOL1_2 | Reverse Primer | AGCTCATCTGCCTCATTC |
| 1408 | STAT1/2 | APOL1 | APOL1_3 | Forward Primer | AGGCCTGGAACGGATTC |
| 1409 | STAT1/2 | APOL1 | APOL1_3 | Probe | AGCTCCGTAAAGCTCTGGACAACCT |
| 1410 | STAT1/2 | APOL1 | APOL1_3 | Reverse Primer | GTCTTTCATGATCATTTGTCTTGC |
| 1411 | STAT1/2 | APOL1 | APOL1_4 | Forward Primer | ACTCCTGCTGACTGATAATG |
| 1412 | STAT1/2 | APOL1 | APOL1_4 | Probe | CTCATTCCTGGGCAGTTCAGCAGC |
| 1413 | STAT1/2 | APOL1 | APOL1_4 | Reverse Primer | AGGTTGTCCAGAGCTTTAC |
| 1414 | STAT1/2 | BID | BID_1 | Forward Primer | CAGAACCTACGCACCTAC |
| 1415 | STAT1/2 | BID | BID_1 | Probe | CCGTTCAGTCCATCCCATTTCTGGC |
| 1416 | STAT1/2 | BID | BID_1 | Reverse Primer | TGACCACATCGAGCTTTAG |
| 1417 | STAT1/2 | BID | BID_2 | Forward Primer | CGTGATGTCTTTCACACAAC |
| 1418 | STAT1/2 | BID | BID_2 | Probe | CCGTTCAGTCCATCCCATTTCTGGC |
| 1419 | STAT1/2 | BID | BID_2 | Reverse Primer | TTAGCCAGTCACACTTCTG |
| 1420 | STAT1/2 | BID | BID_3 | PForward rimer | ACCTACGTGAGGAGCTTA |
| 1421 | STAT1/2 | BID | BID_3 | Probe | CCGTTCAGTCCATCCCATTTCTGGC |
| 1422 | STAT1/2 | BID | BID_3 | Reverse Primer | GCTATACAGCTGTGACCA |
| 1423 | STAT1/2 | BID | BID_4 | Forward Primer | CGTCCTTGCTCCGTGAT |
| 1424 | STAT1/2 | BID | BID_4 | Probe | AACCAGAACCTACGCACCTACGTGA |
| 1425 | STAT1/2 | BID | BID_4 | Reverse Primer | AACTGTCCGTTCAGTCCA |
| 1426 | STAT1/2 | CXCL9 | CXCL9_1 | Forward Primer | CATCTTGCTGGTTCTGATTG |
| 1427 | STAT1/2 | CXCL9 | CXCL9_1 | Probe | TTCCTGCATCAGCACCAACCAAGG |
| 1428 | STAT1/2 | CXCL9 | CXCL9_1 | Reverse Primer | CAAGGATTGTAGGTGGATAGT |
| 1429 | STAT1/2 | CXCL9 | CXCL9_2 | Forward Primer | TCTGATTGGAGTGCAAGG |
| 1430 | STAT1/2 | CXCL9 | CXCL9_2 | Probe | TTCCTGCATCAGCACCAACCAAGG |
| 1431 | STAT1/2 | CXCL9 | CXCL9_2 | Reverse Primer | AGGTCTTTCAAGGATTGTAGG |
| 1432 | STAT1/2 | CXCL9 | CXCL9_3 | Forward primer | AGGGACTATCCACCTACAA |
| 1433 | STAT1/2 | CXCL9 | CXCL9_3 | Reverse primer | GACATGTTTGAACTCCATTCT |
| 1434 | STAT1/2 | CXCL9 | CXCL9_3 | Probe | CCCAAGCCCTTCCTGCGAGAAA |
| 1435 | STAT1/2 | CXCL9 | CXCL9_4 | Forward Primer | GAAAGACCTTAAACAATTTGCC |
| 1436 | STAT1/2 | CXCL9 | CXCL9_4 | Probe | CCAAGCCCTTCCTGCGAGAAA |
| 1437 | STAT1/2 | CXCL9 | CXCL9_4 | Reverse Primer | TCAGTTCCTTCACATCTGC |
| 1438 | STAT1/2 | GBP1 | GBP1_1 | Forward Primer | GTTCAGAAGCTACAAGACCT |
| 1439 | STAT1/2 | GBP1 | GBP1_1 | Probe | TCTGCAGAATCTCTTCAGCCTGTATCCC |
| 1440 | STAT1/2 | GBP1 | GBP1_1 | Reverse Primer | AGTCATAGACTCCTTGGATTTC |
| 1441 | STAT1/2 | GBP1 | GBP1_2 | Forward Primer | GGCGGGAATTTATTCGAAAC |
| 1442 | STAT1/2 | GBP1 | GBP1_2 | Probe | TGTAGCTTCTGAACAAAGAGACGATAGCCC |
| 1443 | STAT1/2 | GBP1 | GBP1_2 | Reverse Primer | CCTTGGATTTCAAGTATGTCTG |
| 1444 | STAT1/2 | GBP1 | GBP1_3 | Forward Primer | AGCTACAAGACCTGAAGAAA |
| 1445 | STAT1/2 | GBP1 | GBP1_3 | Probe | AAGTACTATGAGGAACCGAGGAAGGG |
| 1446 | STAT1/2 | GBP1 | GBP1_3 | Reverse Primer | AGAATCTCTTCAGCCTGTATC |
| 1447 | STAT1/2 | GBP1 | GBP1_4 | Forward primer | ACTATGAGGAACCGAGGA |
| 1448 | STAT1/2 | GBP1 | GBP1_4 | Reverse primer | GGAGAATTGCATCAGTCATAG |
| 1449 | STAT1/2 | GBP1 | GBP1_4 | Probe | ATACAGGCTGAAGAGATTCTGCAGACAT |
| 1450 | STAT1/2 | GNAZ | GNAZ_1 | Forward primer | GAGGTGAAGGGCTGGAT |
| 1451 | STAT1/2 | GNAZ | GNAZ_1 | Probe | TCCAACCCTCCAGCCACTCA |
| 1452 | STAT1/2 | GNAZ | GNAZ_1 | Reverse Primer | GTTGCTGTGGCGATGTT |
| 1453 | STAT1/2 | GNAZ | GNAZ_2 | Forward primer | GCAGATGCTCTGTGCTG |
| 1454 | STAT1/2 | GNAZ | GNAZ_2 | Probe | ACTGTGCATCCAGCCCTTCACC |
| 1455 | STAT1/2 | GNAZ | GNAZ_2 | Reverse Primer | GGCGATGTTGCTGAGTG |
| 1456 | STAT1/2 | GNAZ | GNAZ_3 | Forward primer | CTGGATGCACAGTGGGA |
| 1457 | STAT1/2 | GNAZ | GNAZ_3 | Probe | TCCAGCCACTCAGCAACATCGC |
| 1458 | STAT1/2 | GNAZ | GNAZ_3 | Reverse Primer | GGTTGCTGGTTGCTGTG |
| 1459 | STAT1/2 | GNAZ | GNAZ_4 | Forward primer | CAACCCTCCAGCCACTC |
| 1460 | STAT1/2 | GNAZ | GNAZ_4 | Probe | ACAGCAACCAGCAACCAGACGG |
| 1461 | STAT1/2 | GNAZ | GNAZ_4 | Reverse Primer | CCGTCCGCTTGTGTTTG |
| 1462 | STAT1/2 | IFI6 | IFI6_1 | Forward primer | CTAGCCTCAAGTGATCCTC |
| 1463 | STAT1/2 | IF16 | IFI6_1 | Probe | ATCGTCGGCGCATGCTTGTAATCC |
| 1464 | STAT1/2 | IFI6 | IFI6_1 | Reverse Primer | GGGAGAGTGATAGACAAAGT |
| 1465 | STAT1/2 | IFI6 | IFI6_2 | Forward primer | CTAGAGTGCAGTGGCTATT |
| 1466 | STAT1/2 | IFI6 | IFI6_2 | Reverse primer | GGCGCATGCTTGTAATC |
| 1467 | STAT1/2 | IFI6 | IFI6_2 | Probe | TGCAGCCTCCAACTCCTAGCCT |
| 1468 | STAT1/2 | IFI6 | IFI6_3 | Forward primer | CCTCCCAAGTAGGATTACAAG |
| 1469 | STAT1/2 | IFI6 | IFI6_3 | Probe | CCGACGATGCCCAGAATCCAGAAC |
| 1470 | STAT1/2 | IFI6 | IFI6_3 | Reverse Primer | CTGGGTGAAGTTTATTCTGTTT |
| 1471 | STAT1/2 | IFI6 | IFI6_4 | Forward primer | CACTATATTGTCCAGGCTAGAG |
| 1472 | STAT1/2 | IFI6 | IFI6_4 | Probe | AGTACACTGCAGCCTCCAACTCCT |
| 1473 | STAT1/2 | IFI6 | IFI6_4 | Reverse Primer | GTTGAGACAGGAGGATCAC |
| 1474 | STAT1/2 | IFIT2 | IFIT2_1 | Forward primer | GAGTGCAGCTGCCTGAA |
| 1475 | STAT1/2 | IFIT2 | IFIT2_1 | Reverse primer | GGCTGCTCTCCAAGGAAT |
| 1476 | STAT1/2 | IFIT2 | IFIT2_1 | Probe | AATTCTCAGCTGTTCGGCAGGGC |
| 1477 | STAT1/2 | IFIT2 | IFIT2_2 | Forward primer | AGAGGAAGATTTCTGAAGAGTG |
| 1478 | STAT1/2 | IFIT2 | IFIT2_2 | Probe | TGCCGAACAGCTGAGAATTGCACT |
| 1479 | STAT1/2 | IFIT2 | IFIT2_2 | Reverse Primer | CTCCAAGGAATTCTTATTGTTCTC |
| 1480 | STAT1/2 | IFIT2 | IFIT2_3 | Forward primer | AACCATGAGTGAGAACAATAAG |
| 1481 | STAT1/2 | IFIT2 | IFIT2_3 | Probe | TGGAGAGCAGCCTACGGCAACTAA |
| 1482 | STAT1/2 | IFIT2 | IFIT2_3 | Reverse Primer | CACGATTCTGAAACTCAGTC |
| 1483 | STAT1/2 | IFIT2 | IFIT2_4 | Forward primer | GCCGAACAGCTGAGAAT |
| 1484 | STAT1/2 | IFIT2 | IFIT2_4 | Probe | TGGAGAGCAGCCTACGGCAACTAA |
| 1485 | STAT1/2 | IFIT2 | IFIT2_4 | Reverse Primer | CATCAAGTTCCAGGTGAAATG |
| 1486 | STAT1/2 | IFITM1 | IFITM1_1 | Forward primer | TTCATAGCATTCGCCTACTC |
| 1487 | STAT1/2 | IFITM1 | IFITM1_1 | Probe | TAGGGACAGGAAGATGGTTGGCGA |
| 1488 | STAT1/2 | IFITM1 | IFITM1_1 | Reverse Primer | AGATGTTCAGGCACTTGG |
| 1489 | STAT1/2 | IFITM1 | IFITM1_2 | Forward primer | ACACCCTCTTCTTGAACTG |
| 1490 | STAT1/2 | IFITM1 | IFITM1_2 | Probe | AGCATTCGCCTACTCCGTGAAGTCT |
| 1491 | STAT1/2 | IFITM1 | IFITM1_2 | Reverse Primer | GCCAACCATCTTCCTGT |
| 1492 | STAT1/2 | IFITM1 | IFITM1_3 | Forward primer | CTTCTTGAACTGGTGCTGTC |
| 1493 | STAT1/2 | IFITM1 | IFITM1_3 | Probe | TAGGGACAGGAAGATGGTTGGCGA |
| 1494 | STAT1/2 | IFITM1 | IFITM1_3 | Reverse Primer | AGGGCCCAGATGTTCAG |
| 1495 | STAT1/2 | IFITM1 | IFITM1_4 | Forward primer | CCTGTTCAACACCCTCTT |
| 1496 | STAT1/2 | IFITM1 | IFITM1_4 | Reverse primer | CTGTCCCTAGACTTCACG |
| 1497 | STAT1/2 | IFITM1 | IFITM1_4 | Probe | TCTGGGCTTCATAGCATTCGCCTACT |
| 1498 | STAT1/2 | IRF1 | IRF1_1 | Forward primer | TAAGAGCAAGGCCAAGAG |
| 1499 | STAT1/2 | IRF1 | IRF1_1 | Probe | TGATGGACTCAGCAGCTCCACTCT |
| 1500 | STAT1/2 | IRF1 | IRF1_1 | Reverse Primer | GTAGCCTGGAACTGTGTAG |
| 1501 | STAT1/2 | IRF1 | IRF1_2 | Forward primer | CCACCTCTCACCAAGAAC |
| 1502 | STAT1/2 | IRF1 | IRF1_2 | Reverse primer | GGTATCAGGGCTGGAATC |
| 1503 | STAT1/2 | IRF1 | IRF1_2 | Probe | AGTCGAAGTCCAGCCGAGATGCT |
| 1504 | STAT1/2 | IRF1 | IRF1_3 | Forward primer | ATGCTTCCACCTCTCAC |
| 1505 | STAT1/2 | IRF1 | IRF1_3 | Probe | AAGTCGAAGTCCAGCCGAGATGCT |
| 1506 | STAT1/2 | IRF1 | IRF1_3 | Reverse Primer | CCCACATGACTTCCTCTT |
| 1507 | STAT1/2 | IRF1 | IRF1_4 | Forward primer | AAAGACCAGAGCAGGAAC |
| 1508 | STAT1/2 | IRF1 | IRF1_4 | Probe | TGCTTCCACCTCTCACCAAGAACCA |
| 1509 | STAT1/2 | IRF1 | IRF1_4 | Reverse Primer | GCTGGACTTCGACTTTCT |
| 1510 | STAT1/2 | IRF7 | IRF7_1 | Forward primer | TACCATCTACCTGGGCTT |
| 1511 | STAT1/2 | IRF7 | IRF7_1 | Reverse primer | CAGGGTTCCAGCTTCAC |
| 1512 | STAT1/2 | IRF7 | IRF7_1 | Probe | CCAAGGAGAAGAGCCTGGTCCT |
| 1513 | STAT1/2 | IRF7 | IRF7_2 | Forward primer | AAGGAGAAGAGCCTGGT |
| 1514 | STAT1/2 | IRF7 | IRF7_2 | Probe | AGCGTGAGGGTGTGTCTTCCCT |
| 1515 | STAT1/2 | IRF7 | IRF7_2 | Reverse Primer | CTGAGGCTGCTGCTATC |
| 1516 | STAT1/2 | IRF7 | IRF7_3 | Forward primer | GAGGCCCAAGGAGAAGA |
| 1517 | STAT1/2 | IRF7 | IRF7_3 | Probe | ACAGCCAGGGTTCCAGCTTCAC |
| 1518 | STAT1/2 | IRF7 | IRF7_3 | Reverse Primer | TGCTGCTATCCAGGGAA |
| 1519 | STAT1/2 | IRF7 | IRF7_4 | Forward primer | CCCACGCTATACCATCTAC |
| 1520 | STAT1/2 | IRF7 | IRF7_4 | Probe | TTCACCAGGACCAGGCTCTTCTCC |
| 1521 | STAT1/2 | IRF7 | IRF7_4 | Reverse Primer | CTATCCAGGGAAGACACAC |
| 1522 | STAT1/2 | IRF9 | IRF9_1 | Forward primer | GAGCCCTACAAGGTGTATC |
| 1523 | STAT1/2 | IRF9 | IRF9_1 | Probe | CCAGCCAGGGACTCAGAAAGTACCA |
| 1524 | STAT1/2 | IRF9 | IRF9_1 | Reverse Primer | ATCCTCTTCCTCCTTCCT |
| 1525 | STAT1/2 | IRF9 | IRF9_2 | Forward primer | CGCATGGATGTTGCTGA |
| 1526 | STAT1/2 | IRF9 | IRF9_2 | Reverse primer | TGATGGTACTTTCTGAGTCC |
| 1527 | STAT1/2 | IRF9 | IRF9_2 | Probe | CAGTTGCTGCCACCAGGAATCGT |
| 1528 | STAT1/2 | IRF9 | IRF9_3 | Forward primer | CACCAGGAATCGTCTCTG |
| 1529 | STAT1/2 | IRF9 | IRF9_3 | Probe | CCAGCCAGGGACTCAGAAAGTACCA |
| 1530 | STAT1/2 | IRF9 | IRF9_3 | Reverse Primer | GACTGAGTGTGCAGTTCT |
| 1531 | STAT1/2 | IRF9 | IRF9_4 | Forward primer | ATGTTGCTGAGCCCTAC |
| 1532 | STAT1/2 | IRF9 | IRF9_4 | Probe | ATCAGTTGCTGCCACCAGGAATCG |
| 1533 | STAT1/2 | IRF9 | IRF9_4 | Reverse Primer | CGCTTTGATGGTACTTTCTG |
| 1534 | STAT1/2 | ISG15 | ISG15_1 | Forward primer | AATGCGACGAACCTCTG |
| 1535 | STAT1/2 | ISG15 | ISG15_1 | Probe | CTGCTGCGGCCCTTGTTATTCCTC |
| 1536 | STAT1/2 | ISG15 | ISG15_1 | Reverse Primer | TCACTTGCTGCTTCAGG |
| 1537 | STAT1/2 | ISG15 | ISG15_2 | Forward primer | TGGGACCTGACGGTGAA |
| 1538 | STAT1/2 | ISG15 | ISG15_2 | Reverse primer | CGATCTTCTGGGTGATCTG |
| 1539 | STAT1/2 | ISG15 | ISG15_2 | Probe | TTCCAGGTGTCCCTGAGCAGCT |
| 1540 | STAT1/2 | ISG15 | ISG15_3 | Forward primer | GCATCCTGGTGAGGAATAA |
| 1541 | STAT1/2 | ISG15 | ISG15_3 | Reverse primer | AGCCAGAACAGGTCGTC |
| 1542 | STAT1/2 | ISG15 | ISG15_2 | Probe | ACCTGAAGCAGCAAGTGAGCGG |
| 1543 | STAT1/2 | ISG15 | ISG15_4 | Forward primer | CAGATCACCCAGAAGATCG |
| 1544 | STAT1/2 | ISG15 | ISG15_4 | Probe | TTCCAGCAGCGTCTGGCTGT |
| 1545 | STAT1/2 | ISG15 | ISG15_4 | Reverse Primer | TTCGTCGCATTTGTCCA |
| 1546 | STAT1/2 | LY6E | LY6E_1 | Forward Primer | TGACTGTGTCTGCTAGTG |
| 1547 | STAT1/2 | LY6E | LY6E_1 | Probe | ACATTTGGCCACAGCCTGAGCA |
| 1548 | STAT1/2 | LY6E | LY6E_1 | Reverse Primer | CAACATTGACGCCTTCTG |
| 1549 | STAT1/2 | LY6E | LY6E_2 | Forward Primer | TCCGACCAGGACAACTA |
| 1550 | STAT1/2 | LY6E | LY6E_2 | Probe | ACATTTGGCCACAGCCTGAGCA |
| 1551 | STAT1/2 | LY6E | LY6E_2 | Reverse Primer | AGCCACACCAACATTGA |
| 1552 | STAT1/2 | LY6E | LY6E_3 | Forward Primer | GACAACTACTGCGTGACT |
| 1553 | STAT1/2 | LY6E | LY6E_3 | Probe | TCACGAGATTCCCAATGCCGGC |
| 1554 | STAT1/2 | LY6E | LY6E_3 | Reverse Primer | AACAGGTCTTGCTCAGG |
| 1555 | STAT1/2 | LY6E | LY6E_4 | Forward Primer | GCAATCTGTACTGCCTGAA |
| 1556 | STAT1/2 | LY6E | LY6E_4 | Probe | TCCGACCAGGACAACTACTGCGT |
| 1557 | STAT1/2 | LY6E | LY6E_4 | Reverse Primer | TGGCCAAATGTCACGAG |
| 1558 | STAT1/2 | OAS1 | OAS1_1 | Forward Primer | CCTGTGTGTGTGTCCAA |
| 1559 | STAT1/2 | OAS1 | OAS1_1 | Reverse primer | CCAGGTCAGCGTCAGAT |
| 1560 | STAT1/2 | OAS1 | OAS1_1 | Probe | AAAGGGTGGCTCCTCAGGCAAG |
| 1561 | STAT1/2 | OAS1 | OAS1_2 | Forward Primer | CCATGCCATTGACATCATC |
| 1562 | STAT1/2 | OAS1 | OAS1_2 | Probe | TCCTACCCTGTGTGTGTGTCCAAGG |
| 1563 | STAT1/2 | OAS1 | OAS1_2 | Reverse Primer | GAGGAGCCACCCTTTAC |
| 1564 | STAT1/2 | OAS1 | OAS1_3 | Forward Primer | TGACATCATCTGTGGGTTC |
| 1565 | STAT1/2 | OAS1 | OAS1_3 | Probe | AGGTGGTAAAGGGTGGCTCCTCA |
| 1566 | STAT1/2 | OAS1 | OAS1_3 | Reverse Primer | GGAAGACAACCAGGTCAG |
| 1567 | STAT1/2 | OAS1 | OAS1_4 | Forward Primer | TGTGTCCAAGGTGGTAAAG |
| 1568 | STAT1/2 | OAS1 | OAS1_4 | Probe | CGATCTGACGCTGACCTGGTTGTCT |
| 1569 | STAT1/2 | OAS1 | OAS1_4 | Reverse Primer | AAGTGGTGAGAGGACTGA |
| 1570 | STAT1/2 | PDCD1 | PDCD1_1 | Forward Primer | CCAGGATGGTTCTTAGACTC |
| 1571 | STAT1/2 | PDCD1 | PDCD1_1 | Probe | TGCAGGTGAAGGTGGCGTTGTC |
| 1572 | STAT1/2 | PDCD1 | PDCD1_1 | Reverse Primer | TCCGATGTGTTGGAGAAG |
| 1573 | STAT1/2 | PDCD1 | PDCD1_2 | Forward Primer | CCTGAGCAGTGGAGAAG |
| 1574 | STAT1/2 | PDCD1 | PDCD1_2 | Probe | TCGTCTGGGCGGTGCTACAACT |
| 1575 | STAT1/2 | PDCD1 | PDCD1_2 | Reverse Primer | GGAGTCTAAGAACCATCCTG |
| 1576 | STAT1/2 | PDCD1 | PDCD1_3 | Forward Primer | TCCAGGCATGCAGATCC |
| 1577 | STAT1/2 | PDCD1 | PDCD1_3 | Probe | TCGTCTGGGCGGTGCTACAACT |
| 1578 | STAT1/2 | PDCD1 | PDCD1_3 | Reverse Primer | GTTCCAGGGCCTGTCTG |
| 1579 | STAT1/2 | PDCD1 | PDCD1_4 | Forward Primer | TCTGGGCGGTGCTACAA |
| 1580 | STAT1/2 | PDCD1 | PDCD1_4 | Probe | TGGCGGCCAGGATGGTTCTTAGA |
| 1581 | STAT1/2 | PDCD1 | PDCD1_4 | Reverse Primer | TGGAGAAGCTGCAGGTGAA |
| 1582 | STAT1/2 | RFPL3 | RFPL3_1 | Forward Primer | CTACAGATGAACCCAAGGAT |
| 1583 | STAT1/2 | RFPL3 | RFPL3_1 | Probe | TGGATGCCGACACAGCCAACAA |
| 1584 | STAT1/2 | RFPL3 | RFPL3_1 | Reverse Primer | TGAGGTCGTCAGAAATGAG |
| 1585 | STAT1/2 | RFPL3 | RFPL3_2 | Forward Primer | GAGCCCAAGCTGAAGAA |
| 1586 | STAT1/2 | RFPL3 | RFPL3_2 | Probe | TTGGATGCCGACACAGCCAACA |
| 1587 | STAT1/2 | RFPL3 | RFPL3_2 | Reverse Primer | GTCAGAAATGAGGAGGAAGT |
| 1588 | STAT1/2 | RFPL3 | RFPL3_3 | Forward Primer | GCTGGTTTCCCACATCAA |
| 1589 | STAT1/2 | RFPL3 | RFPL3_3 | Probe | ACAGATGAACCCAAGGATGCGGAA |
| 1590 | STAT1/2 | RFPL3 | RFPL3_3 | Reverse Primer | CGGCATCCAAGGTCATATC |
| 1591 | STAT1/2 | RFPL3 | RFPL3_4 | Forward Primer | GCTAGAGAGGCTGGTTTC |
| 1592 | STAT1/2 | RFPL3 | RFPL3_4 | Probe | ACAGATGAACCCAAGGATGCGGAA |
| 1593 | STAT1/2 | RFPL3 | RFPL3_4 | Reverse Primer | AGGTCATATCCACTTGGAAC |
| 1594 | STAT1/2 | SSTR3 | SSTR3_1 | Forward Primer | CACAGGATTCCAGCTCTAAA |
| 1595 | STAT1/2 | SSTR3 | SSTR3_1 | Probe | ATAGCTGACTGCTGACCACCCTCC |
| 1596 | STAT1/2 | SSTR3 | SSTR3_1 | Reverse Primer | GACACCGATGATGGATGAA |
| 1597 | STAT1/2 | SSTR3 | SSTR3_2 | Forward Primer | AAAGACGGCACCTCAAT |
| 1598 | STAT1/2 | SSTR3 | SSTR3_2 | Probe | TCCATCATCGGTGTCCACGACCT |
| 1599 | STAT1/2 | SSTR3 | SSTR3_2 | Reverse Primer | AGGCATTCTCAGGTTCTG |
| 1600 | STAT1/2 | SSTR3 | SSTR3_3 | Forward Primer | AAAGTCCCTCCTTCTCAAG |
| 1601 | STAT1/2 | SSTR3 | SSTR3_3 | Probe | ATAGCTGACTGCTGACCACCCTCC |
| 1602 | STAT1/2 | SSTR3 | SSTR3_3 | Reverse Primer | GATGATGGATGAAGCATGTC |
| 1603 | STAT1/2 | SSTR3 | SSTR3_4 | Forward Primer | CAGGCAAGCTTGTGCCA |
| 1604 | STAT1/2 | SSTR3 | SSTR3_4 | Probe | ACGGCACCTCAATTGCAGGCAA |
| 1605 | STAT1/2 | SSTR3 | SSTR3_4 | Reverse Primer | AGGGTGGTCAGCAGTCA |
| 1606 | STAT1/2 | STAT1 | STAT1_1 | Forward Primer | CTGGCACAGTGGTTAGAA |
| 1607 | STAT1/2 | STAT1 | STAT1_1 | Probe | AAGCAAGACTGGGAGCACGCTG |
| 1608 | STAT1/2 | STAT1 | STAT1 | Reverse Primer | GTGACAGGAGGTCATGAAA |
| 1609 | STAT1/2 | STAT1 | STAT1_2 | Forward Primer | CAGGTTCACCAGCTTTATGA |
| 1610 | STAT1/2 | STAT1 | STAT1_2 | Probe | AAAGCAAGACTGGGAGCACGCT |
| 1611 | STAT1/2 | STAT1 | STAT1_2 | Reverse Primer | AAACGGATGGTGGCAAA |
| 1612 | STAT1/2 | STAT1 | STAT1_3 | Forward Primer | AATCAGACAGTACCTGGCA |
| 1613 | STAT1/2 | STAT1 | STAT1_3 | Probe | AAAGCAAGACTGGGAGCACGCT |
| 1614 | STAT1/2 | STAT1 | STAT1_3 | Reverse Primer | CAGCTGTGACAGGAGGT |
| 1615 | STAT1/2 | STAT1 | STAT1_4 | Forward Primer | ACCAGCTTTATGATGACAGTT |
| 1616 | STAT1/2 | STAT1 | STAT1_4 | Reverse Primer | GATGGTGGCAAATGAAACA |
| 1617 | STAT1/2 | STAT1 | STAT1_4 | Probe | AAGCAAGACTGGGAGCACGCTG |
| 1618 | STAT1/2 | TAP1 | TAP1_1 | Forward Primer | GATGCAAACAGCCAGTTAC |
| 1619 | STAT1/2 | TAP1 | TAP1_1 | Probe | TGTACGAAAGCCCTGAGCGGTACT |
| 1620 | STAT1/2 | TAP1 | TAP1_1 | Reverse Primer | GGTGATGAGAAGCACTGA |
| 1621 | STAT1/2 | TAP1 | TAP1_2 | Forward Primer | CGGAAACCGTGTGTACTT |
| 1622 | STAT1/2 | TAP1 | TAP1_2 | Probe | ACCAGTGCCCTGGATGCAAACA |
| 1623 | STAT1/2 | TAP1 | TAP1_2 | Reverse Primer | CTCAGGGCTTTCGTACAG |
| 1624 | STAT1/2 | TAP1 | TAP1_3 | Forward Primer | TGCCCTGCTGCAGAATC |
| 1625 | STAT1/2 | TAP1 | TAP1_3 | Reverse Primer | AGACTTCTTCCAAATACCTGTGG |
| 1626 | STAT1/2 | TAP1 | TAP1_3 | Probe | ATGAGCACCGCTACCTGCACAG |
| 1627 | STAT1/2 | TAP1 | TAP1_4 | Forward Primer | GTGCCCTGGATGCAAAC |
| 1628 | STAT1/2 | TAP1 | TAP1_4 | Probe | AGGGCTTTCGTACAGGAGCTGC |
| 1629 | STAT1/2 | TAP1 | TAP1_4 | Reverse Primer | CACTGAGCGGGAGTACC |
| 1630 | STAT1/2 | USP18 | USP18_1 | Forward Primer | AGCGAGAGTCTTGTGATG |
| 1631 | STAT1/2 | USP18 | USP18_1 | Probe | TTTGCTGTGATTGCGCACGTGG |
| 1632 | STAT1/2 | USP18 | USP18_1 | Reverse Primer | CCGGATGTAGACACAGTAAT |
| 1633 | STAT1/2 | USP18 | USP18_2 | Forward Primer | AGCCAGATCCTTCCAATG |
| 1634 | STAT1/2 | USP18 | USP18_2 | Reverse Primer | TTCCCACGTGCGCAATC |
| 1635 | STAT1/2 | USP18 | USP18_2 | Probe | TGTGATGCTGAGGAGCAGTCTGGA |
| 1636 | STAT1/2 | USP18 | USP18_3 | Forward Primer | CCTTCCAATGAAGCGAGA |
| 1637 | STAT1/2 | USP18 | USP18_3 | Probe | TGTGATGCTGAGGAGCAGTCTGGA |
| 1638 | STAT1/2 | USP18 | USP18_3 | Reverse Primer | CAGTAATGACCGGAGTCTG |
| 1639 | STAT1/2 | USP18 | USP18_4 | Forward Primer | TTTCAGCCAGATCCTTCC |
| 1640 | STAT1/2 | USP18 | USP18_4 | Probe | TGGGAATGGCAGACTCCGGTCA |
| 1641 | STAT1/2 | USP18 | USP18_4 | Reverse Primer | TCCATCCACAGCATTCC |

TABLE 11
Sets of primers and probes for determining the JAK-STAT1/2 cellular
signaling pathway activity.

| NO | | | | | |
|---|---|---|---|---|---|
| SEQ | | | | | |
| ID | Pathway | Gene | Assay | Oligo | Sequence |
| 1642 | NFkB | BIRC3 | BIRC3_1 | Forward primer | CTAGTCAATGATCTTGTGTTAGAC |
| 1643 | NFkB | BIRC3 | BIRC3_1 | Reverse Primer | GGATTGGAATTACACAAGTCAA |
| 1644 | NFkB | BIRC3 | BIRC3_1 | Probe | AGGGAAGAGGAGAGAGAAAGAGCAACTG |
| 1645 | NFkB | BIRC3 | BIRC3_4 | Forward primer | CCTGGAGAAGACCATTCAG |
| 1646 | NFkB | BIRC3 | BIRC3_4 | Probe | ATGCTGCCGTGGAAATGGGCTTTA |
| 1647 | NFkB | BIRC3 | BIRC3_4 | Reverse Primer | CTCTCCAGTTGCTAGGATTT |
| 1648 | NFkB | BIRC3 | BIRC3_2 | Forward primer | CTGCTATCCACATCAGACA |
| 1649 | NFkB | BIRC3 | BIRC3_2 | Probe | ACCTGGAGAAGACCATTCAGAAGATGCA |
| 1650 | NFkB | BIRC3 | BIRC3_2 | Reverse Primer | CACGGCAGCATTAATCAC |
| 1651 | NFkB | BIRC3 | BIRC3_3 | Forward primer | GACTTACTCAATGCAGAAGATG |
| 1652 | NFkB | BIRC3 | BIRC3_3 | Probe | AGGGAAGAGGAGAGAGAAAGAGCAACTG |
| 1653 | NFkB | BIRC3 | BIRC3_3 | Reverse Primer | CCAGGATTGGAATTACACAAG |
| 1654 | NFkB | CCL20 | CCL20_4 | Forward primer | GTGACATCAATGCTATCATCTT |
| 1655 | NFkB | CCL20 | CCL20_4 | Reverse Primer | AGGAGACGCACAATATATTTCA |
| 1656 | NFkB | CCL20 | CCL20_4 | Probe | AGTTGTCTGTGTGCGCAAATCCA |
| 1657 | NFkB | CCL20 | CCL20_1 | Forward primer | CAGACCGTATTCTTCATCCT |
| 1658 | NFkB | CCL20 | CCL20_1 | Probe | TTATTGTGGGCTTCACACGGCAGC |
| 1659 | NFkB | CCL20 | CCL20_1 | Reverse Primer | GATTTGCGCACACAGAC |
| 1660 | NFkB | CCL20 | CCL20_2 | Forward primer | TGCTATCATCTTTCACACAAAG |
| 1661 | NFkB | CCL20 | CCL20_2 | Probe | AGTTGTCTGTGTGCGCAAATCCAA |
| 1662 | NFkB | CCL20 | CCL20_2 | Reverse Primer | TGTCCAATTCCATTCCAGA |
| 1663 | NFkB | CCL20 | CCL20_3 | Forward primer | TATTGTGGGCTTCACACG |
| 1664 | NFkB | CCL20 | CCL20_3 | Probe | TGGCCAATGAAGGCTGTGACATCA |
| 1665 | NFkB | CCL20 | CCL20_3 | Reverse Primer | CGCACAATATATTTCACCCAAG |
| 1666 | NFkB | CCL3 | CCL3_3 | Forward primer | CCACAGAATTTCATAGCTGAC |
| 1667 | NFkB | CCL3 | CCL3_3 | Reverse Primer | GCTTGGTTAGGAAGATGACAC |
| 1668 | NFkB | CCL3 | CCL3_3 | Probe | ACTTTGAGACGAGCAGCCAGTGC |
| 1669 | NFkB | CCL3 | CCL3_1 | Forward primer | GACTACTTTGAGACGAGCA |
| 1670 | NFkB | CCL3 | CCL3_1 | Probe | AGCCCGGTGTCATCTTCCTAACCA |
| 1671 | NFkB | CCL3 | CCL3_1 | Reverse Primer | CCAGGTCGCTGACATATT |
| 1672 | NFkB | CCL3 | CCL3_2 | Forward primer | CGGCAGATTCCACAGAA |
| 1673 | NFkB | CCL3 | CCL3_2 | Probe | TGACTACTTTGAGACGAGCAGCCAGT |
| 1674 | NFkB | CCL3 | CCL3_2 | Reverse Primer | GCTTCGCTTGGTTAGGA |
| 1675 | NFkB | CCL3 | CCL3_4 | Forward Primer | GCAGATTCCACAGAATTTCATAG |
| 1676 | NFkB | CCL3 | CCL3_4 | Probe | AGCCCGGTGTCATCTTCCTAACCA |
| 1677 | NFkB | CCL3 | CCL3_4 | Reverse Primer | GACCCACTCCTCACTGG |
| 1678 | NFkB | CCL4 | CCL4_4 | Forward Primer | TCGCAACTTTGTGGTAGAT |
| 1679 | NFkB | CCL4 | CCL4_4 | Reverse primer | GATTCACTGGGATCAGCAC |
| 1680 | NFkB | CCL4 | CCL4_4 | Probe | TCCCAGCCAGCTGTGGTATTCCA |
| 1681 | NFkB | CCL4 | CCL4_1 | Forward Primer | TGTCCTGTCTCTCCTCAT |
| 1682 | NFkB | CCL4 | CCL4_1 | Probe | TAGTAGCTGCCTTCTGCTCTCCAGC |
| 1683 | NFkB | CCL4 | CCL4_1 | Reverse Primer | CCTCGCGGTGTAAGAAA |
| 1684 | NFkB | CCL4 | CCL4_2 | Forward Primer | AGCTTCCTCGCAACTTT |
| 1685 | NFkB | CCL4 | CCL4_2 | Probe | CAGCCAGCTGTGGTATTCCAAACCAA |
| 1686 | NFkB | CCL4 | CCL4_2 | Reverse Primer | ACAGACTTGCTTGCTTCT |
| 1687 | NFkB | CCL4 | CCL4_3 | Forward Primer | GCTAGTAGCTGCCTTCTG |
| 1688 | NFkB | CCL4 | CCL4_3 | Probe | ACCACAAAGTTGCGAGGAAGCTTCC |
| 1689 | NFkB | CCL4 | CCL4_3 | Reverse Primer | GCTGCTGGTCTCATAGTAAT |
| 1690 | NFkB | CCL5 | CCL5_2 | Forward Primer | CTGTCATCCTCATTGCTACT |
| 1691 | NFkB | CCL5 | CCL5_2 | Reverse primer | GCCACTGGTGTAGAAATACT |
| 1692 | NFkB | CCL5 | CCL5_2 | Probe | TCGGACACCACACCCTGCTGCT |
| 1693 | NFkB | CCL5 | CCL5_1 | Forward Primer | ATTGCTACTGCCCTCTG |
| 1694 | NFkB | CCL5 | CCL5_1 | Reverse primer | GCCACTGGTGTAGAAATACT |
| 1695 | NFkB | CCL5 | CCL5_1 | Probe | TCGGACACCACACCCTGCTGCT |
| 1696 | NFkB | CCL5 | CCL5_4 | Forward Primer | CTCGCTGTCATCCTCATT |
| 1697 | NFkB | CCL5 | CCL5_4 | Probe | ACACCCTGCTGCTTTGCCTACATT |
| 1698 | NFkB | CCL5 | CCL5_4 | Reverse Primer | CTTGCCACTGGTGTAGAA |
| 1699 | NFkB | CCL5 | CCL5_3 | Forward Primer | TCTGCGCTCCTGCATCT |
| 1700 | NFkB | CCL5 | CCL5_3 | Probe | CCATATTCCTCGGACACCACACCCT |
| 1701 | NFkB | CCL5 | CCL5_3 | Reverse Primer | AGTGGGCGGGCAATGTA |
| 1702 | NFkB | CXCL2 | CXCL2_1 | Forward Primer | CATCGCCCATGGTTAAGA |
| 1703 | NFkB | CXCL2 | CXCL2_1 | Probe | TGGCAAATCCAACTGACCAGAAGG |
| 1704 | NFkB | CXCL2 | CXCL2_1 | Reverse Primer | CAGGAACAGCCACCAATA |
| 1705 | NFkB | CXCL2 | CXCL2_2 | Forward Primer | AATGGCAAATCCAACTGAC |
| 1706 | NFkB | CXCL2 | CXCL2_2 | Probe | CCTTCAGGAACAGCCACCAATAAGC |
| 1707 | NFkB | CXCL2 | CXCL2_2 | Reverse Primer | CTGTGTCTCTCTTTCCTCTT |
| 1708 | NFkB | CXCL2 | CXCL2_3 | Forward Primer | CTCAAGAATGGGCAGAAAG |
| 1709 | NFkB | CXCL2 | CXCL2_3 | Probe | CCGCATCGCCCATGGTTAAGAAA |
| 1710 | NFkB | CXCL2 | CXCL2_3 | Reverse Primer | CTTCTGGTCAGTTGGATTTG |
| 1711 | NFkB | CXCL2 | CXCL2_4 | Forward Primer | GCAGAAAGCTTGTCTCAAC |
| 1712 | NFkB | CXCL2 | CXCL2_4 | Probe | CCGCATCGCCCATGGTTAAGAAA |
| 1713 | NFkB | CXCL2 | CXCL2_4 | Reverse Primer | GCTTCCTCCTTCCTTCTG |
| 1714 | NFkB | ICAM1 | ICAM1_3 | Forward Primer | GCTGACGTGTGCAGTAATA |
| 1715 | NFkB | ICAM1 | ICAM1_3 | Reverse primer | CTGGCTTCGTCAGAATCA |
| 1716 | NFkB | ICAM1 | ICAM1_3 | Probe | ACCAGAGCCAGGAGACACTGCA |
| 1717 | NFkB | ICAM1 | ICAM1_1 | Forward Primer | CTGCAGACAGTGACCATCTA |
| 1718 | NFkB | ICAM1 | ICAM1__1 | Probe | AAGGGACCGAGGTGACAGTGAAGT |
| 1719 | NFkB | ICAM1 | ICAM1_1 | Reverse Primer | GCGTCACCTTGGCTCTA |
| 1720 | NFkB | ICAM1 | ICAM1_2 | Forward Primer | AGGAGACACTGCAGACA |
| 1721 | NFkB | ICAM1 | ICAM1_2 | Probe | CGCCGGAAAGCTGTAGATGGTCAC |
| 1722 | NFkB | ICAM1 | ICAM1_2 | Reverse Primer | TTCTGAGACCTCTGGCTT |
| 1723 | NFkB | ICAM1 | ICAM1_4 | Forward Primer | GAACCAGAGCCAGGAGA |
| 1724 | NFkB | ICAM1 | ICAM1_4 | Probe | CGCCCAACGTGATTCTGACGAAGC |
| 1725 | NFkB | ICAM1 | ICAM1_4 | Reverse Primer | TCGGTCCCTTCTGAGAC |
| 1726 | NFkB | IL6 | IL6_1 | Forward Primer | CCTTCCAAAGATGGCTGAA |
| 1727 | NFkB | IL6 | IL6_1 | Probe | TCAATGAGGAGACTTGCCTGGTGA |
| 1728 | NFkB | IL6 | IL6_1 | Reverse Primer | TGTTCCTCACTACTCTCAAATC |
| 1729 | NFkB | IL6 | IL6_2 | Forward Primer | GATGGATGCTTCCAATCTG |
| 1730 | NFkB | IL6 | IL6_2 | Probe | TCAATGAGGAGACTTGCCTGGTGA |
| 1731 | NFkB | IL6 | IL6_2 | Reverse Primer | AAATCTGTTCTGGAGGTACT |
| 1732 | NFkB | IL6 | IL6_3 | Forward Primer | CTTCCAATCTGGATTCAATGAG |
| 1733 | NFkB | IL6 | IL6_3 | Probe | TGAGAGTAGTGAGGAACAAGCCAGA |
| 1734 | NFkB | IL6 | IL6_3 | Reverse Primer | TGTACTCATCTGCACAGC |
| 1735 | NFkB | IL6 | IL6_4 | Forward Primer | CAGCAAAGAGGCACTGG |
| 1736 | NFkB | IL6 | IL6_4 | Probe | ACAACCTGAACCTTCCAAAGATGGC |
| 1737 | NFkB | IL6 | IL6_4 | Reverse Primer | TGAATCCAGATTGGAAGCAT |
| 1738 | NFkB | IRF1 | IRF1_1 | Forward Primer | CCACCTCTCACCAAGAAC |
| 1739 | NFkB | IRF1 | IRF1_1 | Probe | GGTATCAGGGCTGGAATC |
| 1740 | NFkB | IRF1 | IRF1_1 | Reverse Primer | AGTCGAAGTCCAGCCGAGATGCT |
| 1741 | NFkB | IRF1 | IRF1_4 | Forward Primer | TAAGAGCAAGGCCAAGAG |
| 1742 | NFkB | IRF1 | IRF1_4 | Probe | TGATGGACTCAGCAGCTCCACTCT |
| 1743 | NFkB | IRF1 | IRF1_4 | Reverse Primer | GTAGCCTGGAACTGTGTAG |
| 1744 | NFkB | IRF1 | IRF1_2 | Forward Primer | AAAGACCAGAGCAGGAAC |
| 1745 | NFkB | IRF1 | IRF1_2 | Probe | TGCTTCCACCTCTCACCAAGAACCA |
| 1746 | NFkB | IRF1 | IRF1_2 | Reverse Primer | GCTGGACTTCGACTTTCT |
| 1747 | NFkB | IRF1 | IRF1_3 | Forward Primer | ATGCTTCCACCTCTCAC |
| 1748 | NFkB | IRF1 | IRF1_3 | Probe | AAGTCGAAGTCCAGCCGAGATGCT |
| 1749 | NFkB | IRF1 | IRF1_3 | Reverse Primer | CCCACATGACTTCCTCTT |
| 1750 | NFkB | MMP9 | MMP9_4 | Forward Primer | GGAGACCTGAGAACCAATC |
| 1751 | NFkB | MMP9 | MMP9_4 | Probe | GACTCTCCACGCATCTCTG |
| 1752 | NFkB | MMP9 | MMP9_4 | Reverse Primer | AGGCAGCTGGCAGAGGAATACCT |
| 1753 | NFkB | MMP9 | MMP9_1 | Forward Primer | TGGAGACCTGAGAACCAATC |
| 1754 | NFkB | MMP9 | MMP9_1 | Probe | ACCCGAGTGTAACCATAGC |
| 1755 | NFkB | MMP9 | MMP9_1 | Reverse Primer | AGGCAGCTGGCAGAGGAATACCT |
| 1756 | NFkB | MMP9 | MMP9_2 | Forward Primer | TCCACCCTTGTGCTCTT |
| 1757 | NFkB | MMP9 | MMP9_2 | Probe | ACTCTCCACGCATCTCTG |
| 1758 | NFkB | MMP9 | MMP9_2 | Reverse Primer | AACCAATCTCACCGACAGGCAGC |
| 1759 | NFkB | MMP9 | MMP9_3 | Forward Primer | AGAACCAATCTCACCGACAG |
| 1760 | NFkB | MMP9 | MMP9_3 | Probe | CCAGAGATTTCGACTCTCCAC |
| 1761 | NFkB | MMP9 | MMP9_3 | Reverse Primer | TGGTTACACTCGGGTGGCAGAGA |
| 1762 | NFkB | NFkB2 | NFkB2_2 | Forward Primer | AATGGATGGCAGGCCTTT |
| 1763 | NFkB | NFkB2 | NFkB2_2 | Reverse primer | CGCTCAATCTTCATCTTGTG |
| 1764 | NFkB | NFkB2 | NFkB2_2 | Probe | TGCCATTGTGTTCCGGACACCC |
| 1765 | NFkB | NFkB2 | NFkB2_1 | Forward Primer | TCCCACAGATGTGCATAAA |
| 1766 | NFkB | NFkB2 | NFkB2_1 | Probe | TATGCCATTGTGTTCCGGACACCC |
| 1767 | NFkB | NFkB2 | NFkB2_1 | Reverse Primer | TTACAGGCCGCTCAATC |
| 1768 | NFkB | NFkB2 | NFkB2_4 | Forward Primer | CGGTTCTATGAGGATGATGA |
| 1769 | NFkB | NFkB2 | NFkB2_4 | Probe | TGCACATCTGTGGGAGAGAAGTCCC |
| 1770 | NFkB | NFkB2 | NFkB2_4 | Reverse Primer | CGGAACACAATGGCATAC |
| 1771 | NFkB | NFkB2 | NFkB2_3 | Forward Primer | AGGATGATGAGAATGGATGG |
| 1772 | NFkB | NFkB2 | NFkB2_3 | Probe | CACAAGATGAAGATTGAGCGGCCTGT |
| 1773 | NFkB | NFkB2 | NFkB2_3 | Reverse Primer | GTTTCAGTTGCAGAAACACT |
| 1774 | NFkB | PTGS2 | PTGS2_1 | Forward Primer | TGTGTTGACATCCAGATCAC |
| 1775 | NFkB | PTGS2 | PTGS2_1 | Probe | TAGGAGAGGTTAGAGAAGGC |
| 1776 | NFkB | PTGS2 | PTGS2_1 | Reverse Primer | CCACCAACTTACAATGCTGACTATGGCT |
| 1777 | NFkB | PTGS2 | PTGS2_4 | Forward Primer | TTGACAGTCCACCAACTTAC |
| 1778 | NFkB | PTGS2 | PTGS2_4 | Probe | GGAGGAAGGGCTCTAGTATAA |
| 1779 | NFkB | PTGS2 | PTGS2_4 | Reverse Primer | AAGCTGGGAAGCCTTCTCTAACCTCT |
| 1780 | NFkB | PTGS2 | PTGS2_2 | Forward Primer | GTGAATAACATTCCCTTCCTTC |
| 1781 | NFkB | PTGS2 | PTGS2_2 | Probe | TAGCCATAGTCAGCATTGTAA |
| 1782 | NFkB | PTGS2 | PTGS2_2 | Reverse Primer | CCAGATCACATTTGATTGACAGTCCACCA |
| 1783 | NFkB | PTGS2 | PTGS2_3 | Forward Primer | CCAACTTACAATGCTGACTATG |
| 1784 | NFkB | PTGS2 | PTGS2_3 | Probe | CAATCATCAGGCACAGGAG |
| 1785 | NFkB | PTGS2 | PTGS2_3 | Reverse Primer | AAGCTGGGAAGCCTTCTCTAACCTCT |
| 1786 | NFkB | TNF | TNF_3 | Forward Primer | TGCACTTTGGAGTGATCG |
| 1787 | NFkB | TNF | TNF_3 | Reverse primer | GGTTCGAGAAGATGATCTGAC |
| 1788 | NFkB | TNF | TNF_3 | Probe | AGGGACCTCTCTCTAATCAGCCCTCT |
| 1789 | NFkB | TNF | TNF_1 | Forward Primer | AGCCTCTTCTCCTTCCT |
| 1790 | NFkB | TNF | TNF_1 | Probe | TTCTGCCTGCTGCACTTTGGAGTG |
| 1791 | NFkB | TNF | TNF_1 | Reverse Primer | AGAGGGCTGATTAGAGAGA |
| 1792 | NFkB | TNF | TNF_2 | Forward Primer | TCAGATCATCTTCTCGAACC |
| 1793 | NFkB | TNF | TNF_2 | Probe | AGCCCATGTTGTAGCAAACCCTCA |
| 1794 | NFkB | TNF | TNF_2 | Reverse Primer | GGTTATCTCTCAGCTCCAC |
| 1795 | NFkB | TNF | TNF_4 | Forward Primer | CTGCCTGCTGCACTTTG |
| 1796 | NFkB | TNF | TNF_4 | Probe | AGGGACCTCTCTCTAATCAGCCCTCT |
| 1797 | NFkB | TNF | TNF_4 | Reverse Primer | GGCTACAGGCTTGTCACT |
| 1798 | NFkB | TNIP1 | TNIP1_4 | Forward primer | AGAGGAGCTAGTGAAGGA |
| 1799 | NFkB | TNIP1 | TNIP1_4 | Reverse primer | CTGTGACATTTGAGTCCTTTC |
| 1800 | NFkB | TNIP1 | TNIP1_4 | Probe | TCCCACCACCTTCTCCCTCCTT |
| 1801 | NFkB | TNIP1 | TNIP1_1 | Forward primer | AGAGGAGCTAGTGAAGGA |
| 1802 | NFkB | TNIP1 | TNIP1_1 | Probe | TCCCACCACCTTCTCCCTCCTT |
| 1803 | NFkB | TNIP1 | TNIP1_1 | Reverse Primer | GAGATGCTGTGACATTTGAG |
| 1804 | NFkB | TNIP1 | TNIP1_2 | Forward primer | GCTAGTGAAGGACAACGAG |
| 1805 | NFkB | TNIP1 | TNIP1_2 | Probe | TCCCACCACCTTCTCCCTCCTT |
| 1806 | NFkB | TNIP1 | TNIP1_2 | Reverse Primer | CTGTGGGAGATGCTGTG |
| 1807 | NFkB | TNIP1 | TNIP1_3 | Forward primer | CCACCTTCTCCCTCCTT |
| 1808 | NFkB | TNIP1 | TNIP1_3 | Probe | AAATGTCACAGCATCTCCCACAGCC |
| 1809 | NFkB | TNIP1 | TNIP1_3 | Reverse Primer | GTGCTGGCTTGTCACTG |
| 1810 | NFkB | TRAF | TRAF_3 | Forward primer | CTGGAAAGAGAACCCATCTG |
| 1811 | NFkB | TRAF | TRAF_3 | Reverse primer | CTCACGGTTGTTCTGGT |
| 1812 | NFkB | TRAF | TRAF_3 | Probe | AGTATGATGCGCTGCTGCCG |
| 1813 | NFkB | TRAF | TRAF_2 | Forward primer | AACCCATCTGTCGCTCT |
| 1814 | NFkB | TRAF | TRAF_2 | Reverse primer | CTCACGGTTGTTCTGGT |
| 1815 | NFkB | TRAF | TRAF_2 | Probe | AGTATGATGCGCTGCTGCCG |
| 1816 | NFkB | TRAF | TRAF_1 | Forward primer | CTGAGCTTGGAGCAGAG |
| 1817 | NFkB | TRAF | TRAF_1 | Probe | CAGGAAAGTGCCATCGAAGGAGGC |
| 1818 | NFkB | TRAF | TRAF_1 | Reverse Primer | TGGTGACATTGGTGATCTT |
| 1819 | NFkB | TRAF | TRAF_4 | Forward primer | CCCTGGCCACCTCTATC |
| 1820 | NFkB | TRAF | TRAF_4 | Probe | ATCCTGAGCTTGGAGCAGAGGGT |
| 1821 | NFkB | TRAF | TRAF_4 | Reverse Primer | CAGGGCCTGGTCTTTCT |
| 1822 | NFkB | VCAM1 | VCAM1_1 | Forward primer | CTGGAAGAAGCAGAAAGGA |
| 1823 | NFkB | VCAM1 | VCAM1_1 | Probe | CTGAGAGTGTCAAAGAAGGAGACACTGT |
| 1824 | NFkB | VCAM1 | VCAM1_1 | Reverse Primer | TTCCACATGTACAAGAGATGA |
| 1825 | NFkB | VCAM1 | VCAM1_2 | Forward primer | TTTCCTTCTGAGAGTGTCAA |
| 1826 | NFkB | VCAM1 | VCAM1_2 | Probe | ACACTGTCATCATCTCTTGTACATGTGGA |
| 1827 | NFkB | VCAM1 | VCAM1_2 | Reverse Primer | TAGTACTGTGTCTCCTGTCT |
| 1828 | NFkB | VCAM1 | VCAM1_3 | Forward primer | TGAAGGAATTAACCAGGCT |
| 1829 | NFkB | VCAM1 | VCAM1_3 | Probe | TTCCACTTCCTTTCTGCTTCTTCC |
| 1830 | NFkB | VCAM1 | VCAM1_3 | Reverse Primer | CTTTGACACTCTCAGAAGGA |
| 1831 | NFkB | VCAM1 | VCAM1_4 | Forward primer | GCAGAAAGGAAGTGGAATTA |
| 1832 | NFkB | VCAM1 | VCAM1_4 | Probe | TCCTTCTGAGAGTGTCAAAGAAGGAGA |
| 1833 | NFkB | VCAM1 | VCAM1_4 | Reverse Primer | GTACAAGAGATGATGACAGTG |

1. Assembly of primers and probe for determining the activity of the AR cellular signaling pathway, and optionally one or more additional cellular signaling pathway, wherein the assembly of primers and probes comprises three or more sets of primers or probes for determining the expression level of three or more target genes of the AR cellular signaling pathway, wherein said three or more sets of primers and probes are selected from Table 2 of the description,
wherein each primer and/or probe individually has the listed nucleotide sequence identified by the corresponding SEQ ID NO or has a sequence that differs at 1, 2 or 3 positions, wherein said 1, 2 or 3 differences are individually selected from a single base substitution, a single base deletion or a single base addition.

2. Assembly of primers and probes according to claim 1 further comprising primers and probes for determining the activity of the ER cellular signaling pathway, wherein the assembly of primers and probes comprises three or more sets of primers or probes for determining the expression level of three or more target genes of the ER cellular signaling pathway, wherein said three or more sets of primers and probes are selected from Table 1 of the description,
wherein each primer and/or probe individually has the listed nucleotide sequence identified by the corresponding SEQ ID NO or has a sequence that differs at 1, 2 or 3 positions, wherein said 1, 2 or 3 differences are individually selected from a single base substitution, a single base deletion or a single base addition.

3. Assembly of primers and probes according to claim 1 further comprising primers and probes for determining the activity of the PI3K-FOXO cellular signaling pathway, wherein the assembly of primers and probes comprises three or more sets of primers or probes for determining the expression level of three or more target genes of the PI3K-FOXO cellular signaling pathway, wherein said three or more sets of primers and probes are selected from Table 3 of the description,
wherein each primer and/or probe individually has the listed nucleotide sequence identified by the corresponding SEQ ID NO or has a sequence that differs at 1, 2 or 3 positions, wherein said 1, 2 or 3 differences are individually selected from a single base substitution, a single base deletion or a single base addition.

4. Assembly of primers and probes according to claim 1, further comprising primers and probes for determining the activity of the MAPK-AP1 cellular signaling pathway, wherein the assembly of primers and probes comprises three or more sets of primers or probes for determining the expression level of three or more target genes of the MAPK-AP1 cellular signaling pathway, wherein said three or more sets of primers and probes are selected from Table 4 of the description,
wherein each primer and/or probe individually has the listed nucleotide sequence identified by the corresponding SEQ ID NO or has a sequence that differs at 1, 2 or 3 positions, wherein said 1, 2 or 3 differences are individually selected from a single base substitution, a single base deletion or a single base addition.

5. Assembly of primers and probes according to claim 1, further comprising primers and probes for determining the activity of the Notch cellular signaling pathway, wherein the assembly of primers and probes comprises three or more sets of primers or probes for determining the expression level of three or more target genes of the Notch cellular signaling pathway, wherein said three or more sets of primers and probes are selected from Table 5 of the description,
wherein each primer and/or probe individually has the listed nucleotide sequence identified by the corresponding SEQ ID NO or has a sequence that differs at 1, 2 or 3 positions, wherein said 1, 2 or 3 differences are individually selected from a single base substitution, a single base deletion or a single base addition.

6. Assembly of primers and probes according to claim 1, further comprising primers and probes for determining the activity of the Hedgehog (HH) cellular signaling pathway, wherein the assembly of primers and probes comprises three or more sets of primers or probes for determining the expression level of three or more target genes of the HH cellular signaling pathway, wherein said three or more sets of primers and probes are selected from Table 6 of the description,
wherein each primer and/or probe individually has the listed nucleotide sequence identified by the corresponding SEQ ID NO or has a sequence that differs at 1, 2 or 3 positions, wherein said 1, 2 or 3 differences are individually selected from a single base substitution, a single base deletion or a single base addition.

7. Assembly of primers and probes according to claim 1, further comprising primers and probes for determining the activity of the TGFbeta cellular signaling pathway, wherein the assembly of primers and probes comprises three or more sets of primers or probes for determining the expression level of three or more target genes of the TGFbeta cellular signaling pathway, wherein said three or more sets of primers and probes are selected from Table 7 of the description,
wherein each primer and/or probe individually has the listed nucleotide sequence identified by the corresponding SEQ ID NO or has a sequence that differs at 1, 2 or 3 positions, wherein said 1, 2 or 3 differences are individually selected from a single base substitution, a single base deletion or a single base addition.

8. Assembly of primers and probes according to claim 1, further comprising primers and probes for determining the activity of the JAK-STAT1/2 cellular signaling pathway, wherein the assembly of primers and probes comprises three or more sets of primers or probes for determining the expression level of three or more target genes of the JAK-STAT1/2 cellular signaling pathway, wherein said three or more sets of primers and probes are selected from Table 10 of the description,
wherein each primer and/or probe individually has the listed nucleotide sequence identified by the corresponding SEQ ID NO or has a sequence that differs at 1, 2 or 3 positions, wherein said 1, 2 or 3 differences are individually selected from a single base substitution, a single base deletion or a single base addition.

9. Assembly of primers and probes according to claim 1, further comprising primers and probes for determining the activity of the NFkB cellular signaling pathway, wherein the assembly of primers and probes comprises three or more sets of primers or probes for determining the expression level of three or more target genes of the NFkB cellular signaling pathway, wherein said three or more sets of primers and probes are selected from Table 11 of the description,
wherein each primer and/or probe individually has the listed nucleotide sequence identified by the corresponding SEQ ID NO or has a sequence that differs at 1, 2 or 3 positions, wherein said 1, 2 or 3 differences are individually selected from a single base substitution, a single base deletion or a single base addition.

10. Assembly of primers and probes according to claim 1, wherein all of the primers and probes in the three or more sets of primers and probes in the assembly are identical to the corresponding sequence according to Tables 1 to 7, 10 and 11.

11. A kit of parts for determining the expression levels for a plurality of genes, the kit comprising primers and probes for the amplification and detection of the expression levels of the plurality of genes, wherein the kit comprises an assembly of primers and probes as defined in claim 1, wherein the kit further comprises primers and probes for the amplification and detection of three or more of the reference genes selected from ACTB, ALAS1, B2M, EEF1A1 POLR2A, PUM1, RPLP0, TBP, TPT1 and TUBA1B, preferably wherein said three or more sets of primers and probes are selected from Table 8 of the description, and wherein each primer and/or probe individually has the listed nucleotide sequence identified by the corresponding SEQ ID NO or has a sequence that differs at 1, 2 or 3 positions, wherein said 1, 2 or 3 differences are individually selected from a single base substitution, a single base deletion or a single base addition.

12. Use of the assembly of primers and probes as defined in claim 1 for determining the AR cellular signaling pathway activity, and optionally the cellular signaling pathway activity of one or more cellular signaling pathways selected from the group consisting of: HH, ER, TGFbeta, PI3K-FOXO, Notch, MAPK-AP1, JAK-STAT1/2 and NFkB.

13. Use of a set of three or more primers and probes to determine the expression levels of three or more target genes of a cellular signaling pathway, wherein the set of primers and probe combinations are as defined in claim 1, and
wherein the three or more target genes for the AR cellular signaling pathway are selected from the group consisting of: ABCC4, AR, CREB3L4, DHCR24, ELL2, FKBP5, GUCY1A3, KLK2, KLK3, LRIG1, NDRG1, NKX3.1 (also known as NKX3_1), PLAU, PMEPA1, PPAP2A, PRKACB 2, SGK1, and TMPRSS2;
wherein the three or more target genes for the ER cellular signaling pathway are selected from the group consisting of: AP1B1, CA12, CDH26, CELSR2, CTSD, ERBB2, ESR1, GREB1, HSPB1, IGFBP4, MYC, NRIP1, PDZK1, PGR, RARA, SGK3, SOD1, TFF1, WISP2, and XBP1;
the three or more target genes for the PI3K-FOXO cellular signaling pathway are selected from the group consisting of: AGRP, BCL2L11, BCL6, BNIP3, BTG1, CAT, CAV1, CCND1, CCNG2, CDKN1A, CDKN1B, ESR1, FBXO32, FOXO3, GADD45A, INSR, MXI1, SOD2, TNFSF10;
wherein the three or more target genes for the MAPK-AP1 cellular signaling pathway are selected from the group consisting of: BCL2L11, CCND1, DDIT3, EGFR, ENPP2, EZR, GLRX, MMP1, MMP3, MMP9, PLAU, PLAUR, PTGS2, SERPINE1, TIMP1, TP53, VEGFD, and VIM;
wherein the three or more target genes for the Notch cellular signaling pathway are selected from the group consisting of: CD44, EPHB3, FABP7, HES1, HES4, HES5, HEY1, HEY2, MYC, NOX1, NRARP, PIN1, PLXND1, and SOX9;
wherein the three or more target genes for the HH cellular signaling pathway are selected from the group consisting of: CFLAR, FOXM1, FYN, GLI1, HHIP, MYCN, NKX2-2, PTCH1, PTCH2, RAB34, SPP1, TCEA2, and TSC22D1;
wherein the three or more target genes for the TGFbeta cellular signaling pathway are selected from the group consisting of: ANGPTL4, CDKN1A, CTGF, GADD45A, GADD45B, ID1, IL11, JUNB, MMP2, MMP9, PDGFB, SERPINE1, SGK1, SKIL, SMAD4, SMAD7, SNAI1, TIMP1, and VEGFA;
wherein the three or more target genes for the WNT cellular signaling pathway are selected from the group consisting of: CEMIP, AXIN2, CD44, RNF43, MYC, TBX3, TDGF1, SOX9, ASCL2, CXCL8, SP5, ZNRF3, EPHB2, LGR5, EPHB3, KLF6, CCND1, DEFA6, and FZD7;
wherein the three or more target genes for the PR cellular signaling pathway are selected from the group consisting of: AGRP, BCL2L11, BCL6, BNIP3, BTG1, CAT, CAV1, CCND1, CCND2, CCNG2, CDKN1A, CDKN1B, ESR1, FASLG, FBXO32, GADD45A, INSR, MXI1, NOS3, PCK1, POMC, PPARGC1A, PRDX3, RBL2, SOD2 and TNFSF10;
wherein the three or more target genes for the NFkB cellular signaling pathway are selected from the group consisting of: BIRC3, CCL3, CCL4, CCL5, CCL20, CXCL2, ICAM1, IL6, IRF1, MMP9, NFKB2, PTGS2, TNF, TNIP1, TRAF1, and VCAM1;
wherein the three or more target genes for the JAK-STAT1/2 cellular signaling pathway are selected from the group consisting of: APOL1, BID, CXCL9, GBP1, GNAZ, IFI6, IFIT2, IFITM1, IRF1, IRF7, IRF9, ISG15, LY6E, OAS1, PDCD1, RFPL3, SSTR3, STAT1, TAP1 and USP18;
wherein the three or more target genes for the JAK-STAT3 cellular signaling pathway are selected from the group consisting of: AKT1, BCL2, BCL2L1, BIRC5, CCND1, CD274, CDKNIA, CRP, FGF2, FOS, FSCN1, FSCN2, FSCN3, HIFIA, HSP90AA1, HSP90AB1, HSP90B1, HSPA1A, HSPA1B, ICAM1, IFNG, IL10, JunB, MCL1, MMP1, MMP3, MMP9, MUC1, MYC, NOS2, POU2F1, PTGS2, SAA1, STAT1, TIMP1, TNFRSF1B, TWIST1, VIM and ZEB1.

14. A method for designing primers and probes for the detection of the expression levels of target genes of a cellular signaling pathway suitable for determining the activity of the AR cellular signaling pathway and optionally one or more additional cellular signaling pathways, the method comprising:
designing for a target gene of the AR cellular signaling pathway and optionally one or more additional cellular signaling pathway a forward primer and a reverse primer such that:
the forward and reverse primer have a GC content between 35% and 69%, preferably between 35% and 65%;
the forward and reverse primer have a melting temperature between 50 and 71 degrees Celsius, preferably between 58 and 64 degrees Celsius;
the forward and reverse primer have a length between 16 and 25 nucleotides, preferably between 17 and 24 nucleotides;
wherein the amplification product, when using the forward and reverse primers in a PCR amplification reaction, has a size between 60 and 240 base pairs, preferably between 65 and 150 base pairs, and preferably wherein the amplicon product is intron spanning;
designing the probe such that:
the probe used for detection of an amplification product comprises a binding part which is complementary to a part of the amplification product, the binding part further having the following characteristics:
the binding part of the probe has a GC content between 35% and 69%, preferably between 40% and 60%;
the binding part of the probe has a melting temperature between 56 and 72 degrees Celsius, preferably between 64 and 72 degrees Celsius;
the binding part of the probe has a length between 17 and 31 nucleotides, preferably between 18 and 30 nucleotides; and
the binding part of the probe does not have a G at the 5′ part.

15. A method of determining the AR cellular signaling pathway activity and optionally one or more additional cellular signaling pathway activity or activities, by simultaneously determining the expression level of six or more genes in a sample, the method comprising simultaneously amplifying six or more gene products using a polymerase chain reaction to generate a plurality of amplification products, followed by the detection of the plurality of amplification products using a plurality of probes,
wherein the polymerase chain reaction uses, for each amplification product, a forward and a reverse primer which have the following characteristics:
the forward and reverse primer have a GC content between 35% and 69%, preferably between 35% and 65%;
the forward and reverse primer have a melting temperature between 50 and 71 degrees Celsius, preferably between 58 and 64 degrees Celsius;
the forward and reverse primer have a length between 16 and 25 nucleotides, preferably between 17 and 24 nucleotides;
wherein the amplification products have a size between 60 and 240 base pairs, preferably between 65 and 150 base pairs, and preferably wherein the amplicon product is intron spanning,
wherein each of the probes used for detection of an amplification product comprises a binding part which is complementary to a part of the amplification product, the binding part further having the following characteristics:
the binding part of the probe has a GC content between 35% and 69%, preferably between 40% and 60%;
the binding part of the probe has a melting temperature between 56 and 72 degrees Celsius, preferably between 64 and 72 degrees Celsius;
the binding part of the probe has a length between 17 and 31 nucleotides, preferably between 18 and 30 nucleotides;
the binding part of the probe does not have a G at the 5′ part,
wherein the expression levels are used in a method for determining the AR cellular signaling pathway and optionally one or more cellular signaling pathway activities selected from the group consisting of: WNT, HH, ER, PR, PR, TGFbeta, NFkB, STAT1/2, STAT3, PI3K-FOXO, Notch, MAPK-AP1, and
wherein the primers and probes amplify and detect of the expression levels of three or more of the reference genes selected from: ACTB, ALAS1, B2M, EEF1A1 POLR2A, PUM1, RPLP0, TBP, TPT1 and TUBA1B, and
wherein the primers and probes further amplify and detect the expression levels of three or more target genes for the AR cellular signaling pathway and optionally one or more cellular signaling pathways selected from the group consisting of: ER, PI3K-FOXO, MAPK-AP1, HH, Notch, TGFbeta, WNT, PR, NFkB, JAK-STAT1/2, JAK-STAT3,
wherein the three or more target genes for the AR cellular signaling pathway are selected from the group consisting of: ABCC4, AR, CREB3L4, DHCR24, ELL2, FKBP5, GUCY1A3, KLK2, KLK3, LRIG1, NDRG1, NKX3.1 (also known as NKX3_1), PLAU, PMEPA1, PPAP2A, PRKACB 2, SGK1, and TMPRSS2;
wherein the three or more target genes for the ER cellular signaling pathway are selected from the group consisting of: AP1B1, CA12, CDH26, CELSR2, CTSD, ERBB2, ESR1, GREB1, HSPB1, IGFBP4, MYC, NRIP1, PDZK1, PGR, RARA, SGK3, SOD1, TFF1, WISP2, and XBP1;
the three or more target genes for the PI3K-FOXO cellular signaling pathway are selected from the group consisting of: AGRP, BCL2L11, BCL6, BNIP3, BTG1, CAT, CAV1, CCND1, CCNG2, CDKN1A, CDKN1B, ESR1, FBXO32, FOXO3, GADD45A, INSR, MXI1, SOD2, TNFSF10;
wherein the three or more target genes for the MAPK-AP1 cellular signaling pathway are selected from the group consisting of: BCL2L11, CCND1, DDIT3, EGFR, ENPP2, EZR, GLRX, MMP1, MMP3, MMP9, PLAU, PLAUR, PTGS2, SERPINE1, TIMP1, TP53, VEGFD, and VIM;
wherein the three or more target genes for the Notch cellular signaling pathway are selected from the group consisting of: CD44, EPHB3, FABP7, HES1, HES4, HES5, HEY1, HEY2, MYC, NOX1, NRARP, PIN1, PLXND1, and SOX9;
wherein the three or more target genes for the HH cellular signaling pathway are selected from the group consisting of: CFLAR, FOXM1, FYN, GLI1, HHIP, MYCN, NKX2-2, PTCH1, PTCH2, RAB34, SPP1, TCEA2, and TSC22D1;
wherein the three or more target genes for the TGFbeta cellular signaling pathway are selected from the group consisting of: ANGPTL4, CDKN1A, CTGF, GADD45A, GADD45B, ID1, IL11, JUNB, MMP2, MMP9, PDGFB, SERPINE1, SGK1, SKIL, SMAD4, SMAD7, SNAI1, TIMP1, and VEGFA;
wherein the three or more target genes for the WNT cellular signaling pathway are selected from the group consisting of: CEMIP, AXIN2, CD44, RNF43, MYC, TBX3, TDGF1, SOX9, ASCL2, CXCL8, SP5, ZNRF3, EPHB2, LGR5, EPHB3, KLF6, CCND1, DEFA6, and FZD7;
wherein the three or more target genes for the PR cellular signaling pathway are selected from the group consisting of: AGRP, BCL2L11, BCL6, BNIP3, BTG1, CAT, CAV1, CCND1, CCND2, CCNG2, CDKN1A, CDKN1B, ESR1, FASLG, FBXO32, GADD45A, INSR, MXI1, NOS3, PCK1, POMC, PPARGC1A, PRDX3, RBL2, SOD2 and TNFSF10;
wherein the three or more target genes for the NFkB cellular signaling pathway are selected from the group consisting of: BIRC3, CCL3, CCL4, CCL5, CCL20, CXCL2, ICAM1, IL6, IRF1, MMP9, NFKB2, PTGS2, TNF, TNIP1, TRAF1, and VCAM1;
wherein the three or more target genes for the JAK-STAT1/2 cellular signaling pathway are selected from the group consisting of: APOL1, BID, CXCL9, GBP1, GNAZ, IFI6, IFIT2, IFITM1, IRF1, IRF7, IRF9, ISG15, LY6E, OAS1, PDCD1, RFPL3, SSTR3, STAT1, TAP1 and USP18;
wherein the three or more target genes for the JAK-STAT3 cellular signaling pathway are selected from the group consisting of: AKT1, BCL2, BCL2L1, BIRC5, CCND1, CD274, CDKNIA, CRP, FGF2, FOS, FSCN1, FSCN2, FSCN3, HIFIA, HSP90AA1, HSP90AB1, HSP90B1, HSPA1A, HSPA1B, ICAM1, IFNG, IL10, JunB, MCL1, MMP1, MMP3, MMP9, MUC1, MYC, NOS2, POU2F1, PTGS2, SAA1, STAT1, TIMP1, TNFRSF1B, TWIST1, VIM and ZEB1,
wherein the primers and probes are able to amplify and detect the respective genes under the following reaction conditions:
50 mM monovalent salt;
400 nM forward primer
400 nM reverse primer
3.0 mM divalent salt, preferably the divalent salt being Mg2+;
100 nM probe; and
0.8 mM dNTP.
